(19) United States
(12) Patent Application Publication
(10) Pub. No.: US 2016/0213633 A1
(43) Pub. Date: Jul. 28, 2016

(54) METHOD OF INDUCING ANTI-GLATIRAMER ACETATE ANTIBODY RESPONSE

(71) Applicants: David Ladkani, Jerusalem (IL); Natalia Ashtamker, Modiin (IL); Shlomo Bakshi, Moshav Salit (IL); Bracha Timan, Petch Tikva (IL)

(72) Inventors: David Ladkani, Jerusalem (IL); Natalia Ashtamker, Modiin (IL); Shlomo Bakshi, Moshav Salit (IL); Bracha Timan, Petch Tikva (IL)

(73) Assignee: Teva Pharmaceutical Industries, Ltd., Petach-Tikva (IL)

(21) Appl. No.: 14/800,014

(22) Filed: Jul. 15, 2015

Related U.S. Application Data
(60) Provisional application No. 62/109,033, filed on Jan. 28, 2015.

(51) Int. Cl. A61K 31/198; G01N 33/68; A61K 9/00

(52) U.S. Cl. CPC A61K 31/198; A61K 9/0019; G01N 33/6854; G01N 2800/52; G01N 2800/285

(57) ABSTRACT

The present invention provides methods of inducing anti-glatiramer acetate specific antibodies in a human subject afflicted with multiple sclerosis, comprising administration to the human subject of three subcutaneous injections of a 40 mg/ml dose of glatiramer acetate per week for at least 12 months, such that the level of anti-GA specific antibodies in the blood or serum of the human subject i) increases for up to about 6 months after an initial 40 mg/ml dose of glatiramer acetate of the administration is administered to the human subject; ii) increases at a greater rate during the first month after the initial 40 mg/ml dose of glatiramer acetate compared to the rate of increase after the first month to the third month after the initial 40 mg/ml dose of glatiramer acetate; iii) peaks at about 3 or about 6 months or between about 3 and about 6 months after the initial 40 mg/ml dose of glatiramer acetate; and iv) is higher than baseline at least about 12 months after the initial 40 mg/ml dose of glatiramer acetate, wherein the 40 mg/ml dose of glatiramer acetate is in a prefilled syringe containing 1 ml of an aqueous pharmaceutical solution of 40 mg/ml of glatiramer acetate and 40 mg/ml mannitol, and wherein the aqueous pharmaceutical solution has a pH in the range of 5.5 to 7.0.

This application claims the benefit of U.S. Provisional Application No. 62/109,033, filed Jan. 28, 2015, the contents of which are hereby incorporated by reference.

Throughout this application, various publications are referenced, including referenced by Arabic numerals. Full citations for publications referenced in Arabid numerals may be found listed at the end of the specification immediately preceding the claims. The disclosures of all referenced publications in their entireties are hereby incorporated by reference into this application in order to more fully describe the state of the art to which this invention pertains.

### BACKGROUND OF THE INVENTION

Multiple sclerosis (MS) is a chronic, debilitating autoimmune disease of the central nervous system (CNS) with either relapsing-remitting (RR) or progressive course leading to neurologic deterioration and disability. At time of initial diagnosis, RRMS is the most common form of the disease (1) which is characterized by unpredictable acute episodes of neurological dysfunction (relapses), followed by variable recovery and periods of clinical stability. The vast majority of RRMS patients eventually develop secondary progressive (SP) disease with or without superimposed relapses. Around 15% of patients develop a sustained deterioration of their neurological function from the beginning; this form is called primary progressive (PP) MS. Patients who have experienced a single clinical event (Clinically Isolated Syndrome or “CIS”) and who show lesion dissemination on subsequent magnetic resonance imaging (MRI) scans according to McDonald's criteria, are also considered as having relapsing MS (2).

With a prevalence that varies considerably around the world, MS is the most common cause of chronic neurological disability in young adults (3, 4). Anderson et al. estimated that there were about 350,000 physician-diagnosed patients with MS in the United States in 1990 (approx. 140 per 100,000 population) (5). It is estimated that about 2.5 million individuals are affected worldwide (6). In general, there has been a trend toward an increasing prevalence and incidence of MS worldwide, but the reasons for this trend are not fully understood (5).

Current therapeutic approaches consist of i) symptomatic treatment ii) treatment of acute relapses with corticosteroids and iii) treatment aimed to modify the course of the disease. Currently approved therapies target the inflammatory processes of the disease. Most of them are considered to act as immunomodulators but their mechanisms of action have not been completely elucidated. Immunosuppressants or cytotoxic agents are also used in some patients after failure of conventional therapies. Several medications have been approved and clinically ascertained as efficacious for the treatment of RR-MS; including BETASERON®, AVONEX® and REBIF®, which are derivatives of the cytokine interferon beta (IFNB), whose mechanism of action in MS is generally attributed to its immunomodulatory effects, antagonizing pro-inflammatory reactions and inducing suppressor cells. Other approved drugs for the treatment of MS include Mitoxantrone and Natalizumab (7).

Copaxone® (Teva Pharmaceutical Industries Ltd.) is indicated for the treatment of patients with relapsing forms of multiple sclerosis. Copaxone® is a clear, colorless to slightly yellow, sterile, nonpyrogenic solution for subcutaneous injection (8). Each 1 mL of Copaxone® solution contains 20 mg or 40 mg of the active ingredient, glatiramer acetate (GA), the inactive ingredient, 40 mg of mannitol (8).

GA, the active ingredient of Copaxone®, consists of the acetate salts of synthetic polypeptides, containing four naturally occurring amino acids: L-glutamic acid, L-alanine, L-tyrosine, and L-lysine with an average molar fraction of 0.141, 0.427, 0.095, and 0.338, respectively. Glatiramer acetate is identified by specific antibodies (8).

GA elicits anti-inflammatory as well as neuroprotective effects in various animal models of chronic inflammatory and neurodegenerative diseases (9-13) and has been shown to be safe and effective in reducing relapses and delaying neurologic disability in MS patients following long-term treatment (14).

The mechanisms underlying GA therapeutic activity are not fully elucidated, but GA activity on immune cells has been well demonstrated. GA appears to act as an altered peptide ligand (APL) of encephalitogenic epitopes within myelin basic protein (MBP) (15) and demonstrates cross-reactivity with MBP at the humoral and cellular levels (16-22). The unique antigenic sequences of the GA polypeptide mixture compete with myelin antigens for binding to MHC class II molecules on antigen presenting cells (APCs) and presentation to the T cell receptor (TCR), resulting in the induction of anergy or deletion of autoreactive MBP-reactive T cells and proliferation of GA-reactive T cells. At initiation of Copaxone® treatment, GA-reactive CD4+ T-cell lines from MS patients secrete both pro-inflammatory T helper type 1 (Th1) and anti-inflammatory Th2 cytokines (20, 23), but continued exposure to Copaxone® induces a shift in GA-reactive T cells toward the Th2 phenotype (20, 22, 24-27). In MS patients treated with daily subcutaneous Copaxone 20 mg/ml, anti-GA antibody peaked at 3 months after initiation of treatment, decreasing at 6 months and remaining low, and IgG1 antibody levels were 2-3 fold higher than those of IgG2 (28).

Copaxone® also increases the number and suppressive capacity of CD4+CD25+FOXP3+ regulatory T cells, which are functionally impaired in MS patients (29-31). Furthermore, treatment leads to antigen-nonspecific modulation of APC function. Copaxon® treatment promotes development of anti-inflammatory type II monocytes characterized by an increase in interleukin (IL)-10 and transforming growth factor-beta (TGF-β) and decreased production of IL-12 and tumor necrosis factor (TNF) (32).

### SUMMARY OF THE INVENTION

The present invention provides a method of inducing anti-glatiramer acetate (GA) specific antibodies in a human subject afflicted with multiple sclerosis, comprising administration to the human subject of three subcutaneous injections of a 40 mg/ml dose of glatiramer acetate per week for at least 12 months, such that the level of anti-GA specific antibodies in the blood or serum of the human subject
i) increases for up to about 6 months after an initial 40 mg/ml dose of glatiramer acetate of the administration is administered to the human subject;
ii) increases at a greater rate during the first month after the initial 40 mg/ml dose of glatiramer acetate compared to the rate of increase after the first month to the third month after the initial 40 mg/ml dose of glatiramer acetate;
iii) peaks at about 3 or about 6 months or between about 3 and about 6 months after the initial 40 mg/ml dose of glatiramer acetate; and
iv) is higher than baseline at least about 12 months after the initial 40 mg/ml dose of glatiramer acetate.

The present invention provides a method of producing a glatiramer acetate response profile for a human subject afflicted with multiple sclerosis comprising the steps of:
a) obtaining blood or serum samples periodically collected from a human subject afflicted with multiple sclerosis who is administered three subcutaneous injections of a 40 mg/ml dose of glatiramer acetate per week for at least about 12 or at least about 24 months after the initial 40 mg/ml dose of glatiramer acetate is administered;
b) assaying whether the level of anti-glatiramer acetate (GA) specific antibodies in the blood or serum of the human subject
   i) increases for up to about 6 months after the initial 40 mg/ml dose of glatiramer acetate;
   ii) increases at a greater rate during the first month after the initial 40 mg/ml dose of glatiramer acetate compared to the rate of increase after the first month to the third month after the initial 40 mg/ml dose of glatiramer acetate;
   iii) peaks at about 3 or about 6 months or between about 3 and about 6 months after the initial 40 mg/ml dose of glatiramer acetate; and
   iv) is higher than baseline about 12 and about 24 months after the initial 40 mg/ml dose of glatiramer acetate,
c) producing the glatiramer acetate response profile of the human subject comprising the level of anti-GA specific antibodies determined in step b).

The present invention provides a method of inducing anti-glatiramer acetate (GA) specific antibodies in a human subject afflicted with multiple sclerosis, comprising administration to the human subject of three subcutaneous injections of a 40 mg/ml dose of glatiramer acetate per week for at least 12 months, such that the level of anti-GA specific antibodies in the blood or serum of the human subject
i) increases for up to 6 months after an initial 40 mg/ml dose of glatiramer acetate of the administration is administered to the human subject;
ii) increases at a greater rate during the first month after the initial 40 mg/ml dose of glatiramer acetate compared to the rate of increase after the first month to the third month after the initial 40 mg/ml dose of glatiramer acetate;
iii) peaks at 3 or 6 months or between 3 and 6 months after the initial 40 mg/ml dose of glatiramer acetate; and
iv) is higher than baseline at least 12 months after the initial 40 mg/ml dose of glatiramer acetate,
wherein the 40 mg/ml dose of glatiramer acetate is in a prefilled syringe containing 1 ml of an aqueous pharmaceutical solution of 40 mg/ml of glatiramer acetate and 40 mg/ml mannitol, and wherein the aqueous pharmaceutical solution
   a) has a viscosity in the range of 2.0-3.5 cPa; or
   b) has an osmolality in the range of 270-330 mosmol/Kg.

### BRIEF DESCRIPTION OF THE DRAWINGS

FIG. 1. Anti-GA specific antibody response profile in patients who were treated with Copaxone® 20 mg/ml subcutaneously daily for 9 months. Anti-GA specific antibodies were detected using enzyme-linked immunosorbant assay (ELISA). The line representing cut-point value crosses the Y axis at 7.1(RA %).

FIG. 2. Anti-GA specific antibody response in patients who were treated with Copaxone® 20 mg/ml subcutaneously daily for 2 years. Anti-GA specific antibodies were detected using either (A) radioimmunoassay (RIA), and (B) ELISA techniques.

FIG. 3. Anti-GA specific antibody response in patients who were treated with Copaxone® 40 mg/ml subcutaneously three times per week for 2 years. The line representing cut-point value crosses the Y axis at 1.4(RA %).

### DETAILED DESCRIPTION OF THE INVENTION

The present invention provides a method of inducing anti-glatiramer acetate (GA) specific antibodies in a human subject afflicted with multiple sclerosis, comprising administration to the human subject of three subcutaneous injections of a 40 mg/ml dose of glatiramer acetate per week for at least 12 months, such that the level of anti-GA specific antibodies in the blood or serum of the human subject
i) increases for up to about 6 months after an initial 40 mg/ml dose of glatiramer acetate of the administration is administered to the human subject;
ii) increases at a greater rate during the first month after the initial 40 mg/ml dose of glatiramer acetate compared to the rate of increase after the first month to the third month after the initial 40 mg/ml dose of glatiramer acetate;
iii) peaks at about 3 or about 6 months or between about 3 and about 6 months after the initial 40 mg/ml dose of glatiramer acetate; and
iv) is higher than baseline at least about 12 months after the initial 40 mg/ml dose of glatiramer acetate.

In some embodiments, the level of anti-GA specific antibodies in the blood or serum of the human subject increases for about 3 to about 6 months after the initial 40 mg/ml dose of glatiramer acetate.

In some embodiments, three subcutaneous injections of a 40 mg/ml dose of glatiramer acetate per week are administered to the human subject for at least 18 months, and the level of anti-GA specific antibodies in the blood or serum of the human subject is higher than baseline about 18 months after the initial 40 mg/ml dose of glatiramer acetate.

In some embodiments, three subcutaneous injections of a 40 mg/ml dose of glatiramer acetate per week are administered to the human subject for at least 24 months, and the level of anti-GA specific antibodies in the blood or serum of the human subject is higher than baseline about 24 months after the initial 40 mg/ml dose of glatiramer acetate.

In some embodiments, the anti-GA specific antibodies are other than IgM or IgE antibodies.

In some embodiments, the level of anti-GA specific antibodies in the blood or serum of the human subject increases
i) to at least about 25, about 30, about 35, about 40, about 45, about 50, about 55, about 60, about 65, about 67.5, about 70, about 75, about 80, about 25 to about 50, about 29 to about 106, about 29 to about 60, about 31 to about 60, about 35 to about 70, about 39 to about 71, about 50 to about 75, about 75 to about 80, or about 50 to about 80 RA % within about 1, about 3, about 6, about 9, about 12, about 18, or about 24 months after the initial 40 mg/ml dose of glatiramer acetate, as measured by an enzyme-linked immunosorbent assay (ELISA);
ii) by at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 25.9 to about 975, or about 100 to about 200-fold increase in RA % over baseline within about 1, about 3, about 6, about 9, about 12, about 18, or about 24 months after the initial 40 mg/ml dose of glatiramer acetate, as measured by ELISA; or
iii) to at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150 or about 100 to about 200-fold over baseline.

In some embodiments, the level of anti-GA specific antibodies in the blood or serum of the human subject increases to at least about 34, about 35, or about 35 to about 70 RA % within about 1 month after the initial 40 mg/ml dose of glatiramer acetate, as measured by ELISA.

In some embodiments, the level of anti-GA specific antibodies in the blood or serum of the human subject increases to at least about 67.5 or about 29 to about 106 RA % within about 3 months after the initial 40 mg/ml dose of glatiramer acetate, as measured by ELISA.

In some embodiments, the level of anti-GA specific antibodies in the blood or serum of the human subject increases to at least about 40 or about 39 to about 71 RA % within about 6 months after the initial 40 mg/ml dose of glatiramer acetate, as measured by ELISA.

In some embodiments, the level of anti-GA specific antibodies in the blood or serum of the human subject increases to at least about 30 or about 31 to about 60 RA % within about 9 months after the initial 40 mg/ml dose of glatiramer acetate, as measured by ELISA.

In some embodiments, the level of anti-GA specific antibodies in the blood or serum of the human subject increases to at least about 30 or about 30 to about 60 RA % within about 12 months after the initial 40 mg/ml dose of glatiramer acetate, as measured by ELISA.

In some embodiments, the level of anti-GA specific antibodies in the blood or serum of the human subject increases to at least about 25 or about 25 to about 50 RA % within about 18 months after the initial 40 mg/ml dose of glatiramer acetate, as measured by ELISA.

In some embodiments, the level of anti-GA specific antibodies in the blood or serum of the human subject increases to at least about 25 or about 25 to about 50 RA % within about 24 months after the initial 40 mg/ml dose of glatiramer acetate, as measured by ELISA.

The present invention provides a method of producing a glatiramer acetate response profile for a human subject afflicted with multiple sclerosis comprising the steps of:
a) obtaining blood or serum samples periodically collected from a human subject afflicted with multiple sclerosis who is administered three subcutaneous injections of a 40 mg/ml dose of glatiramer acetate per week for at least about 12 or at least about 24 months after the initial 40 mg/ml dose of glatiramer acetate is administered;
b) assaying whether the level of anti-glatiramer acetate (GA) specific antibodies in the blood or serum of the human subject
   i) increases for up to about 6 months after the initial 40 mg/ml dose of glatiramer acetate;
   ii) increases at a greater rate during the first month after the initial 40 mg/ml dose of glatiramer acetate compared to the rate of increase after the first month to the third month after the initial 40 mg/ml dose of glatiramer acetate;
   iii) peaks at about 3 or about 6 months or between about 3 and about 6 months after the initial 40 mg/ml dose of glatiramer acetate; and
   iv) is higher than baseline about 12 and about 24 months after the initial 40 mg/ml dose of glatiramer acetate,
c) producing the glatiramer acetate response profile of the human subject comprising the level of anti-GA specific antibodies determined in step b).

In some embodiments, the method comprises assaying whether the level of anti-GA specific antibodies in the blood or serum of the human subject increases for about 3 to about 6 months after the initial 40 mg/ml dose of glatiramer acetate.

In some embodiments, the method further comprises assaying whether the anti-GA specific antibodies are other than IgM or IgE antibodies, and the glatiramer acetate response profile of the human subject produced in step c) identifies the anti-GA specific antibodies as other than IgM or IgG antibodies.

In some embodiments, the glatiramer acetate response profile is a written glatiramer acetate response profile report.

In some embodiments, step b) comprises assaying whether the level of anti-GA specific antibodies in the blood or serum of the human subject increases
i) to at least about 25, about 30, about 35, about 40, about 45, about 50, about 55, about 60, about 65, about 67.5, about 70, about 75, about 80, about 25 to about 50, about 29 to about 106, about 29 to about 60, about 31 to about 60, about 35 to about 70, about 39 to about 71, about 50 to about 75, about 75 to about 80, or about 50 to about 80 RA % within about 1, about 3, about 6, about 9, about 12, about 18, or about 24 months after the initial 40 mg/ml dose of glatiramer acetate, as measured by an enzyme-linked immunosorbent assay (ELISA);
ii) by at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 25.9 to about 975, or about 100 to about 200-fold increase in RA % over baseline within about 1, about 3, about 6, about 9, about 12, about 18, or about 24 months after the initial 40 mg/ml dose of glatiramer acetate, as measured by ELISA; or
iii) to at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150 or about 100 to about 200-fold over baseline.

In some embodiments, the level of anti-GA specific antibodies in the blood or serum of the human subject increases to at least about 34, about 35, or about 35 to about 70 RA % within about 1 month after the initial 40 mg/ml dose of glatiramer acetate, as measured by ELISA.

In some embodiments, the level of anti-GA specific antibodies in the blood or serum of the human subject increases to at least about 67.5 or about 29 to about 106 RA % within about 3 months after the initial 40 mg/ml dose of glatiramer acetate, as measured by ELISA.

In some embodiments, the level of anti-GA specific antibodies in the blood or serum of the human subject increases to at least about 40 or about 39 to about 71 RA % within about 6 months after the initial 40 mg/ml dose of glatiramer acetate, as measured by ELISA.

In some embodiments, the level of anti-GA specific antibodies in the blood or serum of the human subject increases to at least about 30 or about 31 to about 60 RA % within about 9 months after the initial 40 mg/ml dose of glatiramer acetate, as measured by ELISA.

In some embodiments, the level of anti-GA specific antibodies in the blood or serum of the human subject increases to at least about 30 or about 30 to about 60 RA % within about 12 months after the initial 40 mg/ml-dose of glatiramer acetate, as measured by ELISA.

In some embodiments, the level of anti-GA specific antibodies in the blood or serum of the human subject increases to at least about 25 or about 25 to about 50 RA % within about 18 months after the initial 40 mg/ml dose of glatiramer acetate, as measured by ELISA.

In some embodiments, the level of anti-GA specific antibodies in the blood or serum of the human subject increases to at least about 25 or about 25 to about 50 RA % within about 24 months after the initial 40 mg/ml dose of glatiramer acetate, as measured by ELISA.

In some embodiments, the three subcutaneous injections are on three days each week selected from the group consisting of day 1, day 3 and day 5; day 1, day 3 and day 6; day 1, day 4 and day 6; day 2, day 4 and day 6; day 2, day 4 and day 7; 2, day 5 and day 7; and day 3, day 5 and day 7.

In some embodiments, the glatiramer acetate is present in 1 ml of a pharmaceutical composition in a prefilled syringe for self-administration by the human subject.

In some embodiments, the pharmaceutical composition further comprises mannitol and has a pH in the range of 5.5 to 7.0.

In some embodiments, the 40 mg/ml dose of glatiramer acetate is in a prefilled syringe containing 40 mg of glatiramer acetate and 40 mg mannitol.

In some embodiments, the prefilled syringe contains 1 ml of an aqueous pharmaceutical solution of 40 mg/ml of glatiramer acetate and 40 mg/ml mannitol.

In some embodiments, the aqueous pharmaceutical solution
a) has a viscosity in the range of 2.0-3.5 cPa; or
b) has an osmolality in the range of 270-330 mosmol/Kg.

In some embodiments, the aqueous pharmaceutical solution
a) has a viscosity in the range of 2.2-3.0 cPa; or
b) has an osmolality in the range of 275-325 mosmol/Kg.

In some embodiments, the 40 mg/ml dose of glatiramer acetate is an aqueous pharmaceutical solution comprising 40 mg/ml glatiramer acetate and 40 mg/ml mannitol, wherein the aqueous pharmaceutical solution
a) has a viscosity in the range of 2.0-3.5 cPa; or
b) has an osmolality in the range of 275-325 mosmol/Kg.

In some embodiments, the aqueous pharmaceutical solution has a viscosity in the range of 2.0-3.5 cPa.

In some embodiments, the aqueous pharmaceutical solution has a viscosity in the range of 2.61-2.92 cPa.

In some embodiments, the aqueous pharmaceutical solution has an osmolality in the range of 275-325 mosmol/Kg.

In some embodiments, the aqueous pharmaceutical solution has an osmolality in the range of 300-303 mosmol/Kg.

In some embodiments, the human subject is suffering from relapsing-remitting multiple sclerosis (RRMS).

In some embodiments, the human subject is suffering from multiple sclerosis other than RRMS.

In some embodiments, the human subject is suffering from progressive-relapsing multiple sclerosis, secondary progressive multiple sclerosis, or primary progressive multiple sclerosis.

In some embodiments, the human subject has previously received administration of a 20 mg/ml dose of glatiramer acetate.

In some embodiments, the human subject is a naïve subject or has been previously administered a multiple sclerosis drug other than glatiramer acetate.

In some embodiments, the multiple sclerosis drug other than glatiramer acetate is interferon β-1a, interferon β-1b, mitoxantrone, natalizumab, fingolimod, teriflunomide, or dimethyl fumarate.

In some embodiments, the human subject has a genotype comprising:
one or more A alleles at the location of one or more single nucleotide polymorphisms (SNPs) selected from the group consisting of: kgp10152733, kgp10224254, kgp10305127, kgp10351364, kgp10372946, kgp10404633, kgp10564659, kgp10591989, kgp10594414, kgp10619195, kgp10620244, kgp10633631, kgp10974833, kgp11002881, kgp11285862, kgp11328629, kgp11407560, kgp11514107, kgp11627530, kgp11702474, kgp11711524, kgp11768533, kgp11804835, kgp12083934, kgp12182745, kgp12230354, kgp1224440, kgp124162, kgp12557319, kgp1371881, kgp1699628, kgp1753445, kgp1779254, kgp1786079, kgp18379774, kgp18525257, kgp20163979, kgp2023214, kgp20478926, kgp21171930, kgp2262166, kgp22778566, kgp2465184, kgp24753470, kgp25191871, kgp25216186, kgp25952891, kgp26026546, kgp26533576, kgp27500525, kgp27571222, kgp28532436, kgp28586329, kgp28817122, kgp2958113, kgp29794723, kgp30282494, kgp304921, kgp3205849, kgp3218351, kgp3276689, kgp337461, kgp345301, kgp355027, kgp355723, kgp3593828, kgp3812034, kgp3951463, kgp4162414, kgp4223880, kgp4418535, kgp4543470, kgp4573213, kgp4634875, kgp4755147, kgp4842590, kgp485316, kgp5068397, kgp5334779, kgp5483926, kgp5564995, kgp5869992, kgp5908616, kgp6032617, kgp6038357, kgp6076976, kgp6091119, kgp6127371, kgp61811, kgp6214351, kgp6228750, kgp6236949, kgp6469620, kgp6505544, kgp6507761, kgp6666134, kgp6700691, kgp6772915, kgp6959492, kgp7077322, kgp7117398, kgp7178233, kgp7186699, kgp7506434, kgp759150, kgp7730397, kgp7802182, kgp7804623, kgp7924485, kgp8030775, kgp8036704, kgp8046214, kgp8106690, kgp8110667, kgp8178358, kgp8200264, kgp8372910, kgp841428, kgp8602316, kgp8615910, kgp8793915, kgp8796185, kgp8990121, kgp9018750, kgp9354462, kgp9368119, kgp9410843, kgp9450430, kgp9530088, kgp9627338, kgp9669946, kgp97310, kgp974569, kgp9806386, kgp9884626, rs10049206, rs10124492, rs10125298, rs10162089, rs10203396, rs10251797, rs10278591, rs10489312, rs10492882, rs10498793, rs10501082, rs10510774, rs10512340, rs10815160, rs10816302, rs10841337, rs11029892, rs11029928, rs11192469, rs11559024, rs11648129, rs12013377, rs13394010, rs13415334, rs1478682, rs1544352, rs1545223, rs1604169, rs1621509, rs1644418, rs17029538, rs17400875, rs17449018, rs17577980, rs1858973, rs1894406, rs1894407, rs197523, rs2058742, rs2071469, rs2071472, rs2139612, rs2241883, rs2309760, rs241440, rs241442, rs241444, rs241445, rs241446, rs241449, rs241453, rs241456, rs2453478, rs2660214, rs2824070, rs2845371, rs2857103, rs2926455, rs343087, rs343092, rs3767955, rs3792135, rs3829539, rs3899755, rs4075692, rs4143493, rs423239, rs4254166, rs4356336, rs4584668, rs4780822, rs4782279, rs5024722, rs6032209, rs6110157, rs623011, rs6497396, rs6845927, rs6895094, rs6899068, rs7024953, rs7028906, rs7029123, rs7062312, rs7187976, rs7191155, rs720176, rs7228827, rs7496451, rs7563131, rs759458, rs7666442, rs7670525, rs7677801, rs7725112, rs7850, rs7862565, rs7948420, rs8035826, rs8053136, rs8055485, rs823829, rs9315047, rs9501224, rs9508832, rs950928, rs9597498, rs9670531, rs9671124, rs9817308, rs9834010, rs9876830 or rs9931211;
one or more C alleles at the location of one or more SNPs selected from the group consisting of: kgp10910719, kgp11077373, kgp11453406, kgp12426624, kgp2045074, kgp22811918, kgp23298674, kgp2709692, kgp28687699, kgp3496814, kgp3669685, kgp3730395, kgp4056892, kgp4370912, kgp5053636, kgp5216209, kgp5292386, kgp6023196, kgp652534, kgp7059449, kgp7189498, kgp7521990, kgp7792268, kgp8303520, kgp9320791, kgp9795732, rs10201643, rs11022778, rs11136970, rs11147439, rs11691553, rs1579771, rs16901784, rs2136408, rs2325911, rs241443, rs2857104, rs3803277, rs3885907, rs4738738, rs4894701, rs502530, rs6032205, rs6687976, rs6718758, rs6835202, rs714342, rs7524868, rs7844274, rs9393727 or rs9671182; or
one or more G alleles at the location of one or more SNPs selected from the group consisting of: kgp10090631, kgp1009249, kgp10412303, kgp10523170, kgp1054273, kgp10558725, kgp10632945, kgp10679353, kgp10788130, kgp10826273, kgp10922969, kgp10948564, kgp10967046, kgp1098237, kgp11010680, kgp11141512, kgp11206453, kgp11210903, kgp1124492, kgp11281589, kgp11356379, kgp11467007, kgp11543962, kgp11580695, kgp11633966, kgp11686146, kgp11843177, kgp12008955, kgp12371757, kgp1285441, kgp13161760, kgp1355977, kgp15390522, kgp1683448, kgp1688752, kgp1912531, kgp19568724, kgp2092817, kgp2245775, kgp22793211, kgp22823022, kgp2282938, kgp2299675, kgp2356388, kgp23672937, kgp23737989, kgp2388352, kgp2391411, kgp24131116, kgp24415534, kgp2446153, kgp2451249, kgp24729706, kgp25543811, kgp25921291, kgp26271158, kgp2638591, kgp26528455, kgp2688306, kgp26995430, kgp270001, kgp2715873, kgp27640141, kgp2788291, kgp2923815, kgp29367521, kgp293787, kgp2959751, kgp297178, kgp3048169, kgp3182607, kgp3202939, kgp3267884, kgp3418770, kgp3450875, kgp3477351, kgp3598409, kgp3651767, kgp3854180, kgp3933330, kgp3984567, kgp4011779, kgp4096263, kgp4127859, kgp4155998, kgp4346717, kgp4420791, kgp4479467, kgp4524468, kgp4559907, kgp4705854, kgp4734301, kgp4812831, kgp487328, kgp4898179, kgp5002011, kgp5014707, kgp5017029, kgp512180, kgp5144181, kgp5159037, kgp5388938, kgp5409955, kgp5440506, kgp5441587, kgp55646, kgp5579170, kgp5680955, kgp6190988, kgp6539666, kgp6567154, kgp6599438, kgp6603796, kgp6737096, kgp6768546, kgp6835138, kgp6996560, kgp7063887, kgp7092772, kgp7121374, kgp7181058, kgp7331172, kgp7416024, kgp7481870, kgp767200, kgp7714238, kgp7747883, kgp8107491, kgp8169636, kgp8174785, kgp8183049, kgp8192546, kgp8335515, kgp8437961, kgp8440036, kgp85534, kgp8599417, kgp8767692, kgp8777935, kgp8817856, kgp8869954, kgp9071686, kgp9078300, kgp9354820, kgp9421884, kgp9551947, kgp9601362, kgp9627406, kgp9699754, kgp971582, kgp9854133, rs1079303, rs10841322, rs10954782, rs11002051, rs11029907, rs11083404, rs11085044, rs11192461, rs1157449, rs12494712, rs12943140, rs13002663, rs13419758, rs1380706, rs1387768, rs1410779, rs1508102, rs1532365, rs16886004, rs16895510, rs16927077, rs16930057, rs17224858, rs17238927, rs17329014, rs17638791, rs1886214, rs1894408, rs196295, rs196341, rs196343, rs1979992, rs1979993, rs2043136, rs2071470, rs2074037, rs2175121, rs241435, rs241447, rs241451, rs241452, rs241454, rs2598360, rs2621321, rs2621323, rs2816838, rs2839117, rs2857101, rs2934491, rs3135388, rs3218328, rs3799383, rs3815822, rs3818675, rs419132, rs4360791, rs4449139, rs4669694, rs4709792, rs4769060, rs4822644, rs484482, rs543122, rs6535882, rs6840089, rs7020402, rs7217872, rs7348267, rs7579987, rs7672014, rs7860748, rs7864679, rs7928078, rs8050872, rs858341, rs931570, rs9346979, rs9376361, rs9579566, rs9913349 or rs9931167, or one or more T alleles at the location of kgp18432055, kgp279772, kgp3991733 or kgp7242489.
The method of any one of claims 1-30, wherein assaying the level of anti-GA specific antibodies comprises an enzyme-linked immunosorbent assay (ELISA) or a radioimmunoassay.
The method of claim 31, wherein assaying the level of anti-GA specific antibodies comprises ELISA.

In some embodiments, the baseline level of anti-GA specific antibodies is
i) the level of anti-GA specific antibodies in a corresponding naïve human subject afflicted with multiple sclerosis;
ii) the level of anti-GA specific antibodies in a corresponding naïve healthy human subject;
iii) the level of anti-GA specific antibodies in the human subject before the human subject has been administered the after the initial 40 mg/ml dose of glatiramer acetate; or
iv) the level of anti-GA specific antibodies in the human subject concurrently with administration of the initial 40 mg/ml dose of glatiramer acetate.

In some embodiments, the baseline level of anti-GA specific antibodies is the RA % for
i) the level of anti-GA specific antibodies in a corresponding naïve human subject afflicted with multiple sclerosis;
ii) the level of anti-GA specific antibodies in a corresponding naïve healthy human subject;
iii) the level of anti-GA specific antibodies in the human subject before the human subject has been administered the after the initial 40 mg/ml dose of glatiramer acetate; or
iv) the level of anti-GA specific antibodies in the human subject concurrently with administration of the initial 40 mg/ml dose of glatiramer acetate.

In some embodiments, the ELISA is solid-phase ELISA.

The present invention provides a method of inducing anti-glatiramer acetate (GA) antibodies in a human subject afflicted with multiple sclerosis, comprising administration to the human subject of three subcutaneous injections of a 40 mg/ml dose of glatiramer acetate per week for at least 12 months, such that the level of anti-GA antibodies in the blood or serum of the human subject
i) increases for up to 6 months after an initial 40 mg/ml dose of glatiramer acetate of the administration is administered to the human subject;
ii) increases at a greater rate during the first month after the initial 40 mg/ml dose of glatiramer acetate compared to the rate of increase after the first month to the third month after the initial 40 mg/ml dose of glatiramer acetate;
iii) peaks at 3 or 6 months or between 3 and 6 months after the initial 40 mg/ml dose of glatiramer acetate; and
iv) is higher than baseline at least 12 months after the initial 40 mg/ml dose of glatiramer acetate,
wherein the 40 mg/ml dose of glatiramer acetate is in a prefilled syringe containing 1 ml of an aqueous pharmaceutical solution of 40 mg/ml of glatiramer acetate and 40 mg/ml mannitol, and wherein the aqueous pharmaceutical solution
   a) has a viscosity in the range of 2.0-3.5 cPa; or
   b) has an osmolality in the range of 270-330 mosmol/Kg.

All combinations of the various elements described herein are within the scope of the invention.

### DEFINITIONS

As used herein, “glatiramer acetate” is a complex mixture of the acetate salts of synthetic polypeptides, containing four naturally occurring amino acids: L-glutamic acid, L-alanine, L-tyrosine, and L-lysine. The peak average molecular weight of glatiramer acetate is between 5,000 and 9,000 daltons. Chemically, glatiramer acetate is designated L-glutamic acid polymer with L-alanine, L-lysine and L-tyrosine, acetate (salt). Its structural formula is:

(Glu,Ala,Lys,Tyr)x.XCH3COOH

(C₅H₉NO₄.C₃H₇NO₂.C₆H₁₄N₂O₂.C₉H₁₁NO₃)ₓ.xC₂H₄O₂

“RA %” or “RA (%)” is calculated by the following equation:

$$RA ( \% ) = \frac{AVG test sample ABS - AVG Blank ABS}{AVG SST ABS - AVG SST Blank ABS} \times 100$$

where “AVG test sample ABS” means average absorbance (ABS) of each test sample and/or control;
where “Blank” is one or more wells coated with GA without human serum sample;
where “AVG Blank ABS” is average absorbance (ABS) of Blank;
where “SST” is System Suitability Test which is one or more wells coated with purified human IgG;
where “AVG SST ABS” is average absorbance (ABS) of the SST; and
where “SST Blank” is one or more wells coated with the blocking buffer only, with neither hIgG nor human serum sample.

Glatiramer acetate may be abbreviated herein as “GA.”

As used herein, “anti-GA antibodies” and “anti-GA specific antibodies” are immunoglobulin (Ig) molecules which specifically recognize GA.

As used herein, the “administration” of glatiramer acetate may be oral, nasal, pulmonary, parenteral, intravenous, intra-articular, transdermal, intradermal, subcutaneous, topical, intramuscular, rectal, intrathecal, intraocular, buccal or by gavage.

As used herein, “Benign Multiple Sclerosis” is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis.

As used herein, “Relapsing-Remitting Multiple Sclerosis” (RRMS) is characterized by patients experiencing sporadic exacerbations or relapses, as well as periods of remission. Lesions and evidence of axonal loss may or may not be visible on MRI for patients with RRMS.

As used herein, “Secondary Progressive Multiple Sclerosis” (SPMS) is characterized by patients having relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.

As used herein, “Primary Progressive Multiple Sclerosis” (PPMS) is characterized by a steady progression of increasing neurological deficits without distinct attacks or remissions. Cerebral lesions, diffuse spinal cord damage and evidence of axonal loss are evident on the MRI of patients with PPMS.

As used herein, “Progressive-Relapsing Multiple Sclerosis” (PRMS) is characterized by having periods of acute exacerbations while proceeding along a course of increasing neurological deficits without remissions. Lesions are evident on MRI of patients suffering from PRMS.

A clinically isolated syndrome (CIS) is a single monosymptomatic attack compatible with MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesions go on to develop MS, while approximately 20 percent have a self-limited process. (29,30) Patients who experience a single clinical attack consistent with MS may have at least one lesion consistent with multiple sclerosis prior to the development of clinically definite multiple sclerosis.

Multiple sclerosis may present with optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

The term relapsing MS includes:
1) patients with RAMS;
2) patients with SPMS and superimposed relapses; and
3) patients with CIS who show lesion dissemination on subsequent MRI scans according to McDonald's criteria.

As used herein, relapsing forms of multiple sclerosis include: Relapsing-remitting multiple sclerosis (RRMS), characterized by unpredictable acute episodes of neurological dysfunction (relapses), followed by variable recovery and periods of clinical stability;

Secondary Progressive MS (SPMS), wherein patients having ARMS develop sustained deterioration with or without relapses superimposed; and

Primary progressive-relapsing multiple sclerosis (PPRMS) or progressive-relapsing multiple sclerosis (PANS), an uncommon form wherein patients developing a progressive deterioration from the beginning can also develop relapses later on.

A clinical relapse, which may also be used herein as “relapse,” “confirmed relapse,” or “clinically defined relapse,” is defined as the appearance of one or more new neurological abnormalities or the reappearance of one or more previously observed neurological abnormalities.

This change in clinical state must last at least 48 hours and be immediately preceded by a relatively stable or improving neurological state of at least 30 days. This criterion is different from the clinical definition of exacerbation “at least 24 hours duration of symptoms,” as detailed in the section “relapse evaluation.”

An event is counted as a relapse only when the subject's symptoms are accompanied by observed objective neurological changes, consistent with:

a) an increase of at least 0.5 in the EDSS score or one grade in the score of two or more of the seven FS; or,

b) two grades in the score of one of FS as compared to the previous evaluation.

The subject must not be undergoing any acute metabolic changes such as fever or other medical abnormality. A change in bowel/bladder function or in cognitive function must not be entirely responsible for the changes in EDSS or FS scores.

As used herein, a “multiple sclerosis drug” is a drug or an agent intended to treat clinically defined MS, CIS, any form of neurodegenerative or demyelinating diseases, or symptoms of any of the above mentioned diseases. “Multiple sclerosis drugs” may include but are not limited to antibodies, immunosuppressants, anti-inflammatory agents, immunomodulators, cytokines, cytotoxic agents and steroids and may include approved drugs, drugs in clinical trial, or alternative treatments, intended to treat clinically defined MS, CIS or any form of neurodegenerative or demyelinating diseases. “Multiple sclerosis drugs” include but are not limited to Interferon and its derivatives (including BETASERON®, AVONEX® and REBIF®), Mitoxantrone and Natalizumab. Agents approved or in-trial for the treatment of other autoimmune diseases, but used in a MS or CIS patient to treat MS or CIS are also defined as multiple sclerosis drugs.

As used herein, a “naïve human” is a human that has not been treated with any multiple sclerosis drug.

As used herein, an “array of testing” for identifying whether a human subject afflicted with multiple sclerosis is a likely responder to glatiramer acetate (GA) therapy includes, but is not limited to, any analytical method test such as in vitro tests or biological assays such as the ex vivo tests. Examples of identifying whether a human subject afflicted with multiple sclerosis is a likely responder to glatiramer acetate (GA) therapy are disclosed in U.S. Pat. Nos. 8,759,302, 8,709,433, and 8,815,511, and U.S. Patent Application No. US 2014-0107208, the disclosures of which are hereby incorporated by reference in their entireties.

As used herein, “about” with regard to a stated number encompasses a range of +10 percent to −10 percent of the stated value. By way of example, about 100 mg therefore includes the range 90-110 mg and therefore also includes 90, 91, 92, 93, 94, 95 96, 97, 98, 99, 100, 101, 102, 103, 104, 105, 106, 107, 108, 109 and 110 mg. Accordingly, about 100 mg includes, in an embodiment, 100 mg.

By any range disclosed herein, it is meant that all hundredth, tenth and integer unit amounts within the range are specifically disclosed as part of the invention. Thus, for example, 1 mg to 50 mg means that 1.1, 1.2 . . . 1.9; and 2, 3 . . . 49 mg unit amounts are included as embodiments of this invention.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### Patient Subpopulation can be Selected Based on Genotypes

As used herein a SNP or “single nucleotide polymorphism” refers to a specific site in the genome where there is a difference in DNA base between individuals.

Several non-limiting examples of databases from which information on SNPs or genes that are associated with human disease can be retrieved include: NCBI resources, The SNP Consortium LTD, NCBI dbSNP database, International HapMap Project, 1000 Genomes Project, Glovar Variation Browser, SNPStats, PharmGKB, GEN-SniP, and SNPedia.

SNPs are identified herein using the rs identifier numbers in accordance with the NCBI dbSNP database, which is publically available at <URL:ncbi.nlm.nih.gov/projects/SNP/> or using the kgp identifier numbers, which were created by Illumina. Genotype at the kgp SNPs can be obtained by using the Illumina genotyping arrays. In addition, SNPs can be identified by the specific location on the chromosome indicated for the specific SNP.

Additional information about identifying SNPs can be obtained from the NCBI database SNP FAQ archive located at <UAL ncbi.nlm.nih.gov/books/NBK3848/> or from literature available on the Illumina website located at <URL: illumina.com/applications/genotyping/literature.ilmn>.

A genotype at a position of SNP (genotype “at a” SNP) may be represented by a single letter which corresponds to the identity of the nucleotide at the SNP, where A represents adenine, T represents thymine, C represents cytosine, and G represents guanine. The identity of two alleles at a single SNP may be represented by a two letter combination of A, T, C, and G, where the first letter of the two letter combination represents one allele and the second letter represents the second allele, and where A represents adenine, T represents thymine, C represents cytosine, and G represents guanine. Thus, a two allele genotype at a SNP can be represented as, for example, AA, AT, AG, AC, TT, TG, TC, GG, GC, or CC. It is understood that AT, AG, AC, TG, TC, and GC are equivalent to TA, GA, CA, GT, CT, and CG, respectively.

SNPs can be used as predictive indicators of the response to GA in subjects afflicted with multiple sclerosis. Presence of SNPs can be detected through obtaining a patient DNA sample and evaluating the patient sample for the presence of one or more SNPs, or for a certain set of SNPs. A patient DNA sample can be extracted, and a SNP can be detected in the sample, through any means known to one of ordinary skill in art. Some non-limiting examples of known techniques include detection via restriction fragment length polymorphism (RFLP) analysis, arrays including but not limited to planar microarrays or bead arrays, sequencing, single strand conformation polymorphism analysis (SSCP), chemical cleavage of mismatch (CCM), Polymerase chain reaction (PCR) and denaturing high performance liquid chromatography (DHPLC).

The genotyping array is a whole genome genotyping array. Whole-genome genotyping arrays are arrays that contain hundreds of thousands to millions of genetic sequences (which may also be named “probes”).

SNP can be detected through PCR amplification and sequencing of the DNA region comprising the SNP. SNPs can be detected using arrays, exemplified by gene chip, including but not limited to DNA arrays or microarrays, DNA chips, and whole genome genotyping arrays, all of which may be for example planar arrays or bead arrays, or a TaqMan open Array. Arrays/Microarrays for detection of genetic polymorphisms, changes or mutations (in general, genetic variations) such as a SNP in a DNA sequence, may comprise a solid surface, typically glass, on which a high number of genetic sequences are deposited (the probes), complementary to the genetic variations to be studied. Using standard robotic printers to apply probes to the array a high density of individual probe features can be obtained, for example probe densities of 600 features per cm² or more can be typically achieved. The positioning of probes on an array is precisely controlled by the printing device (robot, inkjet printer, photolithographic mask etc) and probes are aligned in a grid. The organization of probes on the array facilitates the subsequent identification of specific probe-target interactions. Additionally it is common, but not necessary, to divide the array features into smaller sectors, also grid-shaped, that are subsequently referred to as sub-arrays. Sub-arrays typically comprise 32 individual probe features although lower (e.g. 16) or higher (e.g. 64 or more) features can comprise each sub-array. In some arrays the probes are connected to beads instead of the solid support. Such arrays are called “bead arrays” or “bead CHIPs”.

Detection of SNP can be achieved by hybridization to sequences which specifically recognize the normal and the mutant allele in a fragment of DNA derived from a test sample. Typically, the fragment has been amplified, e.g. by using the polymerase chain reaction (PCR), and labeled e.g. with a fluorescent molecule. A laser can be used to detect bound labeled fragments on the chip and thus an individual who is homozygous for the normal allele can be specifically distinguished from heterozygous individuals (in the case of autosomal dominant conditions then these individuals are referred to as carriers) or those who are homozygous for the mutant allele. The amplification reaction and/or extension reaction is carried out on the microarray or bead itself. For differential hybridization based methods there are a number of methods for analyzing hybridization data for genotyping:
Increase in hybridization level: The hybridization levels of probes complementary to the normal and mutant alleles are compared.
Decrease in hybridization level: Differences in the sequence between a control sample and a test sample can be identified by a decrease in the hybridization level of the totally complementary oligonucleotides with a reference sequence.

A loss approximating 100% is produced in mutant homozygous individuals while there is only an approximately 50% loss in heterozygotes. In Microarrays for examining all the bases of a sequence of “n” nucleotides (“oligonucleotide”) of length in both strands, a minimum of “2n” oligonucleotides that overlap with the previous oligonucleotide in all the sequence except in the nucleotide are necessary. Typically the size of the oligonucleotides is about 25 nucleotides. However it should be appreciated that the oligonucleotide can be any length that is appropriate as would be understood by one of ordinary skill in the art. The increased number of oligonucleotides used to reconstruct the sequence reduces errors derived from fluctuation of the hybridization level.

However, the exact change in sequence cannot be identified with this method; this method is combined with sequencing to identify the mutation. Where amplification or extension is carried out on the microarray or bead itself, three methods are presented by way of example: In the Minisequencing strategy, a mutation specific primer is fixed on the slide and after an extension reaction with fluorescent dideoxynucleotides, the image of the Microarray is captured with a scanner. In the Primer extension strategy, two oligonucleotides are designed for detection of the wild type and mutant sequences respectively. The extension reaction is subsequently carried out with one fluorescently labeled nucleotide and the remaining nucleotides unlabelled. In either case the starting material can be either an RNA sample or a DNA product amplified by PCR. In the Tag arrays strategy, an extension reaction is carried out in solution with specific primers, which carry a determined 5¹ sequence or “tag”. The use of Microarrays with oligonucleotides complementary to these sequences or “tags” allows the capture of the resultant products of the extension.

The following abbreviations listed in Table 1 are used herein.

TABLE 1
Listing of Abbreviations

| Abbreviation | Explanation |
|---|---|
| Abs | Antibodies |
| ABS | Absorbance |
| AVG | Average |
| ELISA | Enzyme-linked Immunosorbent Assay |
| ET | End of Treatment |
| GA | Glatiramer Acetate |
| HRP | Horseradish Peroxidase |
| ICF | Informed consent form |
| IgG | Immunoglobuline G |
| MRI | Magnetic Resonance Imaging |
| MS | Multiple Sclerosis |
| NHS | Normal Human Serum pool |
| NMT | Not More Than |
| OL | Open Label |
| PC | Placebo Controlled |
| QAU | Quality Assurance Unit |
| RA % | Relative Absorbance % |
| PBS | Phosphate Buffered Saline |
| PHS | Positive Human Serum pool |
| RRMS | Relapsing Remitting Multiple Sclerosis |
| SST | System Suitability Test |
| TMB | 3,3,5,5-tetramethylbenzidine |

### EXPERIMENTAL DETAILS

### Example 1

This report presents the results of anti-Glatiramer Acetate (GA) IgG specific antibodies measurement in human serum samples of a cohort of 262 GA-treated patients tested in the bioanalytical Experimental Phases A, B and C of this study. Patients were derived from 1404 patients who participated in the clinical study entitled:

“A multinational, multicenter, randomized, parallel-group study performed in subjects with Relapsing-Remitting Multiple Sclerosis (RRMS) to assess the efficacy, safety and tolerability of Glatiramer Acetate (GA) injection 40 mg administered three times a week compared to placebo in a double-blind design” (8)

Patients were treated with 40 mg GA by subcutaneous injection three times a week (TIW) for 12 months in placebo controlled (PC) phase, and with the same treatment (40 mg GA by subcutaneous injection three times a week) in an open label extension phase.
Placebo-Controlled (PC) phase: 12 months of subcutaneous injections of Glatiramer Acetate 40 mg administered either three times a week or a matching placebo injection.
Open-Label (OL) Extension phase: Subjects completing the PC phase will be offered the opportunity to enter into an open-label (OL) extension phase in which all subjects will continue treatment with GA 40 mg administered three times a week until this dose is commercially available for the treatment of relapsing remitting multiple sclerosis (RRMS) patients or until the development of this GA dose is stopped by the Sponsor. This phase will be of variable duration for each subject, dependent upon when he/she is recruited into the study.

Serum samples, analyzed to detect anti-GA specific IgG antibodies, were collected at months 0 (baseline), 1, 3, 6, 9, 12 (end of PC phase), 18 and 24.

For the measurement of anti-GA specific IgG Abs, an enzyme-linked immunosorbent assay (ELISA) method was used.

Results of 50 baseline samples were used to calculate the study screening cut-point and confirmatory cut-point. The calculated screening cut-point value is 1.4% Relative Absorbance (RA) for this study. The confirmatory cut-point was calculated from OD values of baseline samples spiked with GA relative to the OD values of the same un-spiked samples. The confirmatory cut-point is 50.6%.

All samples of each patient were analyzed in two dilutions ( 1/500 and 1/1,000) to detect the presence of anti-GA IgG Abs. The screening results of the 1/500 dilution are presented as RA %. The determined RA % of 1740 out of the 1809 non-baseline samples was higher than the screening cut-point and these were considered as positive in the screening step.

Sample positivity of each screen positive sample of each patient was confirmed in further testing by comparison to the confirmatory cut-point. In the confirmatory tests, samples were tested both with and without GA spiking samples i.e. samples were spiked with GA reference standard to get a final GA concentration of 125 μg/ml in the 1:500 diluted serum. Sample positivity was verified in 99.9% of screen positive non baseline samples.

Confirmed positive samples were further analyzed in a titration test. Ten-fold serial dilutions of the samples were performed on top of the 500 fold dilution described in the method (i.e. starting from 1/500 sample to dilutions of 1/500,000) and the anti-GA IgG Abs titer of each positive sample was determined.

The bioanalytical study consisted testing all time points of 262 patients. The serum samples were screened for anti-GA IgG Abs. All samples which were found screen positive were tested in confirmatory test. The titer of the anti-GA Abs also were determined from all confirmed positive samples.

### Study Population

### Inclusion Criteria:

Subjects must meet all inclusion criteria in order to be eligible for the study:

1. Subjects must have a confirmed and documented MS diagnosis as defined by the Revised McDonald criteria [Ann Neurol 2005: 58:840-846], with a relapsing-remitting disease course.

2. Subjects must be ambulatory with an EDSS score of 0-5.5 in both screening and baseline visits.

3. Subjects must be in a relapse-free, stable neurological condition and free of corticosteroid treatment [intravenous (IV), intramuscular (IM) and/or per os(PO)] or ACTH (Adrenocorticotropic hormone) 30 days prior to screening (month −1) and between screening and baseline (month 0) visits.

4. Subjects must have experienced one of the following:
At least one documented relapse in the 12 months prior to screening, or
At least two documented relapses in the 24 months prior to screening, or
One documented relapse between 12 and 24 months prior to screening with at least one documented T1-Gd enhancing lesion in an MRI performed within 12 months prior to screening.

5. Subjects must be between 18 and 55 years of age, inclusive.

6. women of child-bearing potential must practice an acceptable method of birth control [acceptable methods of birth control in this study include: surgical sterilization, intrauterine devices, oral contraceptive, contraceptive patch, longacting injectable contraceptive, partner's vasectomy or a double-barrier method (condom or diaphragm with spermicide)].

7. Subjects must be able to sign and date a written informed consent prior to entering the study.

8. Subjects must be willing and able to comply with the protocol requirements for the duration of the study.

Exclusion Criteria:

Any of the following conditions will exclude the subject from entering the study:

1. Subjects with progressive forms of MS.

2. Use of experimental or investigational drugs, and/or participation in drug clinical studies within the 6 months prior to screening.

3. Use of immunosuppressive agents (including Mitoxantrone and Fingolimod) or cytotoxic agents within 6 months prior to the screening visit.

4. Use of natalizumab (Tysabri®) or any other monoclonal antibodies within 2 years prior to screening.

5. Use of cladribine within 2 years prior to screening.

6. Previous treatment with immunomodulators [including IFNβ 1a and 1b, and IV Immunoglobulin (IVIg)] within 2 months prior to screening.

7. Previous use of GA or any other glatiramoid.

8. Chronic (more than 30 consecutive days) systemic (IV, PO or IM) corticosteroid treatment within 6 months prior to screening visit.

9. Previous total body irradiation or total lymphoid irradiation.

10. Previous stem-cell treatment, autologous bone marrow transplantation or allogenic bone marrow transplantation.

11. Pregnancy or breastfeeding.

12. Subjects with a clinically significant or unstable medical or surgical condition that would preclude safe and complete study participation, as determined by medical history, physical exams, ECG, abnormal laboratory tests and chest Xray. Such conditions may include hepatic, renal or metabolic diseases, systemic disease, acute infection, current malignancy or recent history (5 years) of malignancy, major psychiatric disorder, history of drug and/or alcohol abuse and allergies that could be detrimental according to the investigator's judgment.

13. A known history of sensitivity to Gadolinium.

14. Inability to successfully undergo MRI scanning.

15. A known drug hypersensitivity to Mannitol.

16. Subjects who underwent endovascular treatment for Chronic Cerebrospinal Venous Insufficiency (CCSVI).

Study Samples

Test samples were generated in a Phase III, parallel-group, double-blind and open label extension study.

Serum samples to detect anti-GA specific antibodies in Experimental Phases A and B were collected at months 0 (baseline), 1, 3, 6, 9, 12 (end of PC phase), 18 and 24 according to the clinical protocol and laboratory manual for the clinical study.

Serum samples were separated into 3 aliquots with sample volume of 0.6 to 0.8 mL per aliquot. All aliquots from each blood collection point were labeled with the following information: “anti-GA serum”, study number, patient ID, sample ID. In the Experimental Phases A and B, altogether 2133 serum samples (from 270 patients from GA-treated study group) were analyzed for anti-GA IgG Abs (50 baseline samples were used for cut-point and confirmatory cut-point determination).

No factors which could affect the sample integrity were identified during the transfer and storage of samples at the Biological Testing Department.

Patients, the most time point samples of whom were available, were selected for testing. The analyzed samples derived from 270 patients as follows:
1945 samples from 243 patients collected according to clinical protocol at 8 time points, plus an extra sample from patient 601027 at TERMOL/ETOL sampling point
188 samples from 27 patients from 7 time points per patient, except for patient 537113 where baseline sample was whole blood instead of serum, and that sample was not analyzed.

Altogether 261 samples at baseline and 1809 samples at different time point after treatment from 262 patients have reportable results.

### Determination of Anti-GA IgG Antibodies

The purpose was to detect anti-GA specific IgG antibodies in human serum samples obtained from individuals participating in GA clinical studies.

Analysis was performed using ELISA. A microtiter plate is coated with GA (Teva Pharmaceutical Industries, Ltd.) reference standard (RS). After washing the excess unbound antigen and blocking the exposed well surface, the human serum samples (in serial dilutions of 1:500 and 1:1000) are added to the GA RS pre-coated wells. Anti-GA IgG antibodies are subsequently detected using a secondary antibody HRP conjugated mouse anti human IgG. Following washing of the excess unbound antibody-enzyme reagent, a substrate solution is added to the wells and color develops in proportion to the amount of anti-GA IgG antibodies in the serum sample.

The tested human serum samples are considered as at or above a normal range for the presence of the anti-GA IgG antibodies according to a cut-point. The cut-point is determined for each study from untreated human serum (NHS) samples obtained from the untreated individuals of that study (prior to treatment is administered or placebo subjects) or from untreated individuals from a population that is similar as much as possible to the study population.

Reagents are summarized in Table 2.

TABLE 2
Reagents Used in Determination of anti-GA IgG Specific
Antibodies

| REAGENTS | PREPARATION |
|---|---|
| COATING BUFFER | Dissolve the content of one Carbonate-Bicarbonate buffer capsule |
| | in 100 ml of purified water. Mix well on a magnetic stir plate. |
| | Adjust the pH to 9.5-9.6 using 1N HCl. Store up to 24 hours at |
| | 2°-8° C. |
| PHOSPHATE | Dissolve the content of one pouch pre-mixed bag in 1 liter of |
| BUFFERED SALINE | Purified water. |
| (PBS) | Store up to 1 week at 2°-8° C. Alternatively, sterilize PBS by |
| | autoclaving and store unopened up to three months at 2-8° C. |
| BLOCKING BUFFER | PBS solution contains 2% non-fat powdered milk (add 2.0 g of |
| | no-fat powdered milk into 100 ml PBS solution). |
| | Store up to one week at 2°-8° C. |
| WASHING BUFFER | PBS solution contains 0.05% Tween 20 (add 500 μl of Tween 20 |
| (PBST) | into 1000 ml PBS solution). |
| | Store up to one week at 2°-8° C. |
| SAMPLE BUFFER | PBST contains 0.1% gelatin and 1% non-fat powdered milk |
| | (Pre-warm 100 ml PBST solution by a hot plate for at least 5 min |
| | before adding 100 mg gelatin and 1.0 g milk powders), |
| | Store up to one week at 2°-8° C. |
| GLATIRAMER | For example, weigh about 30-40 mg of GA RS lyophilized |
| ACETATE | material and bring to a final concentration of 1.2 mg/ml with |
| REFERENCE | purified water, Determine the absorbance of the primary stock |
| STANDARD (RS) | solution at 275 nm. The concentration of GA is given by the |
| SOLUTION | equstion: |
| (1 MG/ML) | Absorbance_{275 nm} GA 1.0 mg/ml = 1.03 |
| | Based on the determined concentration, correct the volume of the |
| | primary stock solution to obtain a solution of 1 mg/ml. |
| | Divide the GA RS primary stock solution into working aliquots |
| | (250 μl/tube). |
| | Store at (−10° C.)-(−20° C.) for up to nine months. Use working |
| | aliquots once and discard after use. |

Prior to performing an assay, allow buffers to reach room temperature.

Each plate should include the following samples:
System suitability Test (SST)—wells coated with purified human IgG.
SST Blank—wells coated with the blocking buffer only (w/o hIgG and human serum sample). To be subtracted from the SST positive control.
Positive Human Serum (PHS)—positive human serum pool in dilution of 1/500.
Negative Human Serum (NHS)—normal human serum pool in dilution of 1/500.
Blank—wells coated with GA (w/o human serum sample). To be subtracted from the NHS & PHS controls and from the human tested serum samples.
Sample 1-Sample 13—different tested serum samples. Each sample applied in two dilutions ( 1/500 and 1/1000)

All tested human serum samples of different time points from the same individual should be tested (in triplicates), if possible, in the same plate to allow comparison.

For coating, prepare 20 ug/ml GA RS solution by diluting the 200 ul GA RS stock solution in 9.8 ml coating buffer (1:50). Prepare a 1.0 ug/ml dilution of hIgG coating antibody and coat the plate at 100 ul/well.

For washing, empty plate and tap out residual liquid, fill each well with 300 ul washing buffer and then invert plate to empty, tap out residual liquid onto paper towel.

For blocking, add 300 ul/well of blocking buffer to each well, seal the plate and incubate at room temperature for 1 hour, and wash the plates three times.

For sample loading, add 100 ul diluted test samples and/or control sample and/or sample buffer to each well. Seal the plate and incubate for 2 hours at room temperature, empty plate, tap out residual liquid and wash three times.

For detection antibody, prepare 1:4000 dilution of the biotinylated anti-hIgG detection antibody by diluting 2.5 ul biotinylated anti-hIgG antibody to 10 ml of blocking buffer. Prepare 10 ml biotinylated anti-human IgG detection antibody solution per plate and add 100 ul of the prepared detection antibodies to each well. Seal the plate and incubate for 1 hour at room temperature. Then empty plate, tap out residual liquid and wash five times.

For streptavidin conjugated horseradish peroxidase (HRP), prepare working dilution of the streptavidin conjugated HRP in blocking buffer. Then add 100 ul/well of the prepared streptavidin conjugated HRP solution, following shaking the plate for a few seconds. Then incubate the plate for 1 hour at room temperature in the dark, following washing five times.

For enzymatic reaction, TMB (3,3,5,5-tetramethylbenzidine peroxidase) is used. Mix equal volumes of TMB Peroxidase Substrate (Part 1) and Peroxidase Substrate Solution B in a clean glass container immediately prior to use. Dispense 100 ul of TMB solution into each well and incubate the plate for 15-30 minutes in the dark at 37° C. Stop the reaction with 100 ul stop solution per well (1N sulphuric acid) and shake the plate for a few seconds. Read the absorbance (ABS) of each well at 450 nm.

Evaluation of triplicates and outlier rejection are performed using SOP for triplicate analysis and outlier rejection in ELISA test. For each triplicates suspected of having an outlier, record all outlier rejection calculations and conclusion.

For blank subtraction, subtract the calculated average absorbance (ABS) of SST blank from average (AVG) ABS value of the SST. Subtract the calculated AVG ABS of blank from AVG ABS value of each test sample including PHS and NHS control samples. All data points that fall below 0.000 are set equal to 0.000.

To overcome plate-to-plate variability, the results are normalized by dividing the absorbance (ABS) of each test sample (including the controls) with that of the SST. This value is the Relative Absorbance (RA) ratio and is multiplied by 100 expressed in percent. These values are the reported response values of the test. The RA value is calculated according to the following equation:

$$RA ( \% ) = \frac{AVG test sample ABS - AVG Blank ABS}{AVG SST ABS - AVG SST Blank ABS} \times 100$$

AVG test sample means AVG ABS of each test sample and/or controls (PHS & NHS).

The RA values of the normal human serum samples obtained from untreated individuals are used to calculate a cut-point value of the test. The cut-point value is used to distinguish between negative and positive human serum samples. In case that the calculated cut point value of the study is lower than the RA of the detection limit (2*Blank) of the study, the cut point is calculated as follows:

$$RA = \frac{2 * Blank - Blank}{SST - SST Blank} \times 100$$

Plate acceptance criteria is the following:
The AVG ABS of the blank controls should be not more than (NMT) 20% from that of the SST control.
The AVG ABS of the SST should be not less than 0.8 at 450 nm.
The RA of the negative control (NHS) should be less than or equal to the RA value of the calculated cut point value.
The RA of the positive control (PHS) should be more than the RA value of the calculated cut point value.
In case that one of the test results do not comply with the plate acceptance criteria above, the test is not valid and should be repeated.

A tested human serum sample is considered as positive for the presence of anti-GA IgG antibodies only if in the 1/500 dilution its RA value is higher than the calculated cut-point value of that study. The results of the other dilution ( 1/1000) will be reported for monitoring, to allow semi quantitative comparison of the tested serum defined as positive.

### Serum Controls

Negative human serum pool (NHS) was prepared from 50 baseline samples used for cut point determination. Anti-GA IgG Abs-positive human serum pool (PHS) was prepared from anti-GA IgG Ab positive serum samples of 6 MS (Multiple Sclerosis) patients treated with GA obtained from an earlier clinical study.

### Analyses

Each plate included the following controls in triplicate wells:
System Suitability Test (SST)
SST Blank
Positive human serum (PHS)
Negative human serum (NHS)
Reagent Blank

In addition to above controls, PHS spiked with GA to get a final GA concentration of 125 μg/ml (in the 1:500 diluted sample) and PHS with solvent were also tested in the same dilution as test samples during the tests of Confirmatory cut-point determination and Confirmation of samples positivity.

Sample Analyses were Performed as Follows:

A screening cut-point value and confirmatory cut-point determination:

In order to distinguish positive samples from negative ones in the screening step and to confirm the positivity of the samples, a screening cut-point and a confirmatory cut point were determined. Fifty baseline samples were analyzed unspiked and spiked with 125 μg/ml GA (125 μg/ml GA concentration in the 1:500 diluted sample) according to the Bioanalytical Study Plan in 1/500 dilution. Each sample was analyzed 4 times by two analysts (2 runs each) on two different days.

### Sample Screening:

For the screening analysis, all available time point samples of 270 patients from the GA treated study group were tested in two dilutions ( 1/500 and 1/1000) in triplicate wells. All samples of each patient were tested on the same plate, whenever it was possible.

### Confirmation of Sample Positivity:

Each screen positive sample of each patient was tested both without spiking and with spiking with GA (dissolved in purified water). The final GA concentration in the spiked samples after the 1:500 dilution required by the method was 125 μg/ml. Unspiked and spiked samples were tested according to the method but only in 1/500 dilution.

### Anti-GA IgG Abs Titer Determination:

A titration test for determination of anti-GA IgG Abs titer was performed on all samples whose positivity was confirmed. Ten-fold serial dilutions of the samples were carried out (from 1:500 dilution to dilution of 1/500,000) and were tested according to the method.

### Cut-Point Calculations

### Screening Cut-Point Calculation:

The purpose of this study is to determine a screening cut-point for anti-GA IgG At in human serum samples obtained from untreated individuals in the clinical study. Fifty patients were enrolled to this study and were tested for IgG antibody levels at baseline (pre-treatment).

The calculated screening RA % cut-point was 1.4.

Each sample was tested in two replicates and by two analysts. The four values per individual were averaged to produce the data set used in the following calculations.

The ELISA OD 1/500 dilution readings (ABS) were transformed to percent relative absorbance (RA %) using the following equation:

$$RA ( \% ) = \frac{AVG test sample ABS - AVG Blank ABS}{AVG SST ABS - AVG SST Blank ABS} \times 100$$

The initial distribution of RA % was right skewed with some observed outliers. Clearly, this distribution does not follow the normal distribution model. This distribution's shape suggests the use of a log transformation. After applying the log transformation only one outlier was seen and the null hypothesis assuming normal distribution was not rejected (Pv=0.165). Assuming normal distribution, the 95% quantile on the log scale is 0.370. Using the backwards transformation on this value, yields a cut point of 1.447. In conclusion, the calculated cut-point for RA % of anti-GA IgG antibodies is 1.4%.

### Confirmatory Cut-Point Calculation:

The purpose of this study is to determine a confirmatory cut-point for anti-GA IgG Ab in human serum samples obtained from untreated individuals in the clinical study. Fifty patients were enrolled to this study and were tested for IgG antibody levels at baseline (pre-treatment).

The Confirmatory Cut Point (CCP) and % Inhibition were estimated using the nonparametric 99% percentile of In(Unspiked sample avg. OD/Spiked sample avg. OD). The obtained CCP is 2.024 and the obtained % Inhibition Cut Point is 50.589.

The confirmatory assay is intended to test for specificity of antibody binding by comparing the screening sample without spiked GA to a corresponding sample spiked with GA. The confirmatory cut-point factor is calculated to set the false positive rate at 1%. Reported value (RV) were the unspiked sample avg. ODA and the spiked sample avg. OD. RV were obtained from 50 naïve individuals, each measured 4 times over 2 days by 2 analysts.

The natural logarithmic of the ratio between the unspiked sample avg. OD to spiked sample avg. OD (i.e. In(Unspiked sample avg. OD/Spiked sample avg. OD)) was calculated and used for the CCP calculation and % inhibition cut-point.

The % inhibition cut-point is based on the following formula:

% Inhibition=100×(1−(1/CCP))

The four values per individual were averaged to produce the data set used in the CCPs. Since the distribution of In(Unspiked sample avg. OD/Spiked sample avg. OD) is not normally distributed (Shapiro-Wilk pv<0.0001) and the skewness is more than 1 the exp (nonparametric 1-alpha percentile of the set In(Unspiked sample avg. OD/Spiked sample avg. OD)) was used for the calculation of the cut-point.

The distribution of In(Unspiked sample avg. OD/Spiked sample avg. OD) was right skewed with some observed outliers. Clearly, this distribution does not follow the normal distribution (Shapiro-Wilk=0.675752, pv<0.0001), thus the cut-point is calculated as the nonparametric 99% quantile. The CCP (Unspiked/Spiked) at 99% quantile was 2.024 and the % Inhibition Cut Point was 50.589.

### Confirmation of Positivity of Samples

The positivity of a sample for anti-GA IgG antibodies was considered to be confirmed if the calculated percent RA inhibition was higher than the calculated confirmatory cut point (50.6%).

Percent RA inhibition was calculated according to the following equation:

Percent inhibition=100×(1−(RA of spiked)/(RA of unspiked))

99.9% of the tested screen positive non-baseline samples were found to have higher percent inhibition values than the confirmatory cut-point (50.6%).

### Determination of Anti-GA IgG Abs Titer

The titer was calculated using the following equation:

Titer of the sample=−log dilution factor of the last dilution that tests positive

(RA % higher than cut point)

The anti-GA IgG Abs titer was determined for all the confirmed positive samples.

### Determination of Annual Relapse Rate

The annualized relapse rate is defined as the total number of confirmed relapses a patient experienced per year. It is calculated as the total number of confirmed relapses divided by the overall exposure (in years) within the treatment group.

A complete neurological assessment will be performed at months −1 (screening), 0 (baseline), 3, 6, 9, 12 (end of PC phase). In the OL extension phase, a neurological examination will be performed every 6 months. In addition, a neurological examination will be performed at the termination visit of the OL phase.

A complete neurological assessment will also be performed in the follow-up visits for relapses.

The decision as to whether the neurological change is considered a confirmed relapse will be made by the Treating Neurologist/Physician (or Study Neurologist/Physician as applicable), based on the Converted EDSS or on the FS scores, as assessed by the Examining Neurologist/Physician (or Study Neurologist/Physician as applicable).

Follow-up visits to monitor the course of the relapse will be made at the Treating Neurologist/Physician's (or Study Neurologist/Physician as applicable) discretion and in case of ongoing/un-stabilized relapse within one month following the initial relapse, in addition to the assessment at the next scheduled visit, but the neurological assessments will be performed by the Examining Neurologist/Physician (or Study Neurologist/Physician as applicable).

### Viscosity Measurement

The manufacturing process for Copaxone® 40 mg/mL was based on the same process used to produce the marketed Copaxone® 20 mg/mL, except that aqueous Copaxone® 40 mg/mL was filtered at reduced temperature instead of at controlled room temperature prior to being filled into the syringe.

The average viscosity (cPa) of the batches of Copaxone® 40 mg/mL were measured using Rheocalc V2.5 Model LV, Spindle CP40, speed 80 rpm, Shear Rate 600 1/sec, Temperature 25° C.±0.1.

### Osmolality Measurement

The average osmolality (mosmol/Kg) of the batches of Copaxone® 40 mg/mL were measured in triplicates.

### Results

The number of scheduled and analyzed patient number was 270, but results of 262 were reported. The analyses were performed on altogether 690 plates. Thirty seven of these plates failed because of the following reasons:
Relative Absorbance (RA %) value of NHS pool sample was higher than the cut-point on 31 screening plates, on 2 confirmatory plates and on 1 titration plate
the Absorbance (ABS) value of System Suitability Test (SST) sample was higher than the measurement range of the plate reader on 3 confirmatory plates

The samples of the failed plates were retested. All plate control results met the acceptance criteria of the method, in the valid 653 plates of cut-point determination, screening, confirmatory and titration tests.

The average ABS values of the blank controls, i.e. Reagent Blank and SST Blank samples, obtained in 653 plate runs were 0.054 and 0.045, respectively. As shown in Table 3, in all plate runs, the % background of the two blank controls were found to be not more than (NMT) that specified in the method (NMT 20%).

The average ABS of the positive control samples (PHS and SST) were 1.406 and 1.840, respectively. As shown in Table 3, in all 653 plates, the ABS values of the SST were not less than 0.8, as specified in the SST acceptance criterion of the method.

In all the 653 reported plate runs, the RA % values of NHS pool samples were less than or equal to the calculated cut-point (1.4%), and the RA % results of PHS pool samples were higher than the calculated cut-point.

TABLE 3
Results of Plate Controls

| | NHS pool | | Blank | | SST | SST Blank | | PHS | |
|---|---|---|---|---|---|---|---|---|---|
| | ABS | RA % | ABS | % Back | ABS | ABS | % Back | ABS | RA % |
| Mean | 0.064 | 0.5 | 0.054 | 3.0 | 1.840 | 0.045 | 2.5 | 1.406 | 76.2 |
| SD | 0.014 | 0.3 | 0.013 | 0.9 | 0.309 | 0.004 | 0.5 | 0.314 | 17.8 |
| Min | 0.041 | 0.0 | 0.038 | 1.5 | 0.931 | 0.036 | 1.2 | 0.425 | 29.8 |
| Max | 0.155 | 1.4 | 0.151 | 9.4 | 3.510 | 0.088 | 5.5 | 2.634 | 199.6 |

* % Back (% Background) = (ABS value of the blank control/ABS value of the SST) × 100

The results of total of 2070 serum samples from 262 patients were reported for anti-GA IgG Abs. 261 of them were baseline samples, 1809 of them were non-baseline samples (months 1, 3, 6, 9, 12, 18, 24).

The RA % results of the 1/500 diluted samples are shown in Table 4. Based on the RA % values of the 1:500 diluted samples, 1740 out of the 1809 non-baseline samples (96.2%) were found to be higher than the cut-point value (RA % of 1.4).

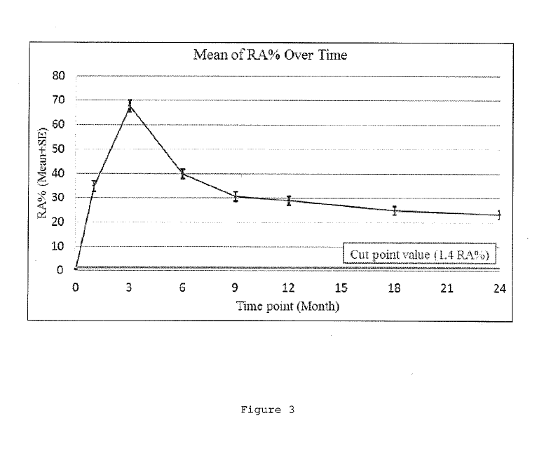

The immunological response profile of the averaged anti-GA IgG response (mean RA % values) in the GA treatment group, is represented in FIG. 3. The total antibody level increased at least 50 to 80 RA % within about 3 months from initiation of treatment. The antibody level peaked between about 3 and about 6 months from initiation of treatment and is higher than baseline about 12 months after the initial 40 mg/ml dose of glatiramer acetate. The antibody level increased at a greater rate during the first month compared to the rate of increase after the first month to the third month. As shown in FIG. 3, the antibody level after the peak remained low but did not reach the baseline level at month 24.

The percent inhibition results and conclusion of confirmatory test are listed in Table 4. The determined titers are also shown in Table 4.

TABLE 4
Results of Screening of the 1/500 Diluted Samples,
Confirmation Test and Anti-GA Antibodies Titer Determination

| | | | | | | | | Confirmed | | |
|---|---|---|---|---|---|---|---|---|---|---|
| | Visit | Screening | Average | Screening | Sample | Confirmation | Inhibition | Positivity | Titration | |
| Patient ID | Name | Plate# | OD | RA % | Positivity | Plate# | (%) | (Yes/No) | Plate# | Titer |
| 130001 | Baseline | SP1 | 0.054 | 0.6 | Negative | | | | | |
| 130001 | Month 1 | SP1 | 0.427 | 23.7 | POSITIVE | ConfP-7 | 99.0 | YES | TP211 | 2.7 |
| 130001 | Month 3 | SP1 | 2.354 | 143.1 | POSITIVE | ConfP-1 | 100.0 | YES | TP5 | 3.7 |
| 130001 | Month 6 | SP1 | 1.265 | 75.6 | POSITIVE | ConfP-7 | 99.2 | YES | TP211 | 3.7 |
| 130001 | Month 9 | SP1 | 0.964 | 57.0 | POSITIVE | ConfP-7 | 99.3 | YES | TP211 | 3.7 |
| 130001 | Month 12 | SP1 | 1.249 | 74.6 | POSITIVE | ConfP-7 | 99.5 | YES | TP211 | 3.7 |
| 130001 | Month 18 | SP1 | 1.752 | 105.8 | POSITIVE | ConfP-7 | 98.5 | YES | TP211 | 3.7 |
| 130001 | Month 24 | SP1 | 1.514 | 91.1 | POSITIVE | ConfP-7 | 98.2 | YES | TP211 | 3.7 |
| 130201 | Baseline | SP2 | 0.049 | 0.1 | Negative | | | | | |
| 130201 | Month 1 | SP2 | 0.675 | 43.6 | POSITIVE | ConfP-7 | 99.3 | YES | TP212 | 3.7 |
| 130201 | Month 3 | SP2 | 1.552 | 104.5 | POSITIVE | ConfP-1 | 99.2 | YES | TP5 | 3.7 |
| 130201 | Month 6 | SP2 | 0.236 | 13.1 | POSITIVE | ConfP-7 | 95.5 | YES | TP212 | 2.7 |
| 130201 | Month 9 | SP2 | 0.824 | 53.9 | POSITIVE | ConfP-7 | 99.5 | YES | TP212 | 3.7 |
| 130201 | Month 12 | SP2 | 1.499 | 100.8 | POSITIVE | ConfP-7 | 99.9 | YES | TP212 | 3.7 |
| 130201 | Month 18 | SP2 | 0.807 | 52.7 | POSITIVE | ConfP-7 | 99.8 | YES | TP212 | 3.7 |
| 130201 | Month 24 | SP2 | 1.016 | 67.3 | POSITIVE | ConfP-7 | 99.5 | YES | TP212 | 3.7 |
| 130304 | Baseline | SP3 | 0.055 | 0.8 | Negative | | | | | |
| 130304 | Month 1 | SP3 | 2.225 | 127.2 | POSITIVE | ConfP-7 | 95.1 | YES | TP213 | 4.7 |
| 130304 | Month 3 | SP3 | 2.522 | 144.5 | POSITIVE | ConfP-1 | 97.7 | YES | TP5 | 4.7 |
| 130304 | Month 6 | SP3 | 2.437 | 139.6 | POSITIVE | ConfP-8 | 90.0 | YES | TP213 | 4.7 |
| 130304 | Month 9 | SP3 | 2.324 | 133.0 | POSITIVE | ConfP-8 | 94.4 | YES | TP213 | 4.7 |
| 130304 | Month 12 | SP3 | 2.230 | 127.5 | POSITIVE | ConfP-8 | 95.4 | YES | TP213 | 4.7 |
| 130304 | Month 18 | SP3 | 2.087 | 119.2 | POSITIVE | ConfP-8 | 94.3 | YES | TP213 | 3.7 |
| 130304 | Month 24 | SP3 | 1.815 | 103.3 | POSITIVE | ConfP-8 | 92.0 | YES | TP213 | 3.7 |
| 130305 | Baseline | SP53 | 0.050 | 0.3 | Negative | | | | | |
| 130305 | Month 1 | SP53 | 0.672 | 31.4 | POSITIVE | ConfP-39 | 98.0 | YES | TP14 | 3.7 |
| 130305 | Month 3 | SP53 | 2.496 | 122.5 | POSITIVE | ConfP-40 | 99.5 | YES | TP14 | 4.7 |
| 130305 | Month 6 | SP53 | 2.606 | 128.0 | POSITIVE | ConfP-40 | 99.5 | YES | TP14 | 4.7 |
| 130305 | Month 9 | SP53 | 2.319 | 113.7 | POSITIVE | ConfP-40 | 99.5 | YES | TP14 | 4.7 |
| 130305 | Month 12 | SP53 | 1.913 | 93.4 | POSITIVE | ConfP-40 | 99.7 | YES | TP14 | 3.7 |
| 130305 | Month 18 | SP53 | 0.224 | 9.0 | POSITIVE | ConfP-40 | 99.2 | YES | TP14 | 2.7 |
| 130305 | Month 24 | SP53 | 1.341 | 64.8 | POSITIVE | ConfP-40 | 99.8 | YES | TP15 | 3.7 |
| 130306 | Baseline | SP53 | 0.045 | 0.1 | Negative | | | | | |
| 130306 | Month 1 | SP53 | 0.370 | 16.3 | POSITIVE | ConfP-40 | 99.1 | YES | TP15 | 3.7 |
| 130306 | Month 3 | SP53 | 1.879 | 91.7 | POSITIVE | ConfP-40 | 99.5 | YES | TP15 | 3.7 |
| 130306 | Month 6 | SP53 | 0.790 | 37.3 | POSITIVE | ConfP-40 | 99.4 | YES | TP15 | 3.7 |
| 130306 | Month 9 | SP54 | 0.284 | 12.0 | POSITIVE | ConfP-41 | 99.5 | YES | TP16 | 3.7 |
| 130306 | Month 12 | SP54 | 0.211 | 8.3 | POSITIVE | ConfP-41 | 98.9 | YES | TP17 | 2.7 |
| 130306 | Month 18 | SP54 | 0.179 | 6.7 | POSITIVE | ConfP-41 | 98.3 | YES | TP17 | 2.7 |
| 130306 | Month 24 | SP54 | 0.102 | 2.9 | POSITIVE | ConfP-41 | 97.1 | YES | TP17 | 2.7 |
| 130307 | Baseline | SP54 | 0.048 | 0.2 | Negative | | | | | |
| 130307 | Month 1 | SP54 | 0.786 | 37.0 | POSITIVE | ConfP-40 | 99.5 | YES | TP15 | 3.7 |
| 130307 | Month 3 | SP54 | 1.511 | 73.2 | POSITIVE | ConfP-40 | 99.5 | YES | TP15 | 3.7 |
| 130307 | Month 6 | SP54 | 0.740 | 34.7 | POSITIVE | ConfP-40 | 99.5 | YES | TP16 | 3.7 |
| 130307 | Month 9 | SP54 | 0.464 | 21.0 | POSITIVE | ConfP-41 | 99.5 | YES | TP16 | 3.7 |
| 130307 | Month 12 | SP54 | 0.822 | 38.8 | POSITIVE | ConfP-41 | 99.7 | YES | TP16 | 3.7 |
| 130307 | Month 18 | SP54 | 0.538 | 24.7 | POSITIVE | ConfP-41 | 99.7 | YES | TP16 | 3.7 |
| 130307 | Month 24 | SP54 | 0.250 | 10.3 | POSITIVE | ConfP-41 | 99.4 | YES | TP16 | 2.7 |
| 131301 | Baseline | SP191 | 0.076 | 0.2 | Negative | | | | | |
| 131301 | Month 1 | SP191 | 0.409 | 13.5 | POSITIVE | ConfP-136 | 99.1 | YES | TP314 | 2.7 |
| 131301 | Month 3 | SP191 | 2.612 | 101.7 | POSITIVE | ConfP-136 | 98.1 | YES | TP290 | 4.7 |
| 131301 | Month 6 | No sample received | | | | | | | | |
| 131301 | Month 9 | SP191 | 0.521 | 18.0 | POSITIVE | ConfP-136 | 99.2 | YES | TP290 | 2.7 |
| 131301 | Month 12 | SP191 | 0.560 | 19.6 | POSITIVE | ConfP-136 | 99.0 | YES | TP290 | 2.7 |
| 131301 | Month 18 | SP191 | 0.571 | 20.0 | POSITIVE | ConfP-136 | 99.4 | YES | TP290 | 2.7 |
| 131301 | Month 24 | SP191 | 0.522 | 18.0 | POSITIVE | ConfP-136 | 99.7 | YES | TP290 | 2.7 |
| 131501 | Baseline | SP3 | 0.046 | 0.2 | Negative | | | | | |
| 131501 | Month 1 | SP3 | 0.308 | 15.5 | POSITIVE | ConfP-8 | 84.8 | YES | TP214 | 2.7 |
| 131501 | Month 3 | SP3 | 1.998 | 114.0 | POSITIVE | ConfP-1 | 100.0 | YES | TP5 | 3.7 |
| 131501 | Month 6 | SP3 | 1.229 | 69.2 | POSITIVE | ConfP-8 | 94.9 | YES | TP214 | 3.7 |
| 131501 | Month 9 | SP4 | 0.863 | 45.8 | POSITIVE | ConfP-8 | 94.4 | YES | TP214 | 3.7 |
| 131501 | Month 12 | SP4 | 0.388 | 19.3 | POSITIVE | ConfP-8 | 90.1 | YES | TP214 | 2.7 |
| 131501 | Month 18 | SP4 | 0.976 | 52.1 | POSITIVE | ConfP-8 | 96.8 | YES | TP214 | 3.7 |
| 131501 | Month 24 | SP4 | 1.944 | 106.0 | POSITIVE | ConfP-8 | 97.8 | YES | TP214 | 3.7 |
| 131602 | Baseline | SP4 | 0.049 | 0.4 | Negative | | | | | |
| 131602 | Month 1 | SP4 | 0.796 | 42.0 | POSITIVE | ConfP-1 | 100.0 | YES | TP5 | 2.7 |
| 131602 | Month 3 | SP4 | 0.270 | 12.7 | POSITIVE | ConfP-8 | 99.1 | YES | TP215 | 2.7 |
| 131602 | Month 6 | SP4 | 0.114 | 4.0 | POSITIVE | ConfP-8 | 96.7 | YES | TP215 | 2.7 |
| 131602 | Month 9 | SP4 | 0.095 | 3.0 | POSITIVE | ConfP-9 | 99.0 | YES | TP215 | 2.7 |
| 131602 | Month 12 | SP4 | 0.062 | 1.1 | Negative | | | | | |
| 131602 | Month 18 | SP4 | 0.122 | 4.5 | POSITIVE | ConfP-9 | 98.6 | YES | TP215 | 2.7 |
| 131602 | Month 24 | SP4 | 0.112 | 3.9 | POSITIVE | ConfP-9 | 97.7 | YES | TP215 | 2.7 |
| 132201 | Baseline | SP55 | 0.044 | 0.3 | Negative | | | | | |
| 132201 | Month 1 | SP55 | 1.466 | 74.6 | POSITIVE | ConfP-41 | 99.4 | YES | TP17 | 3.7 |
| 132201 | Month 3 | SP55 | 1.704 | 87.0 | POSITIVE | ConfP-41 | 99.7 | YES | TP17 | 3.7 |
| 132201 | Month 6 | SP55 | 0.512 | 24.8 | POSITIVE | ConfP-41 | 99.3 | YES | TP17 | 3.7 |
| 132201 | Month 9 | SP55 | 2.268 | 116.5 | POSITIVE | ConfP-41 | 99.7 | YES | TP18 | 4.7 |
| 132201 | Month 12 | SP55 | 1.987 | 101.8 | POSITIVE | ConfP-41 | 99.8 | YES | TP18 | 3.7 |
| 132201 | Month 18 | SP55 | 1.991 | 102.0 | POSITIVE | ConfP-42 | 99.7 | YES | TP18 | 4.7 |
| 132201 | Month 24 | SP55 | 1.786 | 91.3 | POSITIVE | ConfP-42 | 99.7 | YES | TP18 | 4.7 |
| 132701 | Baseline | SP5 | 0.055 | 0.5 | Negative | | | | | |
| 132701 | Month 1 | SP5 | 0.177 | 7.1 | POSITIVE | ConfP-9 | 98.6 | YES | TP215 | 2.7 |
| 132701 | Month 3 | SP5 | 0.794 | 40.6 | POSITIVE | ConfP-1 | 99.7 | YES | TP5 | 3.7 |
| 132701 | Month 6 | SP5 | 0.321 | 14.9 | POSITIVE | ConfP-9 | 99.1 | YES | TP216 | 2.7 |
| 132701 | Month 9 | SP5 | 0.271 | 12.2 | POSITIVE | ConfP-9 | 99.0 | YES | TP216 | 2.7 |
| 132701 | Month 12 | SP5 | 0.190 | 7.9 | POSITIVE | ConfP-9 | 98.6 | YES | TP216 | 2.7 |
| 132701 | Month 18 | SP5 | 0.344 | 16.2 | POSITIVE | ConfP-9 | 99.1 | YES | TP216 | 3.7 |
| 132701 | Month 24 | SP5 | 0.359 | 17.0 | POSITIVE | ConfP-9 | 99.4 | YES | TP216 | 3.7 |
| 133501 | Baseline | SP5 | 0.053 | 0.4 | Negative | | | | | |
| 133501 | Month 1 | SP5 | 1.029 | 53.3 | POSITIVE | ConfP-9 | 97.0 | YES | TP216 | 3.7 |
| 133501 | Month 3 | SP5 | 1.873 | 99.0 | POSITIVE | ConfP-1 | 98.8 | YES | TP6 | 3.7 |
| 133501 | Month 6 | SP5 | 0.917 | 47.2 | POSITIVE | ConfP-9 | 96.7 | YES | TP217 | 3.7 |
| 133501 | Month 9 | SP6 | 0.848 | 41.2 | POSITIVE | ConfP-9 | 96.2 | YES | TP217 | 3.7 |
| 133501 | Month 12 | SP6 | 0.532 | 24.8 | POSITIVE | ConfP-9 | 95.0 | YES | TP217 | 3.7 |
| 133501 | Month 18 | SP6 | 0.638 | 30.3 | POSITIVE | ConfP-10 | 99.2 | YES | TP217 | 3.7 |
| 133501 | Month 24 | SP6 | 0.434 | 19.7 | POSITIVE | ConfP-10 | 98.2 | YES | TP217 | 3.7 |
| 133601 | Baseline | SP6 | 0.046 | 0.0 | Negative | | | | | |
| 133601 | Month 1 | SP6 | 1.063 | 52.4 | POSITIVE | ConfP-10 | 99.8 | YES | TP217 | 3.7 |
| 133601 | Month 3 | SP6 | 2.189 | 111.0 | POSITIVE | ConfP-1 | 99.9 | YES | TP6 | 3.7 |
| 133601 | Month 6 | SP6 | 0.479 | 22.0 | POSITIVE | ConfP-10 | 99.8 | YES | TP218 | 3.7 |
| 133601 | Month 9 | SP6 | 1.152 | 57.0 | POSITIVE | ConfP-10 | 99.7 | YES | TP218 | 3.7 |
| 133601 | Month 12 | SP6 | 0.865 | 42.1 | POSITIVE | ConfP-10 | 99.9 | YES | TP218 | 3.7 |
| 133601 | Month 18 | SP6 | 0.483 | 22.3 | POSITIVE | ConfP-10 | 99.8 | YES | TP218 | 3.7 |
| 133601 | Month 24 | SP6 | 0.926 | 45.3 | POSITIVE | ConfP-10 | 99.7 | YES | TP218 | 3.7 |
| 133903 | Baseline | SP7 | 0.044 | 0.0 | Negative | | | | | |
| 133903 | Month 1 | SP7 | 1.769 | 83.5 | POSITIVE | ConfP-10 | 99.5 | YES | TP218 | 3.7 |
| 133903 | Month 3 | SP7 | 2.409 | 114.5 | POSITIVE | ConfP-1 | 100.0 | YES | TP6 | 3.7 |
| 133903 | Month 6 | SP7 | 1.361 | 63.7 | POSITIVE | ConfP-10 | 99.8 | YES | TP219 | 3.7 |
| 133903 | Month 9 | SP7 | 1.082 | 50.2 | POSITIVE | ConfP-10 | 99.9 | YES | TP219 | 3.7 |
| 133903 | Month 12 | SP7 | 0.909 | 41.8 | POSITIVE | ConfP-10 | 99.7 | YES | TP219 | 3.7 |
| 133903 | Month 18 | SP7 | 1.014 | 46.9 | POSITIVE | ConfP-10 | 99.6 | YES | TP219 | 3.7 |
| 133903 | Month 24 | SP7 | 0.442 | 19.2 | POSITIVE | ConfP-11 | 99.4 | YES | TP219 | 2.7 |
| 134001 | Baseline | SP8 | 0.045 | 0.0 | Negative | | | | | |
| 134001 | Month 1 | SP8 | 0.048 | 0.2 | Negative | | | | | |
| 134001 | Month 3 | SP8 | 0.158 | 5.7 | POSITIVE | ConfP-11 | 97.8 | YES | TP219 | 2.7 |
| 134001 | Month 6 | SP8 | 0.170 | 6.3 | POSITIVE | ConfP-1 | 100.0 | YES | TP6 | 2.7 |
| 134001 | Month 9 | SP8 | 0.069 | 1.2 | Negative | | | | | |
| 134001 | Month 12 | SP8 | 0.059 | 0.7 | Negative | | | | | |
| 134001 | Month 18 | SP8 | 0.063 | 0.9 | Negative | | | | | |
| 134001 | Month 24 | SP8 | 0.050 | 0.3 | Negative | | | | | |
| 134602 | Baseline | SP55 | 0.045 | 0.4 | Negative | | | | | |
| 134602 | Month 1 | SP55 | 0.086 | 2.5 | POSITIVE | ConfP-42 | 97.6 | YES | TP18 | 2.7 |
| 134602 | Month 3 | SP55 | 0.737 | 36.5 | POSITIVE | ConfP-42 | 99.7 | YES | TP18 | 3.7 |
| 134602 | Month 6 | SP55 | 0.114 | 4.0 | POSITIVE | ConfP-42 | 98.4 | YES | TP19 | 2.7 |
| 134602 | Month 9 | SP56 | 0.105 | 2.6 | POSITIVE | ConfP-42 | 98.2 | YES | TP20 | 2.7 |
| 134602 | Month 12 | SP56 | 0.112 | 2.9 | POSITIVE | ConfP-43 | 97.8 | YES | TP20 | 2.7 |
| 134602 | Month 18 | SP56 | 0.128 | 3.6 | POSITIVE | ConfP-43 | 98.4 | YES | TP20 | 2.7 |
| 134602 | Month 24 | SP56 | 0.167 | 5.3 | POSITIVE | ConfP-43 | 96.7 | YES | TP20 | 2.7 |
| 134701 | Baseline | SP56 | 0.049 | 0.2 | Negative | | | | | |
| 134701 | Month 1 | SP56 | 0.125 | 3.5 | POSITIVE | ConfP-42 | 98.6 | YES | TP19 | 2.7 |
| 134701 | Month 3 | SP56 | 1.810 | 76.4 | POSITIVE | ConfP-42 | 99.7 | YES | TP19 | 3.7 |
| 134701 | Month 6 | SP56 | 0.554 | 22.1 | POSITIVE | ConfP-42 | 99.3 | YES | TP19 | 3.7 |
| 134701 | Month 9 | SP56 | 0.852 | 34.9 | POSITIVE | ConfP-42 | 99.7 | YES | TP19 | 3.7 |
| 134701 | Month 12 | SP56 | 2.183 | 92.5 | POSITIVE | ConfP-42 | 99.8 | YES | TP19 | 3.7 |
| 134701 | Month 18 | SP56 | 2.278 | 96.6 | POSITIVE | ConfP-42 | 99.9 | YES | TP20 | 4.7 |
| 134701 | Month 24 | SP56 | 2.069 | 87.6 | POSITIVE | ConfP-42 | 99.8 | YES | TP20 | 3.7 |
| 326401 | Baseline | SP57 | 0.044 | 0.1 | Negative | | | | | |
| 326401 | Month 1 | SP57 | 0.518 | 28.0 | POSITIVE | ConfP-43 | 99.7 | YES | TP21 | 2.7 |
| 326401 | Month 3 | SP57 | 1.443 | 82.6 | POSITIVE | ConfP-43 | 99.0 | YES | TP21 | 3.7 |
| 326401 | Month 6 | SP57 | 0.225 | 10.7 | POSITIVE | ConfP-43 | 99.1 | YES | TP21 | 2.7 |
| 326401 | Month 9 | SP57 | 0.121 | 4.6 | POSITIVE | ConfP-43 | 97.9 | YES | TP21 | 2.7 |
| 326401 | Month 12 | SP57 | 0.079 | 2.1 | POSITIVE | ConfP-43 | 98.4 | YES | TP21 | 2.7 |
| 326401 | Month 18 | SP57 | 0.077 | 2.0 | POSITIVE | ConfP-43 | 95.5 | YES | TP21 | 2.7 |
| 326401 | Month 24 | SP57 | 0.068 | 1.5 | POSITIVE | ConfP-43 | 98.0 | YES | TP22 | 2.7 |
| 327201 | Baseline | SP57 | 0.048 | 0.3 | Negative | | | | | |
| 327201 | Month 1 | SP57 | 0.639 | 35.2 | POSITIVE | ConfP-43 | 99.8 | YES | TP22 | 3.7 |
| 327201 | Month 3 | SP57 | 1.224 | 69.7 | POSITIVE | ConfP-43 | 99.9 | YES | TP22 | 3.7 |
| 327201 | Month 6 | SP57 | 0.610 | 33.5 | POSITIVE | ConfP-43 | 99.6 | YES | TP22 | 3.7 |
| 327201 | Month 9 | SP58 | 0.888 | 46.5 | POSITIVE | ConfP-44 | 99.6 | YES | TP23 | 3.7 |
| 327201 | Month 12 | SP58 | 0.474 | 23.7 | POSITIVE | ConfP-44 | 99.3 | YES | TP24 | 3.7 |
| 327201 | Month 18 | SP58 | 0.581 | 29.6 | POSITIVE | ConfP-44 | 99.6 | YES | TP24 | 3.7 |
| 327201 | Month 24 | SP58 | 0.480 | 24.0 | POSITIVE | ConfP-44 | 99.1 | YES | TP24 | 3.7 |
| 505201 | Baseline | SP59 | 0.049 | 0.2 | Negative | | | | | |
| 505201 | Month 1 | SP59 | 0.241 | 10.9 | POSITIVE | ConfP-44 | 98.3 | YES | TP24 | 2.7 |
| 505201 | Month 3 | SP59 | 0.495 | 25.0 | POSITIVE | ConfP-44 | 98.1 | YES | TP24 | 2.7 |
| 505201 | Month 6 | SP59 | 0.389 | 19.1 | POSITIVE | ConfP-45 | 99.0 | YES | TP24 | 2.7 |
| 505201 | Month 9 | SP59 | 0.093 | 2.6 | POSITIVE | ConfP-45 | 95.6 | YES | TP25 | 2.7 |
| 505201 | Month 12 | SP59 | 0.097 | 2.8 | POSITIVE | ConfP-45 | 94.6 | YES | TP25 | 2.7 |
| 505201 | Month 18 | SP59 | 0.108 | 3.5 | POSITIVE | ConfP-45 | 95.8 | YES | TP25 | 2.7 |
| 505201 | Month 24 | SP59 | 0.077 | 1.7 | POSITIVE | ConfP-45 | 90.0 | YES | TP25 | 2.7 |
| 505203 | Baseline | SP59 | 0.056 | 0.6 | Negative | | | | | |
| 505203 | Month 1 | SP59 | 0.140 | 5.2 | POSITIVE | ConfP-45 | 92.0 | YES | TP25 | 2.7 |
| 505203 | Month 3 | SP59 | 0.371 | 18.1 | POSITIVE | ConfP-45 | 97.0 | YES | TP25 | 2.7 |
| 505203 | Month 6 | SP59 | 0.298 | 14.0 | POSITIVE | ConfP-45 | 98.9 | YES | TP26 | 2.7 |
| 505203 | Month 9 | SP60 | 0.158 | 6.8 | POSITIVE | ConfP-46 | 96.6 | YES | TP27 | 2.7 |
| 505203 | Month 12 | SP60 | 0.106 | 3.7 | POSITIVE | ConfP-46 | 84.6 | YES | TP27 | 2.7 |
| 505203 | Month 18 | SP60 | 1.303 | 75.6 | POSITIVE | ConfP-46 | 99.2 | YES | TP27 | 3.7 |
| 505203 | Month 24 | SP60 | 0.094 | 2.9 | POSITIVE | ConfP-46 | 89.4 | YES | TP27 | 2.7 |
| 505205 | Baseline | SP60 | 0.047 | 0.1 | Negative | | | | | |
| 505205 | Month 1 | SP60 | 2.586 | 152.7 | POSITIVE | ConfP-45 | 98.1 | YES | TP26 | 4.7 |
| 505205 | Month 3 | SP60 | 2.454 | 144.8 | POSITIVE | ConfP-45 | 98.0 | YES | TP26 | 3.7 |
| 505205 | Month 6 | SP60 | 2.265 | 133.4 | POSITIVE | ConfP-45 | 99.0 | YES | TP26 | 3.7 |
| 505205 | Month 9 | SP60 | 2.245 | 132.2 | POSITIVE | ConfP-45 | 99.1 | YES | TP26 | 3.7 |
| 505205 | Month 12 | SP60 | 2.357 | 138.9 | POSITIVE | ConfP-45 | 99.6 | YES | TP26 | 3.7 |
| 505205 | Month 18 | SP60 | 1.400 | 81.4 | POSITIVE | ConfP-46 | 96.8 | YES | TP27 | 3.7 |
| 505205 | Month 24 | SP60 | 1.727 | 101.1 | POSITIVE | ConfP-46 | 98.3 | YES | TP27 | 3.7 |
| 505207 | Baseline | SP61 | 0.046 | 0.2 | Negative | | | | | |
| 505207 | Month 1 | SP61 | 0.109 | 3.2 | POSITIVE | ConfP-46 | 94.3 | YES | TP28 | 2.7 |
| 505207 | Month 3 | SP61 | 1.109 | 51.5 | POSITIVE | ConfP-46 | 96.5 | YES | TP28 | 3.7 |
| 505207 | Month 6 | SP61 | 0.232 | 9.2 | POSITIVE | ConfP-46 | 99.2 | YES | TP28 | 2.7 |
| 505207 | Month 9 | SP61 | 0.089 | 2.3 | POSITIVE | ConfP-46 | 100.0 | YES | TP28 | 2.7 |
| 505207 | Month 12 | SP61 | 0.092 | 2.4 | POSITIVE | ConfP-46 | 100.0 | YES | TP28 | 2.7 |
| 505207 | Month 18 | SP61 | 0.070 | 1.4 | Negative | | | | | |
| 505207 | Month 24 | SP61 | 0.072 | 1.4 | Negative | | | | | |
| 505210 | Baseline | SP61 | 0.048 | 0.3 | Negative | | | | | |
| 505210 | Month 1 | SP61 | 0.278 | 11.4 | POSITIVE | ConfP-46 | 100.0 | YES | TP28 | 2.7 |
| 505210 | Month 3 | SP61 | 0.937 | 43.2 | POSITIVE | ConfP-46 | 100.0 | YES | TP29 | 3.7 |
| 505210 | Month 6 | SP61 | 0.153 | 5.4 | POSITIVE | ConfP-47 | 97.8 | YES | TP29 | 2.7 |
| 505210 | Month 9 | SP62 | 0.353 | 14.1 | POSITIVE | ConfP-131 | 98.0 | YES | TP283 | 3.7 |
| 505210 | Month 12 | SP62 | 0.536 | 22.5 | POSITIVE | ConfP-131 | 94.4 | YES | TP283 | 3.7 |
| 505210 | Month 18 | SP62 | 0.449 | 18.5 | POSITIVE | ConfP-131 | 91.1 | YES | TP283 | 3.7 |
| 505210 | Month 24 | SP62 | 0.313 | 12.2 | POSITIVE | ConfP-131 | 85.3 | YES | TP283 | 3.7 |
| 505214 | Baseline | SP62 | 0.048 | 0.0 | Negative | | | | | |
| 505214 | Month 1 | SP62 | 0.127 | 3.7 | POSITIVE | ConfP-131 | 99.0 | YES | TP283 | 2.7 |
| 505214 | Month 3 | SP62 | 0.699 | 30.0 | POSITIVE | ConfP-131 | 99.7 | YES | TP283 | 3.7 |
| 505214 | Month 6 | SP62 | 0.797 | 34.5 | POSITIVE | ConfP-131 | 99.1 | YES | TP284 | 3.7 |
| 505214 | Month 9 | SP62 | 0.218 | 7.9 | POSITIVE | ConfP-131 | 99.0 | YES | TP284 | 2.7 |
| 505214 | Month 12 | SP62 | 0.406 | 16.5 | POSITIVE | ConfP-131 | 99.3 | YES | TP284 | 3.7 |
| 505214 | Month 18 | SP62 | 0.332 | 13.1 | POSITIVE | ConfP-131 | 99.4 | YES | TP284 | 3.7 |
| 505214 | Month 24 | SP62 | 0.261 | 9.8 | POSITIVE | ConfP-131 | 98.4 | YES | TP284 | 4.7 |
| 505301 | Baseline | SP63 | 0.045 | 0.2 | Negative | | | | | |
| 505301 | Month 1 | SP63 | 1.582 | 93.1 | POSITIVE | ConfP-47 | 99.0 | YES | TP29 | 3.7 |
| 505301 | Month 3 | SP63 | 1.918 | 113.4 | POSITIVE | ConfP-47 | 98.9 | YES | TP29 | 3.7 |
| 505301 | Month 6 | SP63 | 1.315 | 77.0 | POSITIVE | ConfP-47 | 98.5 | YES | TP29 | 3.7 |
| 505301 | Month 9 | SP63 | 0.951 | 55.0 | POSITIVE | ConfP-47 | 99.1 | YES | TP29 | 3.7 |
| 505301 | Month 12 | SP63 | 0.453 | 24.8 | POSITIVE | ConfP-47 | 98.0 | YES | TP30 | 2.7 |
| 505301 | Month 18 | SP63 | 0.509 | 28.2 | POSITIVE | ConfP-47 | 97.4 | YES | TP30 | 2.7 |
| 505301 | Month 24 | SP63 | 0.257 | 13.0 | POSITIVE | ConfP-47 | 92.3 | YES | TP30 | 2.7 |
| 505303 | Baseline | SP63 | 0.045 | 0.2 | Negative | | | | | |
| 505303 | Month 1 | SP63 | 0.121 | 4.8 | POSITIVE | ConfP-47 | 99.3 | YES | TP30 | 2.7 |
| 505303 | Month 3 | SP63 | 1.086 | 63.1 | POSITIVE | ConfP-47 | 99.6 | YES | TP30 | 3.7 |
| 505303 | Month 6 | SP63 | 0.187 | 8.8 | POSITIVE | ConfP-47 | 99.6 | YES | TP30 | 2.7 |
| 505303 | Month 9 | SP64 | 0.423 | 23.5 | POSITIVE | ConfP-48 | 99.2 | YES | TP32 | 2.7 |
| 505303 | Month 12 | SP64 | 0.070 | 1.7 | POSITIVE | ConfP-48 | 91.8 | YES | TP32 | 2.7 |
| 505303 | Month 18 | SP64 | 0.544 | 31.0 | POSITIVE | ConfP-48 | 99.7 | YES | TP32 | 3.7 |
| 505303 | Month 24 | SP64 | 0.072 | 1.8 | POSITIVE | ConfP-48 | 95.7 | YES | TP32 | 2.7 |
| 505306 | Baseline | SP64 | 0.044 | 0.1 | Negative | | | | | |
| 505306 | Month 1 | SP64 | 0.130 | 5.4 | POSITIVE | ConfP-47 | 98.5 | YES | TP31 | 2.7 |
| 505306 | Month 3 | SP64 | 0.748 | 43.6 | POSITIVE | ConfP-47 | 99.3 | YES | TP31 | 3.7 |
| 505306 | Month 6 | SP64 | 0.404 | 22.3 | POSITIVE | ConfP-48 | 98.9 | YES | TP31 | 2.7 |
| 505306 | Month 9 | SP64 | 0.103 | 3.7 | POSITIVE | ConfP-48 | 97.2 | YES | TP31 | 2.7 |
| 505306 | Month 12 | SP64 | 0.080 | 2.3 | POSITIVE | ConfP-48 | 94.7 | YES | TP31 | 2.7 |
| 505306 | Month 18 | SP64 | 0.091 | 3.0 | POSITIVE | ConfP-48 | 94.2 | YES | TP31 | 2.7 |
| 505306 | Month 24 | SP64 | 0.089 | 2.8 | POSITIVE | ConfP-48 | 93.5 | YES | TP32 | 2.7 |
| 505307 | Baseline | SP65 | 0.052 | 0.5 | Negative | | | | | |
| 505307 | Month 1 | SP65 | 2.992 | 137.5 | POSITIVE | ConfP-48 | 98.6 | YES | TP32 | 4.7 |
| 505307 | Month 3 | SP65 | 2.889 | 132.7 | POSITIVE | ConfP-48 | 98.6 | YES | TP33 | 4.7 |
| 505307 | Month 6 | SP65 | 2.061 | 94.1 | POSITIVE | ConfP-48 | 98.8 | YES | TP33 | 3.7 |
| 505307 | Month 9 | SP65 | 1.765 | 80.3 | POSITIVE | ConfP-48 | 98.9 | YES | TP33 | 3.7 |
| 505307 | Month 12 | SP65 | 1.442 | 65.2 | POSITIVE | ConfP-49 | 99.1 | YES | TP37 | 3.7 |
| 505307 | Month 18 | SP65 | 1.222 | 55.0 | POSITIVE | ConfP-49 | 99.1 | YES | TP33 | 3.7 |
| 505307 | Month 24 | SP65 | 0.768 | 33.8 | POSITIVE | ConfP-49 | 99.2 | YES | TP33 | 3.7 |
| 505308 | Baseline | SP65 | 0.048 | 0.3 | Negative | | | | | |
| 505308 | Month 1 | SP65 | 0.058 | 0.7 | Negative | | | | | |
| 505308 | Month 3 | SP65 | 0.338 | 13.8 | POSITIVE | ConfP-49 | 99.1 | YES | TP33 | 2.7 |
| 505308 | Month 6 | SP65 | 1.514 | 68.6 | POSITIVE | ConfP-49 | 99.5 | YES | TP34 | 3.7 |
| 505308 | Month 9 | SP66 | 0.693 | 32.1 | POSITIVE | ConfP-49 | 99.1 | YES | TP35 | 3.7 |
| 505308 | Month 12 | SP66 | 0.330 | 14.0 | POSITIVE | ConfP-50 | 99.6 | YES | TP35 | 2.7 |
| 505308 | Month 18 | SP66 | 1.091 | 51.9 | POSITIVE | ConfP-50 | 99.7 | YES | TP35 | 3.7 |
| 505308 | Month 24 | SP66 | 1.747 | 84.6 | POSITIVE | ConfP-50 | 99.8 | YES | TP35 | 3.7 |
| 505704 | Baseline | SP9 | 0.048 | 0.2 | Negative | | | | | |
| 505704 | Month 1 | SP9 | 1.916 | 97.8 | POSITIVE | ConfP-11 | 98.6 | YES | TP220 | 3.7 |
| 505704 | Month 3 | SP9 | 2.009 | 102.6 | POSITIVE | ConfP-1 | 99.4 | YES | TP6 | 3.7 |
| 505704 | Month 6 | SP9 | 1.274 | 64.2 | POSITIVE | ConfP-11 | 98.0 | YES | TP220 | 2.7 |
| 505704 | Month 9 | SP9 | 0.892 | 44.3 | POSITIVE | ConfP-11 | 99.6 | YES | TP220 | 2.7 |
| 505704 | Month 12 | SP9 | 0.552 | 26.5 | POSITIVE | ConfP-11 | 97.6 | YES | TP220 | 2.7 |
| 505704 | Month 18 | SP9 | 0.546 | 26.2 | POSITIVE | ConfP-11 | 100.0 | YES | TP220 | 2.7 |
| 505704 | Month 24 | SP9 | 0.478 | 22.6 | POSITIVE | ConfP-11 | 98.6 | YES | TP220 | 2.7 |
| 505705 | Baseline | SP197 | 0.094 | 0.3 | Negative | | | | | |
| 505705 | Month 1 | SP197 | 0.832 | 34.0 | POSITIVE | ConfP-145 | 99.2 | YES | TP298 | 3.7 |
| 505705 | Month 3 | SP197 | 1.122 | 47.2 | POSITIVE | ConfP-145 | 99.4 | YES | TP298 | 3.7 |
| 505705 | Month 6 | SP197 | 0.652 | 25.8 | POSITIVE | ConfP-145 | 98.4 | YES | TP298 | 2.7 |
| 505705 | Month 9 | SP197 | 0.359 | 12.4 | POSITIVE | ConfP-145 | 99.2 | YES | TP298 | 2.7 |
| 505705 | Month 12 | SP197 | 0.323 | 10.8 | POSITIVE | ConfP-145 | 99.5 | YES | TP298 | 2.7 |
| 505705 | Month 18 | No sample received | | | | | | | | |
| 505705 | Month 24 | SP199 | 0.161 | 4.5 | POSITIVE | ConfP-145 | 98.8 | YES | TP298 | 2.7 |
| 505706 | Baseline | SP9 | 0.049 | 0.2 | Negative | | | | | |
| 505706 | Month 1 | SP9 | 0.517 | 24.7 | POSITIVE | ConfP-11 | 93.1 | YES | TP221 | 2.7 |
| 505706 | Month 3 | SP9 | 1.273 | 64.2 | POSITIVE | ConfP-1 | 95.1 | YES | TP6 | 3.7 |
| 505706 | Month 6 | SP9 | 0.519 | 24.8 | POSITIVE | ConfP-11 | 95.3 | YES | TP221 | 2.7 |
| 505706 | Month 9 | SP10 | 0.483 | 22.5 | POSITIVE | ConfP-11 | 95.2 | YES | TP221 | 2.7 |
| 505706 | Month 12 | SP10 | 0.413 | 18.9 | POSITIVE | ConfP-11 | 98.6 | YES | TP221 | 2.7 |
| 505706 | Month 18 | SP10 | 0.675 | 32.3 | POSITIVE | ConfP-11 | 98.7 | YES | TP221 | 2.7 |
| 505706 | Month 24 | SP10 | 0.202 | 8.1 | POSITIVE | ConfP-12 | 85.8 | YES | TP221 | 2.7 |
| 505708 | Baseline | SP10 | 0.050 | 0.3 | Negative | | | | | |
| 505708 | Month 1 | SP10 | 0.049 | 0.3 | Negative | | | | | |
| 505708 | Month 3 | SP10 | 1.268 | 62.6 | POSITIVE | ConfP-12 | 98.8 | YES | TP222 | 3.7 |
| 505708 | Month 6 | SP10 | 1.360 | 67.3 | POSITIVE | ConfP-1 | 100.0 | YES | TP7 | 3.7 |
| 505708 | Month 9 | SP10 | 1.238 | 61.1 | POSITIVE | ConfP-12 | 98.5 | YES | TP222 | 3.7 |
| 505708 | Month 12 | SP10 | 0.901 | 43.9 | POSITIVE | ConfP-12 | 98.0 | YES | TP222 | 2.7 |
| 505708 | Month 18 | SP10 | 0.626 | 29.8 | POSITIVE | ConfP-12 | 99.3 | YES | TP222 | 2.7 |
| 505708 | Month 24 | SP10 | 0.357 | 16.0 | POSITIVE | ConfP-12 | 93.4 | YES | TP222 | 2.7 |
| 505712 | Baseline | SP198 | 0.103 | 1.0 | Negative | | | | | |
| 505712 | Month 1 | SP198 | 0.222 | 7.2 | POSITIVE | ConfP-146 | 63.3 | YES | TP310 | 2.7 |
| 505712 | Month 3 | SP198 | 1.112 | 53.2 | POSITIVE | ConfP-146 | 92.3 | YES | TP310 | 2.7 |
| 505712 | Month 6 | SP198 | 0.526 | 22.9 | POSITIVE | ConfP-146 | 81.9 | YES | TP310 | 2.7 |
| 505712 | Month 9 | SP198 | 0.390 | 15.9 | POSITIVE | ConfP-146 | 80.4 | YES | TP310 | 2.7 |
| 505712 | Month 12 | SP198 | 0.403 | 16.5 | POSITIVE | ConfP-146 | 77.9 | YES | TP310 | 2.7 |
| 505712 | Month 18 | No sample received | | | | | | | | |
| 505712 | Month 24 | SP198 | 0.442 | 18.6 | POSITIVE | ConfP-146 | 98.6 | YES | TP310 | 2.7 |
| 505713 | Baseline | SP66 | 0.053 | 0.2 | Negative | | | | | |
| 505713 | Month 1 | SP66 | 0.286 | 11.8 | POSITIVE | ConfP-49 | 98.3 | YES | TP34 | 2.7 |
| 505713 | Month 3 | SP66 | 0.779 | 36.4 | POSITIVE | ConfP-49 | 99.3 | YES | TP34 | 3.7 |
| 505713 | Month 6 | SP66 | 0.316 | 13.3 | POSITIVE | ConfP-49 | 99.2 | YES | TP34 | 2.7 |
| 505713 | Month 9 | SP66 | 0.970 | 45.9 | POSITIVE | ConfP-49 | 98.5 | YES | TP34 | 3.7 |
| 505713 | Month 12 | SP66 | 0.672 | 31.0 | POSITIVE | ConfP-49 | 98.6 | YES | TP34 | 3.7 |
| 505713 | Month 18 | SP66 | 0.765 | 35.7 | POSITIVE | ConfP-49 | 99.0 | YES | TP35 | 3.7 |
| 505713 | Month 24 | SP66 | 0.494 | 22.2 | POSITIVE | ConfP-49 | 99.1 | YES | TP35 | 3.7 |
| 505717 | Baseline | SP67 | 0.059 | 0.7 | Negative | | | | | |
| 505717 | Month 1 | SP67 | 1.815 | 88.8 | POSITIVE | ConfP-50 | 98.9 | YES | TP36 | 3.7 |
| 505717 | Month 3 | SP67 | 1.865 | 91.4 | POSITIVE | ConfP-50 | 99.3 | YES | TP36 | 3.7 |
| 505717 | Month 6 | SP67 | 1.668 | 81.5 | POSITIVE | ConfP-50 | 99.3 | YES | TP36 | 3.7 |
| 505717 | Month 9 | SP67 | 1.158 | 55.9 | POSITIVE | ConfP-50 | 99.1 | YES | TP36 | 3.7 |
| 505717 | Month 12 | SP67 | 1.616 | 78.9 | POSITIVE | ConfP-50 | 99.4 | YES | TP36 | 3.7 |
| 505717 | Month 18 | SP67 | 1.318 | 63.9 | POSITIVE | ConfP-50 | 99.3 | YES | TP36 | 3.7 |
| 505717 | Month 24 | SP67 | 0.676 | 31.6 | POSITIVE | ConfP-50 | 99.6 | YES | TP37 | 3.7 |
| 505802 | Baseline | SP11 | 0.057 | 0.8 | Negative | | | | | |
| 505802 | Month 1 | SP11 | 1.505 | 76.0 | POSITIVE | ConfP-12 | 99.1 | YES | TP222 | 3.7 |
| 505802 | Month 3 | SP11 | 1.557 | 78.7 | POSITIVE | ConfP-12 | 98.3 | YES | TP223 | 3.7 |
| 505802 | Month 6 | SP11 | 1.536 | 77.7 | POSITIVE | ConfP-12 | 99.5 | YES | TP223 | 3.7 |
| 505802 | Month 9 | SP11 | 1.677 | 85.0 | POSITIVE | ConfP-12 | 99.4 | YES | TP223 | 3.7 |
| 505802 | Month 12 | SP11 | 1.735 | 88.0 | POSITIVE | ConfP-12 | 99.5 | YES | TP223 | 3.7 |
| 505802 | Month 18 | SP11 | 1.741 | 88.3 | POSITIVE | ConfP-2 | 100.0 | YES | TP7 | 3.7 |
| 505802 | Month 24 | SP11 | 1.390 | 70.1 | POSITIVE | ConfP-12 | 99.4 | YES | TP223 | 3.7 |
| 505806 | Baseline | SP11 | 0.045 | 0.2 | Negative | | | | | |
| 505806 | Month 1 | SP11 | 1.378 | 69.4 | POSITIVE | ConfP-12 | 99.9 | YES | TP223 | 3.7 |
| 505806 | Month 3 | SP11 | 1.568 | 79.3 | POSITIVE | ConfP-2 | 100.0 | YES | TP7 | 3.7 |
| 505806 | Month 6 | SP11 | 0.717 | 35.1 | POSITIVE | ConfP-13 | 99.4 | YES | TP224 | 2.7 |
| 505806 | Month 9 | SP12 | 0.455 | 21.4 | POSITIVE | ConfP-13 | 98.0 | YES | TP224 | 2.7 |
| 505806 | Month 12 | SP12 | 0.313 | 14.0 | POSITIVE | ConfP-13 | 97.9 | YES | TP224 | 2.7 |
| 505806 | Month 18 | SP12 | 0.239 | 10.2 | POSITIVE | ConfP-13 | 98.1 | YES | TP224 | 2.7 |
| 505806 | Month 24 | SP12 | 0.189 | 7.6 | POSITIVE | ConfP-13 | 96.7 | YES | TP224 | 2.7 |
| 505807 | Baseline | SP12 | 0.088 | 2.3 | POSITIVE | ConfP-13 | 85.9 | YES | TP224 | 2.7 |
| 505807 | Month 1 | SP12 | 0.448 | 21.1 | POSITIVE | ConfP-13 | 91.7 | YES | TP225 | 3.7 |
| 505807 | Month 3 | SP12 | 1.142 | 57.2 | POSITIVE | ConfP-2 | 98.7 | YES | TP7 | 3.7 |
| 505807 | Month 6 | SP12 | 0.756 | 37.1 | POSITIVE | ConfP-13 | 91.3 | YES | TP225 | 3.7 |
| 505807 | Month 9 | SP12 | 0.594 | 28.7 | POSITIVE | ConfP-13 | 86.4 | YES | TP225 | 3.7 |
| 505807 | Month 12 | SP12 | 0.500 | 23.8 | POSITIVE | ConfP-13 | 84.4 | YES | TP225 | 3.7 |
| 505807 | Month 18 | SP12 | 0.378 | 17.4 | POSITIVE | ConfP-13 | 59.5 | YES | TP225 | 3.7 |
| 505807 | Month 24 | SP12 | 0.462 | 21.8 | POSITIVE | ConfP-13 | 58.2 | YES | TP225 | 3.7 |
| 505809 | Baseline | SP13 | 0.041 | 0.0 | Negative | | | | | |
| 505809 | Month 1 | SP13 | 0.597 | 46.1 | POSITIVE | ConfP-13 | 99.2 | YES | TP226 | 3.7 |
| 505809 | Month 3 | SP13 | 1.236 | 99.3 | POSITIVE | ConfP-2 | 100.0 | YES | TP7 | 3.7 |
| 505809 | Month 6 | SP13 | 0.401 | 29.8 | POSITIVE | ConfP-14 | 99.3 | YES | TP226 | 3.7 |
| 505809 | Month 9 | SP13 | 0.685 | 53.5 | POSITIVE | ConfP-14 | 99.6 | YES | TP226 | 3.7 |
| 505809 | Month 12 | SP13 | 0.543 | 41.6 | POSITIVE | ConfP-14 | 99.8 | YES | TP226 | 3.7 |
| 505809 | Month 18 | SP13 | 0.616 | 47.7 | POSITIVE | ConfP-14 | 99.8 | YES | TP226 | 2.7 |
| 505809 | Month 24 | SP13 | 0.156 | 9.4 | POSITIVE | ConfP-14 | 99.3 | YES | TP226 | 2.7 |
| 505810 | Baseline | SP67 | 0.052 | 0.3 | Negative | | | | | |
| 505810 | Month 1 | SP67 | 0.379 | 16.7 | POSITIVE | ConfP-50 | 100.0 | YES | TP37 | 2.7 |
| 505810 | Month 3 | SP67 | 1.056 | 50.7 | POSITIVE | ConfP-50 | 100.0 | YES | TP37 | 3.7 |
| 505810 | Month 6 | SP67 | 0.537 | 24.7 | POSITIVE | ConfP-50 | 99.5 | YES | TP41 | 2.7 |
| 505810 | Month 9 | SP68 | 0.155 | 5.5 | POSITIVE | ConfP-51 | 99.1 | YES | TP38 | 2.7 |
| 505810 | Month 12 | SP68 | 0.081 | 1.7 | POSITIVE | ConfP-51 | 96.9 | YES | TP39 | 2.7 |
| 505810 | Month 18 | SP68 | 0.280 | 12.1 | POSITIVE | ConfP-51 | 99.6 | YES | TP39 | 2.7 |
| 505810 | Month 24 | SP68 | 0.064 | 0.8 | Negative | | | | | |
| 505812 | Baseline | SP68 | 0.067 | 0.9 | Negative | | | | | |
| 505812 | Month 1 | SP68 | 0.218 | 8.8 | POSITIVE | ConfP-51 | 97.8 | YES | TP37 | 2.7 |
| 505812 | Month 3 | SP68 | 1.339 | 67.4 | POSITIVE | ConfP-51 | 99.5 | YES | TP37 | 3.7 |
| 505812 | Month 6 | SP68 | 0.861 | 42.4 | POSITIVE | ConfP-51 | 98.8 | YES | TP38 | 3.7 |
| 505812 | Month 9 | SP68 | 0.191 | 7.4 | POSITIVE | ConfP-51 | 98.1 | YES | TP38 | 2.7 |
| 505812 | Month 12 | SP68 | 0.117 | 3.6 | POSITIVE | ConfP-51 | 96.6 | YES | TP38 | 2.7 |
| 505812 | Month 18 | SP68 | 0.158 | 5.7 | POSITIVE | ConfP-51 | 95.8 | YES | TP38 | 2.7 |
| 505812 | Month 24 | SP68 | 0.129 | 4.2 | POSITIVE | ConfP-51 | 95.1 | YES | TP38 | 2.7 |
| 505813 | Baseline | SP69 | 0.046 | 0.2 | Negative | | | | | |
| 505813 | Month 1 | SP69 | 0.331 | 16.9 | POSITIVE | ConfP-51 | 98.2 | YES | TP39 | 2.7 |
| 505813 | Month 3 | SP69 | 1.425 | 80.8 | POSITIVE | ConfP-51 | 99.2 | YES | TP39 | 3.7 |
| 505813 | Month 6 | SP69 | 0.325 | 16.5 | POSITIVE | ConfP-51 | 98.9 | YES | TP39 | 2.7 |
| 505813 | Month 9 | SP69 | 0.319 | 16.2 | POSITIVE | ConfP-52 | 98.9 | YES | TP39 | 2.7 |
| 505813 | Month 12 | SP69 | 0.262 | 12.9 | POSITIVE | ConfP-52 | 98.6 | YES | TP40 | 2.7 |
| 505813 | Month 18 | SP69 | 0.212 | 9.9 | POSITIVE | ConfP-52 | 98.9 | YES | TP40 | 2.7 |
| 505813 | Month 24 | SP69 | 0.198 | 9.1 | POSITIVE | ConfP-52 | 98.7 | YES | TP40 | 2.7 |
| 505901 | Baseline | SP179 | 0.047 | 0.3 | Negative | | | | | |
| 505901 | Month 1 | SP179 | 1.990 | 108.4 | POSITIVE | ConfP-129 | 99.4 | YES | TP208 | 3.7 |
| 505901 | Month 3 | SP179 | 1.219 | 65.5 | POSITIVE | ConfP-129 | 99.7 | YES | TP208 | 3.7 |
| 505901 | Month 6 | SP179 | 1.048 | 56.0 | POSITIVE | ConfP-129 | 99.7 | YES | TP208 | 3.7 |
| 505901 | Month 9 | SP179 | 0.173 | 7.3 | POSITIVE | ConfP-130 | 97.8 | YES | TP208 | 2.7 |
| 505901 | Month 12 | SP179 | 0.152 | 6.2 | POSITIVE | ConfP-130 | 97.4 | YES | TP209 | 2.7 |
| 505901 | Month 18 | SP179 | 0.216 | 9.7 | POSITIVE | ConfP-130 | 98.0 | YES | TP209 | 2.7 |
| 505901 | Month 24 | SP179 | 0.168 | 7.1 | POSITIVE | ConfP-130 | 95.3 | YES | TP209 | 2.7 |
| 506003 | Baseline | SP69 | 0.046 | 0.2 | Negative | | | | | |
| 506003 | Month 1 | SP69 | 0.594 | 32.3 | POSITIVE | ConfP-52 | 99.4 | YES | TP40 | 2.7 |
| 506003 | Month 3 | SP69 | 1.897 | 108.4 | POSITIVE | ConfP-52 | 99.5 | YES | TP40 | 3.7 |
| 506003 | Month 6 | SP69 | 0.884 | 49.2 | POSITIVE | ConfP-52 | 99.5 | YES | TP40 | 3.7 |
| 506003 | Month 9 | SP70 | 0.400 | 19.0 | POSITIVE | ConfP-53 | 98.8 | YES | TP42 | 2.7 |
| 506003 | Month 12 | SP70 | 0.476 | 23.0 | POSITIVE | ConfP-53 | 98.8 | YES | TP42 | 2.7 |
| 506003 | Month 18 | SP70 | 0.570 | 28.0 | POSITIVE | ConfP-53 | 99.2 | YES | TP42 | 2.7 |
| 506003 | Month 24 | SP70 | 1.109 | 56.7 | POSITIVE | ConfP-53 | 99.6 | YES | TP43 | 3.7 |
| 506101 | Baseline | SP13 | 0.043 | 0.0 | Negative | | | | | |
| 506101 | Month 1 | SP13 | 1.948 | 158.6 | POSITIVE | ConfP-2 | 100.0 | YES | TP7 | 3.7 |
| 506101 | Month 3 | SP13 | 1.087 | 86.9 | POSITIVE | ConfP-14 | 99.7 | YES | TP227 | 3.7 |
| 506101 | Month 6 | SP13 | 0.444 | 33.4 | POSITIVE | ConfP-14 | 99.5 | YES | TP227 | 3.7 |
| 506101 | Month 9 | SP14 | 0.382 | 25.2 | POSITIVE | ConfP-14 | 99.5 | YES | TP227 | 3.7 |
| 506101 | Month 12 | SP14 | 0.252 | 15.6 | POSITIVE | ConfP-14 | 98.6 | YES | TP227 | 2.7 |
| 506101 | Month 18 | SP14 | 0.234 | 14.3 | POSITIVE | ConfP-14 | 98.9 | YES | TP227 | 2.7 |
| 506101 | Month 24 | SP14 | 0.387 | 25.6 | POSITIVE | ConfP-14 | 99.4 | YES | TP227 | 2.7 |
| 506102 | Baseline | SP14 | 0.047 | 0.5 | Negative | | | | | |
| 506102 | Month 1 | SP14 | 0.104 | 4.7 | POSITIVE | ConfP-14 | 95.8 | YES | TP228 | 2.7 |
| 506102 | Month 3 | SP14 | 0.476 | 32.1 | POSITIVE | ConfP-2 | 100.0 | YES | TP8 | 2.7 |
| 506102 | Month 6 | SP14 | 0.394 | 26.1 | POSITIVE | ConfP-14 | 99.1 | YES | TP228 | 3.7 |
| 506102 | Month 9 | SP14 | 0.221 | 13.3 | POSITIVE | ConfP-15 | 98.3 | YES | TP228 | 2.7 |
| 506102 | Month 12 | SP14 | 0.101 | 4.5 | POSITIVE | ConfP-15 | 97.7 | YES | TP228 | 2.7 |
| 506102 | Month 18 | SP14 | 0.087 | 3.5 | POSITIVE | ConfP-15 | 95.7 | YES | TP228 | 2.7 |
| 506102 | Month 24 | SP14 | 0.078 | 2.8 | POSITIVE | ConfP-15 | 93.8 | YES | TP228 | 2.7 |
| 506105 | Baseline | SP15 | 0.044 | 0.0 | Negative | | | | | |
| 506105 | Month 1 | SP15 | 0.230 | 13.1 | POSITIVE | ConfP-15 | 99.4 | YES | TP229 | 3.7 |
| 506105 | Month 3 | SP15 | 1.122 | 76.4 | POSITIVE | ConfP-2 | 100.0 | YES | TP8 | 3.7 |
| 506105 | Month 6 | SP15 | 0.592 | 38.8 | POSITIVE | ConfP-15 | 99.2 | YES | TP229 | 3.7 |
| 506105 | Month 9 | SP15 | 0.228 | 13.0 | POSITIVE | ConfP-15 | 98.5 | YES | TP229 | 2.7 |
| 506105 | Month 12 | SP15 | 0.266 | 15.7 | POSITIVE | ConfP-15 | 99.3 | YES | TP229 | 3.7 |
| 506105 | Month 18 | SP15 | 0.173 | 9.1 | POSITIVE | ConfP-15 | 99.3 | YES | TP229 | 2.7 |
| 506105 | Month 24 | SP15 | 0.340 | 20.9 | POSITIVE | ConfP-15 | 99.8 | YES | TP229 | 3.7 |
| 506106 | Baseline | SP15 | 0.043 | 0.0 | Negative | | | | | |
| 506106 | Month 1 | SP15 | 0.081 | 2.6 | POSITIVE | ConfP-15 | 98.6 | YES | TP230 | 2.7 |
| 506106 | Month 3 | SP15 | 0.180 | 9.6 | POSITIVE | ConfP-2 | 100.0 | YES | TP8 | 2.7 |
| 506106 | Month 6 | SP15 | 0.099 | 3.8 | POSITIVE | ConfP-15 | 97.3 | YES | TP230 | 2.7 |
| 506106 | Month 9 | SP16 | 0.154 | 7.4 | POSITIVE | ConfP-15 | 99.1 | YES | TP230 | 2.7 |
| 506106 | Month 12 | SP16 | 0.088 | 2.7 | POSITIVE | ConfP-16 | 98.4 | YES | TP230 | 2.7 |
| 506106 | Month 18 | SP16 | 0.104 | 3.9 | POSITIVE | ConfP-16 | 98.2 | YES | TP230 | 2.7 |
| 506106 | Month 24 | SP16 | 0.066 | 1.2 | Negative | | | | | |
| 506107 | Baseline | SP16 | 0.052 | 0.2 | Negative | | | | | |
| 506107 | Month 1 | SP16 | 1.301 | 87.7 | POSITIVE | ConfP-16 | 99.0 | YES | TP230 | 3.7 |
| 506107 | Month 3 | SP16 | 1.871 | 127.6 | POSITIVE | ConfP-2 | 99.1 | YES | TP8 | 3.7 |
| 506107 | Month 6 | SP16 | 1.098 | 73.5 | POSITIVE | ConfP-16 | 98.6 | YES | TP231 | 3.7 |
| 506107 | Month 9 | SP16 | 0.991 | 66.0 | POSITIVE | ConfP-16 | 98.4 | YES | TP231 | 3.7 |
| 506107 | Month 12 | SP16 | 0.593 | 38.1 | POSITIVE | ConfP-16 | 98.3 | YES | TP231 | 3.7 |
| 506107 | Month 18 | SP16 | 0.573 | 36.7 | POSITIVE | ConfP-16 | 98.6 | YES | TP231 | 3.7 |
| 506107 | Month 24 | SP16 | 0.469 | 29.4 | POSITIVE | ConfP-16 | 98.5 | YES | TP231 | 3.7 |
| 506109 | Baseline | SP17 | 0.070 | 1.9 | POSITIVE | ConfP-16 | 92.9 | YES | TP231 | 2.7 |
| 506109 | Month 1 | SP17 | 1.506 | 113.6 | POSITIVE | ConfP-2 | 100.0 | YES | TP8 | 3.7 |
| 506109 | Month 3 | SP17 | 1.206 | 90.3 | POSITIVE | ConfP-16 | 99.8 | YES | TP232 | 3.7 |
| 506109 | Month 6 | SP17 | 0.365 | 24.9 | POSITIVE | ConfP-16 | 99.7 | YES | TP232 | 3.7 |
| 506109 | Month 9 | SP17 | 0.226 | 14.1 | POSITIVE | ConfP-16 | 99.7 | YES | TP232 | 3.7 |
| 506109 | Month 12 | SP17 | 0.177 | 10.3 | POSITIVE | ConfP-16 | 99.1 | YES | TP232 | 2.7 |
| 506109 | Month 18 | SP17 | 0.201 | 12.1 | POSITIVE | ConfP-17 | 99.7 | YES | TP232 | 3.7 |
| 506109 | Month 24 | SP17 | 0.122 | 6.0 | POSITIVE | ConfP-17 | 99.0 | YES | TP232 | 2.7 |
| 506111 | Baseline | SP70 | 0.073 | 1.6 | POSITIVE | ConfP-52 | 33.3 | No | | |
| 506111 | Month 1 | SP70 | 0.714 | 35.7 | POSITIVE | ConfP-52 | 99.8 | YES | TP41 | 3.7 |
| 506111 | Month 3 | SP70 | 1.509 | 78.0 | POSITIVE | ConfP-52 | 99.5 | YES | TP41 | 3.7 |
| 506111 | Month 6 | SP70 | 0.846 | 42.7 | POSITIVE | ConfP-52 | 99.1 | YES | TP41 | 3.7 |
| 506111 | Month 9 | SP70 | 0.523 | 25.5 | POSITIVE | ConfP-52 | 99.2 | YES | TP41 | 2.7 |
| 506111 | Month 12 | SP70 | 0.903 | 45.7 | POSITIVE | ConfP-52 | 99.3 | YES | TP42 | 3.7 |
| 506111 | Month 18 | SP70 | 0.842 | 42.5 | POSITIVE | ConfP-53 | 99.2 | YES | TP42 | 2.7 |
| 506111 | Month 24 | SP70 | 0.424 | 20.3 | POSITIVE | ConfP-53 | 98.9 | YES | TP42 | 2.7 |
| 506301 | Baseline | SP71 | 0.046 | 0.2 | Negative | | | | | |
| 506301 | Month 1 | SP71 | 1.500 | 70.9 | POSITIVE | ConfP-53 | 99.4 | YES | TP43 | 3.7 |
| 506301 | Month 3 | SP71 | 2.163 | 103.2 | POSITIVE | ConfP-53 | 99.2 | YES | TP43 | 3.7 |
| 506301 | Month 6 | SP71 | 1.943 | 92.5 | POSITIVE | ConfP-53 | 99.1 | YES | TP43 | 3.7 |
| 506301 | Month 9 | SP71 | 1.386 | 65.4 | POSITIVE | ConfP-53 | 98.6 | YES | TP43 | 3.7 |
| 506301 | Month 12 | SP71 | 0.890 | 41.2 | POSITIVE | ConfP-53 | 98.2 | YES | TP43 | 3.7 |
| 506301 | Month 18 | SP71 | 1.988 | 94.6 | POSITIVE | ConfP-53 | 98.8 | YES | TP44 | 3.7 |
| 506301 | Month 24 | SP71 | 1.279 | 60.2 | POSITIVE | ConfP-53 | 98.1 | YES | TP44 | 3.7 |
| 506302 | Baseline | SP71 | 0.077 | 1.7 | POSITIVE | ConfP-54 | 44.4 | No | | |
| 506302 | Month 1 | SP71 | 1.083 | 50.6 | POSITIVE | ConfP-54 | 98.6 | YES | TP44 | 3.7 |
| 506302 | Month 3 | SP71 | 1.496 | 70.7 | POSITIVE | ConfP-54 | 99.1 | YES | TP44 | 3.7 |
| 506302 | Month 6 | SP71 | 0.897 | 41.6 | POSITIVE | ConfP-54 | 98.2 | YES | TP44 | 2.7 |
| 506302 | Month 9 | SP72 | 0.825 | 37.7 | POSITIVE | ConfP-54 | 98.3 | YES | TP46 | 2.7 |
| 506302 | Month 12 | SP72 | 0.697 | 31.5 | POSITIVE | ConfP-54 | 97.8 | YES | TP46 | 2.7 |
| 506302 | Month 18 | SP72 | 0.802 | 36.6 | POSITIVE | ConfP-55 | 98.3 | YES | TP46 | 2.7 |
| 506302 | Month 24 | SP72 | 0.873 | 40.0 | POSITIVE | ConfP-55 | 98.5 | YES | TP46 | 2.7 |
| 506303 | Baseline | SP72 | 0.047 | 0.2 | Negative | | | | | |
| 506303 | Month 1 | SP72 | 0.257 | 10.4 | POSITIVE | ConfP-54 | 99.1 | YES | TP45 | 2.7 |
| 506303 | Month 3 | SP72 | 2.161 | 102.0 | POSITIVE | ConfP-54 | 99.4 | YES | TP45 | 3.7 |
| 506303 | Month 6 | SP72 | 1.024 | 47.3 | POSITIVE | ConfP-54 | 98.6 | YES | TP45 | 3.7 |
| 506303 | Month 9 | SP72 | 0.561 | 25.0 | POSITIVE | ConfP-54 | 98.6 | YES | TP45 | 2.7 |
| 506303 | Month 12 | SP72 | 0.336 | 14.2 | POSITIVE | ConfP-54 | 98.2 | YES | TP45 | 2.7 |
| 506303 | Month 18 | SP72 | 0.722 | 32.7 | POSITIVE | ConfP-54 | 98.7 | YES | TP45 | 2.7 |
| 506303 | Month 24 | SP72 | 0.340 | 14.3 | POSITIVE | ConfP-54 | 98.6 | YES | TP46 | 2.7 |
| 506305 | Baseline | SP73 | 0.045 | 0.1 | Negative | | | | | |
| 506305 | Month 1 | SP73 | 0.561 | 23.1 | POSITIVE | ConfP-55 | 98.5 | YES | TP46 | 2.7 |
| 506305 | Month 3 | SP73 | 2.236 | 97.5 | POSITIVE | ConfP-55 | 98.6 | YES | TP47 | 3.7 |
| 506305 | Month 6 | SP73 | 1.256 | 53.9 | POSITIVE | ConfP-55 | 99.0 | YES | TP47 | 3.7 |
| 506305 | Month 9 | SP73 | 0.548 | 22.5 | POSITIVE | ConfP-55 | 99.0 | YES | TP47 | 2.7 |
| 506305 | Month 12 | SP73 | 0.843 | 35.6 | POSITIVE | ConfP-55 | 98.9 | YES | TP47 | 2.7 |
| 506305 | Month 18 | SP73 | 0.135 | 4.1 | POSITIVE | ConfP-55 | 97.3 | YES | TP47 | 2.7 |
| 506305 | Month 24 | SP73 | 0.135 | 4.1 | POSITIVE | ConfP-55 | 94.7 | YES | TP47 | 2.7 |
| 506401 | Baseline | SP73 | 0.044 | 0.1 | Negative | | | | | |
| 506401 | Month 1 | SP73 | 2.512 | 109.7 | POSITIVE | ConfP-55 | 99.4 | YES | TP48 | 4.7 |
| 506401 | Month 3 | SP73 | 1.988 | 86.5 | POSITIVE | ConfP-55 | 99.6 | YES | TP48 | 3.7 |
| 506401 | Month 6 | SP73 | 0.315 | 12.1 | POSITIVE | ConfP-55 | 99.0 | YES | TP48 | 2.7 |
| 506401 | Month 9 | SP74 | 0.297 | 12.5 | POSITIVE | ConfP-56 | 98.9 | YES | TP49 | 2.7 |
| 506401 | Month 12 | SP74 | 0.345 | 14.8 | POSITIVE | ConfP-56 | 98.8 | YES | TP49 | 2.7 |
| 506401 | Month 18 | SP74 | 0.481 | 21.4 | POSITIVE | ConfP-56 | 98.9 | YES | TP50 | 2.7 |
| 506401 | Month 24 | SP74 | 0.839 | 38.9 | POSITIVE | ConfP-56 | 99.5 | YES | TP50 | 3.7 |
| 513201 | Baseline | SP75 | 0.054 | 0.5 | Negative | | | | | |
| 513201 | Month 1 | SP75 | 0.143 | 5.0 | POSITIVE | ConfP-57 | 98.3 | YES | TP51 | 2.7 |
| 513201 | Month 3 | SP75 | 0.859 | 40.6 | POSITIVE | ConfP-57 | 98.5 | YES | TP51 | 3.7 |
| 513201 | Month 6 | SP75 | 0.429 | 19.2 | POSITIVE | ConfP-57 | 95.6 | YES | TP51 | 3.7 |
| 513201 | Month 9 | SP76 | 0.330 | 14.0 | POSITIVE | ConfP-58 | 96.8 | YES | TP53 | 2.7 |
| 513201 | Month 12 | SP76 | 0.105 | 3.0 | POSITIVE | ConfP-58 | 97.5 | YES | TP53 | 2.7 |
| 513201 | Month 18 | SP76 | 0.132 | 4.3 | POSITIVE | ConfP-58 | 90.3 | YES | TP53 | 2.7 |
| 513201 | Month 24 | SP76 | 0.087 | 2.1 | POSITIVE | ConfP-58 | 89.5 | YES | TP53 | 2.7 |
| 513202 | Baseline | SP76 | 0.046 | 0.1 | Negative | | | | | |
| 513202 | Month 1 | SP76 | 2.059 | 98.5 | POSITIVE | ConfP-57 | 99.0 | YES | TP52 | 3.7 |
| 513202 | Month 3 | SP76 | 1.115 | 52.3 | POSITIVE | ConfP-57 | 98.9 | YES | TP52 | 3.7 |
| 513202 | Month 6 | SP76 | 0.698 | 32.0 | POSITIVE | ConfP-57 | 99.4 | YES | TP52 | 3.7 |
| 513202 | Month 9 | SP76 | 0.282 | 11.6 | POSITIVE | ConfP-57 | 98.8 | YES | TP52 | 2.7 |
| 513202 | Month 12 | SP76 | 0.323 | 13.6 | POSITIVE | ConfP-57 | 98.5 | YES | TP52 | 2.7 |
| 513202 | Month 18 | SP76 | 0.493 | 21.9 | POSITIVE | ConfP-57 | 94.5 | YES | TP52 | 2.7 |
| 513202 | Month 24 | SP76 | 0.113 | 3.4 | POSITIVE | ConfP-58 | 96.7 | YES | TP53 | 2.7 |
| 513204 | Baseline | SP77 | 0.045 | 0.2 | Negative | | | | | |
| 513204 | Month 1 | SP77 | 0.132 | 6.3 | POSITIVE | ConfP-58 | 100.0 | YES | TP53 | 2.7 |
| 513204 | Month 3 | SP77 | 0.302 | 18.2 | POSITIVE | ConfP-58 | 100.0 | YES | TP54 | 2.7 |
| 513204 | Month 6 | SP77 | 0.073 | 2.2 | POSITIVE | ConfP-58 | 100.0 | YES | TP54 | 2.7 |
| 513204 | Month 9 | SP77 | 0.070 | 2.0 | POSITIVE | ConfP-58 | 96.4 | YES | TP54 | 2.7 |
| 513204 | Month 12 | SP77 | 0.096 | 3.8 | POSITIVE | ConfP-58 | 100.0 | YES | TP54 | 2.7 |
| 513204 | Month 18 | SP77 | 0.058 | 1.1 | Negative | | | | | |
| 513204 | Month 24 | SP77 | 0.086 | 3.1 | POSITIVE | ConfP-58 | 100.0 | YES | TP54 | 2.7 |
| 522001 | Baseline | SP183 | 0.061 | 1.3 | Negative | | | | | |
| 522001 | Month 1 | SP183 | 0.747 | 47.4 | POSITIVE | ConfP-132 | 98.2 | YES | TP285 | 3.7 |
| 522001 | Month 3 | SP183 | 0.910 | 58.4 | POSITIVE | ConfP-132 | 97.4 | YES | TP286 | 3.7 |
| 522001 | Month 6 | SP183 | 0.894 | 57.3 | POSITIVE | ConfP-132 | 96.8 | YES | TP286 | 3.7 |
| 522001 | Month 9 | SP183 | 0.905 | 58.0 | POSITIVE | ConfP-132 | 97.6 | YES | TP286 | 3.7 |
| 522001 | Month 12 | SP183 | 0.951 | 61.1 | POSITIVE | ConfP-132 | 96.8 | YES | TP286 | 3.7 |
| 522001 | Month 18 | SP183 | 0.606 | 37.9 | POSITIVE | ConfP-132 | 96.3 | YES | TP286 | 3.7 |
| 522001 | Month 24 | SP183 | 0.419 | 25.4 | POSITIVE | ConfP-132 | 95.9 | YES | TP286 | 3.7 |
| 522201 | Baseline | SP77 | 0.050 | 0.6 | Negative | | | | | |
| 522201 | Month 1 | SP77 | 0.415 | 26.1 | POSITIVE | ConfP-58 | 100.0 | YES | TP54 | 2.7 |
| 522201 | Month 3 | SP77 | 1.716 | 117.2 | POSITIVE | ConfP-58 | 99.7 | YES | TP55 | 3.7 |
| 522201 | Month 6 | SP77 | 0.370 | 23.0 | POSITIVE | ConfP-59 | 99.7 | YES | TP55 | 2.7 |
| 522201 | Month 9 | SP78 | 0.452 | 25.3 | POSITIVE | ConfP-59 | 99.5 | YES | TP56 | 3.7 |
| 522201 | Month 12 | SP78 | 0.651 | 37.5 | POSITIVE | ConfP-59 | 99.0 | YES | TP56 | 3.7 |
| 522201 | Month 18 | SP78 | 0.511 | 28.9 | POSITIVE | ConfP-59 | 99.2 | YES | TP56 | 3.7 |
| 522201 | Month 24 | SP78 | 0.536 | 30.5 | POSITIVE | ConfP-59 | 99.0 | YES | TP57 | 3.7 |
| 522205 | Baseline | SP78 | 0.045 | 0.4 | Negative | | | | | |
| 522205 | Month 1 | SP78 | 0.265 | 13.8 | POSITIVE | ConfP-59 | 99.6 | YES | TP55 | 2.7 |
| 522205 | Month 3 | SP78 | 0.809 | 47.2 | POSITIVE | ConfP-59 | 99.7 | YES | TP55 | 3.7 |
| 522205 | Month 6 | SP78 | 0.426 | 23.7 | POSITIVE | ConfP-59 | 99.4 | YES | TP55 | 2.7 |
| 522205 | Month 9 | SP78 | 0.306 | 16.4 | POSITIVE | ConfP-59 | 99.7 | YES | TP55 | 2.7 |
| 522205 | Month 12 | SP78 | 0.402 | 22.2 | POSITIVE | ConfP-59 | 99.6 | YES | TP56 | 3.7 |
| 522205 | Month 18 | SP78 | 0.471 | 26.5 | POSITIVE | ConfP-59 | 99.1 | YES | TP56 | 2.7 |
| 522205 | Month 24 | SP78 | 0.351 | 19.1 | POSITIVE | ConfP-59 | 99.3 | YES | TP56 | 3.7 |
| 522401 | Baseline | SP79 | 0.046 | 0.0 | Negative | | | | | |
| 522401 | Month 1 | SP79 | 0.689 | 35.0 | POSITIVE | ConfP-59 | 99.5 | YES | TP57 | 2.7 |
| 522401 | Month 3 | SP79 | 1.833 | 97.6 | POSITIVE | ConfP-60 | 99.5 | YES | TP57 | 3.7 |
| 522401 | Month 6 | SP79 | 1.579 | 83.7 | POSITIVE | ConfP-60 | 99.6 | YES | TP57 | 3.7 |
| 522401 | Month 9 | SP79 | 0.929 | 48.2 | POSITIVE | ConfP-60 | 99.5 | YES | TP57 | 3.7 |
| 522401 | Month 12 | SP79 | 0.284 | 12.9 | POSITIVE | ConfP-60 | 99.7 | YES | TP57 | 2.7 |
| 522401 | Month 18 | SP79 | 0.621 | 31.3 | POSITIVE | ConfP-60 | 99.6 | YES | TP58 | 2.7 |
| 522401 | Month 24 | SP79 | 0.274 | 12.4 | POSITIVE | ConfP-60 | 99.7 | YES | TP58 | 2.7 |
| 522402 | Baseline | SP79 | 0.045 | 0.0 | Negative | | | | | |
| 522402 | Month 1 | SP79 | 0.070 | 1.2 | Negative | | | | | |
| 522402 | Month 3 | SP79 | 1.364 | 72.0 | POSITIVE | ConfP-60 | 99.7 | YES | TP58 | 3.7 |
| 522402 | Month 6 | SP79 | 0.387 | 18.5 | POSITIVE | ConfP-60 | 99.5 | YES | TP58 | 2.7 |
| 522402 | Month 9 | SP80 | 0.208 | 9.8 | POSITIVE | ConfP-61 | 99.0 | YES | TP59 | 2.7 |
| 522402 | Month 12 | SP80 | 0.122 | 4.6 | POSITIVE | ConfP-61 | 99.4 | YES | TP59 | 2.7 |
| 522402 | Month 18 | SP80 | 0.083 | 2.3 | POSITIVE | ConfP-61 | 99.0 | YES | TP59 | 2.7 |
| 522402 | Month 24 | SP80 | 0.057 | 0.7 | Negative | | | | | |
| 522403 | Baseline | SP80 | 0.055 | 0.6 | Negative | | | | | |
| 522403 | Month 1 | SP80 | 0.079 | 2.0 | POSITIVE | ConfP-60 | 63.2 | YES | TP58 | 2.7 |
| 522403 | Month 3 | SP80 | 0.861 | 49.2 | POSITIVE | ConfP-60 | 97.1 | YES | TP58 | 3.7 |
| 522403 | Month 6 | SP80 | 0.129 | 5.1 | POSITIVE | ConfP-60 | 90.2 | YES | TP59 | 2.7 |
| 522403 | Month 9 | SP80 | 0.125 | 4.8 | POSITIVE | ConfP-60 | 91.8 | YES | TP59 | 2.7 |
| 522403 | Month 12 | SP80 | 0.199 | 9.3 | POSITIVE | ConfP-60 | 96.1 | YES | TP59 | 2.7 |
| 522403 | Month 18 | SP80 | 0.069 | 1.4 | Negative | | | | | |
| 522403 | Month 24 | SP80 | 0.046 | 0.1 | Negative | | | | | |
| 522404 | Baseline | SP81 | 0.053 | 0.4 | Negative | | | | | |
| 522404 | Month 1 | SP81 | 0.843 | 28.6 | POSITIVE | ConfP-61 | 99.6 | YES | TP60 | 3.7 |
| 522404 | Month 3 | SP81 | 1.845 | 64.3 | POSITIVE | ConfP-61 | 99.7 | YES | TP60 | 3.7 |
| 522404 | Month 6 | SP81 | 0.704 | 23.6 | POSITIVE | ConfP-61 | 99.0 | YES | TP60 | 2.7 |
| 522404 | Month 9 | SP81 | 0.586 | 19.4 | POSITIVE | ConfP-61 | 99.5 | YES | TP60 | 2.7 |
| 522404 | Month 12 | SP81 | 1.650 | 57.3 | POSITIVE | ConfP-61 | 99.6 | YES | TP60 | 3.7 |
| 522404 | Month 18 | SP81 | 0.467 | 15.2 | POSITIVE | ConfP-61 | 99.4 | YES | TP60 | 2.7 |
| 522404 | Month 24 | SP81 | 0.658 | 22.0 | POSITIVE | ConfP-61 | 99.5 | YES | TP61 | 2.7 |
| 522410 | Baseline | SP81 | 0.052 | 0.4 | Negative | | | | | |
| 522410 | Month 1 | SP81 | 0.157 | 4.1 | POSITIVE | ConfP-61 | 99.1 | YES | TP61 | 2.7 |
| 522410 | Month 3 | SP81 | 0.299 | 9.2 | POSITIVE | ConfP-61 | 99.6 | YES | TP61 | 2.7 |
| 522410 | Month 6 | SP81 | 0.232 | 6.8 | POSITIVE | ConfP-61 | 97.2 | YES | TP61 | 2.7 |
| 522410 | Month 9 | SP82 | 0.296 | 10.8 | POSITIVE | ConfP-62 | 98.4 | YES | TP62 | 2.7 |
| 522410 | Month 12 | SP82 | 0.590 | 23.3 | POSITIVE | ConfP-62 | 99.1 | YES | TP63 | 2.7 |
| 522410 | Month 18 | SP82 | 0.636 | 25.3 | POSITIVE | ConfP-62 | 98.6 | YES | TP63 | 2.7 |
| 522410 | Month 24 | SP82 | 0.707 | 28.3 | POSITIVE | ConfP-62 | 97.8 | YES | TP63 | 2.7 |
| 536601 | Baseline | SP82 | 0.053 | 0.5 | Negative | | | | | |
| 536601 | Month 1 | SP82 | 1.161 | 47.7 | POSITIVE | ConfP-62 | 99.8 | YES | TP61 | 3.7 |
| 536601 | Month 3 | SP82 | 1.243 | 51.1 | POSITIVE | ConfP-62 | 99.7 | YES | TP61 | 3.7 |
| 536601 | Month 6 | SP82 | 0.294 | 10.7 | POSITIVE | ConfP-62 | 99.0 | YES | TP62 | 2.7 |
| 536601 | Month 9 | SP82 | 0.122 | 3.4 | POSITIVE | ConfP-62 | 98.9 | YES | TP62 | 2.7 |
| 536601 | Month 12 | SP82 | 0.110 | 2.9 | POSITIVE | ConfP-62 | 98.6 | YES | TP62 | 2.7 |
| 536601 | Month 18 | SP82 | 0.090 | 2.0 | POSITIVE | ConfP-62 | 97.7 | YES | TP62 | 2.7 |
| 536601 | Month 24 | SP82 | 0.103 | 2.6 | POSITIVE | ConfP-62 | 96.7 | YES | TP62 | 2.7 |
| 536603 | Baseline | SP83 | 0.046 | 0.0 | Negative | | | | | |
| 536603 | Month 1 | SP83 | 0.096 | 2.3 | POSITIVE | ConfP-62 | 95.9 | YES | TP63 | 2.7 |
| 536603 | Month 3 | SP83 | 0.862 | 38.0 | POSITIVE | ConfP-62 | 98.6 | YES | TP63 | 3.7 |
| 536603 | Month 6 | SP83 | 0.643 | 27.8 | POSITIVE | ConfP-63 | 99.5 | YES | TP63 | 2.7 |
| 536603 | Month 9 | SP83 | 0.346 | 13.9 | POSITIVE | ConfP-63 | 99.8 | YES | TP64 | 3.7 |
| 536603 | Month 12 | SP83 | 0.275 | 10.6 | POSITIVE | ConfP-63 | 99.4 | YES | TP64 | 2.7 |
| 536603 | Month 18 | SP83 | 0.227 | 8.4 | POSITIVE | ConfP-63 | 99.2 | YES | TP64 | 2.7 |
| 536603 | Month 24 | SP83 | 0.246 | 9.3 | POSITIVE | ConfP-63 | 98.8 | YES | TP64 | 2.7 |
| 536604 | Baseline | SP185 | 0.077 | 0.2 | Negative | | | | | |
| 536604 | Month 1 | No sample received | | | | | | | | |
| 536604 | Month 3 | SP185 | 0.744 | 33.0 | POSITIVE | ConfP-137 | 99.7 | YES | TP314 | 3.7 |
| 536604 | Month 6 | SP185 | 0.941 | 42.6 | POSITIVE | ConfP-137 | 98.7 | YES | TP290 | 3.7 |
| 536601 | Month 9 | SP185 | 0.800 | 35.7 | POSITIVE | ConfP-137 | 98.0 | YES | TP291 | 2.7 |
| 536604 | Month 12 | SP185 | 0.469 | 19.5 | POSITIVE | ConfP-137 | 97.4 | YES | TP291 | 2.7 |
| 536604 | Month 18 | SP185 | 0.283 | 10.3 | POSITIVE | ConfP-137 | 98.4 | YES | TP291 | 2.7 |
| 536604 | Month 24 | SP185 | 0.246 | 8.5 | POSITIVE | ConfP-137 | 97.2 | YES | TP291 | 2.7 |
| 536701 | Baseline | SP83 | 0.046 | 0.0 | Negative | | | | | |
| 536701 | Month 1 | SP83 | 0.370 | 15.1 | POSITIVE | ConfP-63 | 99.5 | YES | TP64 | 2.7 |
| 536701 | Month 3 | SP83 | 1.432 | 64.6 | POSITIVE | ConfP-63 | 99.8 | YES | TP64 | 3.7 |
| 536701 | Month 6 | SP83 | 0.866 | 38.2 | POSITIVE | ConfP-63 | 99.5 | YES | TP65 | 3.7 |
| 536701 | Month 9 | SP84 | 0.467 | 20.4 | POSITIVE | ConfP-64 | 99.3 | YES | TP66 | 3.7 |
| 536701 | Month 12 | SP84 | 0.530 | 23.4 | POSITIVE | ConfP-64 | 99.3 | YES | TP66 | 3.7 |
| 536701 | Month 18 | SP84 | 0.581 | 25.9 | POSITIVE | ConfP-64 | 97.2 | YES | TP66 | 3.7 |
| 536701 | Month 24 | SP84 | 0.568 | 25.3 | POSITIVE | ConfP-64 | 97.6 | YES | TP66 | 3.7 |
| 536702 | Baseline | SP84 | 0.050 | 0.4 | Negative | | | | | |
| 536702 | Month 1 | SP84 | 0.519 | 22.9 | POSITIVE | ConfP-63 | 99.1 | YES | TP65 | 3.7 |
| 536702 | Month 3 | SP84 | 1.842 | 86.4 | POSITIVE | ConfP-63 | 99.8 | YES | TP65 | 3.7 |
| 536702 | Month 6 | SP84 | 0.638 | 28.6 | POSITIVE | ConfP-63 | 99.5 | YES | TP65 | 3.7 |
| 536702 | Month 9 | SP84 | 0.787 | 35.8 | POSITIVE | ConfP-63 | 99.2 | YES | TP65 | 3.7 |
| 536702 | Month 12 | SP84 | 0.605 | 27.0 | POSITIVE | ConfP-63 | 99.5 | YES | TP65 | 3.7 |
| 536702 | Month 18 | SP84 | 0.344 | 14.5 | POSITIVE | ConfP-64 | 98.8 | YES | TP66 | 2.7 |
| 536702 | Month 24 | SP84 | 0.428 | 18.5 | POSITIVE | ConfP-64 | 99.4 | YES | TP66 | 2.7 |
| 536703 | Baseline | SP85 | 0.046 | 0.1 | Negative | | | | | |
| 536703 | Month 1 | SP85 | 2.417 | 118.9 | POSITIVE | ConfP-64 | 97.9 | YES | TP67 | 4.7 |
| 536703 | Month 3 | SP85 | 2.358 | 115.9 | POSITIVE | ConfP-64 | 98.2 | YES | TP67 | 4.7 |
| 536703 | Month 6 | SP85 | 1.351 | 65.5 | POSITIVE | ConfP-64 | 99.4 | YES | TP67 | 3.7 |
| 536703 | Month 9 | SP85 | 0.649 | 30.3 | POSITIVE | ConfP-64 | 99.3 | YES | TP67 | 3.7 |
| 536703 | Month 12 | SP85 | 0.398 | 17.7 | POSITIVE | ConfP-64 | 99.1 | YES | TP67 | 2.7 |
| 536703 | Month 18 | SP85 | 0.492 | 22.4 | POSITIVE | ConfP-64 | 98.5 | YES | TP67 | 3.7 |
| 536703 | Month 24 | SP85 | 0.477 | 21.7 | POSITIVE | ConfP-64 | 98.6 | YES | TP68 | 2.7 |
| 536704 | Baseline | SP85 | 0.044 | 0.0 | Negative | | | | | |
| 536704 | Month 1 | SP85 | 0.049 | 0.2 | Negative | | | | | |
| 536704 | Month 3 | SP85 | 0.334 | 14.5 | POSITIVE | ConfP-65 | 96.3 | YES | TP68 | 2.7 |
| 536704 | Month 6 | SP85 | 0.444 | 20.0 | POSITIVE | ConfP-65 | 99.1 | YES | TP68 | 3.7 |
| 536704 | Month 9 | SP86 | 0.134 | 4.9 | POSITIVE | ConfP-65 | 97.6 | YES | TP69 | 2.7 |
| 536704 | Month 12 | SP86 | 0.184 | 7.7 | POSITIVE | ConfP-65 | 97.8 | YES | TP69 | 2.7 |
| 536704 | Month 18 | SP86 | 0.072 | 1.6 | POSITIVE | ConfP-65 | 92.2 | YES | TP69 | 2.7 |
| 536704 | Month 24 | SP86 | 0.086 | 2.3 | POSITIVE | ConfP-65 | 97.1 | YES | TP69 | 2.7 |
| 536707 | Baseline | SP86 | 0.052 | 0.5 | Negative | | | | | |
| 536707 | Month 1 | SP86 | 0.060 | 0.9 | Negative | | | | | |
| 536707 | Month 3 | SP86 | 0.282 | 13.0 | POSITIVE | ConfP-65 | 99.2 | YES | TP68 | 3.7 |
| 536707 | Month 6 | SP86 | 0.176 | 7.2 | POSITIVE | ConfP-65 | 100.0 | YES | TP68 | 2.7 |
| 536707 | Month 9 | SP184 | 0.322 | 17.7 | POSITIVE | ConfP-132 | 97.5 | YES | TP287 | 2.7 |
| 536707 | Month 12 | SP86 | 0.526 | 26.2 | POSITIVE | ConfP-65 | 98.2 | YES | TP68 | 3.7 |
| 536707 | Month 18 | SP86 | 0.261 | 11.8 | POSITIVE | ConfP-65 | 93.9 | YES | TP69 | 2.7 |
| 536707 | Month 24 | SP86 | 0.367 | 17.6 | POSITIVE | ConfP-65 | 97.2 | YES | TP69 | 2.7 |
| 536708 | Baseline | SP87 | 0.050 | 0.4 | Negative | | | | | |
| 536708 | Month 1 | SP87 | 0.933 | 54.1 | POSITIVE | ConfP-65 | 99.5 | YES | TP70 | 3.7 |
| 536708 | Month 3 | SP87 | 2.002 | 119.2 | POSITIVE | ConfP-65 | 99.1 | YES | TP70 | 3.7 |
| 536708 | Month 6 | SP87 | 0.980 | 57.0 | POSITIVE | ConfP-66 | 97.6 | YES | TP70 | 3.7 |
| 536708 | Month 9 | SP87 | 0.623 | 35.2 | POSITIVE | ConfP-66 | 98.2 | YES | TP70 | 3.7 |
| 536708 | Month 12 | SP87 | 0.354 | 18.9 | POSITIVE | ConfP-66 | 98.2 | YES | TP70 | 2.7 |
| 536708 | Month 18 | SP87 | 0.709 | 40.5 | POSITIVE | ConfP-66 | 98.9 | YES | TP70 | 3.7 |
| 536708 | Month 24 | SP87 | 0.212 | 10.2 | POSITIVE | ConfP-66 | 96.6 | YES | TP71 | 2.7 |
| 536709 | Baseline | SP87 | 0.047 | 0.2 | Negative | | | | | |
| 536709 | Month 1 | SP87 | 1.695 | 100.5 | POSITIVE | ConfP-66 | 98.7 | YES | TP71 | 3.7 |
| 536709 | Month 3 | SP87 | 2.290 | 136.7 | POSITIVE | ConfP-66 | 98.6 | YES | TP71 | 4.7 |
| 536709 | Month 6 | SP87 | 2.086 | 124.3 | POSITIVE | ConfP-66 | 99.3 | YES | TP71 | 3.7 |
| 536709 | Month 9 | SP88 | 1.818 | 96.4 | POSITIVE | ConfP-67 | 99.6 | YES | TP72 | 4.7 |
| 536709 | Month 12 | SP88 | 1.500 | 79.0 | POSITIVE | ConfP-67 | 99.5 | YES | TP73 | 3.7 |
| 536709 | Month 18 | SP88 | 1.221 | 63.7 | POSITIVE | ConfP-67 | 98.4 | YES | TP73 | 3.7 |
| 536709 | Month 24 | SP88 | 0.679 | 34.0 | POSITIVE | ConfP-67 | 98.9 | YES | TP73 | 3.7 |
| 536804 | Baseline | SP88 | 0.046 | 0.0 | Negative | | | | | |
| 536804 | Month 1 | SP88 | 1.682 | 89.0 | POSITIVE | ConfP-66 | 99.5 | YES | TP71 | 3.7 |
| 536804 | Month 3 | SP88 | 1.305 | 68.3 | POSITIVE | ConfP-66 | 99.7 | YES | TP71 | 3.7 |
| 536804 | Month 6 | SP88 | 0.824 | 41.9 | POSITIVE | ConfP-66 | 99.5 | YES | TP72 | 3.7 |
| 536804 | Month 9 | SP88 | 0.514 | 24.9 | POSITIVE | ConfP-66 | 99.6 | YES | TP72 | 3.7 |
| 536804 | Month 12 | SP88 | 0.454 | 21.7 | POSITIVE | ConfP-66 | 98.5 | YES | TP72 | 3.7 |
| 536804 | Month 18 | SP88 | 0.332 | 15.0 | POSITIVE | ConfP-67 | 98.2 | YES | TP72 | 2.7 |
| 536804 | Month 24 | SP88 | 0.383 | 17.8 | POSITIVE | ConfP-67 | 98.3 | YES | TP72 | 3.7 |
| 536805 | Baseline | SP102 | 0.052 | 0.8 | Negative | | | | | |
| 536805 | Month 1 | SP102 | 0.391 | 29.6 | POSITIVE | ConfP-77 | 98.4 | YES | TP94 | 2.7 |
| 536805 | Month 3 | SP102 | 1.787 | 148.0 | POSITIVE | ConfP-77 | 98.0 | YES | TP94 | 3.7 |
| 536805 | Month 6 | SP102 | 1.298 | 106.5 | POSITIVE | ConfP-77 | 98.5 | YES | TP94 | 3.7 |
| 536805 | Month 9 | SP102 | 0.975 | 79.1 | POSITIVE | ConfP-77 | 99.1 | YES | TP94 | 3.7 |
| 536805 | Month 12 | SP102 | 0.786 | 63.1 | POSITIVE | ConfP-77 | 98.6 | YES | TP94 | 3.7 |
| 536805 | Month 18 | SP102 | 0.137 | 8.1 | POSITIVE | ConfP-77 | 96.9 | YES | TP95 | 2.7 |
| 536805 | Month 24 | SP102 | 0.063 | 1.8 | POSITIVE | ConfP-77 | 90.5 | YES | TP95 | 2.7 |
| 536806 | Baseline | SP101 | 0.044 | 0.2 | Negative | | | | | |
| 536806 | Month 1 | SP101 | 0.050 | 0.7 | Negative | | | | | |
| 536806 | Month 3 | SP101 | 0.530 | 41.6 | POSITIVE | ConfP-76 | 98.3 | YES | TP93 | 3.7 |
| 536806 | Month 6 | SP101 | 1.068 | 87.5 | POSITIVE | ConfP-76 | 99.0 | YES | TP94 | 3.7 |
| 536806 | Month 9 | SP102 | 0.841 | 67.8 | POSITIVE | ConfP-77 | 99.4 | YES | TP95 | 3.7 |
| 536806 | Month 12 | SP102 | 0.875 | 70.7 | POSITIVE | ConfP-77 | 99.5 | YES | TP95 | 3.7 |
| 536806 | Month 18 | SP102 | 0.395 | 29.9 | POSITIVE | ConfP-77 | 99.4 | YES | TP95 | 3.7 |
| 536806 | Month 24 | SP102 | 0.310 | 22.7 | POSITIVE | ConfP-77 | 99.7 | YES | TP95 | 2.7 |
| 536807 | Baseline | SP101 | 0.047 | 0.4 | Negative | | | | | |
| 536807 | Month 1 | SP101 | 0.382 | 29.0 | POSITIVE | ConfP-76 | 99.0 | YES | TP92 | 3.7 |
| 536807 | Month 3 | SP101 | 0.916 | 74.6 | POSITIVE | ConfP-76 | 99.0 | YES | TP92 | 3.7 |
| 536807 | Month 6 | SP101 | 0.876 | 71.2 | POSITIVE | ConfP-76 | 98.6 | YES | TP93 | 3.7 |
| 536807 | Month 9 | SP101 | 0.492 | 38.4 | POSITIVE | ConfP-76 | 98.9 | YES | TP93 | 3.7 |
| 536807 | Month 12 | SP101 | 0.735 | 59.1 | POSITIVE | ConfP-76 | 99.4 | YES | TP93 | 3.7 |
| 536807 | Month 18 | SP101 | 0.395 | 30.1 | POSITIVE | ConfP-76 | 99.1 | YES | TP93 | 3.7 |
| 536807 | Month 24 | SP101 | 0.875 | 71.1 | POSITIVE | ConfP-76 | 98.3 | YES | TP93 | 3.7 |
| 536808 | Baseline | SP104 | 0.050 | 0.0 | Negative | | | | | |
| 536808 | Month 1 | SP104 | 1.719 | 133.7 | POSITIVE | ConfP-78 | 98.0 | YES | TP97 | 4.7 |
| 536808 | Month 3 | SP104 | 2.295 | 179.8 | POSITIVE | ConfP-78 | 98.1 | YES | TP97 | 4.7 |
| 536808 | Month 6 | SP104 | 1.797 | 139.9 | POSITIVE | ConfP-78 | 99.7 | YES | TP98 | 3.7 |
| 536808 | Month 9 | SP104 | 2.153 | 168.4 | POSITIVE | ConfP-78 | 99.7 | YES | TP98 | 4.7 |
| 536808 | Month 12 | SP104 | 0.772 | 57.8 | POSITIVE | ConfP-78 | 99.7 | YES | TP98 | 3.7 |
| 536808 | Month 18 | SP104 | 2.103 | 164.4 | POSITIVE | ConfP-79 | 97.4 | YES | TP98 | 4.7 |
| 536808 | Month 24 | SP104 | 2.181 | 170.7 | POSITIVE | ConfP-79 | 97.4 | YES | TP98 | 4.7 |
| 536811 | Baseline | SP103 | 0.048 | 0.5 | Negative | | | | | |
| 536811 | Month 1 | SP103 | 0.138 | 7.6 | POSITIVE | ConfP-78 | 99.3 | YES | TP97 | 2.7 |
| 536811 | Month 3 | SP103 | 0.363 | 25.5 | POSITIVE | ConfP-78 | 99.4 | YES | TP97 | 3.7 |
| 536811 | Month 6 | SP103 | 0.176 | 10.7 | POSITIVE | ConfP-78 | 98.6 | YES | TP97 | 2.7 |
| 536811 | Month 9 | SP104 | 0.120 | 5.5 | POSITIVE | ConfP-79 | 94.6 | YES | TP98 | 2.7 |
| 536811 | Month 12 | SP104 | 0.071 | 1.6 | POSITIVE | ConfP-79 | 91.3 | YES | TP99 | 2.7 |
| 536811 | Month 18 | SP104 | 0.068 | 1.4 | Negative | | | | | |
| 536811 | Month 24 | SP104 | 0.066 | 1.2 | Negative | | | | | |
| 536901 | Baseline | SP17 | 0.047 | 0.2 | Negative | | | | | |
| 536901 | Month 1 | SP17 | 0.371 | 25.3 | POSITIVE | ConfP-17 | 99.8 | YES | TP233 | 3.7 |
| 536901 | Month 3 | SP17 | 1.293 | 97.0 | POSITIVE | ConfP-2 | 100.0 | YES | TP8 | 3.7 |
| 536901 | Month 6 | SP17 | 0.244 | 15.5 | POSITIVE | ConfP-17 | 99.7 | YES | TP233 | 3.7 |
| 536901 | Month 9 | SP18 | 0.202 | 11.8 | POSITIVE | ConfP-17 | 98.0 | YES | TP233 | 3.7 |
| 536901 | Month 12 | SP18 | 0.340 | 22.3 | POSITIVE | ConfP-17 | 99.7 | YES | TP233 | 3.7 |
| 536901 | Month 18 | SP18 | 0.390 | 26.1 | POSITIVE | ConfP-17 | 100.0 | YES | TP233 | 3.7 |
| 536901 | Month 24 | SP18 | 0.325 | 21.2 | POSITIVE | ConfP-17 | 99.5 | YES | TP233 | 3.7 |
| 536902 | Baseline | SP18 | 0.049 | 0.2 | Negative | | | | | |
| 536902 | Month 1 | SP18 | 0.939 | 67.8 | POSITIVE | ConfP-17 | 99.6 | YES | TP234 | 3.7 |
| 536902 | Month 3 | SP18 | 1.248 | 91.3 | POSITIVE | ConfP-2 | 100.0 | YES | TP9 | 3.7 |
| 536902 | Month 6 | SP18 | 0.633 | 44.6 | POSITIVE | ConfP-17 | 99.4 | YES | TP234 | 3.7 |
| 536902 | Month 9 | SP18 | 0.987 | 71.5 | POSITIVE | ConfP-17 | 99.8 | YES | TP234 | 3.7 |
| 536902 | Month 12 | SP18 | 0.341 | 22.4 | POSITIVE | ConfP-17 | 99.0 | YES | TP234 | 3.7 |
| 536902 | Month 18 | SP18 | 0.292 | 18.7 | POSITIVE | ConfP-17 | 98.2 | YES | TP234 | 3.7 |
| 536902 | Month 24 | SP18 | 0.194 | 11.2 | POSITIVE | ConfP-18R | 98.4 | YES | TP234 | 2.7 |
| 536905 | Baseline | SP103 | 0.048 | 0.5 | Negative | | | | | |
| 536905 | Month 1 | SP103 | 0.136 | 7.5 | POSITIVE | ConfP-77 | 95.6 | YES | TP96 | 2.7 |
| 536905 | Month 3 | SP103 | 0.185 | 11.4 | POSITIVE | ConfP-78 | 97.2 | YES | TP96 | 2.7 |
| 536905 | Month 6 | SP103 | 0.077 | 2.8 | POSITIVE | ConfP-78 | 94.4 | YES | TP96 | 2.7 |
| 536905 | Month 9 | SP103 | 0.083 | 3.3 | POSITIVE | ConfP-78 | 88.2 | YES | TP96 | 2.7 |
| 536905 | Month 12 | SP103 | 0.075 | 2.6 | POSITIVE | ConfP-78 | 95.8 | YES | TP96 | 2.7 |
| 536905 | Month 18 | SP103 | 0.066 | 1.9 | POSITIVE | ConfP-78 | 90.9 | YES | TP96 | 2.7 |
| 536905 | Month 24 | SP103 | 0.060 | 1.4 | Negative | | | | | |
| 536906 | Baseline | SP97 | 0.058 | 1.2 | Negative | | | | | |
| 536906 | Month 1 | SP97 | 0.260 | 14.8 | POSITIVE | ConfP-73 | 99.5 | YES | TP86 | 2.7 |
| 536906 | Month 3 | SP97 | 0.431 | 26.4 | POSITIVE | ConfP-73 | 99.2 | YES | TP86 | 2.7 |
| 536906 | Month 6 | SP97 | 0.814 | 52.4 | POSITIVE | ConfP-73 | 98.6 | YES | TP86 | 2.7 |
| 536906 | Month 9 | SP97 | 0.474 | 29.3 | POSITIVE | ConfP-73 | 98.6 | YES | TP86 | 2.7 |
| 536906 | Month 12 | SP97 | 0.243 | 13.7 | POSITIVE | ConfP-73 | 98.3 | YES | TP86 | 2.7 |
| 536906 | Month 18 | SP97 | 0.161 | 8.1 | POSITIVE | ConfP-73 | 98.3 | YES | TP87 | 2.7 |
| 536906 | Month 24 | SP97 | 0.175 | 9.1 | POSITIVE | ConfP-73 | 98.5 | YES | TP87 | 2.7 |
| 536909 | Baseline | SP97 | 0.045 | 0.3 | Negative | | | | | |
| 536909 | Month 1 | SP97 | 0.107 | 4.5 | POSITIVE | ConfP-73 | 93.4 | YES | TP87 | 2.7 |
| 536909 | Month 3 | SP97 | 0.319 | 18.8 | POSITIVE | ConfP-73 | 97.7 | YES | TP87 | 3.7 |
| 536909 | Month 6 | SP97 | 0.163 | 8.3 | POSITIVE | ConfP-74 | 95.7 | YES | TP87 | 3.7 |
| 536909 | Month 9 | SP98 | 0.191 | 8.6 | POSITIVE | ConfP-74 | 95.3 | YES | TP89 | 3.7 |
| 536909 | Month 12 | SP98 | 0.185 | 8.3 | POSITIVE | ConfP-74 | 97.4 | YES | TP89 | 2.7 |
| 536909 | Month 18 | SP98 | 0.120 | 4.4 | POSITIVE | ConfP-74 | 94.9 | YES | TP89 | 2.7 |
| 536909 | Month 24 | SP98 | 0.101 | 3.3 | POSITIVE | ConfP-74 | 94.2 | YES | TP89 | 2.7 |
| 536912 | Baseline | SP98 | 0.046 | 0.1 | Negative | | | | | |
| 536912 | Month 1 | SP98 | 0.079 | 2.0 | POSITIVE | ConfP-74 | 100.0 | YES | TP87 | 2.7 |
| 536912 | Month 3 | SP98 | 0.721 | 40.0 | POSITIVE | ConfP-74 | 99.9 | YES | TP88 | 3.7 |
| 536912 | Month 6 | SP98 | 0.334 | 17.1 | POSITIVE | ConfP-74 | 100.0 | YES | TP88 | 2.7 |
| 536912 | Month 9 | SP98 | 0.327 | 16.7 | POSITIVE | ConfP-74 | 100.0 | YES | TP88 | 2.7 |
| 536912 | Month 12 | SP98 | 0.217 | 10.2 | POSITIVE | ConfP-74 | 100.0 | YES | TP88 | 2.7 |
| 536912 | Month 18 | SP98 | 0.248 | 12.0 | POSITIVE | ConfP-74 | 100.0 | YES | TP88 | 2.7 |
| 536912 | Month 24 | SP98 | 0.718 | 39.8 | POSITIVE | ConfP-74 | 100.0 | YES | TP88 | 3.7 |
| 536913 | Baseline | SP92 | 0.048 | 0.2 | Negative | | | | | |
| 536913 | Month 1 | SP92 | 0.988 | 51.7 | POSITIVE | ConfP-69 | 99.6 | YES | TP78 | 3.7 |
| 536913 | Month 3 | SP92 | 1.847 | 98.8 | POSITIVE | ConfP-69 | 98.6 | YES | TP78 | 4.7 |
| 536913 | Month 6 | SP92 | 1.645 | 87.7 | POSITIVE | ConfP-69 | 98.9 | YES | TP78 | 4.7 |
| 536913 | Month 9 | SP92 | 1.668 | 89.0 | POSITIVE | ConfP-69 | 99.5 | YES | TP78 | 3.7 |
| 536913 | Month 12 | SP92 | 1.062 | 55.8 | POSITIVE | ConfP-69 | 99.4 | YES | TP78 | 3.7 |
| 536913 | Month 18 | SP92 | 0.864 | 44.9 | POSITIVE | ConfP-69 | 99.5 | YES | TP78 | 3.7 |
| 536913 | Month 24 | SP92 | 1.012 | 53.0 | POSITIVE | ConfP-70 | 99.7 | YES | TP79 | 3.7 |
| 536914 | Baseline | SP91 | 0.045 | 0.0 | Negative | | | | | |
| 536914 | Month 1 | SP91 | 0.905 | 45.3 | POSITIVE | ConfP-69 | 99.6 | YES | TP76 | 3.7 |
| 536914 | Month 3 | SP91 | 2.243 | 115.8 | POSITIVE | ConfP-69 | 99.1 | YES | TP77 | 4.7 |
| 536914 | Month 6 | SP91 | 0.979 | 49.2 | POSITIVE | ConfP-69 | 99.4 | YES | TP77 | 3.7 |
| 536914 | Month 9 | SP91 | 0.616 | 30.1 | POSITIVE | ConfP-69 | 98.9 | YES | TP77 | 3.7 |
| 536914 | Month 12 | SP91 | 0.512 | 24.6 | POSITIVE | ConfP-69 | 96.5 | YES | TP77 | 3.7 |
| 536914 | Month 18 | SP91 | 0.272 | 12.0 | POSITIVE | ConfP-69 | 98.8 | YES | TP77 | 3.7 |
| 536914 | Month 24 | SP91 | 0.114 | 3.6 | POSITIVE | ConfP-69 | 96.1 | YES | TP77 | 2.7 |
| 536916 | Baseline | SP90 | 0.047 | 0.1 | Negative | | | | | |
| 536916 | Month 1 | SP90 | 0.074 | 1.5 | POSITIVE | ConfP-68 | 94.1 | YES | TP75 | 2.7 |
| 536916 | Month 3 | SP90 | 0.128 | 4.4 | POSITIVE | ConfP-68 | 97.8 | YES | TP75 | 2.7 |
| 536916 | Month 6 | SP90 | 0.097 | 2.8 | POSITIVE | ConfP-68 | 97.3 | YES | TP75 | 2.7 |
| 536916 | Month 9 | SP90 | 0.142 | 5.1 | POSITIVE | ConfP-68 | 85.1 | YES | TP75 | 2.7 |
| 536916 | Month 12 | SP90 | 0.067 | 1.2 | Negative | | | | | |
| 536916 | Month 18 | SP90 | 0.089 | 2.3 | POSITIVE | ConfP-68 | 91.8 | YES | TP75 | 2.7 |
| 536916 | Month 24 | SP90 | 0.455 | 21.7 | POSITIVE | ConfP-68 | 98.2 | YES | TP76 | 3.7 |
| 537001 | Baseline | SP89 | 0.067 | 1.4 | Negative | | | | | |
| 537001 | Month 1 | SP89 | 1.337 | 71.1 | POSITIVE | ConfP-68 | 98.5 | YES | TP74 | 3.7 |
| 537001 | Month 3 | SP89 | 2.025 | 108.9 | POSITIVE | ConfP-68 | 99.2 | YES | TP74 | 4.7 |
| 537001 | Month 6 | SP89 | 1.667 | 89.2 | POSITIVE | ConfP-68 | 99.5 | YES | TP75 | 4.7 |
| 537001 | Month 9 | SP90 | 1.401 | 71.8 | POSITIVE | ConfP-68 | 99.1 | YES | TP76 | 3.7 |
| 537001 | Month 12 | SP90 | 1.069 | 54.2 | POSITIVE | ConfP-68 | 99.2 | YES | TP76 | 3.7 |
| 537001 | Month 18 | SP90 | 1.504 | 77.2 | POSITIVE | ConfP-68 | 99.4 | YES | TP76 | 3.7 |
| 537001 | Month 24 | SP90 | 1.672 | 86.1 | POSITIVE | ConfP-68 | 99.2 | YES | TP76 | 3.7 |
| 537002 | Baseline | SP89 | 0.044 | 0.1 | Negative | | | | | |
| 537002 | Month 1 | SP89 | 0.525 | 26.5 | POSITIVE | ConfP-67 | 99.3 | YES | TP73 | 2.7 |
| 537002 | Month 3 | SP89 | 0.875 | 45.7 | POSITIVE | ConfP-67 | 99.5 | YES | TP73 | 3.7 |
| 537002 | Month 6 | SP89 | 0.624 | 32.0 | POSITIVE | ConfP-67 | 98.9 | YES | TP73 | 3.7 |
| 537002 | Month 9 | SP89 | 0.510 | 25.7 | POSITIVE | ConfP-67 | 99.4 | YES | TP74 | 3.7 |
| 537002 | Month 12 | SP89 | 0.264 | 12.2 | POSITIVE | ConfP-67 | 99.1 | YES | TP74 | 2.7 |
| 537002 | Month 18 | SP89 | 0.315 | 15.0 | POSITIVE | ConfP-67 | 99.1 | YES | TP74 | 2.7 |
| 537002 | Month 24 | SP89 | 0.182 | 7.7 | POSITIVE | ConfP-67 | 98.6 | YES | TP74 | 2.7 |
| 537003 | Baseline | SP93 | 0.044 | 0.1 | Negative | | | | | |
| 537003 | Month 1 | SP93 | 0.332 | 12.7 | POSITIVE | ConfP-70 | 99.1 | YES | TP79 | 3.7 |
| 537003 | Month 3 | SP93 | 1.936 | 83.1 | POSITIVE | ConfP-70 | 99.0 | YES | TP79 | 4.7 |
| 537003 | Month 6 | SP93 | 1.045 | 44.0 | POSITIVE | ConfP-70 | 99.4 | YES | TP79 | 3.7 |
| 537003 | Month 9 | SP93 | 0.184 | 6.2 | POSITIVE | ConfP-70 | 98.3 | YES | TP79 | 2.7 |
| 537003 | Month 12 | SP93 | 0.809 | 33.7 | POSITIVE | ConfP-70 | 98.9 | YES | TP79 | 3.7 |
| 537003 | Month 18 | SP93 | 0.285 | 10.7 | POSITIVE | ConfP-70 | 98.6 | YES | TP80 | 3.7 |
| 537003 | Month 24 | SP93 | 0.097 | 2.4 | POSITIVE | ConfP-70 | 92.2 | YES | TP80 | 2.7 |
| 537007 | Baseline | SP93 | 0.044 | 0.1 | Negative | | | | | |
| 537007 | Month 1 | SP93 | 0.184 | 6.2 | POSITIVE | ConfP-70 | 97.9 | YES | TP80 | 3.7 |
| 537007 | Month 3 | SP93 | 1.188 | 50.3 | POSITIVE | ConfP-70 | 98.7 | YES | TP80 | 3.7 |
| 537007 | Month 6 | SP93 | 0.770 | 31.9 | POSITIVE | ConfP-70 | 98.2 | YES | TP80 | 3.7 |
| 537007 | Month 9 | SP94 | 1.136 | 47.8 | POSITIVE | ConfP-71 | 98.8 | YES | TP82 | 3.7 |
| 537007 | Month 12 | SP94 | 0.849 | 35.3 | POSITIVE | ConfP-71 | 97.8 | YES | TP82 | 3.7 |
| 537007 | Month 18 | SP94 | 0.484 | 19.3 | POSITIVE | ConfP-71 | 96.9 | YES | TP82 | 3.7 |
| 537007 | Month 24 | SP94 | 0.263 | 9.7 | POSITIVE | ConfP-71 | 97.1 | YES | TP82 | 3.7 |
| 537008 | Baseline | SP94 | 0.048 | 0.3 | Negative | | | | | |
| 537008 | Month 1 | SP94 | 0.596 | 24.2 | POSITIVE | ConfP-70 | 94.8 | YES | TP80 | 3.7 |
| 537008 | Month 3 | SP94 | 0.873 | 36.3 | POSITIVE | ConfP-70 | 92.2 | YES | TP81 | 3.7 |
| 537008 | Month 6 | SP94 | 0.682 | 28.0 | POSITIVE | ConfP-71 | 96.2 | YES | TP81 | 3.7 |
| 537008 | Month 9 | SP94 | 0.555 | 22.4 | POSITIVE | ConfP-71 | 96.4 | YES | TP81 | 3.7 |
| 537008 | Month 12 | SP94 | 0.344 | 13.2 | POSITIVE | ConfP-71 | 94.6 | YES | TP81 | 3.7 |
| 537008 | Month 18 | SP94 | 0.263 | 9.7 | POSITIVE | ConfP-71 | 92.9 | YES | TP81 | 3.7 |
| 537008 | Month 24 | SP94 | 0.418 | 16.4 | POSITIVE | ConfP-71 | 93.0 | YES | TP81 | 3.7 |
| 537010 | Baseline | SP95 | 0.052 | 0.4 | Negative | | | | | |
| 537010 | Month 1 | SP95 | 1.681 | 68.3 | POSITIVE | ConfP-71 | 99.5 | YES | TP82 | 3.7 |
| 537010 | Month 3 | SP95 | 1.010 | 40.3 | POSITIVE | ConfP-71 | 99.4 | YES | TP82 | 3.7 |
| 537010 | Month 6 | SP95 | 0.323 | 11.7 | POSITIVE | ConfP-71 | 98.0 | YES | TP83 | 3.7 |
| 537010 | Month 9 | SP95 | 0.534 | 20.5 | POSITIVE | ConfP-71 | 99.1 | YES | TP83 | 3.7 |
| 537010 | Month 12 | SP95 | 0.690 | 27.0 | POSITIVE | ConfP-72 | 99.2 | YES | TP83 | 3.7 |
| 537010 | Month 18 | SP95 | 0.407 | 15.2 | POSITIVE | ConfP-72 | 99.1 | YES | TP83 | 3.7 |
| 537010 | Month 24 | SP95 | 0.370 | 13.6 | POSITIVE | ConfP-72 | 99.3 | YES | TP83 | 3.7 |
| 537012 | Baseline | SP95 | 0.050 | 0.3 | Negative | | | | | |
| 537012 | Month 1 | SP95 | 0.099 | 2.3 | POSITIVE | ConfP-72 | 96.9 | YES | TP83 | 2.7 |
| 537012 | Month 3 | SP95 | 1.460 | 59.1 | POSITIVE | ConfP-72 | 99.0 | YES | TP84 | 3.7 |
| 537012 | Month 6 | SP95 | 0.528 | 20.2 | POSITIVE | ConfP-72 | 98.3 | YES | TP84 | 3.7 |
| 537012 | Month 9 | SP96 | 0.205 | 7.5 | POSITIVE | ConfP-73 | 98.6 | YES | TP85 | 2.7 |
| 537012 | Month 12 | SP96 | 0.210 | 7.7 | POSITIVE | ConfP-73 | 99.0 | YES | TP85 | 2.7 |
| 537012 | Month 18 | SP96 | 0.149 | 4.9 | POSITIVE | ConfP-73 | 97.2 | YES | TP85 | 2.7 |
| 537012 | Month 24 | SP96 | 0.137 | 4.3 | POSITIVE | ConfP-73 | 98.4 | YES | TP86 | 2.7 |
| 537013 | Baseline | SP96 | 0.047 | 0.1 | Negative | | | | | |
| 537013 | Month 1 | SP96 | 0.709 | 30.8 | POSITIVE | ConfP-72 | 99.4 | YES | TP84 | 3.7 |
| 537013 | Month 3 | SP96 | 0.596 | 25.6 | POSITIVE | ConfP-72 | 98.9 | YES | TP84 | 3.7 |
| 537013 | Month 6 | SP96 | 0.537 | 22.8 | POSITIVE | ConfP-72 | 98.9 | YES | TP84 | 3.7 |
| 537013 | Month 9 | SP96 | 0.363 | 14.8 | POSITIVE | ConfP-72 | 98.7 | YES | TP84 | 3.7 |
| 537013 | Month 12 | SP96 | 0.185 | 6.5 | POSITIVE | ConfP-72 | 98.7 | YES | TP85 | 2.7 |
| 537013 | Month 18 | SP96 | 0.113 | 3.2 | POSITIVE | ConfP-72 | 95.5 | YES | TP85 | 2.7 |
| 537013 | Month 24 | SP96 | 0.125 | 3.8 | POSITIVE | ConfP-72 | 94.0 | YES | TP85 | 2.7 |
| 537015 | Baseline | SP99 | 0.044 | 0.0 | Negative | | | | | |
| 537015 | Month 1 | SP99 | 1.349 | 73.5 | POSITIVE | ConfP-74 | 88.1 | YES | TP89 | 3.7 |
| 537015 | Month 3 | SP99 | 1.439 | 78.6 | POSITIVE | ConfP-75 | 99.3 | YES | TP89 | 3.7 |
| 537015 | Month 6 | SP99 | 0.485 | 24.7 | POSITIVE | ConfP-75 | 99.4 | YES | TP90 | 3.7 |
| 537015 | Month 9 | SP99 | 0.694 | 36.5 | POSITIVE | ConfP-75 | 99.5 | YES | TP90 | 3.7 |
| 537015 | Month 12 | SP99 | 0.354 | 17.3 | POSITIVE | ConfP-75 | 99.2 | YES | TP90 | 2.7 |
| 537015 | Month 18 | SP99 | 0.373 | 18.4 | POSITIVE | ConfP-75 | 99.4 | YES | TP90 | 2.7 |
| 537015 | Month 24 | SP99 | 0.077 | 1.6 | POSITIVE | ConfP-75 | 94.9 | YES | TP90 | 2.7 |
| 537101 | Baseline | SP188 | 0.095 | 0.0 | Negative | | | | | |
| 537101 | Month 1 | SP188 | 0.171 | 3.3 | POSITIVE | ConfP-140 | 97.4 | YES | TP299 | 2.7 |
| 537101 | Month 3 | No sample received | | | | | | | | |
| 537101 | Month 6 | SP199 | 1.170 | 60.8 | POSITIVE | ConfP-140 | 99.2 | YES | TP299 | 3.7 |
| 537101 | Month 9 | SP199 | 0.667 | 32.7 | POSITIVE | ConfP-140 | 98.7 | YES | TP299 | 2.7 |
| 537101 | Month 12 | SP188 | 0.633 | 24.1 | POSITIVE | ConfP-140 | 98.4 | YES | TP299 | 2.7 |
| 537101 | Month 18 | SP188 | 0.548 | 20.3 | POSITIVE | ConfP-140 | 98.6 | YES | TP299 | 2.7 |
| 537101 | Month 24 | SP196 | 0.910 | 35.1 | POSITIVE | ConfP-140 | 98.1 | YES | TP299 | 2.7 |
| 537102 | Baseline | SP189 | 0.094 | 1.3 | Negative | | | | | |
| 537102 | Month 1 | SP189 | 0.791 | 32.7 | POSITIVE | ConfP-134 | 100.0 | YES | TP291 | 3.7 |
| 537102 | Month 3 | No sample received | | | | | | | | |
| 537102 | Month 6 | SP189 | 0.912 | 38.2 | POSITIVE | ConfP-134 | 99.6 | YES | TP291 | 2.7 |
| 537102 | Month 9 | SP189 | 0.293 | 10.2 | POSITIVE | ConfP-134 | 100.0 | YES | TP317 | 2.7 |
| 537102 | Month 12 | SP189 | 0.435 | 16.6 | POSITIVE | ConfP-134 | 100.0 | YES | TP314 | 2.7 |
| 537102 | Month 18 | SP189 | 0.760 | 31.3 | POSITIVE | ConfP-134 | 100.0 | YES | TP314 | 2.7 |
| 537102 | Month 24 | SP196 | 1.055 | 41.1 | POSITIVE | ConfP-134 | 99.7 | YES | TP314 | 2.7 |
| 537106 | Baseline | SP189 | 0.077 | 0.5 | Negative | | | | | |
| 537106 | Month 1 | SP189 | 1.621 | 70.1 | POSITIVE | ConfP-134 | 99.9 | YES | TP314 | 3.7 |
| 537106 | Month 3 | No sample received | | | | | | | | |
| 537106 | Month 6 | SP189 | 2.539 | 111.5 | POSITIVE | ConfP-134 | 99.4 | YES | TP292 | 3.7 |
| 537106 | Month 9 | SP189 | 2.453 | 107.7 | POSITIVE | ConfP-134 | 99.6 | YES | TP292 | 4.7 |
| 537106 | Month 12 | SP189 | 2.449 | 107.5 | POSITIVE | ConfP-134 | 99.6 | YES | TP292 | 3.7 |
| 537106 | Month 18 | SP189 | 2.239 | 98.0 | POSITIVE | ConfP-134 | 99.6 | YES | TP292 | 3.7 |
| 537106 | Month 24 | SP189 | 2.020 | 88.1 | POSITIVE | ConfP-134 | 99.6 | YES | TP292 | 3.7 |
| 537109 | Baseline | SP19 | 0.043 | 0.0 | Negative | | | | | |
| 537109 | Month 1 | SP19 | 0.199 | 12.0 | POSITIVE | ConfP-18R | 99.5 | YES | TP235 | 2.7 |
| 537109 | Month 3 | SP19 | 0.548 | 41.2 | POSITIVE | ConfP-2 | 100.0 | YES | TP9 | 3.7 |
| 537109 | Month 6 | SP19 | 0.484 | 35.9 | POSITIVE | ConfP-18R | 99.6 | YES | TP235 | 3.7 |
| 537109 | Month 9 | SP19 | 0.238 | 15.3 | POSITIVE | ConfP-18R | 99.6 | YES | TP235 | 2.7 |
| 537109 | Month 12 | SP19 | 0.168 | 9.4 | POSITIVE | ConfP-18R | 99.3 | YES | TP235 | 2.7 |
| 537109 | Month 18 | SP19 | 0.146 | 7.6 | POSITIVE | ConfP-18R | 99.2 | YES | TP235 | 2.7 |
| 537109 | Month 24 | SP19 | 0.224 | 14.1 | POSITIVE | ConfP-18R | 99.5 | YES | TP235 | 2.7 |
| 537111 | Baseline | SP19 | 0.044 | 0.0 | Negative | | | | | |
| 537111 | Month 1 | SP19 | 0.147 | 7.7 | POSITIVE | ConfP-18R | 99.0 | YES | TP236 | 2.7 |
| 537111 | Month 3 | SP19 | 0.393 | 28.3 | POSITIVE | ConfP-3 | 100.0 | YES | TP9 | 2.7 |
| 537111 | Month 6 | SP19 | 0.104 | 4.1 | POSITIVE | ConfP-18R | 98.0 | YES | TP236 | 2.7 |
| 537111 | Month 9 | SP20 | 0.085 | 3.3 | POSITIVE | ConfP-18R | 98.0 | YES | TP236 | 2.7 |
| 537111 | Month 12 | SP20 | 0.060 | 1.2 | Negative | | | | | |
| 537111 | Month 18 | SP20 | 0.066 | 1.7 | POSITIVE | ConfP-18R | 97.8 | YES | TP236 | 2.7 |
| 537111 | Month 24 | SP20 | 0.070 | 2.1 | POSITIVE | ConfP-18R | 97.5 | YES | TP236 | 2.7 |
| 537113 | Baseline | Whole blod sample received instead of serum, not analysed. | | | | | | | | |
| 537113 | Month 1 | No sample received | | | | | | | | |
| 537113 | Month 3 | SP185 | 0.780 | 34.7 | POSITIVE | ConfP-137 | 99.1 | YES | TP296 | 3.7 |
| 537113 | Month 6 | SP185 | 0.240 | 8.2 | POSITIVE | ConfP-137 | 97.3 | YES | TP296 | 2.7 |
| 537113 | Month 9 | SP185 | 0.166 | 4.6 | POSITIVE | ConfP-137 | 96.9 | YES | TP296 | 2.7 |
| 537113 | Month 12 | SP185 | 0.253 | 8.9 | POSITIVE | ConfP-137 | 98.7 | YES | TP296 | 2.7 |
| 537113 | Month 18 | SP185 | 0.176 | 5.1 | POSITIVE | ConfP-137 | 96.0 | YES | TP296 | 2.7 |
| 537113 | Month 24 | SP185 | 0.342 | 13.2 | POSITIVE | ConfP-137 | 99.3 | YES | TP296 | 2.7 |
| 537114 | Baseline | SP20 | 0.062 | 1.4 | Negative | | | | | |
| 537114 | Month 1 | SP20 | 0.640 | 50.9 | POSITIVE | ConfP-18R | 98.3 | YES | TP236 | 3.7 |
| 537114 | Month 3 | SP20 | 1.359 | 112.6 | POSITIVE | ConfP-3 | 99.4 | YES | TP9 | 3.7 |
| 537114 | Month 6 | SP20 | 1.129 | 92.9 | POSITIVE | ConfP-19R | 98.6 | YES | TP237 | 3.7 |
| 537114 | Month 9 | SP20 | 0.299 | 21.7 | POSITIVE | ConfP-19R | 98.1 | YES | TP237 | 2.7 |
| 537114 | Month 12 | SP20 | 1.024 | 83.9 | POSITIVE | ConfP-19R | 98.6 | YES | TP237 | 3.7 |
| 537114 | Month 18 | SP20 | 0.830 | 67.2 | POSITIVE | ConfP-19R | 98.2 | YES | TP237 | 3.7 |
| 537114 | Month 24 | SP20 | 0.757 | 61.0 | POSITIVE | ConfP-19R | 97.0 | YES | TP237 | 3.7 |
| 537116 | Baseline | SP21 | 0.045 | 0.1 | Negative | | | | | |
| 537116 | Month 1 | SP21 | 0.059 | 1.0 | Negative | | | | | |
| 537116 | Month 3 | SP21 | 0.748 | 44.9 | POSITIVE | ConfP-3 | 99.3 | YES | TP9 | 3.7 |
| 537116 | Month 6 | SP21 | 0.571 | 33.6 | POSITIVE | ConfP-19R | 99.5 | YES | TP237 | 3.7 |
| 537116 | Month 9 | SP21 | 0.378 | 21.3 | POSITIVE | ConfP-19R | 99.4 | YES | TP238 | 3.7 |
| 537116 | Month 12 | SP21 | 0.321 | 17.7 | POSITIVE | ConfP-19R | 99.4 | YES | TP238 | 2.7 |
| 537116 | Month 18 | SP21 | 0.466 | 26.9 | POSITIVE | ConfP-19R | 99.4 | YES | TP238 | 3.7 |
| 537116 | Month 24 | SP21 | 0.339 | 18.9 | POSITIVE | ConfP-19R | 99.2 | YES | TP238 | 2.7 |
| 537117 | Baseline | SP21 | 0.045 | 0.1 | Negative | | | | | |
| 537117 | Month 1 | SP21 | 0.407 | 23.2 | POSITIVE | ConfP-19R | 99.1 | YES | TP238 | 3.7 |
| 537117 | Month 3 | SP21 | 0.761 | 45.7 | POSITIVE | ConfP-3 | 100.0 | YES | TP9 | 3.7 |
| 537117 | Month 6 | SP21 | 0.493 | 28.7 | POSITIVE | ConfP-19R | 99.0 | YES | TP238 | 3.7 |
| 537117 | Month 9 | SP22 | 0.219 | 12.7 | POSITIVE | ConfP-19R | 96.7 | YES | TP239 | 2.7 |
| 537117 | Month 12 | SP22 | 0.131 | 6.3 | POSITIVE | ConfP-20R | 97.4 | YES | TP239 | 2.7 |
| 537117 | Month 18 | SP22 | 0.108 | 4.6 | POSITIVE | ConfP-20R | 96.1 | YES | TP239 | 2.7 |
| 537117 | Month 24 | SP22 | 0.076 | 2.3 | POSITIVE | ConfP-20R | 93.7 | YES | TP239 | 2.7 |
| 537118 | Baseline | SP22 | 0.046 | 0.1 | Negative | | | | | |
| 537118 | Month 1 | SP22 | 0.135 | 6.6 | POSITIVE | ConfP-20R | 97.9 | YES | TP239 | 2.7 |
| 537118 | Month 3 | SP22 | 1.355 | 95.6 | POSITIVE | ConfP-3 | 100.0 | YES | TP10 | 3.7 |
| 537118 | Month 6 | SP22 | 0.252 | 15.1 | POSITIVE | ConfP-20R | 99.5 | YES | TP239 | 2.7 |
| 537118 | Month 9 | SP22 | 0.409 | 26.5 | POSITIVE | ConfP-20R | 99.2 | YES | TP240 | 3.7 |
| 537118 | Month 12 | SP22 | 0.590 | 39.8 | POSITIVE | ConfP-20R | 99.6 | YES | TP240 | 3.7 |
| 537118 | Month 18 | SP22 | 0.146 | 7.4 | POSITIVE | ConfP-20R | 98.5 | YES | TP240 | 2.7 |
| 537118 | Month 24 | SP22 | 0.196 | 11.0 | POSITIVE | ConfP-20R | 99.2 | YES | TP240 | 2.7 |
| 537121 | Baseline | SP99 | 0.047 | 0.0 | Negative | | | | | |
| 537121 | Month 1 | SP99 | 0.074 | 1.5 | POSITIVE | ConfP-75 | 100.0 | YES | TP90 | 2.7 |
| 537121 | Month 3 | SP99 | 1.878 | 103.4 | POSITIVE | ConfP-75 | 97.7 | YES | TP91R | 4.7 |
| 537121 | Month 6 | SP99 | 0.922 | 49.4 | POSITIVE | ConfP-75 | 99.6 | YES | TP91R | 3.7 |
| 537121 | Month 9 | SP100 | 0.373 | 19.3 | POSITIVE | ConfP-76 | 98.9 | YES | TP92 | 3.7 |
| 537121 | Month 12 | SP100 | 0.317 | 16.1 | POSITIVE | ConfP-76 | 98.9 | YES | TP92 | 3.7 |
| 537121 | Month 18 | SP100 | 0.055 | 0.8 | Negative | | | | | |
| 537121 | Month 24 | SP100 | 0.367 | 19.0 | POSITIVE | ConfP-76 | 99.0 | YES | TP92 | 3.7 |
| 537123 | Baseline | SP100 | 0.049 | 0.4 | Negative | | | | | |
| 537123 | Month 1 | SP100 | 0.222 | 10.5 | POSITIVE | ConfP-75 | 98.7 | YES | TP91R | 2.7 |
| 537123 | Month 3 | SP100 | 0.203 | 9.4 | POSITIVE | ConfP-75 | 98.3 | YES | TP91R | 2.7 |
| 537123 | Month 6 | SP100 | 0.111 | 4.0 | POSITIVE | ConfP-75 | 96.7 | YES | TP91R | 2.7 |
| 537123 | Month 9 | SP100 | 0.079 | 2.2 | POSITIVE | ConfP-75 | 89.7 | YES | TP91R | 2.7 |
| 537123 | Month 12 | SP100 | 0.073 | 1.8 | POSITIVE | ConfP-76 | 88.0 | YES | TP92 | 2.7 |
| 537123 | Month 18 | SP100 | 0.064 | 1.3 | Negative | | | | | |
| 537123 | Month 24 | SP100 | 0.052 | 0.6 | Negative | | | | | |
| 537125 | Baseline | SP105R | 0.044 | 0.1 | Negative | | | | | |
| 537125 | Month 1 | SP105R | 1.517 | 95.7 | POSITIVE | ConfP-79 | 99.4 | YES | TP99 | 3.7 |
| 537125 | Month 3 | SP105R | 1.239 | 77.7 | POSITIVE | ConfP-79 | 99.5 | YES | TP99 | 3.7 |
| 537125 | Month 6 | SP105R | 0.411 | 23.9 | POSITIVE | ConfP-79 | 99.3 | YES | TP99 | 2.7 |
| 537125 | Month 9 | SP105R | 0.264 | 14.4 | POSITIVE | ConfP-79 | 97.6 | YES | TP99 | 2.7 |
| 537125 | Month 12 | SP105R | 0.332 | 18.8 | POSITIVE | ConfP-79 | 99.0 | YES | TP99 | 2.7 |
| 537125 | Month 18 | SP105R | 0.307 | 17.2 | POSITIVE | ConfP-79 | 99.6 | YES | TP100 | 2.7 |
| 537125 | Month 24 | SP105R | 0.349 | 19.9 | POSITIVE | ConfP-79 | 99.4 | YES | TP100 | 2.7 |
| 537130 | Baseline | SP195 | 0.141 | 3.2 | POSITIVE | ConfP-147 | 65.5 | YES | TP315 | <2.7 |
| 537130 | Month 1 | SP195 | 1.135 | 43.0 | POSITIVE | ConfP-144 | 99.2 | YES | TP300 | 2.7 |
| 537130 | Month 3 | SP195 | 2.284 | 89.0 | POSITIVE | ConfP-144 | 99.7 | YES | TP300 | 3.7 |
| 537130 | Month 6 | SP195 | 2.029 | 78.7 | POSITIVE | ConfP-144 | 99.6 | YES | TP300 | 3.7 |
| 537130 | Month 9 | SP195 | 1.133 | 42.9 | POSITIVE | ConfP-144 | 98.6 | YES | TP300 | 2.7 |
| 537130 | Month 12 | No sample received | | | | | | | | |
| 537130 | Month 18 | SP195 | 1.288 | 49.1 | POSITIVE | ConfP-144 | 99.3 | YES | TP300 | 2.7 |
| 537130 | Month 24 | SP195 | 0.944 | 35.3 | POSITIVE | ConfP-144 | 99.2 | YES | TP300 | 2.7 |
| 537131 | Baseline | SP195 | 0.071 | 0.4 | Negative | | | | | |
| 537131 | Month 1 | SP195 | 0.538 | 19.1 | POSITIVE | ConfP-144 | 98.9 | YES | TP301 | 2.7 |
| 537131 | Month 3 | SP195 | 0.877 | 32.6 | POSITIVE | ConfP-144 | 99.7 | YES | TP301 | 2.7 |
| 537131 | Month 6 | SP195 | 0.432 | 14.8 | POSITIVE | ConfP-144 | 98.6 | YES | TP301 | 2.7 |
| 537131 | Month 9 | SP195 | 0.194 | 5.3 | POSITIVE | ConfP-144 | 97.3 | YES | TP301 | 2.7 |
| 537131 | Month 12 | No sample received | | | | | | | | |
| 537131 | Month 18 | SP195 | 0.247 | 7.4 | POSITIVE | ConfP-144 | 93.7 | YES | TP301 | 2.7 |
| 537131 | Month 24 | SP196 | 0.290 | 9.5 | POSITIVE | ConfP-144 | 97.9 | YES | TP301 | 2.7 |
| 537132 | Baseline | SP105R | 0.045 | 0.2 | Negative | | | | | |
| 537132 | Month 1 | SP105R | 0.057 | 1.0 | Negative | | | | | |
| 537132 | Month 3 | SP105R | 0.457 | 26.9 | POSITIVE | ConfP-79 | 98.3 | YES | TP100 | 2.7 |
| 537132 | Month 6 | SP105R | 0.379 | 21.9 | POSITIVE | ConfP-79 | 98.8 | YES | TP100 | 2.7 |
| 537132 | Month 9 | SP106R | 0.387 | 20.2 | POSITIVE | ConfP-80 | 98.4 | YES | TP101 | 2.7 |
| 537132 | Month 12 | SP106R | 0.483 | 25.9 | POSITIVE | ConfP-80 | 98.0 | YES | TP102 | 3.7 |
| 537132 | Month 18 | SP106R | 0.143 | 6.0 | POSITIVE | ConfP-80 | 96.6 | YES | TP102 | 2.7 |
| 537132 | Month 24 | SP106R | 0.223 | 10.6 | POSITIVE | ConfP-80 | 96.8 | YES | TP102 | 2.7 |
| 537201 | Baseline | SP23 | 0.044 | 0.1 | Negative | | | | | |
| 537201 | Month 1 | SP23 | 1.830 | 113.8 | POSITIVE | ConfP-20R | 99.4 | YES | TP240 | 3.7 |
| 537201 | Month 3 | SP23 | 1.973 | 122.9 | POSITIVE | ConfP-3 | 99.7 | YES | TP10 | 3.7 |
| 537201 | Month 6 | SP23 | 1.481 | 91.6 | POSITIVE | ConfP-20R | 99.2 | YES | TP240 | 3.7 |
| 537201 | Month 9 | SP23 | 1.199 | 73.6 | POSITIVE | ConfP-20R | 99.4 | YES | TP241 | 3.7 |
| 537201 | Month 12 | SP23 | 1.180 | 72.4 | POSITIVE | ConfP-20R | 99.6 | YES | TP241 | 3.7 |
| 537201 | Month 18 | SP23 | 1.507 | 93.2 | POSITIVE | ConfP-21 | 99.1 | YES | TP241 | 3.7 |
| 537201 | Month 24 | SP23 | 1.808 | 112.4 | POSITIVE | ConfP-21 | 99.5 | YES | TP241 | 3.7 |
| 537203 | Baseline | SP23 | 0.051 | 0.5 | Negative | | | | | |
| 537203 | Month 1 | SP23 | 0.148 | 6.7 | POSITIVE | ConfP-21 | 92.5 | YES | TP241 | 2.7 |
| 537203 | Month 3 | SP23 | 1.483 | 91.7 | POSITIVE | ConfP-3 | 99.4 | YES | TP10 | 3.7 |
| 537203 | Month 6 | SP23 | 1.339 | 82.5 | POSITIVE | ConfP-21 | 98.8 | YES | TP241 | 3.7 |
| 537203 | Month 9 | SP24 | 1.026 | 73.2 | POSITIVE | ConfP-21 | 99.2 | YES | TP242 | 3.7 |
| 537203 | Month 12 | SP24 | 0.921 | 65.3 | POSITIVE | ConfP-21 | 99.3 | YES | TP242 | 3.7 |
| 537203 | Month 18 | SP24 | 0.677 | 47.1 | POSITIVE | ConfP-21 | 98.5 | YES | TP242 | 3.7 |
| 537203 | Month 24 | SP24 | 0.604 | 41.6 | POSITIVE | ConfP-21 | 98.1 | YES | TP242 | 3.7 |
| 537205 | Baseline | SP106R | 0.047 | 0.4 | Negative | | | | | |
| 537205 | Month 1 | SP106R | 1.973 | 113.0 | POSITIVE | ConfP-80 | 97.0 | YES | TP100 | 3.7 |
| 537205 | Month 3 | SP106R | 2.510 | 144.5 | POSITIVE | ConfP-80 | 96.1 | YES | TP100 | 4.7 |
| 537205 | Month 6 | SP106R | 2.366 | 136.0 | POSITIVE | ConfP-80 | 98.5 | YES | TP101 | 4.7 |
| 537205 | Month 9 | SP106R | 2.200 | 126.3 | POSITIVE | ConfP-80 | 98.1 | YES | TP101 | 3.7 |
| 537205 | Month 12 | SP106R | 2.263 | 130.0 | POSITIVE | ConfP-80 | 98.6 | YES | TP101 | 4.7 |
| 537205 | Month 18 | SP106R | 1.786 | 102.1 | POSITIVE | ConfP-80 | 99.1 | YES | TP101 | 3.7 |
| 537205 | Month 24 | SP106R | 1.479 | 84.1 | POSITIVE | ConfP-80 | 99.0 | YES | TP101 | 3.7 |
| 537206 | Baseline | SP107R | 0.047 | 0.4 | Negative | | | | | |
| 537206 | Month 1 | SP107R | 0.070 | 2.4 | POSITIVE | ConfP-80 | 84.0 | YES | TP102 | <2.7 |
| 537206 | Month 3 | SP107R | 0.411 | 31.7 | POSITIVE | ConfP-80 | 98.8 | YES | TP102 | 2.7 |
| 537206 | Month 6 | SP107R | 0.554 | 43.9 | POSITIVE | ConfP-81 | 99.3 | YES | TP102 | 2.7 |
| 537206 | Month 9 | SP107R | 0.366 | 27.8 | POSITIVE | ConfP-81 | 99.5 | YES | TP103 | 2.7 |
| 537206 | Month 12 | SP107R | 0.408 | 31.4 | POSITIVE | ConfP-81 | 99.2 | YES | TP103 | 2.7 |
| 537206 | Month 18 | SP107R | 0.312 | 23.2 | POSITIVE | ConfP-81 | 98.6 | YES | TP103 | 2.7 |
| 537206 | Month 24 | SP107R | 0.189 | 12.6 | POSITIVE | ConfP-81 | 97.8 | YES | TP103 | 2.7 |
| 537207 | Baseline | SP107R | 0.048 | 0.5 | Negative | | | | | |
| 537207 | Month 1 | SP107R | 0.266 | 19.2 | POSITIVE | ConfP-81 | 98.5 | YES | TP103 | 2.7 |
| 537207 | Month 3 | SP107R | 0.448 | 34.8 | POSITIVE | ConfP-81 | 99.4 | YES | TP103 | 3.7 |
| 537207 | Month 6 | SP107R | 0.485 | 38.0 | POSITIVE | ConfP-81 | 99.4 | YES | TP104 | 3.7 |
| 537207 | Month 9 | SP108R | 0.490 | 32.5 | POSITIVE | ConfP-82R | 99.1 | YES | TP105 | 2.7 |
| 537207 | Month 12 | SP108R | 0.475 | 31.4 | POSITIVE | ConfP-82R | 99.0 | YES | TP105 | 3.7 |
| 537207 | Month 18 | SP108R | 0.348 | 22.2 | POSITIVE | ConfP-82R | 99.1 | YES | TP105 | 2.7 |
| 537207 | Month 24 | SP108R | 0.154 | 8.1 | POSITIVE | ConfP-82R | 97.5 | YES | TP105 | 2.7 |
| 537208 | Baseline | SP108R | 0.057 | 1.1 | Negative | | | | | |
| 537208 | Month 1 | SP108R | 0.818 | 56.3 | POSITIVE | ConfP-81 | 98.9 | YES | TP104 | 3.7 |
| 537208 | Month 3 | SP108R | 1.616 | 114.1 | POSITIVE | ConfP-81 | 99.5 | YES | TP104 | 3.7 |
| 537208 | Month 6 | SP108R | 0.256 | 15.5 | POSITIVE | ConfP-81 | 98.6 | YES | TP104 | 2.7 |
| 537208 | Month 9 | SP108R | 1.523 | 107.4 | POSITIVE | ConfP-81 | 99.4 | YES | TP104 | 3.7 |
| 537208 | Month 12 | SP108R | 0.520 | 34.7 | POSITIVE | ConfP-81 | 99.0 | YES | TP104 | 3.7 |
| 537208 | Month 18 | SP108R | 0.259 | 15.7 | POSITIVE | ConfP-82R | 97.8 | YES | TP105 | 2.7 |
| 537208 | Month 24 | SP108R | 0.566 | 38.0 | POSITIVE | ConfP-82R | 98.7 | YES | TP105 | 3.7 |
| 537210 | Baseline | SP109R | 0.046 | 0.2 | Negative | | | | | |
| 537210 | Month 1 | SP109R | 0.104 | 4.1 | POSITIVE | ConfP-82R | 98.0 | YES | TP106 | 2.7 |
| 537210 | Month 3 | SP109R | 1.557 | 100.6 | POSITIVE | ConfP-82R | 99.4 | YES | TP106 | 3.7 |
| 537210 | Month 6 | SP109R | 0.988 | 62.8 | POSITIVE | ConfP-82R | 99.3 | YES | TP106 | 3.7 |
| 537210 | Month 9 | SP109R | 0.727 | 45.4 | POSITIVE | ConfP-82R | 99.6 | YES | TP106 | 3.7 |
| 537210 | Month 12 | SP109R | 0.639 | 39.6 | POSITIVE | ConfP-82R | 99.4 | YES | TP106 | 3.7 |
| 537210 | Month 18 | SP109R | 0.336 | 19.5 | POSITIVE | ConfP-82R | 99.2 | YES | TP106 | 2.7 |
| 537210 | Month 24 | SP109R | 0.340 | 19.7 | POSITIVE | ConfP-82R | 98.8 | YES | TP107 | 3.7 |
| 537214 | Baseline | SP109R | 0.044 | 0.1 | Negative | | | | | |
| 537214 | Month 1 | SP109R | 0.044 | 0.1 | Negative | | | | | |
| 537214 | Month 3 | SP109R | 0.112 | 4.6 | POSITIVE | ConfP-83R | 97.5 | YES | TP107 | 2.7 |
| 537214 | Month 6 | SP109R | 0.152 | 7.2 | POSITIVE | ConfP-83R | 98.7 | YES | TP107 | 2.7 |
| 537214 | Month 9 | SP110 | 0.064 | 1.0 | Negative | | | | | |
| 537214 | Month 12 | SP110 | 0.054 | 0.6 | Negative | | | | | |
| 537214 | Month 18 | SP110 | 0.084 | 1.8 | POSITIVE | ConfP-83R | 96.4 | YES | TP108 | 2.7 |
| 537214 | Month 24 | SP110 | 0.070 | 1.2 | Negative | | | | | |
| 537215 | Baseline | SP110 | 0.057 | 0.7 | Negative | | | | | |
| 537215 | Month 1 | SP110 | 1.955 | 82.1 | POSITIVE | ConfP-83R | 97.0 | YES | TP107 | 3.7 |
| 537215 | Month 3 | SP110 | 1.347 | 56.0 | POSITIVE | ConfP-83R | 93.3 | YES | TP107 | 3.7 |
| 537215 | Month 6 | SP110 | 1.054 | 43.5 | POSITIVE | ConfP-83R | 91.9 | YES | TP107 | 3.7 |
| 537215 | Month 9 | SP110 | 1.834 | 76.9 | POSITIVE | ConfP-83R | 95.6 | YES | TP108 | 4.7 |
| 537215 | Month 12 | SP110 | 1.834 | 76.9 | POSITIVE | ConfP-83R | 97.4 | YES | TP108 | 4.7 |
| 537215 | Month 18 | SP110 | 1.579 | 66.0 | POSITIVE | ConfP-83R | 97.2 | YES | TP108 | 3.7 |
| 537215 | Month 24 | SP110 | 1.257 | 52.2 | POSITIVE | ConfP-83R | 97.6 | YES | TP108 | 3.7 |
| 537216 | Baseline | SP111 | 0.068 | 1.2 | Negative | | | | | |
| 537216 | Month 1 | SP111 | 0.073 | 1.4 | Negative | | | | | |
| 537216 | Month 3 | SP111 | 0.883 | 39.9 | POSITIVE | ConfP-83R | 99.0 | YES | TP108 | 3.7 |
| 537216 | Month 6 | SP111 | 0.510 | 22.2 | POSITIVE | ConfP-83R | 98.2 | YES | TP109 | 3.7 |
| 537216 | Month 9 | SP111 | 0.167 | 5.9 | POSITIVE | ConfP-83R | 95.7 | YES | TP109 | 2.7 |
| 537216 | Month 12 | SP111 | 0.113 | 3.3 | POSITIVE | ConfP-84 | 94.2 | YES | TP109 | 2.7 |
| 537216 | Month 18 | SP111 | 0.121 | 3.7 | POSITIVE | ConfP-84 | 96.4 | YES | TP109 | 2.7 |
| 537216 | Month 24 | SP111 | 0.167 | 5.9 | POSITIVE | ConfP-84 | 97.7 | YES | TP109 | 2.7 |
| 537305 | Baseline | SP24 | 0.047 | 0.0 | Negative | | | | | |
| 537305 | Month 1 | SP24 | 0.509 | 34.5 | POSITIVE | ConfP-21 | 98.3 | YES | TP242 | 3.7 |
| 537305 | Month 3 | SP24 | 1.644 | 119.5 | POSITIVE | ConfP-3 | 99.5 | YES | TP10 | 3.7 |
| 537305 | Month 6 | SP24 | 0.526 | 35.8 | POSITIVE | ConfP-21 | 95.5 | YES | TP242 | 3.7 |
| 537305 | Month 9 | SP24 | 0.431 | 28.7 | POSITIVE | ConfP-21 | 97.5 | YES | TP243 | 2.7 |
| 537305 | Month 12 | SP24 | 0.196 | 11.1 | POSITIVE | ConfP-21 | 97.0 | YES | TP243 | 2.7 |
| 537305 | Month 18 | SP24 | 0.201 | 11.5 | POSITIVE | ConfP-21 | 93.8 | YES | TP243 | 2.7 |
| 537305 | Month 24 | SP24 | 0.123 | 5.6 | POSITIVE | ConfP-22 | 93.2 | YES | TP243 | 2.7 |
| 537307 | Baseline | SP25 | 0.046 | 0.2 | Negative | | | | | |
| 537307 | Month 1 | SP25 | 2.628 | 129.4 | POSITIVE | ConfP-22 | 93.5 | YES | TP243 | 4.7 |
| 537307 | Month 3 | SP25 | 2.636 | 129.8 | POSITIVE | ConfP-3 | 98.1 | YES | TP10 | 3.7 |
| 537307 | Month 6 | SP25 | 2.355 | 115.7 | POSITIVE | ConfP-22 | 96.8 | YES | TP243 | 3.7 |
| 537307 | Month 9 | SP25 | 2.035 | 99.7 | POSITIVE | ConfP-22 | 97.2 | YES | TP244 | 3.7 |
| 537307 | Month 12 | SP25 | 1.690 | 82.4 | POSITIVE | ConfP-22 | 95.8 | YES | TP244 | 3.7 |
| 537307 | Month 18 | SP25 | 1.381 | 67.0 | POSITIVE | ConfP-22 | 93.8 | YES | TP244 | 3.7 |
| 537307 | Month 24 | SP25 | 1.594 | 77.6 | POSITIVE | ConfP-22 | 86.1 | YES | TP244 | 3.7 |
| 537309 | Baseline | SP25 | 0.044 | 0.1 | Negative | | | | | |
| 537309 | Month 1 | SP25 | 2.075 | 101.7 | POSITIVE | ConfP-22 | 99.5 | YES | TP244 | 3.7 |
| 537309 | Month 3 | SP25 | 2.411 | 118.5 | POSITIVE | ConfP-3 | 99.7 | YES | TP10 | 3.7 |
| 537309 | Month 6 | SP25 | 1.527 | 74.3 | POSITIVE | ConfP-22 | 99.2 | YES | TP244 | 3.7 |
| 537309 | Month 9 | SP26 | 1.548 | 76.7 | POSITIVE | ConfP-22 | 99.4 | YES | TP245 | 3.7 |
| 537309 | Month 12 | SP26 | 1.607 | 79.7 | POSITIVE | ConfP-22 | 99.7 | YES | TP245 | 3.7 |
| 537309 | Month 18 | SP26 | 0.918 | 44.5 | POSITIVE | ConfP-22 | 99.3 | YES | TP245 | 3.7 |
| 537309 | Month 24 | SP26 | 0.788 | 37.8 | POSITIVE | ConfP-22 | 98.8 | YES | TP245 | 3.7 |
| 537310 | Baseline | SP26 | 0.052 | 0.2 | Negative | | | | | |
| 537310 | Month 1 | SP26 | 0.656 | 31.1 | POSITIVE | ConfP-23 | 99.3 | YES | TP245 | 2.7 |
| 537310 | Month 3 | SP26 | 1.685 | 83.7 | POSITIVE | ConfP-3 | 99.8 | YES | TP11 | 3.7 |
| 537310 | Month 6 | SP26 | 1.569 | 77.8 | POSITIVE | ConfP-23 | 99.6 | YES | TP245 | 3.7 |
| 537310 | Month 9 | SP26 | 0.619 | 29.2 | POSITIVE | ConfP-23 | 99.3 | YES | TP246 | 3.7 |
| 537310 | Month 12 | SP26 | 1.021 | 49.7 | POSITIVE | ConfP-23 | 99.6 | YES | TP246 | 3.7 |
| 537310 | Month 18 | SP26 | 1.475 | 73.0 | POSITIVE | ConfP-23 | 99.7 | YES | TP246 | 3.7 |
| 537310 | Month 24 | SP26 | 0.331 | 14.5 | POSITIVE | ConfP-23 | 98.6 | YES | TP246 | 2.7 |
| 537312 | Baseline | SP27 | 0.044 | 0.0 | Negative | | | | | |
| 537312 | Month 1 | SP27 | 0.873 | 40.7 | POSITIVE | ConfP-23 | 95.9 | YES | TP246 | 3.7 |
| 537312 | Month 3 | SP27 | 2.052 | 98.4 | POSITIVE | ConfP-23 | 98.1 | YES | TP246 | 3.7 |
| 537312 | Month 6 | SP27 | 2.463 | 118.6 | POSITIVE | ConfP-3 | 99.7 | YES | TP11 | 3.7 |
| 537312 | Month 9 | SP27 | 2.174 | 104.4 | POSITIVE | ConfP-23 | 98.9 | YES | TP247 | 3.7 |
| 537312 | Month 12 | SP27 | 1.963 | 94.1 | POSITIVE | ConfP-23 | 98.7 | YES | TP247 | 3.7 |
| 537312 | Month 18 | SP27 | 1.743 | 83.3 | POSITIVE | ConfP-23 | 98.7 | YES | TP247 | 3.7 |
| 537312 | Month 24 | SP27 | 1.395 | 66.2 | POSITIVE | ConfP-23 | 97.6 | YES | TP247 | 3.7 |
| 537313 | Baseline | SP111 | 0.067 | 1.1 | Negative | | | | | |
| 537313 | Month 1 | SP111 | 0.148 | 5.0 | POSITIVE | ConfP-84 | 99.2 | YES | TP109 | 2.7 |
| 537313 | Month 3 | SP111 | 0.682 | 30.4 | POSITIVE | ConfP-84 | 99.5 | YES | TP110 | 3.7 |
| 537313 | Month 6 | SP111 | 0.321 | 13.2 | POSITIVE | ConfP-84 | 99.3 | YES | TP110 | 3.7 |
| 537313 | Month 9 | SP112 | 0.172 | 7.7 | POSITIVE | ConfP-85 | 98.2 | YES | TP111 | 3.7 |
| 537313 | Month 12 | SP112 | 0.101 | 3.5 | POSITIVE | ConfP-85 | 98.1 | YES | TP111 | 2.7 |
| 537313 | Month 18 | SP112 | 0.080 | 2.3 | POSITIVE | ConfP-85 | 93.3 | YES | TP111 | 2.7 |
| 537313 | Month 24 | SP112 | 0.073 | 1.8 | POSITIVE | ConfP-85 | 93.4 | YES | TP112 | 2.7 |
| 537315 | Baseline | SP112 | 0.063 | 1.2 | Negative | | | | | |
| 537315 | Month 1 | SP112 | 0.300 | 15.3 | POSITIVE | ConfP-84 | 99.4 | YES | TP110 | 3.7 |
| 537315 | Month 3 | SP112 | 0.387 | 20.4 | POSITIVE | ConfP-84 | 99.4 | YES | TP110 | 3.7 |
| 537315 | Month 6 | SP112 | 0.104 | 3.7 | POSITIVE | ConfP-84 | 99.0 | YES | TP110 | 2.7 |
| 537315 | Month 9 | SP112 | 0.093 | 3.0 | POSITIVE | ConfP-84 | 98.9 | YES | TP110 | 2.7 |
| 537315 | Month 12 | SP112 | 0.101 | 3.5 | POSITIVE | ConfP-84 | 98.1 | YES | TP111 | 2.7 |
| 537315 | Month 18 | SP112 | 0.105 | 3.7 | POSITIVE | ConfP-84 | 98.8 | YES | TP111 | 2.7 |
| 537315 | Month 24 | SP112 | 0.111 | 4.1 | POSITIVE | ConfP-84 | 98.3 | YES | TP111 | 2.7 |
| 537316 | Baseline | SP113R | 0.049 | 0.0 | Negative | | | | | |
| 537316 | Month 1 | SP113R | 0.079 | 1.4 | Negative | | | | | |
| 537316 | Month 3 | SP113R | 0.518 | 26.7 | POSITIVE | ConfP-85 | 98.4 | YES | TP112 | 3.7 |
| 537316 | Month 6 | SP113R | 0.385 | 19.1 | POSITIVE | ConfP-85 | 98.6 | YES | TP112 | 3.7 |
| 537316 | Month 9 | SP113R | 0.118 | 3.6 | POSITIVE | ConfP-85 | 96.1 | YES | TP112 | 2.7 |
| 537316 | Month 12 | SP113R | 0.155 | 5.8 | POSITIVE | ConfP-85 | 97.0 | YES | TP112 | 2.7 |
| 537316 | Month 18 | SP113R | 0.256 | 11.6 | POSITIVE | ConfP-85 | 98.1 | YES | TP112 | 2.7 |
| 537316 | Month 24 | SP113R | 0.101 | 2.7 | POSITIVE | ConfP-85 | 96.4 | YES | TP113 | 2.7 |
| 537317 | Baseline | SP113R | 0.053 | 0.0 | Negative | | | | | |
| 537317 | Month 1 | SP113R | 0.091 | 2.1 | POSITIVE | ConfP-85 | 18.7 | No | | |
| 537317 | Month 3 | SP113R | 0.353 | 17.2 | POSITIVE | ConfP-85 | 47.0 | No | | |
| 537317 | Month 6 | SP113R | 0.275 | 12.7 | POSITIVE | ConfP-85 | 99.6 | YES | TP113 | 2.7 |
| 537317 | Month 9 | SP114R | 0.280 | 13.4 | POSITIVE | ConfP-86 | 80.6 | YES | TP114 | 2.7 |
| 537317 | Month 12 | SP114R | 0.196 | 8.7 | POSITIVE | ConfP-86 | 85.9 | YES | TP115 | 2.7 |
| 537317 | Month 18 | SP114R | 0.201 | 8.9 | POSITIVE | ConfP-86 | 88.5 | YES | TP115 | 2.7 |
| 537317 | Month 24 | SP114R | 0.386 | 19.4 | POSITIVE | ConfP-86 | 89.2 | YES | TP115 | 3.7 |
| 537319 | Baseline | SP114R | 0.045 | 0.2 | Negative | | | | | |
| 537319 | Month 1 | SP114R | 0.089 | 2.6 | POSITIVE | ConfP-86 | 83.1 | YES | TP287 | 2.7 |
| 537319 | Month 3 | SP114R | 0.906 | 48.6 | POSITIVE | ConfP-86 | 97.0 | YES | TP287 | 3.7 |
| 537319 | Month 6 | SP114R | 0.745 | 39.6 | POSITIVE | ConfP-86 | 97.1 | YES | TP114 | 3.7 |
| 537319 | Month 9 | SP114R | 0.874 | 46.8 | POSITIVE | ConfP-86 | 97.4 | YES | TP114 | 3.7 |
| 537319 | Month 12 | SP114R | 0.825 | 44.1 | POSITIVE | ConfP-86 | 94.9 | YES | TP114 | 3.7 |
| 537319 | Month 18 | SP114R | 0.411 | 20.8 | POSITIVE | ConfP-86 | 94.3 | YES | TP114 | 3.7 |
| 537319 | Month 24 | SP114R | 0.678 | 35.8 | POSITIVE | ConfP-86 | 91.4 | YES | TP114 | 2.7 |
| 537321 | Baseline | SP115R | 0.047 | 0.0 | Negative | | | | | |
| 537321 | Month 1 | SP115R | 1.948 | 121.7 | POSITIVE | ConfP-86 | 98.5 | YES | TP115 | 4.7 |
| 537321 | Month 3 | SP115R | 2.107 | 131.9 | POSITIVE | ConfP-86 | 96.9 | YES | TP115 | 4.7 |
| 537321 | Month 6 | SP115R | 1.817 | 113.3 | POSITIVE | ConfP-87 | 98.0 | YES | TP115 | 4.7 |
| 537321 | Month 9 | SP115R | 1.625 | 101.0 | POSITIVE | ConfP-87 | 98.2 | YES | TP116 | 4.7 |
| 537321 | Month 12 | SP115R | 1.002 | 61.0 | POSITIVE | ConfP-87 | 97.5 | YES | TP116 | 4.7 |
| 537321 | Month 18 | SP115R | 0.583 | 34.1 | POSITIVE | ConfP-87 | 97.0 | YES | TP116 | 3.7 |
| 537321 | Month 24 | SP115R | 0.756 | 45.2 | POSITIVE | ConfP-87 | 96.0 | YES | TP116 | 4.7 |
| 537401 | Baseline | SP27 | 0.046 | 0.1 | Negative | | | | | |
| 537401 | Month 1 | SP27 | 2.021 | 96.9 | POSITIVE | ConfP-3 | 99.3 | YES | TP11 | 3.7 |
| 537401 | Month 3 | SP27 | 1.006 | 47.2 | POSITIVE | ConfP-23 | 98.8 | YES | TP247 | 3.7 |
| 537401 | Month 6 | SP27 | 0.400 | 17.5 | POSITIVE | ConfP-24 | 98.6 | YES | TP247 | 2.7 |
| 537401 | Month 9 | SP28 | 0.312 | 13.8 | POSITIVE | ConfP-24 | 96.9 | YES | TP248 | 2.7 |
| 537401 | Month 12 | SP28 | 0.360 | 16.3 | POSITIVE | ConfP-24 | 99.1 | YES | TP248 | 2.7 |
| 537401 | Month 18 | SP28 | 0.375 | 17.1 | POSITIVE | ConfP-24 | 98.7 | YES | TP248 | 2.7 |
| 537401 | Month 24 | SP28 | 0.133 | 4.6 | POSITIVE | ConfP-24 | 95.6 | YES | TP248 | 2.7 |
| 537403 | Baseline | SP28 | 0.043 | 0.0 | Negative | | | | | |
| 537403 | Month 1 | SP28 | 0.047 | 0.2 | Negative | | | | | |
| 537403 | Month 3 | SP28 | 0.553 | 26.2 | POSITIVE | ConfP-4 | 100.0 | YES | TP11 | 2.7 |
| 537403 | Month 6 | SP28 | 0.553 | 26.2 | POSITIVE | ConfP-24 | 99.3 | YES | TP248 | 2.7 |
| 537403 | Month 9 | SP28 | 0.174 | 6.7 | POSITIVE | ConfP-24 | 99.3 | YES | TP248 | 2.7 |
| 537403 | Month 12 | SP28 | 0.118 | 3.9 | POSITIVE | ConfP-24 | 97.0 | YES | TP249 | 2.7 |
| 537403 | Month 18 | SP28 | 0.071 | 1.4 | Negative | | | | | |
| 537403 | Month 24 | SP28 | 0.060 | 0.9 | Negative | | | | | |
| 537405 | Baseline | SP115R | 0.066 | 1.0 | Negative | | | | | |
| 537405 | Month 1 | SP115R | 0.232 | 11.6 | POSITIVE | ConfP-87 | 97.2 | YES | TP116 | 3.7 |
| 537405 | Month 3 | SP115R | 1.425 | 88.1 | POSITIVE | ConfP-87 | 98.8 | YES | TP116 | 4.7 |
| 537405 | Month 6 | SP115R | 0.560 | 32.6 | POSITIVE | ConfP-87 | 97.5 | YES | TP117 | 3.7 |
| 537405 | Month 9 | SP116R | 0.453 | 24.0 | POSITIVE | ConfP-88 | 96.3 | YES | TP118 | 3.7 |
| 537405 | Month 12 | SP116R | 0.359 | 18.5 | POSITIVE | ConfP-88 | 97.8 | YES | TP118 | 3.7 |
| 537405 | Month 18 | SP116R | 0.332 | 16.9 | POSITIVE | ConfP-88 | 97.4 | YES | TP118 | 3.7 |
| 537405 | Month 24 | SP116R | 0.204 | 9.5 | POSITIVE | ConfP-88 | 95.1 | YES | TP118 | 2.7 |
| 537407 | Baseline | SP116R | 0.045 | 0.2 | Negative | | | | | |
| 537407 | Month 1 | SP116R | 1.034 | 57.9 | POSITIVE | ConfP-87 | 99.1 | YES | TP117 | 3.7 |
| 537407 | Month 3 | SP116R | 1.484 | 84.2 | POSITIVE | ConfP-87 | 99.2 | YES | TP117 | 3.7 |
| 537407 | Month 6 | SP116R | 0.815 | 45.2 | POSITIVE | ConfP-87 | 99.0 | YES | TP117 | 3.7 |
| 537407 | Month 9 | SP116R | 0.810 | 44.9 | POSITIVE | ConfP-87 | 98.8 | YES | TP117 | 3.7 |
| 537407 | Month 12 | SP116R | 0.480 | 25.6 | POSITIVE | ConfP-87 | 96.1 | YES | TP117 | 3.7 |
| 537407 | Month 18 | SP116R | 0.398 | 20.8 | POSITIVE | ConfP-88 | 98.4 | YES | TP118 | 3.7 |
| 537407 | Month 24 | SP116R | 0.530 | 28.5 | POSITIVE | ConfP-88 | 98.6 | YES | TP118 | 3.7 |
| 537408 | Baseline | SP117R | 0.052 | 0.5 | Negative | | | | | |
| 537408 | Month 1 | SP117R | 0.115 | 3.9 | POSITIVE | ConfP-88 | 95.7 | YES | TP119 | 2.7 |
| 537408 | Month 3 | SP117R | 0.751 | 38.3 | POSITIVE | ConfP-88 | 98.8 | YES | TP119 | 3.7 |
| 537408 | Month 6 | SP117R | 0.222 | 9.7 | POSITIVE | ConfP-88 | 97.3 | YES | TP119 | 2.7 |
| 537408 | Month 9 | SP117R | 0.144 | 5.5 | POSITIVE | ConfP-88 | 96.7 | YES | TP119 | 2.7 |
| 537408 | Month 12 | SP117R | 0.114 | 3.8 | POSITIVE | ConfP-88 | 94.6 | YES | TP119 | 2.7 |
| 537408 | Month 18 | SP117R | 0.137 | 5.1 | POSITIVE | ConfP-88 | 97.1 | YES | TP119 | 2.7 |
| 537408 | Month 24 | SP117R | 0.117 | 4.0 | POSITIVE | ConfP-88 | 94.8 | YES | TP120 | 2.7 |
| 537410 | Baseline | SP117R | 0.052 | 0.5 | Negative | | | | | |
| 537410 | Month 1 | SP117R | 0.346 | 16.4 | POSITIVE | ConfP-89 | 98.6 | YES | TP120 | 2.7 |
| 537410 | Month 3 | SP117R | 2.235 | 118.7 | POSITIVE | ConfP-89 | 99.5 | YES | TP120 | 4.7 |
| 537410 | Month 6 | SP117R | 2.016 | 106.8 | POSITIVE | ConfP-89 | 99.7 | YES | TP120 | 3.7 |
| 537410 | Month 9 | SP118R | 1.157 | 62.9 | POSITIVE | ConfP-89 | 99.6 | YES | TP121 | 3.7 |
| 537410 | Month 12 | SP118R | 1.067 | 57.8 | POSITIVE | ConfP-89 | 99.6 | YES | TP122 | 3.7 |
| 537410 | Month 18 | SP118R | 0.518 | 26.8 | POSITIVE | ConfP-89 | 98.9 | YES | TP122 | 3.7 |
| 537410 | Month 24 | SP118R | 0.775 | 41.3 | POSITIVE | ConfP-90 | 99.4 | YES | TP122 | 3.7 |
| 537412 | Baseline | SP118R | 0.044 | 0.1 | Negative | | | | | |
| 537412 | Month 1 | SP118R | 0.311 | 15.1 | POSITIVE | ConfP-89 | 98.9 | YES | TP120 | 2.7 |
| 537412 | Month 3 | SP118R | 1.019 | 55.1 | POSITIVE | ConfP-89 | 99.6 | YES | TP120 | 3.7 |
| 537412 | Month 6 | SP118R | 0.392 | 19.7 | POSITIVE | ConfP-89 | 99.4 | YES | TP121 | 2.7 |
| 537412 | Month 9 | SP118R | 0.209 | 9.4 | POSITIVE | ConfP-89 | 98.9 | YES | TP121 | 2.7 |
| 537412 | Month 12 | SP118R | 0.310 | 15.1 | POSITIVE | ConfP-89 | 99.4 | YES | TP121 | 2.7 |
| 537412 | Month 18 | SP118R | 0.227 | 10.4 | POSITIVE | ConfP-89 | 98.2 | YES | TP121 | 2.7 |
| 537412 | Month 24 | SP118R | 0.341 | 16.8 | POSITIVE | ConfP-89 | 99.4 | YES | TP121 | 2.7 |
| 537414 | Baseline | SP119R2 | 0.044 | 0.3 | Negative | | | | | |
| 537414 | Month 1 | SP119R2 | 0.188 | 10.2 | POSITIVE | ConfP-90 | 98.1 | YES | TP122 | 2.7 |
| 537414 | Month 3 | SP119R2 | 1.118 | 73.8 | POSITIVE | ConfP-90 | 98.9 | YES | TP122 | 3.7 |
| 537414 | Month 6 | SP119R2 | 0.300 | 17.8 | POSITIVE | ConfP-90 | 99.4 | YES | TP122 | 2.7 |
| 537414 | Month 9 | SP119R2 | 0.450 | 28.1 | POSITIVE | ConfP-90 | 98.7 | YES | TP123 | 3.7 |
| 537414 | Month 2 | SP119R2 | 0.230 | 13.1 | POSITIVE | ConfP-90 | 99.4 | YES | TP123 | 3.7 |
| 537414 | Month 18 | SP119R2 | 0.058 | 1.3 | Negative | | | | | |
| 537414 | Month 24 | SP119R2 | 0.046 | 0.5 | Negative | | | | | |
| 537502 | Baseline | SP119R2 | 0.045 | 0.4 | Negative | | | | | |
| 537502 | Month 1 | SP119R2 | 0.790 | 51.3 | POSITIVE | ConfP-90 | 99.3 | YES | TP123 | 3.7 |
| 537502 | Month 3 | SP119R2 | 0.282 | 16.6 | POSITIVE | ConfP-90 | 98.5 | YES | TP123 | 2.7 |
| 537502 | Month 6 | SP119R2 | 0.504 | 31.8 | POSITIVE | ConfP-90 | 97.7 | YES | TP123 | 3.7 |
| 537502 | Month 9 | SP120R2 | 0.370 | 20.9 | POSITIVE | ConfP-132 | 92.8 | YES | TP287 | 3.7 |
| 537502 | Month 12 | SP120R2 | 0.296 | 16.2 | POSITIVE | ConfP-91 | 96.9 | YES | TP125 | 3.7 |
| 537502 | Month 18 | SP120R2 | 0.731 | 43.9 | POSITIVE | ConfP-91 | 91.3 | YES | TP125 | 3.7 |
| 537502 | Month 24 | SP120R2 | 0.654 | 39.0 | POSITIVE | ConfP-91 | 80.4 | YES | TP125 | 3.7 |
| 537504 | Baseline | SP120R2 | 0.043 | 0.0 | Negative | | | | | |
| 537504 | Month 1 | SP120R2 | 0.303 | 16.6 | POSITIVE | ConfP-90 | 94.4 | YES | TP123 | 2.7 |
| 537504 | Month 3 | SP120R2 | 0.577 | 34.1 | POSITIVE | ConfP-90 | 96.1 | YES | TP124 | 3.7 |
| 537504 | Month 6 | SP120R2 | 0.374 | 21.1 | POSITIVE | ConfP-90 | 93.9 | YES | TP124 | 3.7 |
| 537504 | Month 9 | SP120R2 | 0.045 | 0.1 | Negative | | | | | |
| 537504 | Month 12 | SP120R2 | 0.267 | 14.3 | POSITIVE | ConfP-91 | 91.4 | YES | TP124 | 2.7 |
| 537504 | Month 18 | SP120R2 | 0.154 | 7.1 | POSITIVE | ConfP-91 | 82.8 | YES | TP124 | 2.7 |
| 537504 | Month 24 | SP120R2 | 0.111 | 4.3 | POSITIVE | ConfP-91 | 77.9 | YES | TP124 | 2.7 |
| 537506 | Baseline | SP121R | 0.044 | 0.1 | Negative | | | | | |
| 537506 | Month 1 | SP121R | 0.183 | 10.4 | POSITIVE | ConfP-91 | 96.0 | YES | TP125 | 2.7 |
| 537506 | Month 3 | SP121R | 0.463 | 31.1 | POSITIVE | ConfP-91 | 98.3 | YES | TP125 | 3.7 |
| 537506 | Month 6 | SP121R | 0.239 | 14.5 | POSITIVE | ConfP-91 | 96.3 | YES | TP125 | 2.7 |
| 537506 | Month 9 | SP121R | 0.242 | 14.8 | POSITIVE | ConfP-91 | 96.2 | YES | TP126 | 2.7 |
| 537506 | Month 12 | SP121R | 0.116 | 5.5 | POSITIVE | ConfP-91 | 94.0 | YES | TP126 | 2.7 |
| 537506 | Month 18 | SP121R | 0.153 | 8.2 | POSITIVE | ConfP-91 | 93.4 | YES | TP126 | 2.7 |
| 537506 | Month 24 | SP121R | 0.096 | 4.0 | POSITIVE | ConfP-91 | 91.8 | YES | TP126 | 2.7 |
| 537508 | Baseline | SP121R | 0.046 | 0.3 | Negative | | | | | |
| 537508 | Month 1 | SP121R | 0.226 | 13.6 | POSITIVE | ConfP-92 | 96.3 | YES | TP126 | 2.7 |
| 537508 | Month 3 | SP121R | 0.993 | 70.2 | POSITIVE | ConfP-92 | 99.0 | YES | TP126 | 3.7 |
| 537508 | Month 6 | SP121R | 0.634 | 43.7 | POSITIVE | ConfP-92 | 98.7 | YES | TP127 | 3.7 |
| 537508 | Month 9 | SP122R | 0.044 | 0.1 | Negative | | | | | |
| 537508 | Month 12 | SP122R | 0.334 | 19.7 | POSITIVE | ConfP-92 | 97.2 | YES | TP128 | 3.7 |
| 537508 | Month 18 | SP122R | 0.128 | 5.7 | POSITIVE | ConfP-92 | 97.9 | YES | TP128 | 2.7 |
| 537508 | Month 24 | SP122R | 0.467 | 28.7 | POSITIVE | ConfP-92 | 97.8 | YES | TP128 | 3.7 |
| 537511 | Baseline | SP122R | 0.047 | 0.3 | Negative | | | | | |
| 537511 | Month 1 | SP122R | 0.704 | 44.7 | POSITIVE | ConfP-92 | 98.5 | YES | TP127 | 3.7 |
| 537511 | Month 3 | SP122R | 1.357 | 88.8 | POSITIVE | ConfP-92 | 99.4 | YES | TP127 | 3.7 |
| 537511 | Month 6 | SP122R | 0.472 | 29.0 | POSITIVE | ConfP-92 | 98.7 | YES | TP127 | 3.7 |
| 537511 | Month 9 | SP122R | 0.637 | 40.2 | POSITIVE | ConfP-92 | 98.0 | YES | TP127 | 3.7 |
| 537511 | Month 12 | SP122R | 0.293 | 16.9 | POSITIVE | ConfP-92 | 96.6 | YES | TP127 | 3.7 |
| 537511 | Month 18 | SP122R | 0.215 | 11.6 | POSITIVE | ConfP-92 | 97.1 | YES | TP128 | 2.7 |
| 537511 | Month 24 | SP122R | 0.215 | 11.6 | POSITIVE | ConfP-92 | 95.8 | YES | TP128 | 2.7 |
| 537512 | Baseline | SP184 | 0.055 | 0.7 | Negative | | | | | |
| 537512 | Baseline | SP184 | 0.063 | 1.2 | Negative | | | | | |
| 537512 | Month 1 | SP123R | 2.277 | 130.2 | POSITIVE | ConfP-93 | 98.8 | YES | TP128 | 4.7 |
| 537512 | Month 3 | SP123R | 1.188 | 66.8 | POSITIVE | ConfP-93 | 99.2 | YES | TP129 | 3.7 |
| 537512 | Month 6 | SP123R | 0.294 | 14.7 | POSITIVE | ConfP-93 | 98.3 | YES | TP129 | 3.7 |
| 537512 | Month 9 | SP123R | 0.044 | 0.1 | Negative | | | | | |
| 537512 | Month 12 | SP123R | 0.169 | 7.4 | POSITIVE | ConfP-93 | 96.1 | YES | TP129 | 2.7 |
| 537512 | Month 18 | SP123R | 0.288 | 14.3 | POSITIVE | ConfP-93 | 98.2 | YES | TP129 | 2.7 |
| 537512 | Month 24 | SP123R | 0.203 | 9.4 | POSITIVE | ConfP-93 | 97.6 | YES | TP129 | 2.7 |
| 537606 | Baseline | SP192 | 0.084 | 0.4 | Negative | | | | | |
| 537606 | Month 1 | SP192 | 0.943 | 37.7 | POSITIVE | ConfP-141 | 98.8 | YES | TP302 | 2.7 |
| 537606 | Month 3 | SP192 | 2.490 | 105.0 | POSITIVE | ConfP-141 | 98.9 | YES | TP302 | 3.7 |
| 537606 | Month 6 | SP192 | 2.232 | 93.7 | POSITIVE | ConfP-141 | 99.6 | YES | TP302 | 3.7 |
| 537606 | Month 9 | No sample received | | | | | | | | |
| 537606 | Month 12 | SP192 | 2.162 | 90.7 | POSITIVE | ConfP-141 | 99.7 | YES | TP302 | 3.7 |
| 537606 | Month 18 | SP192 | 0.357 | 12.3 | POSITIVE | ConfP-141 | 96.2 | YES | TP302 | 2.7 |
| 537606 | Month 24 | SP196 | 1.231 | 48.3 | POSITIVE | ConfP-141 | 99.2 | YES | TP302 | 2.7 |
| 537901 | Baseline | SP29 | 0.052 | 0.6 | Negative | | | | | |
| 537901 | Month 1 | SP29 | 0.708 | 42.0 | POSITIVE | ConfP-24 | 99.5 | YES | TP249 | 3.7 |
| 537901 | Month 3 | SP29 | 1.177 | 71.7 | POSITIVE | ConfP-4 | 100.0 | YES | TP11 | 3.7 |
| 537901 | Month 6 | SP29 | 0.294 | 15.9 | POSITIVE | ConfP-24 | 99.1 | YES | TP249 | 2.7 |
| 537901 | Month 9 | SP29 | 0.226 | 11.6 | POSITIVE | ConfP-24 | 98.6 | YES | TP249 | 2.7 |
| 537901 | Month 12 | SP29 | 0.462 | 26.5 | POSITIVE | ConfP-24 | 98.9 | YES | TP249 | 2.7 |
| 537901 | Month 18 | SP29 | 0.266 | 14.1 | POSITIVE | ConfP-24 | 98.9 | YES | TP249 | 2.7 |
| 537901 | Month 24 | SP29 | 0.430 | 24.5 | POSITIVE | ConfP-25 | 99.6 | YES | TP250 | 2.7 |
| 537902 | Baseline | SP186 | 0.097 | 1.1 | Negative | | | | | |
| 537902 | Month 1 | No sample received | | | | | | | | |
| 537902 | Month 3 | SP186 | 1.048 | 45.6 | POSITIVE | ConfP-138 | 99.4 | YES | TP315 | 3.7 |
| 537902 | Month 6 | SP186 | 0.236 | 7.6 | POSITIVE | ConfP-138 | 98.6 | YES | TP293 | 2.7 |
| 537902 | Month 9 | SP186 | 0.167 | 4.4 | POSITIVE | ConfP-138 | 100.0 | YES | TP293 | 2.7 |
| 537902 | Month 12 | SP186 | 0.299 | 10.6 | POSITIVE | ConfP-138 | 100.0 | YES | TP293 | 2.7 |
| 537902 | Month 18 | SP186 | 0.173 | 4.7 | POSITIVE | ConfP-138 | 100.0 | YES | TP293 | 2.7 |
| 537902 | Month 24 | SP186 | 0.196 | 5.8 | POSITIVE | ConfP-138 | 100.0 | YES | TP293 | 2.7 |
| 537903 | Baseline | SP193 | 0.102 | 1.4 | Negative | | | | | |
| 537903 | Month 1 | SP193 | 0.247 | 7.1 | POSITIVE | ConfP-142 | 73.3 | YES | TP303 | 2.7 |
| 537903 | Month 3 | SP193 | 2.724 | 104.8 | POSITIVE | ConfP-142 | 99.8 | YES | TP303 | 3.7 |
| 537903 | Month 6 | SP193 | 2.486 | 95.4 | POSITIVE | ConfP-142 | 99.5 | YES | TP303 | 3.7 |
| 537903 | Month 9 | No sample received | | | | | | | | |
| 537903 | Month 12 | SP193 | 2.906 | 112.0 | POSITIVE | ConfP-142 | 98.8 | YES | TP303 | 3.7 |
| 537903 | Month 18 | SP193 | 2.250 | 86.1 | POSITIVE | ConfP-142 | 98.8 | YES | TP303 | 3.7 |
| 537903 | Month 24 | SP193 | 2.582 | 99.2 | POSITIVE | ConfP-142 | 99.3 | YES | TP303 | 3.7 |
| 537906 | Baseline | SP29 | 0.049 | 0.4 | Negative | | | | | |
| 537906 | Month 1 | SP29 | 0.611 | 35.9 | POSITIVE | ConfP-25 | 99.8 | YES | TP250 | 3.7 |
| 537906 | Month 3 | SP29 | 1.546 | 95.0 | POSITIVE | ConfP-4 | 100.0 | YES | TP11 | 3.7 |
| 537906 | Month 6 | SP29 | 0.565 | 33.0 | POSITIVE | ConfP-25 | 98.8 | YES | TP250 | 2.7 |
| 537906 | Month 9 | SP30 | 0.266 | 14.8 | POSITIVE | ConfP-25 | 99.1 | YES | TP250 | 2.7 |
| 537906 | Month 12 | SP30 | 0.298 | 17.0 | POSITIVE | ConfP-25 | 99.0 | YES | TP250 | 2.7 |
| 537906 | Month 18 | SP30 | 0.151 | 7.0 | POSITIVE | ConfP-25 | 98.2 | YES | TP250 | 2.7 |
| 537906 | Month 24 | SP30 | 0.200 | 10.3 | POSITIVE | ConfP-25 | 99.2 | YES | TP251 | 2.7 |
| 537910 | Baseline | SP124R | 0.064 | 1.3 | Negative | | | | | |
| 537910 | Month 1 | SP124R | 0.317 | 16.2 | POSITIVE | ConfP-93 | 97.0 | YES | TP130 | 2.7 |
| 537910 | Month 3 | SP124R | 2.004 | 115.1 | POSITIVE | ConfP-93 | 99.3 | YES | TP130 | 4.7 |
| 537910 | Month 6 | SP124R | 0.332 | 17.1 | POSITIVE | ConfP-93 | 97.3 | YES | TP130 | 2.7 |
| 537910 | Month 9 | SP124R | 0.176 | 7.9 | POSITIVE | ConfP-93 | 96.1 | YES | TP130 | 2.7 |
| 537910 | Month 12 | SP124R | 0.376 | 19.6 | POSITIVE | ConfP-94 | 97.6 | YES | TP131 | 2.7 |
| 537910 | Month 18 | SP124R | 0.291 | 14.7 | POSITIVE | ConfP-94 | 97.9 | YES | TP131 | 2.7 |
| 537910 | Month 24 | SP124R | 0.227 | 10.9 | POSITIVE | ConfP-94 | 96.1 | YES | TP131 | 2.7 |
| 537911 | Baseline | SP123R | 0.049 | 0.4 | Negative | | | | | |
| 537911 | Month 1 | SP123R | 0.507 | 27.1 | POSITIVE | ConfP-93 | 98.4 | YES | TP129 | 3.7 |
| 537911 | Month 3 | SP123R | 0.813 | 44.9 | POSITIVE | ConfP-93 | 98.9 | YES | TP130 | 3.7 |
| 537911 | Month 6 | SP123R | 0.343 | 17.5 | POSITIVE | ConfP-93 | 96.9 | YES | TP130 | 3.7 |
| 537911 | Month 9 | SP124R | 0.135 | 5.5 | POSITIVE | ConfP-94 | 90.4 | YES | TP131 | 2.7 |
| 537911 | Month 12 | SP124R | 0.104 | 3.7 | POSITIVE | ConfP-94 | 91.1 | YES | TP131 | 2.7 |
| 537911 | Month 18 | SP124R | 0.166 | 7.3 | POSITIVE | ConfP-94 | 95.3 | YES | TP131 | 2.7 |
| 537911 | Month 24 | SP124R | 0.093 | 3.0 | POSITIVE | ConfP-94 | 81.5 | YES | TP132 | 2.7 |
| 537912 | Baseline | SP125R | 0.053 | 0.7 | Negative | | | | | |
| 537912 | Month 1 | SP125R | 0.562 | 37.0 | POSITIVE | ConfP-94 | 96.3 | YES | TP132 | 2.7 |
| 537912 | Month 3 | SP125R | 1.967 | 137.3 | POSITIVE | ConfP-94 | 98.4 | YES | TP132 | 3.7 |
| 537912 | Month 6 | SP125R | 1.673 | 116.3 | POSITIVE | ConfP-94 | 99.0 | YES | TP132 | 3.7 |
| 537912 | Month 9 | SP125R | 1.101 | 75.5 | POSITIVE | ConfP-94 | 98.4 | YES | TP132 | 3.7 |
| 537912 | Month 12 | SP125R | 0.854 | 57.9 | POSITIVE | ConfP-94 | 98.1 | YES | TP132 | 3.7 |
| 537912 | Month 18 | SP125R | 0.390 | 24.8 | POSITIVE | ConfP-94 | 98.4 | YES | TP133 | 2.7 |
| 537912 | Month 24 | SP125R | 0.264 | 15.8 | POSITIVE | ConfP-95 | 97.1 | YES | TP133 | 2.7 |
| 538101 | Baseline | SP125R | 0.054 | 0.8 | Negative | | | | | |
| 538101 | Month 1 | SP125R | 0.064 | 1.5 | POSITIVE | ConfP-95 | 82.4 | YES | TP133 | 2.7 |
| 538101 | Month 3 | SP125R | 0.066 | 1.6 | POSITIVE | ConfP-95 | 83.3 | YES | TP133 | <2.7 |
| 538101 | Month 6 | SP125R | 1.065 | 72.9 | POSITIVE | ConfP-95 | 99.0 | YES | TP133 | 3.7 |
| 538101 | Month 9 | SP126R | 0.198 | 10.9 | POSITIVE | ConfP-95 | 99.1 | YES | TP135 | 2.7 |
| 538101 | Month 12 | SP126R | 0.275 | 16.3 | POSITIVE | ConfP-96 | 99.5 | YES | TP135 | 2.7 |
| 538101 | Month 18 | SP126R | 0.149 | 7.5 | POSITIVE | ConfP-96 | 97.8 | YES | TP135 | 2.7 |
| 538101 | Month 24 | SP126R | 0.366 | 22.7 | POSITIVE | ConfP-96 | 99.2 | YES | TP135 | 2.7 |
| 538102 | Baseline | SP126R | 0.066 | 1.7 | POSITIVE | ConfP-95 | 68.2 | YES | TP133 | 2.7 |
| 538102 | Month 1 | SP126R | 0.566 | 36.7 | POSITIVE | ConfP-95 | 96.0 | YES | TP134 | 2.7 |
| 538102 | Month 3 | SP126R | 2.112 | 145.1 | POSITIVE | ConfP-95 | 98.9 | YES | TP134 | 3.7 |
| 538102 | Month 6 | SP126R | 0.342 | 21.0 | POSITIVE | ConfP-95 | 98.5 | YES | TP134 | 2.7 |
| 538102 | Month 9 | SP126R | 1.004 | 67.4 | POSITIVE | ConfP-95 | 96.5 | YES | TP134 | 3.7 |
| 538102 | Month 12 | SP126R | 1.812 | 124.0 | POSITIVE | ConfP-95 | 99.4 | YES | TP134 | 3.7 |
| 538102 | Month 18 | SP126R | 1.376 | 93.5 | POSITIVE | ConfP-95 | 97.4 | YES | TP134 | 3.7 |
| 538102 | Month 24 | SP126R | 1.217 | 82.3 | POSITIVE | ConfP-95 | 98.3 | YES | TP135 | 3.7 |
| 538201 | Baseline | SP30 | 0.046 | 0.0 | Negative | | | | | |
| 538201 | Month 1 | SP30 | 0.146 | 6.7 | POSITIVE | ConfP-25 | 98.5 | YES | TP251 | 2.7 |
| 538201 | Month 3 | SP30 | 0.559 | 34.6 | POSITIVE | ConfP-4 | 99.5 | YES | TP12 | 3.7 |
| 538201 | Month 6 | SP30 | 0.177 | 8.8 | POSITIVE | ConfP-25 | 97.4 | YES | TP251 | 2.7 |
| 538201 | Month 9 | SP30 | 0.159 | 7.6 | POSITIVE | ConfP-25 | 97.1 | YES | TP251 | 2.7 |
| 538201 | Month 12 | SP30 | 0.109 | 4.2 | POSITIVE | ConfP-25 | 96.0 | YES | TP251 | 2.7 |
| 538201 | Month 18 | SP30 | 0.064 | 1.1 | Negative | | | | | |
| 538201 | Month 24 | SP30 | 0.064 | 1.1 | Negative | | | | | |
| 538202 | Baseline | SP31 | 0.054 | 0.7 | Negative | | | | | |
| 538202 | Month 1 | SP31 | 0.520 | 31.5 | POSITIVE | ConfP-25 | 99.0 | YES | TP251 | 3.7 |
| 538202 | Month 3 | SP31 | 1.377 | 88.1 | POSITIVE | ConfP-4 | 100.0 | YES | TP12 | 3.7 |
| 538202 | Month 6 | SP31 | 0.623 | 38.3 | POSITIVE | ConfP-25 | 98.8 | YES | TP252 | 2.7 |
| 538202 | Month 9 | SP31 | 0.711 | 44.1 | POSITIVE | ConfP-26 | 99.2 | YES | TP252 | 3.7 |
| 538202 | Month 12 | SP31 | 0.606 | 37.1 | POSITIVE | ConfP-26 | 98.4 | YES | TP252 | 2.7 |
| 538202 | Month 18 | SP31 | 0.473 | 28.4 | POSITIVE | ConfP-26 | 98.9 | YES | TP252 | 2.7 |
| 538202 | Month 24 | SP31 | 0.531 | 32.2 | POSITIVE | ConfP-26 | 99.0 | YES | TP252 | 2.7 |
| 538203 | Baseline | SP127 | 0.047 | 0.4 | Negative | | | | | |
| 538203 | Month 1 | SP127 | 0.052 | 0.8 | Negative | | | | | |
| 538203 | Month 3 | SP127 | 0.171 | 9.0 | POSITIVE | ConfP-96 | 97.0 | YES | TP135 | 2.7 |
| 538203 | Month 6 | SP127 | 0.245 | 14.1 | POSITIVE | ConfP-96 | 97.3 | YES | TP136 | 2.7 |
| 538203 | Month 9 | SP127 | 0.176 | 9.3 | POSITIVE | ConfP-96 | 97.6 | YES | TP136 | 2.7 |
| 538203 | Month 12 | SP127 | 0.219 | 12.3 | POSITIVE | ConfP-96 | 94.6 | YES | TP136 | 2.7 |
| 538203 | Month 18 | SP127 | 0.238 | 13.6 | POSITIVE | ConfP-96 | 96.8 | YES | TP136 | 2.7 |
| 538203 | Month 24 | SP127 | 0.261 | 15.2 | POSITIVE | ConfP-96 | 97.4 | YES | TP136 | 2.7 |
| 543501 | Baseline | SP31 | 0.053 | 0.6 | Negative | | | | | |
| 543501 | Month 1 | SP31 | 0.081 | 2.4 | POSITIVE | ConfP-26 | 97.4 | YES | TP252 | 2.7 |
| 543501 | Month 3 | SP31 | 0.313 | 17.8 | POSITIVE | ConfP-26 | 99.5 | YES | TP253 | 2.7 |
| 543501 | Month 6 | SP31 | 0.555 | 33.8 | POSITIVE | ConfP-4 | 100.0 | YES | TP12 | 2.7 |
| 543501 | Month 9 | SP32 | 0.146 | 7.2 | POSITIVE | ConfP-26 | 99.0 | YES | TP253 | 2.7 |
| 543501 | Month 12 | SP32 | 0.122 | 5.5 | POSITIVE | ConfP-26 | 98.5 | YES | TP253 | 2.7 |
| 543501 | Month 18 | SP32 | 0.280 | 16.7 | POSITIVE | ConfP-26 | 99.1 | YES | TP253 | 2.7 |
| 543501 | Month 24 | SP32 | 0.216 | 12.1 | POSITIVE | ConfP-26 | 66.7 | YES | TP253 | 2.7 |
| 543505 | Baseline | SP190 | 0.077 | 0.1 | Negative | | | | | |
| 543505 | Month 1 | SP190 | 0.177 | 4.0 | POSITIVE | ConfP-135 | 100.0 | YES | TP315 | 2.7 |
| 543505 | Month 3 | No sample received | | | | | | | | |
| 543505 | Month 6 | SP190 | 0.226 | 6.0 | POSITIVE | ConfP-135 | 100.0 | YES | TP317 | 2.7 |
| 543505 | Month 9 | SP190 | 0.156 | 3.2 | POSITIVE | ConfP-135 | 100.0 | YES | TP294 | <2.7 |
| 543505 | Month 12 | SP190 | 0.134 | 2.3 | POSITIVE | ConfP-135 | 100.0 | YES | TP294 | <2.7 |
| 543505 | Month 18 | SP190 | 0.180 | 4.2 | POSITIVE | ConfP-135 | 100.0 | YES | TP294 | 2.7 |
| 543505 | Month 24 | SP190 | 0.196 | 4.8 | POSITIVE | ConfP-135 | 100.0 | YES | TP294 | 2.7 |
| 551001 | Baseline | SP190 | 0.091 | 0.6 | Negative | | | | | |
| 551001 | Month 1 | SP190 | 0.477 | 15.9 | POSITIVE | ConfP-147 | 98.6 | YES | TP315 | 2.7 |
| 551001 | Month 3 | No sample received | | | | | | | | |
| 551001 | Month 6 | SP190 | 1.134 | 42.0 | POSITIVE | ConfP-135 | 100.0 | YES | TP294 | 2.7 |
| 551001 | Month 9 | SP190 | 1.047 | 38.5 | POSITIVE | ConfP-135 | 97.2 | YES | TP294 | 2.7 |
| 551001 | Month 12 | SP190 | 1.250 | 46.6 | POSITIVE | ConfP-135 | 95.5 | YES | TP295 | 3.7 |
| 551001 | Month 18 | SP190 | 0.676 | 23.8 | POSITIVE | ConfP-135 | 92.0 | YES | TP295 | 2.7 |
| 551001 | Month 24 | SP196 | 1.342 | 52.9 | POSITIVE | ConfP-135 | 100.0 | YES | TP295 | 2.7 |
| 551002 | Baseline | SP127 | 0.057 | 1.1 | Negative | | | | | |
| 551002 | Month 1 | SP127 | 1.593 | 107.0 | POSITIVE | ConfP-96 | 99.4 | YES | TP136 | 3.7 |
| 551002 | Month 3 | SP127 | 0.896 | 58.9 | POSITIVE | ConfP-96 | 98.9 | YES | TP137 | 3.7 |
| 551002 | Month 6 | SP127 | 0.640 | 41.3 | POSITIVE | ConfP-96 | 97.4 | YES | TP137 | 3.7 |
| 551002 | Month 9 | SP128 | 0.793 | 44.9 | POSITIVE | ConfP-97 | 97.4 | YES | TP138 | 3.7 |
| 551002 | Month 12 | SP128 | 0.753 | 42.5 | POSITIVE | ConfP-97 | 98.9 | YES | TP138 | 3.7 |
| 551002 | Month 18 | SP128 | 1.147 | 66.0 | POSITIVE | ConfP-97 | 98.8 | YES | TP138 | 3.7 |
| 551002 | Month 24 | SP128 | 0.824 | 46.7 | POSITIVE | ConfP-97 | 98.4 | YES | TP139 | 3.7 |
| 551005 | Baseline | SP128 | 0.056 | 0.9 | Negative | | | | | |
| 551005 | Month 1 | SP128 | 0.340 | 17.9 | POSITIVE | ConfP-96 | 98.6 | YES | TP137 | 2.7 |
| 551005 | Month 3 | SP128 | 2.372 | 139.2 | POSITIVE | ConfP-97 | 99.4 | YES | TP137 | 4.7 |
| 551005 | Month 6 | SP128 | 0.777 | 43.9 | POSITIVE | ConfP-97 | 98.5 | YES | TP137 | 3.7 |
| 551005 | Month 9 | SP128 | 0.893 | 50.9 | POSITIVE | ConfP-97 | 98.6 | YES | TP137 | 3.7 |
| 551005 | Month 12 | SP128 | 1.659 | 96.6 | POSITIVE | ConfP-97 | 99.3 | YES | TP138 | 3.7 |
| 551005 | Month 18 | SP128 | 0.571 | 31.6 | POSITIVE | ConfP-97 | 98.1 | YES | TP138 | 3.7 |
| 551005 | Month 24 | SP128 | 0.390 | 20.8 | POSITIVE | ConfP-97 | 96.9 | YES | TP138 | 3.7 |
| 551206 | Baseline | SP186 | 0.083 | 0.5 | Negative | | | | | |
| 551206 | Month 1 | No sample received | | | | | | | | |
| 551206 | Month 3 | SP186 | 1.784 | 80.1 | POSITIVE | ConfP-138 | 99.6 | YES | TP295 | 3.7 |
| 551206 | Month 6 | SP186 | 0.947 | 40.9 | POSITIVE | ConfP-138 | 99.4 | YES | TP295 | 2.7 |
| 551206 | Month 9 | SP186 | 0.498 | 19.9 | POSITIVE | ConfP-138 | 98.4 | YES | TP295 | 2.7 |
| 551206 | Month 12 | SP186 | 0.473 | 18.7 | POSITIVE | ConfP-138 | 100.0 | YES | TP297 | 2.7 |
| 551206 | Month 18 | SP196 | 0.555 | 20.4 | POSITIVE | ConfP-138 | 99.1 | YES | TP297 | 2.7 |
| 551206 | Month 24 | SP196 | 0.464 | 16.7 | POSITIVE | ConfP-138 | 98.0 | YES | TP297 | 2.7 |
| 551207 | Baseline | SP187 | 0.073 | 0.0 | Negative | | | | | |
| 551207 | Month 1 | No sample received | | | | | | | | |
| 551207 | Month 3 | SP187 | 1.426 | 63.4 | POSITIVE | ConfP-139 | 100.0 | YES | TP315 | 3.7 |
| 551207 | Month 6 | SP187 | 1.063 | 46.3 | POSITIVE | ConfP-139 | 99.7 | YES | TP297 | 3.7 |
| 551207 | Month 9 | SP187 | 0.365 | 13.5 | POSITIVE | ConfP-139 | 99.3 | YES | TP297 | 2.7 |
| 551207 | Month 12 | SP187 | 0.162 | 3.9 | POSITIVE | ConfP-139 | 100.0 | YES | TP297 | 2.7 |
| 551207 | Month 18 | SP199 | 0.584 | 28.1 | POSITIVE | ConfP-139 | 99.1 | YES | TP315 | 2.7 |
| 551207 | Month 24 | SP187 | 0.145 | 3.1 | POSITIVE | ConfP-139 | 95.2 | YES | TP316 | 2.7 |
| 551208 | Baseline | SP187 | 0.083 | 0.2 | Negative | | | | | |
| 551208 | Month 1 | No sample received | | | | | | | | |
| 551208 | Month 3 | SP187 | 0.812 | 34.5 | POSITIVE | ConfP-139 | 99.8 | YES | TP304 | 2.7 |
| 551208 | Month 6 | SP199 | 0.426 | 19.3 | POSITIVE | ConfP-139 | 100.0 | YES | TP304 | 2.7 |
| 551208 | Month 9 | SP199 | 0.707 | 34.9 | POSITIVE | ConfP-139 | 98.4 | YES | TP304 | 2.7 |
| 551208 | Month 12 | SP187 | 0.511 | 20.4 | POSITIVE | ConfP-139 | 97.6 | YES | TP304 | 2.7 |
| 551208 | Month 18 | SP187 | 0.299 | 10.4 | POSITIVE | ConfP-139 | 98.4 | YES | TP304 | 2.7 |
| 551208 | Month 24 | SP196 | 0.642 | 24.0 | POSITIVE | ConfP-139 | 98.4 | YES | TP304 | 2.7 |
| 571001 | Baseline | SP129 | 0.049 | 0.5 | Negative | | | | | |
| 571001 | Month 1 | SP129 | 0.539 | 28.7 | POSITIVE | ConfP-97 | 98.1 | YES | TP139 | 3.7 |
| 571001 | Month 3 | SP129 | 0.452 | 23.7 | POSITIVE | ConfP-97 | 97.6 | YES | TP139 | 3.7 |
| 571001 | Month 6 | SP129 | 0.402 | 20.8 | POSITIVE | ConfP-97 | 98.3 | YES | TP139 | 2.7 |
| 571001 | Month 9 | SP129 | 0.092 | 2.9 | POSITIVE | ConfP-98 | 92.7 | YES | TP139 | 2.7 |
| 571001 | Month 12 | SP129 | 0.146 | 6.1 | POSITIVE | ConfP-98 | 96.0 | YES | TP139 | 2.7 |
| 571001 | Month 18 | SP129 | 0.154 | 6.5 | POSITIVE | ConfP-98 | 96.0 | YES | TP140 | 2.7 |
| 571001 | Month 24 | SP129 | 0.105 | 3.7 | POSITIVE | ConfP-98 | 96.1 | YES | TP140 | 2.7 |
| 571002 | Baseline | SP129 | 0.051 | 0.6 | Negative | | | | | |
| 571002 | Month 1 | SP129 | 1.999 | 112.9 | POSITIVE | ConfP-98 | 98.2 | YES | TP140 | 3.7 |
| 571002 | Month 3 | SP129 | 1.045 | 57.9 | POSITIVE | ConfP-98 | 99.1 | YES | TP140 | 3.7 |
| 571002 | Month 6 | SP129 | 0.127 | 5.0 | POSITIVE | ConfP-98 | 97.8 | YES | TP140 | 2.7 |
| 571002 | Month 9 | SP130 | 0.432 | 23.0 | POSITIVE | ConfP-99 | 98.8 | YES | TP142 | 2.7 |
| 571002 | Month 12 | SP130 | 0.727 | 40.4 | POSITIVE | ConfP-99 | 98.8 | YES | TP142 | 3.7 |
| 571002 | Month 18 | SP130 | 0.675 | 37.4 | POSITIVE | ConfP-99 | 98.8 | YES | TP142 | 2.7 |
| 571002 | Month 24 | SP130 | 1.119 | 63.5 | POSITIVE | ConfP-99 | 99.5 | YES | TP142 | 3.7 |
| 571101 | Baseline | SP197 | 0.101 | 0.6 | Negative | | | | | |
| 571101 | Month 1 | SP197 | 1.117 | 46.9 | POSITIVE | ConfP-145 | 98.9 | YES | TP305 | 2.7 |
| 571101 | Month 3 | SP197 | 2.893 | 127.9 | POSITIVE | ConfP-145 | 99.0 | YES | TP305 | 3.7 |
| 571101 | Month 6 | SP197 | 3.019 | 133.6 | POSITIVE | ConfP-145 | 98.5 | YES | TP305 | 3.7 |
| 571101 | Month 9 | SP197 | 2.113 | 92.3 | POSITIVE | ConfP-145 | 99.3 | YES | TP305 | 3.7 |
| 571101 | Month 12 | No sample received | | | | | | | | |
| 571101 | Month 18 | SP197 | 0.738 | 29.7 | POSITIVE | ConfP-145 | 96.0 | YES | TP305 | 2.7 |
| 571101 | Month 24 | SP197 | 1.124 | 47.3 | POSITIVE | ConfP-145 | 99.3 | YES | TP305 | 2.7 |
| 571103 | Baseline | SP130 | 0.056 | 0.9 | Negative | | | | | |
| 571103 | Month 1 | SP130 | 1.050 | 59.5 | POSITIVE | ConfP-98 | 98.6 | YES | TP140 | 3.7 |
| 571103 | Month 3 | SP130 | 2.181 | 126.1 | POSITIVE | ConfP-98 | 97.9 | YES | TP141 | 3.7 |
| 571103 | Month 6 | SP130 | 1.502 | 86.1 | POSITIVE | ConfP-98 | 96.4 | YES | TP287 | 3.7 |
| 571103 | Month 9 | SP130 | 0.607 | 33.4 | POSITIVE | ConfP-98 | 94.7 | YES | TP141 | 3.7 |
| 571103 | Month 12 | SP130 | 0.380 | 20.0 | POSITIVE | ConfP-98 | 94.3 | YES | TP287 | 2.7 |
| 571103 | Month 18 | SP130 | 0.536 | 29.2 | POSITIVE | ConfP-98 | 96.9 | YES | TP141 | 2.7 |
| 571103 | Month 24 | SP130 | 0.184 | 8.4 | POSITIVE | ConfP-99 | 94.9 | YES | TP288 | 3.7 |
| 571104 | Baseline | SP131 | 0.047 | 0.3 | Negative | | | | | |
| 571104 | Month 1 | SP131 | 0.364 | 21.2 | POSITIVE | ConfP-99 | 99.0 | YES | TP142 | 2.7 |
| 571104 | Month 3 | SP131 | 1.527 | 97.6 | POSITIVE | ConfP-99 | 99.5 | YES | TP142 | 4.7 |
| 571104 | Month 6 | SP131 | 0.685 | 42.3 | POSITIVE | ConfP-99 | 98.9 | YES | TP143 | 3.7 |
| 571104 | Month 9 | SP131 | 0.441 | 26.2 | POSITIVE | ConfP-99 | 98.8 | YES | TP143 | 3.7 |
| 571104 | Month 12 | SP131 | 0.659 | 40.6 | POSITIVE | ConfP-99 | 98.8 | YES | TP143 | 3.7 |
| 571104 | Month 18 | SP131 | 0.773 | 48.1 | POSITIVE | ConfP-99 | 99.1 | YES | TP143 | 3.7 |
| 571104 | Month 24 | SP131 | 0.553 | 33.6 | POSITIVE | ConfP-99 | 98.9 | YES | TP143 | 3.7 |
| 571203 | Baseline | SP131 | 0.045 | 0.2 | Negative | | | | | |
| 571203 | Month 1 | SP131 | 0.997 | 62.8 | POSITIVE | ConfP-99 | 99.3 | YES | TP143 | 3.7 |
| 571203 | Month 3 | SP131 | 1.270 | 80.7 | POSITIVE | ConfP-100 | 97.6 | YES | TP144 | 3.7 |
| 571203 | Month 6 | SP131 | 0.648 | 39.8 | POSITIVE | ConfP-100 | 98.5 | YES | TP144 | 3.7 |
| 571203 | Month 9 | SP132 | 0.493 | 26.4 | POSITIVE | ConfP-100 | 99.0 | YES | TP145 | 3.7 |
| 571203 | Month 12 | SP132 | 0.203 | 9.4 | POSITIVE | ConfP-100 | 98.3 | YES | TP145 | 2.7 |
| 571203 | Month 18 | SP132 | 0.152 | 6.4 | POSITIVE | ConfP-100 | 98.0 | YES | TP145 | 2.7 |
| 571203 | Month 24 | SP132 | 0.089 | 2.8 | POSITIVE | ConfP-100 | 96.1 | YES | TP146 | 2.7 |
| 571204 | Baseline | SP132 | 0.052 | 0.6 | Negative | | | | | |
| 571204 | Month 1 | SP132 | 0.270 | 13.4 | POSITIVE | ConfP-100 | 97.6 | YES | TP144 | 2.7 |
| 571204 | Month 3 | SP132 | 1.202 | 68.0 | POSITIVE | ConfP-100 | 99.4 | YES | TP144 | 3.7 |
| 571204 | Month 6 | SP132 | 0.592 | 32.2 | POSITIVE | ConfP-100 | 98.7 | YES | TP144 | 3.7 |
| 571204 | Month 9 | SP132 | 0.735 | 40.6 | POSITIVE | ConfP-100 | 99.3 | YES | TP144 | 3.7 |
| 571204 | Month 12 | SP132 | 0.630 | 34.5 | POSITIVE | ConfP-100 | 99.0 | YES | TP145 | 3.7 |
| 571204 | Month 18 | SP132 | 0.358 | 18.5 | POSITIVE | ConfP-100 | 98.9 | YES | TP145 | 2.7 |
| 571204 | Month 24 | SP132 | 0.378 | 19.7 | POSITIVE | ConfP-100 | 98.7 | YES | TP145 | 2.7 |
| 571207 | Baseline | SP133 | 0.045 | 0.2 | Negative | | | | | |
| 571207 | Month 1 | SP133 | 0.044 | 0.1 | Negative | | | | | |
| 571207 | Month 3 | SP133 | 0.271 | 12.6 | POSITIVE | ConfP-101 | 99.3 | YES | TP146 | 2.7 |
| 571207 | Month 6 | SP133 | 0.167 | 6.9 | POSITIVE | ConfP-101 | 98.6 | YES | TP146 | 2.7 |
| 571207 | Month 9 | SP133 | 0.146 | 5.7 | POSITIVE | ConfP-101 | 97.5 | YES | TP146 | 2.7 |
| 571207 | Month 12 | SP133 | 0.122 | 4.4 | POSITIVE | ConfP-101 | 97.6 | YES | TP146 | 2.7 |
| 571207 | Month 18 | SP133 | 0.073 | 1.7 | POSITIVE | ConfP-101 | 90.3 | YES | TP146 | 2.7 |
| 571207 | Month 24 | SP133 | 0.060 | 1.0 | Negative | | | | | |
| 571208 | Baseline | SP133 | 0.048 | 0.3 | Negative | | | | | |
| 571208 | Month 1 | SP133 | 0.105 | 3.5 | POSITIVE | ConfP-101 | 96.3 | YES | TP147 | 2.7 |
| 571208 | Month 3 | SP133 | 0.245 | 11.2 | POSITIVE | ConfP-101 | 99.0 | YES | TP147 | 2.7 |
| 571208 | Month 6 | SP133 | 0.079 | 2.0 | POSITIVE | ConfP-101 | 93.8 | YES | TP147 | 2.7 |
| 571208 | Month 9 | SP134 | 0.128 | 5.4 | POSITIVE | ConfP-102 | 99.2 | YES | TP148 | 2.7 |
| 571208 | Month 12 | SP134 | 0.097 | 3.5 | POSITIVE | ConfP-102 | 97.9 | YES | TP148 | 2.7 |
| 571208 | Month 18 | SP134 | 0.067 | 1.6 | POSITIVE | ConfP-102 | 96.2 | YES | TP149 | 2.7 |
| 571208 | Month 24 | SP134 | 0.065 | 1.5 | POSITIVE | ConfP-102 | 100.0 | YES | TP149 | 2.7 |
| 582601 | Baseline | SP134 | 0.047 | 0.3 | Negative | | | | | |
| 582601 | Month 1 | SP134 | 0.341 | 18.9 | POSITIVE | ConfP-101 | 97.6 | YES | TP147 | 2.7 |
| 582601 | Month 3 | SP134 | 0.690 | 41.1 | POSITIVE | ConfP-101 | 98.6 | YES | TP147 | 3.7 |
| 582601 | Month 6 | SP134 | 0.253 | 13.4 | POSITIVE | ConfP-101 | 95.3 | YES | TP147 | 2.7 |
| 582601 | Month 9 | SP134 | 0.234 | 12.2 | POSITIVE | ConfP-101 | 95.0 | YES | TP148 | 2.7 |
| 582601 | Month 12 | SP134 | 0.090 | 3.0 | POSITIVE | ConfP-101 | 86.4 | YES | TP148 | 2.7 |
| 582601 | Month 18 | SP134 | 0.201 | 10.1 | POSITIVE | ConfP-102 | 95.3 | YES | TP148 | 2.7 |
| 582601 | Month 24 | SP134 | 0.180 | 8.7 | POSITIVE | ConfP-102 | 95.6 | YES | TP148 | 2.7 |
| 582603 | Baseline | SP135R | 0.047 | 0.2 | Negative | | | | | |
| 582603 | Month 1 | SP135R | 0.069 | 1.3 | Negative | | | | | |
| 582603 | Month 3 | SP135R | 1.032 | 50.6 | POSITIVE | ConfP-102 | 99.5 | YES | TP149 | 3.7 |
| 582603 | Month 6 | SP135R | 0.448 | 20.7 | POSITIVE | ConfP-102 | 98.8 | YES | TP149 | 3.7 |
| 582603 | Month 9 | SP135R | 0.307 | 13.5 | POSITIVE | ConfP-102 | 99.2 | YES | TP149 | 2.7 |
| 582603 | Month 12 | SP135R | 0.210 | 8.5 | POSITIVE | ConfP-102 | 98.3 | YES | TP149 | 2.7 |
| 582603 | Month 18 | SP135R | 0.109 | 3.4 | POSITIVE | ConfP-102 | 97.0 | YES | TP150 | 2.7 |
| 582603 | Month 24 | SP135R | 0.084 | 2.1 | POSITIVE | ConfP-102 | 95.7 | YES | TP150 | 2.7 |
| 582605 | Baseline | SP135R | 0.056 | 0.7 | Negative | | | | | |
| 582605 | Month 1 | SP135R | 0.704 | 33.8 | POSITIVE | ConfP-102 | 98.5 | YES | TP150 | 3.7 |
| 582605 | Month 3 | SP135R | 0.255 | 10.8 | POSITIVE | ConfP-103 | 95.1 | YES | TP150 | 2.7 |
| 582605 | Month 6 | SP135R | 0.130 | 4.4 | POSITIVE | ConfP-103 | 88.7 | YES | TP150 | 2.7 |
| 582605 | Month 9 | SP136R | 0.109 | 3.5 | POSITIVE | ConfP-103 | 93.4 | YES | TP152 | 2.7 |
| 582605 | Month 12 | SP136R | 0.086 | 2.3 | POSITIVE | ConfP-103 | 87.2 | YES | TP152 | 2.7 |
| 582605 | Month 18 | SP136R | 0.069 | 1.4 | Negative | | | | | |
| 582605 | Month 24 | SP136R | 0.092 | 2.6 | POSITIVE | ConfP-103 | 94.6 | YES | TP152 | 2.7 |
| 582606 | Baseline | SP136R | 0.046 | 0.2 | Negative | | | | | |
| 582606 | Month 1 | SP136R | 0.420 | 19.6 | POSITIVE | ConfP-103 | 99.0 | YES | TP150 | 2.7 |
| 582606 | Month 3 | SP136R | 1.235 | 61.7 | POSITIVE | ConfP-103 | 99.0 | YES | TP151 | 3.7 |
| 582606 | Month 6 | SP136R | 0.445 | 20.8 | POSITIVE | ConfP-103 | 99.0 | YES | TP151 | 2.7 |
| 582606 | Month 9 | SP136R | 0.616 | 29.7 | POSITIVE | ConfP-103 | 99.5 | YES | TP151 | 3.7 |
| 582606 | Month 12 | SP136R | 0.689 | 33.5 | POSITIVE | ConfP-103 | 99.5 | YES | TP151 | 3.7 |
| 582606 | Month 18 | SP136R | 0.323 | 14.5 | POSITIVE | ConfP-103 | 99.5 | YES | TP151 | 2.7 |
| 582606 | Month 24 | SP136R | 0.265 | 11.5 | POSITIVE | ConfP-103 | 99.5 | YES | TP151 | 2.7 |
| 582609 | Baseline | SP137R | 0.061 | 0.8 | Negative | | | | | |
| 582609 | Month 1 | SP137R | 1.335 | 65.0 | POSITIVE | ConfP-103 | 91.4 | YES | TP152 | 3.7 |
| 582609 | Month 3 | SP137R | 1.483 | 72.4 | POSITIVE | ConfP-104 | 98.9 | YES | TP152 | 3.7 |
| 582609 | Month 6 | SP137R | 0.268 | 11.2 | POSITIVE | ConfP-104 | 94.4 | YES | TP152 | 2.7 |
| 582609 | Month 9 | SP137R | 0.341 | 14.9 | POSITIVE | ConfP-104 | 97.4 | YES | TP153 | 2.7 |
| 582609 | Month 12 | SP137R | 0.610 | 28.5 | POSITIVE | ConfP-104 | 98.2 | YES | TP153 | 2.7 |
| 582609 | Month 18 | SP137R | 0.745 | 35.3 | POSITIVE | ConfP-104 | 97.6 | YES | TP153 | 3.7 |
| 582609 | Month 24 | SP137R | 0.932 | 44.7 | POSITIVE | ConfP-104 | 98.5 | YES | TP153 | 3.7 |
| 582611 | Baseline | SP137R | 0.048 | 0.2 | Negative | | | | | |
| 582611 | Month 1 | SP137R | 0.387 | 17.2 | POSITIVE | ConfP-104 | 98.7 | YES | TP153 | 2.7 |
| 582611 | Month 3 | SP137R | 1.241 | 60.3 | POSITIVE | ConfP-104 | 99.6 | YES | TP153 | 3.7 |
| 582611 | Month 6 | SP137R | 0.454 | 20.6 | POSITIVE | ConfP-104 | 99.3 | YES | TP154 | 3.7 |
| 582611 | Month 9 | SP138R | 0.438 | 19.9 | POSITIVE | ConfP-105 | 99.3 | YES | TP155 | 3.7 |
| 582611 | Month 12 | SP138R | 0.255 | 10.7 | POSITIVE | ConfP-105 | 99.6 | YES | TP155 | 3.7 |
| 582611 | Month 18 | SP138R | 0.367 | 16.3 | POSITIVE | ConfP-105 | 98.9 | YES | TP155 | 3.7 |
| 582611 | Month 24 | SP138R | 0.150 | 5.4 | POSITIVE | ConfP-105 | 97.7 | YES | TP155 | 3.7 |
| 582613 | Baseline | SP138R | 0.051 | 0.4 | Negative | | | | | |
| 582613 | Month 1 | SP138R | 0.122 | 4.0 | POSITIVE | ConfP-104 | 92.9 | YES | TP154 | 2.7 |
| 582613 | Month 3 | SP138R | 0.230 | 9.4 | POSITIVE | ConfP-104 | 93.6 | YES | TP154 | 2.7 |
| 582613 | Month 6 | SP138R | 0.099 | 2.8 | POSITIVE | ConfP-104 | 93.6 | YES | TP154 | 2.7 |
| 582613 | Month 9 | SP138R | 0.076 | 1.7 | POSITIVE | ConfP-104 | 86.3 | YES | TP154 | 2.7 |
| 582613 | Month 12 | SP138R | 0.082 | 2.0 | POSITIVE | ConfP-105 | 93.1 | YES | TP154 | 2.7 |
| 582613 | Month 18 | SP138R | 0.066 | 1.2 | Negative | | | | | |
| 582613 | Month 24 | SP138R | 0.074 | 1.6 | POSITIVE | ConfP-105 | 92.7 | YES | TP155 | 2.7 |
| 582614 | Baseline | SP139R | 0.044 | 0.2 | Negative | | | | | |
| 582614 | Month 1 | SP139R | 0.180 | 8.4 | POSITIVE | ConfP-105 | 97.7 | YES | TP155 | 3.7 |
| 582614 | Month 3 | SP139R | 0.485 | 27.0 | POSITIVE | ConfP-105 | 99.3 | YES | TP156 | 3.7 |
| 582614 | Month 6 | SP139R | 0.120 | 4.8 | POSITIVE | ConfP-105 | 97.2 | YES | TP156 | 2.7 |
| 582614 | Month 9 | SP139R | 0.087 | 2.8 | POSITIVE | ConfP-105 | 96.8 | YES | TP156 | 2.7 |
| 582614 | Month 12 | SP139R | 0.109 | 4.1 | POSITIVE | ConfP-105 | 98.6 | YES | TP156 | 2.7 |
| 582614 | Month 18 | SP139R | 0.148 | 6.5 | POSITIVE | ConfP-105 | 98.8 | YES | TP156 | 2.7 |
| 582614 | Month 24 | SP139R | 0.085 | 2.7 | POSITIVE | ConfP-105 | 94.5 | YES | TP156 | 2.7 |
| 582615 | Baseline | SP139R | 0.044 | 0.2 | Negative | | | | | |
| 582615 | Month 1 | SP139R | 0.146 | 6.4 | POSITIVE | ConfP-106 | 98.6 | YES | TP157 | 3.7 |
| 582615 | Month 3 | SP139R | 0.387 | 21.0 | POSITIVE | ConfP-106 | 99.3 | YES | TP157 | 2.7 |
| 582615 | Month 6 | SP139R | 0.206 | 10.0 | POSITIVE | ConfP-106 | 99.6 | YES | TP157 | 2.7 |
| 582615 | Month 9 | SP140R | 0.287 | 15.1 | POSITIVE | ConfP-106 | 98.9 | YES | TP158 | 2.7 |
| 582615 | Month 12 | SP140R | 0.143 | 6.3 | POSITIVE | ConfP-106 | 98.4 | YES | TP158 | 2.7 |
| 582615 | Month 18 | SP140R | 0.127 | 5.4 | POSITIVE | ConfP-106 | 98.4 | YES | TP159 | 2.7 |
| 582615 | Month 24 | SP140R | 0.139 | 6.1 | POSITIVE | ConfP-107 | 97.6 | YES | TP159 | 2.7 |
| 582802 | Baseline | SP193 | 0.096 | 1.1 | Negative | | | | | |
| 582802 | Month 1 | SP193 | 2.023 | 77.2 | POSITIVE | ConfP-142 | 99.6 | YES | TP306 | 3.7 |
| 582802 | Month 3 | SP193 | 1.789 | 67.9 | POSITIVE | ConfP-142 | 99.5 | YES | TP306 | 3.7 |
| 582802 | Month 6 | SP193 | 1.409 | 52.9 | POSITIVE | ConfP-142 | 99.1 | YES | TP306 | 3.7 |
| 582802 | Month 9 | No sample received | | | | | | | | |
| 582802 | Month 12 | SP193 | 1.201 | 44.7 | POSITIVE | ConfP-142 | 99.3 | YES | TP306 | 2.7 |
| 582802 | Month 18 | SP193 | 1.010 | 37.2 | POSITIVE | ConfP-142 | 99.1 | YES | TP306 | 2.7 |
| 582802 | Month 24 | SP196 | 0.451 | 16.1 | POSITIVE | ConfP-142 | 97.9 | YES | TP306 | 2.7 |
| 582804 | Baseline | SP194 | 0.095 | 1.4 | Negative | | | | | |
| 582804 | Month 1 | SP194 | 0.707 | 25.7 | POSITIVE | ConfP-143 | 98.6 | YES | TP307 | 2.7 |
| 582804 | Month 3 | SP194 | 1.682 | 64.5 | POSITIVE | ConfP-143 | 99.9 | YES | TP307 | 3.7 |
| 582804 | Month 6 | SP194 | 0.794 | 29.2 | POSITIVE | ConfP-143 | 99.8 | YES | TP307 | 2.7 |
| 582804 | Month 9 | No sample received | | | | | | | | |
| 582804 | Month 12 | SP194 | 0.645 | 23.3 | POSITIVE | ConfP-143 | 100.0 | YES | TP307 | 2.7 |
| 582804 | Month 18 | SP194 | 0.868 | 32.1 | POSITIVE | ConfP-143 | 98.7 | YES | TP307 | 2.7 |
| 582804 | Month 24 | SP194 | 0.997 | 37.3 | POSITIVE | ConfP-143 | 99.0 | YES | TP307 | 2.7 |
| 582807 | Baseline | SP140R | 0.044 | 0.3 | Negative | | | | | |
| 582807 | Month 1 | SP140R | 1.951 | 116.4 | POSITIVE | ConfP-106 | 98.9 | YES | TP157 | 4.7 |
| 582807 | Month 3 | SP140R | 1.730 | 103.0 | POSITIVE | ConfP-106 | 98.9 | YES | TP157 | 4.7 |
| 582807 | Month 6 | SP140R | 0.924 | 53.9 | POSITIVE | ConfP-106 | 98.9 | YES | TP157 | 3.7 |
| 582807 | Month 9 | SP140R | 0.565 | 32.0 | POSITIVE | ConfP-106 | 98.9 | YES | TP158 | 3.7 |
| 582807 | Month 12 | SP140R | 0.590 | 33.6 | POSITIVE | ConfP-106 | 99.4 | YES | TP158 | 3.7 |
| 582807 | Month 18 | SP140R | 0.536 | 30.3 | POSITIVE | ConfP-106 | 99.1 | YES | TP158 | 3.7 |
| 582807 | Month 24 | SP140R | 0.483 | 27.0 | POSITIVE | ConfP-106 | 98.6 | YES | TP158 | 3.7 |
| 582810 | Baseline | SP198 | 0.092 | 0.5 | Negative | | | | | |
| 582810 | Month 1 | SP198 | 1.619 | 79.4 | POSITIVE | ConfP-146 | 100.0 | YES | TP313 | 3.7 |
| 582810 | Month 3 | SP198 | 1.559 | 76.3 | POSITIVE | ConfP-146 | 100.0 | YES | TP313 | 3.7 |
| 582810 | Month 6 | SP198 | 1.086 | 51.8 | POSITIVE | ConfP-146 | 100.0 | YES | TP313 | 2.7 |
| 582810 | Month 9 | SP198 | 0.843 | 39.3 | POSITIVE | ConfP-146 | 100.0 | YES | TP313 | 2.7 |
| 582810 | Month 12 | SP198 | 0.776 | 35.8 | POSITIVE | ConfP-146 | 100.0 | YES | TP313 | 2.7 |
| 582810 | Month 18 | No sample received | | | | | | | | |
| 582810 | Month 24 | SP199 | 0.358 | 15.5 | POSITIVE | ConfP-146 | 100.0 | YES | TP313 | 2.7 |
| 582811 | Baseline | SP141R | 0.049 | 0.4 | Negative | | | | | |
| 582811 | Month 1 | SP141R | 0.599 | 33.4 | POSITIVE | ConfP-107 | 99.6 | YES | TP159 | 3.7 |
| 582811 | Month 3 | SP141R | 1.334 | 77.4 | POSITIVE | ConfP-107 | 99.5 | YES | TP159 | 3.7 |
| 582811 | Month 6 | SP141R | 0.872 | 49.7 | POSITIVE | ConfP-107 | 99.5 | YES | TP159 | 3.7 |
| 582811 | Month 9 | SP141R | 0.450 | 24.4 | POSITIVE | ConfP-107 | 98.8 | YES | TP159 | 2.7 |
| 582811 | Month 12 | SP141R | 0.443 | 24.0 | POSITIVE | ConfP-107 | 98.6 | YES | TP160 | 3.7 |
| 582811 | Month 18 | SP141R | 0.375 | 20.0 | POSITIVE | ConfP-107 | 98.4 | YES | TP160 | 3.7 |
| 582811 | Month 24 | SP141R | 0.228 | 11.1 | POSITIVE | ConfP-107 | 97.6 | YES | TP160 | 2.7 |
| 582812 | Baseline | SP191 | 0.083 | 0.5 | Negative | | | | | |
| 582812 | Month 1 | SP191 | 0.808 | 29.5 | POSITIVE | ConfP-136 | 100.0 | YES | TP312 | 2.7 |
| 582812 | Month 3 | SP191 | 2.054 | 79.4 | POSITIVE | ConfP-136 | 99.6 | YES | TP312 | 3.7 |
| 582812 | Month 6 | No sample received | | | | | | | | |
| 582812 | Month 9 | SP191 | 0.886 | 32.6 | POSITIVE | ConfP-136 | 99.6 | YES | TP312 | 2.7 |
| 582812 | Month 12 | SP191 | 0.620 | 22.0 | POSITIVE | ConfP-136 | 100.0 | YES | TP312 | 2.7 |
| 582812 | Month 18 | SP191 | 0.609 | 21.5 | POSITIVE | ConfP-136 | 98.8 | YES | TP312 | 2.7 |
| 582812 | Month 24 | SP196 | 1.373 | 54.2 | POSITIVE | ConfP-136 | 99.7 | YES | TP312 | 2.7 |
| 582902 | Baseline | SP141R | 0.051 | 0.5 | Negative | | | | | |
| 582902 | Month 1 | SP141R | 0.416 | 22.4 | POSITIVE | ConfP-107 | 99.3 | YES | TP160 | 2.7 |
| 582902 | Month 3 | SP141R | 1.944 | 114.0 | POSITIVE | ConfP-107 | 97.8 | YES | TP160 | 4.7 |
| 582902 | Month 6 | SP141R | 1.050 | 60.4 | POSITIVE | ConfP-107 | 98.3 | YES | TP160 | 3.7 |
| 582902 | Month 9 | SP142R | 0.404 | 22.2 | POSITIVE | ConfP-108 | 98.0 | YES | TP162 | 2.7 |
| 582902 | Month 12 | SP142R | 0.322 | 17.2 | POSITIVE | ConfP-108 | 98.6 | YES | TP162 | 2.7 |
| 582902 | Month 18 | SP142R | 0.435 | 24.1 | POSITIVE | ConfP-108 | 97.5 | YES | TP162 | 2.7 |
| 582902 | Month 24 | SP142R | 0.203 | 9.9 | POSITIVE | ConfP-108 | 97.8 | YES | TP162 | 2.7 |
| 582906 | Baseline | SP142R | 0.044 | 0.1 | Negative | | | | | |
| 582906 | Month 1 | SP142R | 0.090 | 2.9 | POSITIVE | ConfP-107 | 93.9 | YES | TP161 | 2.7 |
| 582906 | Month 3 | SP142R | 0.491 | 27.5 | POSITIVE | ConfP-107 | 99.4 | YES | TP161 | 3.7 |
| 582906 | Month 6 | SP142R | 0.210 | 10.3 | POSITIVE | ConfP-108 | 98.2 | YES | TP161 | 2.7 |
| 582906 | Month 9 | SP142R | 0.127 | 5.2 | POSITIVE | ConfP-108 | 98.5 | YES | TP161 | 2.7 |
| 582906 | Month 12 | SP142R | 0.062 | 1.2 | Negative | | | | | |
| 582906 | Month 18 | SP142R | 0.130 | 5.4 | POSITIVE | ConfP-108 | 98.4 | YES | TP161 | 2.7 |
| 582906 | Month 24 | SP142R | 0.084 | 2.6 | POSITIVE | ConfP-108 | 95.6 | YES | TP161 | 2.7 |
| 582907 | Baseline | SP143R | 0.050 | 0.5 | Negative | | | | | |
| 582907 | Month 1 | SP143R | 0.096 | 3.4 | POSITIVE | ConfP-108 | 93.3 | YES | TP162 | 2.7 |
| 582907 | Month 3 | SP143R | 0.314 | 17.1 | POSITIVE | ConfP-108 | 98.7 | YES | TP162 | 2.7 |
| 582907 | Month 6 | SP143R | 0.098 | 3.5 | POSITIVE | ConfP-108 | 95.8 | YES | TP163 | 2.7 |
| 582907 | Month 9 | SP143R | 0.059 | 1.1 | Negative | | | | | |
| 582907 | Month 12 | SP143R | 0.125 | 5.2 | POSITIVE | ConfP-108 | 98.5 | YES | TP163 | 2.7 |
| 582907 | Month 18 | SP143R | 0.056 | 0.9 | Negative | | | | | |
| 582907 | Month 24 | SP143R | 0.074 | 2.0 | POSITIVE | ConfP-108 | 87.5 | YES | TP163 | 2.7 |
| 582908 | Baseline | SP143R | 0.049 | 0.4 | Negative | | | | | |
| 582908 | Month 1 | SP143R | 0.092 | 3.1 | POSITIVE | ConfP-109 | 98.5 | YES | TP163 | 2.7 |
| 582908 | Month 3 | SP143R | 0.522 | 28.2 | POSITIVE | ConfP-109 | 99.8 | YES | TP163 | 3.7 |
| 582908 | Month 6 | SP143R | 0.480 | 27.6 | POSITIVE | ConfP-109 | 99.8 | YES | TP163 | 2.7 |
| 582908 | Month 9 | SP144R | 0.153 | 6.8 | POSITIVE | ConfP-109 | 99.3 | YES | TP165 | 2.7 |
| 582908 | Month 12 | SP144R | 0.088 | 2.8 | POSITIVE | ConfP-109 | 92.7 | YES | TP165 | 2.7 |
| 582908 | Month 18 | SP144R | 0.074 | 2.0 | POSITIVE | ConfP-110 | 86.2 | YES | TP165 | 2.7 |
| 582908 | Month 24 | SP144R | 0.059 | 1.0 | Negative | | | | | |
| 582911 | Baseline | SP144R | 0.068 | 1.6 | POSITIVE | ConfP-109 | 86.4 | YES | TP164 | 2.7 |
| 583911 | Month 1 | SP144R | 0.117 | 4.6 | POSITIVE | ConfP-109 | 95.6 | YES | TP164 | 2.7 |
| 582911 | Month 3 | SP144R | 1.322 | 78.0 | POSITIVE | ConfP-109 | 99.7 | YES | TP164 | 3.7 |
| 582911 | Month 6 | SP144R | 1.322 | 78.0 | POSITIVE | ConfP-109 | 99.5 | YES | TP164 | 3.7 |
| 582911 | Month 9 | SP144R | 0.306 | 16.1 | POSITIVE | ConfP-109 | 98.7 | YES | TP164 | 2.7 |
| 582911 | Month 12 | SP144R | 0.920 | 53.5 | POSITIVE | ConfP-109 | 99.8 | YES | TP164 | 3.7 |
| 582911 | Month 18 | SP144R | 0.177 | 8.2 | POSITIVE | ConfP-109 | 99.4 | YES | TP165 | 2.7 |
| 582911 | Month 24 | SP144R | 0.526 | 29.5 | POSITIVE | ConfP-109 | 99.6 | YES | TP165 | 2.7 |
| 582917 | Baseline | SP145 | 0.048 | 0.3 | Negative | | | | | |
| 582917 | Month 1 | SP145 | 1.211 | 61.8 | POSITIVE | ConfP-110 | 99.3 | YES | TP165 | 3.7 |
| 582917 | Month 3 | SP145 | 1.641 | 84.5 | POSITIVE | ConfP-110 | 99.5 | YES | TP166 | 3.7 |
| 582917 | Month 6 | SP145 | 1.187 | 60.5 | POSITIVE | ConfP-110 | 99.0 | YES | TP166 | 3.7 |
| 582917 | Month 9 | SP145 | 1.807 | 93.3 | POSITIVE | ConfP-110 | 98.9 | YES | TP166 | 3.7 |
| 582917 | Month 12 | SP145 | 1.861 | 96.1 | POSITIVE | ConfP-110 | 99.4 | YES | TP166 | 3.7 |
| 582917 | Month 18 | SP145 | 0.357 | 16.6 | POSITIVE | ConfP-110 | 97.9 | YES | TP166 | 2.7 |
| 582917 | Month 24 | SP145 | 0.232 | 10.0 | POSITIVE | ConfP-110 | 95.8 | YES | TP288 | 2.7 |
| 583101 | Baseline | SP145 | 0.049 | 0.4 | Negative | | | | | |
| 583101 | Month 1 | SP145 | 1.359 | 69.6 | POSITIVE | ConfP-110 | 99.8 | YES | TP167 | 3.7 |
| 583101 | Month 3 | SP145 | 1.176 | 59.9 | POSITIVE | ConfP-110 | 99.7 | YES | TP167 | 3.7 |
| 583101 | Month 6 | SP145 | 1.061 | 53.9 | POSITIVE | ConfP-110 | 99.8 | YES | TP167 | 3.7 |
| 583101 | Month 9 | SP146 | 0.361 | 17.7 | POSITIVE | ConfP-111 | 99.2 | YES | TP168 | 3.7 |
| 583101 | Month 12 | SP146 | 0.577 | 29.7 | POSITIVE | ConfP-111 | 99.5 | YES | TP169 | 3.7 |
| 583101 | Month 18 | SP146 | 1.063 | 56.7 | POSITIVE | ConfP-111 | 99.5 | YES | TP169 | 2.7 |
| 583101 | Month 24 | SP146 | 0.430 | 21.6 | POSITIVE | ConfP-111 | 99.3 | YES | TP169 | 2.7 |
| 583104 | Baseline | SP146 | 0.083 | 2.3 | POSITIVE | ConfP-110 | 82.5 | YES | TP167 | 2.7 |
| 583104 | Month 1 | SP146 | 0.604 | 31.2 | POSITIVE | ConfP-110 | 98.9 | YES | TP167 | 2.7 |
| 583104 | Month 3 | SP146 | 1.985 | 107.8 | POSITIVE | ConfP-111 | 99.1 | YES | TP167 | 3.7 |
| 583104 | Month 6 | SP146 | 0.759 | 39.8 | POSITIVE | ConfP-111 | 97.7 | YES | TP168 | 3.7 |
| 583104 | Month 9 | SP146 | 0.122 | 4.5 | POSITIVE | ConfP-111 | 94.0 | YES | TP168 | 2.7 |
| 583104 | Month 12 | SP146 | 0.099 | 3.2 | POSITIVE | ConfP-111 | 87.9 | YES | TP168 | 2.7 |
| 583104 | Month 18 | SP146 | 0.648 | 33.7 | POSITIVE | ConfP-111 | 98.0 | YES | TP168 | 3.7 |
| 583104 | Month 24 | SP146 | 0.326 | 15.8 | POSITIVE | ConfP-111 | 95.0 | YES | TP168 | 3.7 |
| 583105 | Baseline | SP147 | 0.045 | 0.1 | Negative | | | | | |
| 583105 | Month 1 | SP147 | 0.675 | 34.0 | POSITIVE | ConfP-111 | 99.6 | YES | TP169 | 3.7 |
| 583105 | Month 3 | SP147 | 2.489 | 131.7 | POSITIVE | ConfP-111 | 99.7 | YES | TP169 | 4.7 |
| 583105 | Month 6 | SP147 | 1.752 | 92.0 | POSITIVE | ConfP-111 | 99.8 | YES | TP169 | 3.7 |
| 583105 | Month 9 | SP147 | 1.047 | 54.0 | POSITIVE | ConfP-112 | 99.3 | YES | TP170 | 3.7 |
| 583105 | Month 12 | SP147 | 0.932 | 47.8 | POSITIVE | ConfP-112 | 99.5 | YES | TP170 | 3.7 |
| 583105 | Month 18 | SP147 | 0.493 | 24.2 | POSITIVE | ConfP-112 | 99.3 | YES | TP170 | 3.7 |
| 583105 | Month 24 | SP147 | 0.330 | 15.4 | POSITIVE | ConfP-112 | 98.2 | YES | TP170 | 2.7 |
| 583601 | Baseline | SP147 | 0.047 | 0.2 | Negative | | | | | |
| 583601 | Month 1 | SP147 | 0.277 | 12.5 | POSITIVE | ConfP-112 | 95.1 | YES | TP170 | 2.7 |
| 583601 | Month 3 | SP147 | 1.553 | 81.3 | POSITIVE | ConfP-112 | 98.1 | YES | TP170 | 3.7 |
| 583601 | Month 6 | SP147 | 1.006 | 51.8 | POSITIVE | ConfP-112 | 97.7 | YES | TP171 | 3.7 |
| 583601 | Month 9 | SP148 | 0.675 | 33.4 | POSITIVE | ConfP-113 | 97.8 | YES | TP172 | 2.7 |
| 583601 | Month 12 | SP148 | 0.298 | 13.5 | POSITIVE | ConfP-113 | 95.5 | YES | TP172 | 2.7 |
| 583601 | Month 18 | SP148 | 0.208 | 8.8 | POSITIVE | ConfP-113 | 94.7 | YES | TP172 | 2.7 |
| 583601 | Month 24 | SP148 | 0.119 | 4.1 | POSITIVE | ConfP-113 | 86.4 | YES | TP172 | 2.7 |
| 583603 | Baseline | SP148 | 0.047 | 0.3 | Negative | | | | | |
| 583603 | Month 1 | SP148 | 0.098 | 3.0 | POSITIVE | ConfP-112 | 93.0 | YES | TP171 | 2.7 |
| 583603 | Month 3 | SP148 | 0.133 | 4.8 | POSITIVE | ConfP-112 | 97.2 | YES | TP171 | 2.7 |
| 583603 | Month 6 | SP148 | 0.119 | 4.1 | POSITIVE | ConfP-112 | 97.6 | YES | TP171 | 2.7 |
| 583603 | Month 9 | SP148 | 0.175 | 7.1 | POSITIVE | ConfP-112 | 98.9 | YES | TP171 | 2.7 |
| 583603 | Month 12 | SP148 | 0.253 | 11.2 | POSITIVE | ConfP-112 | 99.3 | YES | TP171 | 2.7 |
| 583603 | Month 18 | SP148 | 0.222 | 9.5 | POSITIVE | ConfP-112 | 98.7 | YES | TP172 | 2.7 |
| 583603 | Month 24 | SP148 | 0.185 | 7.6 | POSITIVE | ConfP-113 | 98.6 | YES | TP172 | 2.7 |
| 583604 | Baseline | SP149 | 0.044 | 0.0 | Negative | | | | | |
| 583604 | Month 1 | SP149 | 0.288 | 16.7 | POSITIVE | ConfP-113 | 98.1 | YES | TP173 | 2.7 |
| 583604 | Month 3 | SP149 | 0.566 | 35.8 | POSITIVE | ConfP-113 | 99.4 | YES | TP173 | 3.7 |
| 583604 | Month 6 | SP149 | 0.441 | 27.2 | POSITIVE | ConfP-113 | 98.9 | YES | TP173 | 2.7 |
| 583604 | Month 9 | SP149 | 0.222 | 12.2 | POSITIVE | ConfP-113 | 98.0 | YES | TP173 | 2.7 |
| 583604 | Month 12 | SP149 | 0.224 | 12.3 | POSITIVE | ConfP-113 | 97.0 | YES | TP173 | 2.7 |
| 583604 | Month 18 | SP149 | 0.091 | 3.2 | POSITIVE | ConfP-113 | 96.2 | YES | TP174 | 2.7 |
| 583604 | Month 24 | SP149 | 0.137 | 6.4 | POSITIVE | ConfP-113 | 98.3 | YES | TP174 | 2.7 |
| 583701 | Baseline | SP32 | 0.047 | 0.1 | Negative | | | | | |
| 583701 | Month 1 | SP32 | 0.062 | 1.2 | Negative | | | | | |
| 583701 | Month 3 | SP32 | 0.327 | 20.0 | POSITIVE | ConfP-4 | 100.0 | YES | TP12 | 2.7 |
| 583701 | Month 6 | SP32 | 0.075 | 2.1 | POSITIVE | ConfP-26 | 96.9 | YES | TP253 | 2.7 |
| 583701 | Month 9 | SP32 | 0.188 | 10.1 | POSITIVE | ConfP-26 | 99.2 | YES | TP254 | 2.7 |
| 583701 | Month 12 | SP32 | 0.074 | 2.1 | POSITIVE | ConfP-26 | 85.0 | YES | TP254 | 2.7 |
| 583701 | Month 18 | SP32 | 0.315 | 19.1 | POSITIVE | ConfP-27 | 99.7 | YES | TP254 | 2.7 |
| 583701 | Month 24 | SP32 | 0.115 | 5.0 | POSITIVE | ConfP-27 | 98.4 | YES | TP254 | 2.7 |
| 583703 | Baseline | SP149 | 0.045 | 0.1 | Negative | | | | | |
| 583703 | Month 1 | SP149 | 0.112 | 4.7 | POSITIVE | ConfP-114 | 97.6 | YES | TP174 | 2.7 |
| 583703 | Month 3 | SP149 | 0.276 | 15.9 | POSITIVE | ConfP-114 | 99.2 | YES | TP174 | 2.7 |
| 583703 | Month 6 | SP149 | 0.149 | 7.2 | POSITIVE | ConfP-114 | 99.2 | YES | TP174 | 2.7 |
| 583703 | Month 9 | SP150 | 0.206 | 9.9 | POSITIVE | ConfP-114 | 97.9 | YES | TP176 | 3.7 |
| 583703 | Month 12 | SP150 | 0.115 | 4.3 | POSITIVE | ConfP-114 | 96.3 | YES | TP176 | 2.7 |
| 583703 | Month 18 | SP150 | 0.115 | 4.3 | POSITIVE | ConfP-114 | 97.4 | YES | TP176 | 2.7 |
| 583703 | Month 24 | SP150 | 0.089 | 2.8 | POSITIVE | ConfP-115 | 93.0 | YES | TP176 | 2.7 |
| 583706 | Baseline | SP150 | 0.045 | 0.1 | Negative | | | | | |
| 583706 | Month 1 | SP150 | 0.374 | 20.2 | POSITIVE | ConfP-114 | 97.4 | YES | TP174 | 2.7 |
| 583706 | Month 3 | SP150 | 0.545 | 30.7 | POSITIVE | ConfP-114 | 98.4 | YES | TP175 | 3.7 |
| 583706 | Month 6 | SP150 | 0.389 | 21.1 | POSITIVE | ConfP-114 | 98.1 | YES | TP175 | 3.7 |
| 583706 | Month 9 | SP150 | 0.239 | 11.9 | POSITIVE | ConfP-114 | 97.4 | YES | TP175 | 4.7 |
| 583706 | Month 12 | SP150 | 0.389 | 21.1 | POSITIVE | ConfP-114 | 98.4 | YES | TP175 | 3.7 |
| 583706 | Month 18 | SP150 | 0.251 | 12.7 | POSITIVE | ConfP-114 | 97.2 | YES | TP175 | 3.7 |
| 583706 | Month 24 | SP150 | 0.255 | 12.9 | POSITIVE | ConfP-114 | 97.9 | YES | TP175 | 3.7 |
| 583708 | Baseline | SP151 | 0.067 | 1.4 | Negative | | | | | |
| 583708 | Month 1 | SP151 | 1.316 | 71.4 | POSITIVE | ConfP-115 | 98.9 | YES | TP176 | 3.7 |
| 583708 | Month 3 | SP151 | 0.923 | 49.4 | POSITIVE | ConfP-115 | 99.0 | YES | TP176 | 3.7 |
| 583708 | Month 6 | SP151 | 1.062 | 57.1 | POSITIVE | ConfP-115 | 99.3 | YES | TP177 | 3.7 |
| 583708 | Month 9 | SP151 | 0.246 | 11.4 | POSITIVE | ConfP-115 | 96.8 | YES | TP177 | 2.7 |
| 583708 | Month 12 | SP151 | 0.320 | 15.6 | POSITIVE | ConfP-115 | 98.1 | YES | TP177 | 2.7 |
| 583708 | Month 18 | SP151 | 0.691 | 36.4 | POSITIVE | ConfP-115 | 99.0 | YES | TP177 | 3.7 |
| 583708 | Month 24 | SP151 | 0.840 | 44.7 | POSITIVE | ConfP-115 | 98.8 | YES | TP177 | 3.7 |
| 600902 | Baseline | SP33 | 0.044 | 0.0 | Negative | | | | | |
| 600902 | Month 1 | SP33 | 0.123 | 4.8 | POSITIVE | ConfP-27 | 98.8 | YES | TP254 | 2.7 |
| 600902 | Month 3 | SP33 | 1.054 | 62.0 | POSITIVE | ConfP-4 | 100.0 | YES | TP12 | 3.7 |
| 600902 | Month 6 | SP33 | 0.370 | 20.0 | POSITIVE | ConfP-27 | 99.2 | YES | TP254 | 2.7 |
| 600902 | Month 9 | SP33 | 0.318 | 16.8 | POSITIVE | ConfP-27 | 99.5 | YES | TP255 | 3.7 |
| 600902 | Month 12 | SP33 | 0.200 | 9.6 | POSITIVE | ConfP-27 | 99.4 | YES | TP255 | 2.7 |
| 600902 | Month 18 | SP33 | 0.289 | 15.0 | POSITIVE | ConfP-27 | 99.2 | YES | TP255 | 2.7 |
| 600902 | Month 24 | SP33 | 0.210 | 10.2 | POSITIVE | ConfP-27 | 99.0 | YES | TP255 | 2.7 |
| 600904 | Baseline | SP33 | 0.043 | 0.0 | Negative | | | | | |
| 600904 | Month 1 | SP33 | 0.445 | 24.6 | POSITIVE | ConfP-27 | 99.4 | YES | TP255 | 3.7 |
| 600904 | Month 3 | SP33 | 1.101 | 64.9 | POSITIVE | ConfP-4 | 99.9 | YES | TP12 | 3.7 |
| 600904 | Month 6 | SP33 | 0.873 | 50.9 | POSITIVE | ConfP-27 | 99.3 | YES | TP255 | 3.7 |
| 600904 | Month 9 | SP34 | 0.604 | 35.6 | POSITIVE | ConfP-27 | 99.3 | YES | TP256 | 3.7 |
| 600904 | Month 12 | SP34 | 0.452 | 26.0 | POSITIVE | ConfP-27 | 99.2 | YES | TP256 | 3.7 |
| 600904 | Month 18 | SP34 | 0.359 | 20.1 | POSITIVE | ConfP-27 | 98.7 | YES | TP256 | 3.7 |
| 600904 | Month 24 | SP34 | 0.370 | 20.8 | POSITIVE | ConfP-28 | 99.7 | YES | TP256 | 3.7 |
| 600905 | Baseline | SP34 | 0.043 | 0.0 | Negative | | | | | |
| 600905 | Month 1 | SP34 | 1.080 | 65.9 | POSITIVE | ConfP-28 | 99.7 | YES | TP256 | 4.7 |
| 600905 | Month 3 | SP34 | 1.300 | 79.9 | POSITIVE | ConfP-4 | 100.0 | YES | TP13 | 3.7 |
| 600905 | Month 6 | SP34 | 0.324 | 17.9 | POSITIVE | ConfP-28 | 99.0 | YES | TP256 | 3.7 |
| 600905 | Month 9 | SP34 | 0.314 | 17.2 | POSITIVE | ConfP-28 | 98.6 | YES | TP257 | 3.7 |
| 600905 | Month 12 | SP34 | 0.279 | 15.0 | POSITIVE | ConfP-28 | 98.7 | YES | TP257 | 2.7 |
| 600905 | Month 18 | SP34 | 0.597 | 35.2 | POSITIVE | ConfP-28 | 96.7 | YES | TP257 | 3.7 |
| 600905 | Month 24 | SP34 | 0.129 | 5.5 | POSITIVE | ConfP-28 | 93.7 | YES | TP257 | 2.7 |
| 600906 | Baseline | SP35 | 0.052 | 0.6 | Negative | | | | | |
| 600906 | Month 1 | SP35 | 0.111 | 4.1 | POSITIVE | ConfP-28 | 99.2 | YES | TP257 | 3.7 |
| 600906 | Month 3 | SP35 | 0.904 | 51.4 | POSITIVE | ConfP-4 | 99.3 | YES | TP13 | 3.7 |
| 600906 | Month 6 | SP35 | 0.519 | 28.4 | POSITIVE | ConfP-28 | 99.2 | YES | TP257 | 3.7 |
| 600906 | Month 9 | SP35 | 0.302 | 15.5 | POSITIVE | ConfP-28 | 99.2 | YES | TP258 | 2.7 |
| 600906 | Month 12 | SP35 | 0.266 | 13.4 | POSITIVE | ConfP-28 | 99.2 | YES | TP258 | 2.7 |
| 600906 | Month 18 | SP35 | 0.293 | 15.0 | POSITIVE | ConfP-28 | 99.4 | YES | TP258 | 3.7 |
| 600906 | Month 24 | SP35 | 0.241 | 11.9 | POSITIVE | ConfP-28 | 99.3 | YES | TP258 | 2.7 |
| 600909 | Baseline | SP35 | 0.043 | 0.1 | Negative | | | | | |
| 600909 | Month 1 | SP35 | 0.838 | 47.5 | POSITIVE | ConfP-4 | 100.0 | YES | TP13 | 3.7 |
| 600909 | Month 3 | SP35 | 0.152 | 6.6 | POSITIVE | ConfP-29 | 98.9 | YES | TP258 | 2.7 |
| 600909 | Month 6 | SP35 | 0.204 | 9.7 | POSITIVE | ConfP-29 | 98.1 | YES | TP258 | 2.7 |
| 600909 | Month 9 | SP36 | 0.201 | 9.1 | POSITIVE | ConfP-29 | 99.5 | YES | TP259 | 2.7 |
| 600909 | Month 12 | SP36 | 0.117 | 4.3 | POSITIVE | ConfP-29 | 99.2 | YES | TP259 | 2.7 |
| 600909 | Month 18 | SP36 | 0.344 | 17.3 | POSITIVE | ConfP-29 | 99.7 | YES | TP259 | 3.7 |
| 600909 | Month 24 | SP36 | 0.121 | 4.5 | POSITIVE | ConfP-29 | 99.3 | YES | TP259 | 2.7 |
| 600914 | Baseline | SP36 | 0.174 | 7.5 | POSITIVE | ConfP-29 | 12.4 | No | | |
| 600914 | Month 1 | SP36 | 0.322 | 16.0 | POSITIVE | ConfP-29 | 63.8 | YES | TP259 | 2.7 |
| 600914 | Month 3 | SP36 | 0.403 | 20.7 | POSITIVE | ConfP-4 | 100.0 | YES | TP13 | 2.7 |
| 600914 | Month 6 | SP36 | 0.225 | 10.5 | POSITIVE | ConfP-29 | 61.1 | YES | TP260 | 2.7 |
| 600914 | Month 9 | SP36 | 0.198 | 8.9 | POSITIVE | ConfP-29 | 56.8 | YES | TP260 | 2.7 |
| 600914 | Month 12 | SP36 | 0.217 | 10.0 | POSITIVE | ConfP-29 | 58.5 | YES | TP260 | 2.7 |
| 600914 | Month 18 | SP36 | 0.283 | 13.8 | POSITIVE | ConfP-29 | 60.2 | YES | TP260 | 2.7 |
| 600914 | Month 24 | SP36 | 0.302 | 14.9 | POSITIVE | ConfP-29 | 72.6 | YES | TP260 | 2.7 |
| 600915 | Baseline | SP37 | 0.044 | 0.1 | Negative | | | | | |
| 600915 | Month 1 | SP37 | 0.294 | 22.4 | POSITIVE | ConfP-30 | 99.5 | YES | TP260 | 3.7 |
| 600915 | Month 3 | SP37 | 0.957 | 81.5 | POSITIVE | ConfP-5 | 100.0 | YES | TP1 | 3.7 |
| 600915 | Month 6 | SP37 | 0.146 | 9.2 | POSITIVE | ConfP-30 | 99.6 | YES | TP261 | 2.7 |
| 600915 | Month 9 | SP37 | 0.472 | 38.3 | POSITIVE | ConfP-30 | 99.7 | YES | TP261 | 3.7 |
| 600915 | Month 12 | SP37 | 0.493 | 40.1 | POSITIVE | ConfP-30 | 99.8 | YES | TP261 | 3.7 |
| 600915 | Month 18 | SP37 | 0.141 | 8.7 | POSITIVE | ConfP-30 | 99.6 | YES | TP261 | 2.7 |
| 600915 | Month 24 | SP37 | 0.182 | 12.4 | POSITIVE | ConfP-30 | 99.3 | YES | TP261 | 2.7 |
| 600919 | Baseline | SP37 | 0.056 | 1.2 | Negative | | | | | |
| 600919 | Month 1 | SP37 | 0.268 | 20.1 | POSITIVE | ConfP-30 | 76.7 | YES | TP261 | 3.7 |
| 600919 | Month 3 | SP37 | 1.507 | 130.6 | POSITIVE | ConfP-5 | 99.3 | YES | TP1 | 3.7 |
| 600919 | Month 6 | SP37 | 0.454 | 36.7 | POSITIVE | ConfP-30 | 88.2 | YES | TP262 | 3.7 |
| 600919 | Month 9 | SP38 | 0.456 | 34.8 | POSITIVE | ConfP-30 | 91.8 | YES | TP262 | 2.7 |
| 600919 | Month 12 | SP38 | 0.677 | 53.4 | POSITIVE | ConfP-30 | 95.1 | YES | TP262 | 3.7 |
| 600919 | Month 18 | SP38 | 0.230 | 15.8 | POSITIVE | ConfP-30 | 89.0 | YES | TP262 | 2.7 |
| 600919 | Month 24 | SP38 | 0.227 | 15.5 | POSITIVE | ConfP-30 | 91.6 | YES | TP262 | 2.7 |
| 600923 | Baseline | ConfP- | 0.082 | 0.3 | Negative | | | | | |
| | | 147 | | | | | | | | |
| 600923 | Baseline | ConfP- | 0.086 | 0.6 | Negative | | | | | |
| | | 147 | | | | | | | | |
| 600923 | Month 1 | SP194 | 0.932 | 34.7 | POSITIVE | ConfP-143 | 99.3 | YES | TP311 | 2.7 |
| 600923 | Month 3 | SP194 | 2.378 | 92.1 | POSITIVE | ConfP-147 | 99.5 | YES | TP311 | 3.7 |
| 600923 | Month 6 | SP194 | 0.934 | 34.7 | POSITIVE | ConfP-143 | 99.1 | YES | TP311 | 2.7 |
| 600923 | Month 9 | No sample received | | | | | | | | |
| 600923 | Month 12 | SP194 | 0.324 | 10.5 | POSITIVE | ConfP-143 | 99.4 | YES | TP311 | 2.7 |
| 600923 | Month 18 | SP194 | 0.359 | 11.9 | POSITIVE | ConfP-143 | 100.0 | YES | TP311 | 2.7 |
| 600923 | Month 24 | SP196 | 1.780 | 71.0 | POSITIVE | ConfP-143 | 99.5 | YES | TP311 | 3.7 |
| 600929 | Baseline | SP38 | 0.045 | 0.2 | Negative | | | | | |
| 600929 | Month 1 | SP38 | 0.205 | 13.6 | POSITIVE | ConfP-30 | 97.2 | YES | TP262 | 2.7 |
| 600929 | Month 3 | SP38 | 1.439 | 117.6 | POSITIVE | ConfP-31 | 93.8 | YES | TP263 | 3.7 |
| 600929 | Month 6 | SP38 | 1.494 | 122.2 | POSITIVE | ConfP-5 | 96.7 | YES | TP1 | 3.7 |
| 600929 | Month 9 | SP38 | 0.705 | 55.8 | POSITIVE | ConfP-31 | 94.7 | YES | TP263 | 3.7 |
| 600929 | Month 12 | SP38 | 0.855 | 68.4 | POSITIVE | ConfP-31 | 95.4 | YES | TP263 | 3.7 |
| 600929 | Month 18 | SP38 | 0.717 | 56.8 | POSITIVE | ConfP-31 | 80.9 | YES | TP263 | 3.7 |
| 600929 | Month 24 | SP38 | 1.035 | 83.6 | POSITIVE | ConfP-31 | 66.8 | YES | TP263 | 3.7 |
| 600933 | Baseline | SP39 | 0.044 | 0.0 | Negative | | | | | |
| 600933 | Month 1 | SP39 | 0.046 | 0.0 | Negative | | | | | |
| 600933 | Month 3 | SP39 | 0.174 | 10.4 | POSITIVE | ConfP-31 | 99.7 | YES | TP263 | 2.7 |
| 600933 | Month 6 | SP39 | 0.204 | 12.8 | POSITIVE | ConfP-5 | 100.0 | YES | TP1 | 2.7 |
| 600933 | Month 9 | SP39 | 0.097 | 4.1 | POSITIVE | ConfP-31 | 99.0 | YES | TP264 | 2.7 |
| 600933 | Month 12 | SP39 | 0.072 | 2.1 | POSITIVE | ConfP-31 | 98.5 | YES | TP264 | 2.7 |
| 600933 | Month 18 | SP39 | 0.053 | 0.6 | Negative | | | | | |
| 600933 | Month 24 | SP39 | 0.085 | 3.2 | POSITIVE | ConfP-31 | 100.0 | YES | TP264 | 2.7 |
| 601002 | Baseline | SP39 | 0.044 | 0.0 | Negative | | | | | |
| 601002 | Month 1 | SP39 | 0.794 | 60.8 | POSITIVE | ConfP-31 | 99.6 | YES | TP264 | 3.7 |
| 601002 | Month 3 | SP39 | 0.903 | 69.7 | POSITIVE | ConfP-5 | 98.8 | YES | TP1 | 3.7 |
| 601002 | Month 6 | SP39 | 0.403 | 29.0 | POSITIVE | ConfP-31 | 98.9 | YES | TP264 | 2.7 |
| 601002 | Month 9 | SP40 | 0.308 | 21.7 | POSITIVE | ConfP-31 | 98.7 | YES | TP264 | 2.7 |
| 601002 | Month 12 | SP40 | 0.312 | 22.0 | POSITIVE | ConfP-31 | 99.1 | YES | TP265 | 2.7 |
| 601002 | Month 18 | SP40 | 0.293 | 20.5 | POSITIVE | ConfP-32 | 98.8 | YES | TP265 | 2.7 |
| 601002 | Month 24 | SP40 | 0.237 | 15.9 | POSITIVE | ConfP-32 | 98.8 | YES | TP265 | 2.7 |
| 601003 | Baseline | SP40 | 0.050 | 0.4 | Negative | | | | | |
| 601003 | Month 1 | SP40 | 0.253 | 17.2 | POSITIVE | ConfP-5 | 100.0 | YES | TP1 | 2.7 |
| 601003 | Month 3 | SP40 | 0.221 | 14.5 | POSITIVE | ConfP-32 | 99.1 | YES | TP265 | 2.7 |
| 601003 | Month 6 | SP40 | 0.077 | 2.6 | POSITIVE | ConfP-32 | 95.3 | YES | TP265 | 2.7 |
| 601003 | Month 9 | SP40 | 0.096 | 4.2 | POSITIVE | ConfP-32 | 97.3 | YES | TP265 | 2.7 |
| 601003 | Month 12 | SP40 | 0.142 | 8.0 | POSITIVE | ConfP-32 | 97.8 | YES | TP266 | 2.7 |
| 601003 | Month 18 | SP40 | 0.108 | 5.2 | POSITIVE | ConfP-32 | 98.4 | YES | TP266 | 2.7 |
| 601003 | Month 24 | SP40 | 0.103 | 4.8 | POSITIVE | ConfP-32 | 98.9 | YES | TP266 | 2.7 |
| 601004 | Baseline | SP41 | 0.049 | 0.2 | Negative | | | | | |
| 601004 | Month 1 | SP41 | 1.168 | 53.0 | POSITIVE | ConfP-32 | 99.8 | YES | TP266 | 3.7 |
| 601004 | Month 3 | SP41 | 1.892 | 87.1 | POSITIVE | ConfP-5 | 99.6 | YES | TP2 | 3.7 |
| 601004 | Month 6 | SP41 | 1.129 | 51.2 | POSITIVE | ConfP-32 | 99.9 | YES | TP266 | 2.7 |
| 601004 | Month 9 | SP41 | 1.128 | 51.1 | POSITIVE | ConfP-32 | 99.6 | YES | TP266 | 2.7 |
| 601004 | Month 12 | No sample received | | | | | | | | |
| 601004 | Month 18 | SP41 | 0.767 | 34.1 | POSITIVE | ConfP-32 | 99.8 | YES | TP267 | 2.7 |
| 601004 | Month 24 | SP41 | 0.245 | 9.5 | POSITIVE | ConfP-32 | 98.9 | YES | TP267 | 2.7 |
| 601005 | Baseline | SP41 | 0.052 | 0.4 | Negative | | | | | |
| 601005 | Month 1 | SP41 | 1.343 | 61.2 | POSITIVE | ConfP-33 | 98.3 | YES | TP267 | 3.7 |
| 601005 | Month 3 | SP41 | 1.341 | 61.2 | POSITIVE | ConfP-5 | 100.0 | YES | TP2 | 3.7 |
| 601005 | Month 6 | SP41 | 0.357 | 14.8 | POSITIVE | ConfP-33 | 98.7 | YES | TP267 | 2.7 |
| 601005 | Month 9 | SP42 | 0.359 | 14.4 | POSITIVE | ConfP-40 | 97.8 | YES | TP267 | 2.7 |
| 601005 | Month 12 | SP42 | 0.428 | 17.7 | POSITIVE | ConfP-33 | 99.2 | YES | TP267 | 2.7 |
| 601005 | Month 18 | SP42 | 0.427 | 17.7 | POSITIVE | ConfP-33 | 98.8 | YES | TP268 | 2.7 |
| 601005 | Month 24 | SP42 | 0.216 | 7.6 | POSITIVE | ConfP-33 | 93.5 | YES | TP268 | 2.7 |
| 601008 | Baseline | SP42 | 0.065 | 0.3 | Negative | | | | | |
| 601008 | Month 1 | SP42 | 0.827 | 36.8 | POSITIVE | ConfP-33 | 99.0 | YES | TP268 | 2.7 |
| 601008 | Month 3 | SP42 | 2.219 | 103.4 | POSITIVE | ConfP-5 | 99.8 | YES | TP2 | 3.7 |
| 601008 | Month 6 | SP42 | 1.591 | 73.4 | POSITIVE | ConfP-33 | 99.5 | YES | TP268 | 3.7 |
| 601008 | Month 9 | SP42 | 1.477 | 67.9 | POSITIVE | ConfP-33 | 99.7 | YES | TP268 | 3.7 |
| 601008 | Month 12 | SP42 | 1.320 | 60.4 | POSITIVE | ConfP-33 | 99.8 | YES | TP268 | 3.7 |
| 601008 | Month 18 | SP42 | 1.361 | 62.4 | POSITIVE | ConfP-33 | 99.7 | YES | TP269 | 3.7 |
| 601008 | Month 24 | SP42 | 1.909 | 88.6 | POSITIVE | ConfP-33 | 99.6 | YES | TP269 | 3.7 |
| 601009 | Baseline | SP43 | 0.047 | 0.2 | Negative | | | | | |
| 601009 | Month 1 | SP43 | 0.273 | 12.8 | POSITIVE | ConfP-33 | 99.2 | YES | TP269 | 2.7 |
| 601009 | Month 3 | SP43 | 1.941 | 106.0 | POSITIVE | ConfP-5 | 99.9 | YES | TP2 | 3.7 |
| 601009 | Month 6 | SP43 | 0.881 | 46.8 | POSITIVE | ConfP-34 | 100.0 | YES | TP269 | 2.7 |
| 601009 | Month 9 | SP43 | 0.362 | 17.8 | POSITIVE | ConfP-34 | 100.0 | YES | TP269 | 2.7 |
| 601009 | Month 12 | SP43 | 0.265 | 12.3 | POSITIVE | ConfP-34 | 100.0 | YES | TP269 | 2.7 |
| 601009 | Month 18 | SP43 | 0.215 | 9.6 | POSITIVE | ConfP-34 | 99.5 | YES | TP270 | 2.7 |
| 601009 | Month 24 | SP43 | 0.300 | 14.3 | POSITIVE | ConfP-34 | 100.0 | YES | TP270 | 2.7 |
| 601011 | Baseline | SP43 | 0.056 | 0.7 | Negative | | | | | |
| 601011 | Month 1 | SP43 | 0.135 | 5.1 | POSITIVE | ConfP-34 | 95.9 | YES | TP270 | 2.7 |
| 601011 | Month 3 | SP43 | 1.281 | 69.1 | POSITIVE | ConfP-5 | 99.6 | YES | TP2 | 3.7 |
| 601011 | Month 6 | SP43 | 1.022 | 54.6 | POSITIVE | ConfP-34 | 99.1 | YES | TP270 | 3.7 |
| 601011 | Month 9 | SP44 | 0.707 | 39.3 | POSITIVE | ConfP-34 | 99.4 | YES | TP270 | 2.7 |
| 601011 | Month 12 | SP44 | 0.416 | 22.0 | POSITIVE | ConfP-34 | 100.0 | YES | TP270 | 2.7 |
| 601011 | Month 18 | SP44 | 0.361 | 18.8 | POSITIVE | ConfP-34 | 98.8 | YES | TP271 | 2.7 |
| 601011 | Month 24 | SP44 | 0.341 | 17.6 | POSITIVE | ConfP-34 | 98.0 | YES | TP271 | 2.7 |
| 601013 | Baseline | SP44 | 0.050 | 0.3 | Negative | | | | | |
| 601013 | Month 1 | SP44 | 0.275 | 13.7 | POSITIVE | ConfP-34 | 98.2 | YES | TP271 | 2.7 |
| 601013 | Month 3 | SP44 | 0.699 | 38.8 | POSITIVE | ConfP-34 | 100.0 | YES | TP271 | 2.7 |
| 601013 | Month 6 | SP44 | 0.970 | 54.9 | POSITIVE | ConfP-5 | 99.7 | YES | TP2 | 2.7 |
| 601013 | Month 9 | SP44 | 0.752 | 42.0 | POSITIVE | ConfP-35 | 99.4 | YES | TP271 | 2.7 |
| 601013 | Month 12 | SP44 | 0.396 | 20.8 | POSITIVE | ConfP-35 | 99.4 | YES | TP271 | 2.7 |
| 601013 | Month 18 | SP44 | 0.947 | 53.6 | POSITIVE | ConfP-35 | 99.5 | YES | TP272 | 2.7 |
| 601013 | Month 24 | SP44 | 0.539 | 29.3 | POSITIVE | ConfP-35 | 99.8 | YES | TP272 | 2.7 |
| 601015 | Baseline | SP45 | 0.045 | 0.0 | Negative | | | | | |
| 601015 | Month 1 | SP45 | 0.409 | 18.0 | POSITIVE | ConfP-35 | 99.5 | YES | TP272 | 3.7 |
| 601015 | Month 3 | SP45 | 2.180 | 105.4 | POSITIVE | ConfP-5 | 99.7 | YES | TP3 | 3.7 |
| 601015 | Month 6 | SP45 | 1.342 | 64.0 | POSITIVE | ConfP-35 | 98.2 | YES | TP272 | 3.7 |
| 601015 | Month 9 | SP45 | 0.975 | 45.9 | POSITIVE | ConfP-35 | 98.2 | YES | TP272 | 3.7 |
| 601015 | Month 12 | SP45 | 0.478 | 21.4 | POSITIVE | ConfP-35 | 97.8 | YES | TP272 | 2.7 |
| 601015 | Month 18 | SP45 | 0.552 | 25.0 | POSITIVE | ConfP-35 | 98.5 | YES | TP273 | 2.7 |
| 601015 | Month 24 | SP45 | 0.270 | 11.1 | POSITIVE | ConfP-35 | 97.7 | YES | TP273 | 2.7 |
| 601017 | Baseline | SP151 | 0.049 | 0.4 | Negative | | | | | |
| 601017 | Month 1 | SP151 | 0.165 | 6.9 | POSITIVE | ConfP-115 | 90.1 | YES | TP177 | 2.7 |
| 601017 | Month 3 | SP151 | 1.888 | 103.4 | POSITIVE | ConfP-115 | 98.9 | YES | TP178 | 3.7 |
| 601017 | Month 6 | SP151 | 1.004 | 53.9 | POSITIVE | ConfP-115 | 97.8 | YES | TP178 | 3.7 |
| 601017 | Month 9 | SP152 | 1.108 | 59.6 | POSITIVE | ConfP-116 | 95.7 | YES | TP179 | 3.7 |
| 601017 | Month 12 | SP152 | 2.003 | 109.7 | POSITIVE | ConfP-116 | 98.0 | YES | TP179 | 4.7 |
| 601017 | Month 18 | SP152 | 0.403 | 20.1 | POSITIVE | ConfP-116 | 91.0 | YES | TP179 | 3.7 |
| 601017 | Month 24 | SP152 | 0.183 | 7.8 | POSITIVE | ConfP-116 | 80.5 | YES | TP179 | 2.7 |
| 601019 | Baseline | SP152 | 0.056 | 0.7 | Negative | | | | | |
| 601019 | Month 1 | SP152 | 0.044 | 0.1 | Negative | | | | | |
| 601019 | Month 3 | SP152 | 1.269 | 68.6 | POSITIVE | ConfP-115 | 99.4 | YES | TP178 | 3.7 |
| 601019 | Month 6 | SP152 | 0.677 | 35.5 | POSITIVE | ConfP-115 | 98.1 | YES | TP178 | 3.7 |
| 601019 | Month 9 | SP152 | 0.512 | 26.2 | POSITIVE | ConfP-116 | 98.7 | YES | TP178 | 3.7 |
| 601019 | Month 12 | SP152 | 0.566 | 29.3 | POSITIVE | ConfP-116 | 99.5 | YES | TP178 | 3.7 |
| 601019 | Month 18 | SP152 | 0.317 | 15.3 | POSITIVE | ConfP-116 | 99.1 | YES | TP179 | 3.7 |
| 601019 | Month 24 | SP152 | 0.488 | 24.9 | POSITIVE | ConfP-116 | 98.8 | YES | TP179 | 3.7 |
| 601020 | Baseline | SP153 | 0.046 | 0.2 | Negative | | | | | |
| 601020 | Month 1 | SP153 | 0.113 | 5.0 | POSITIVE | ConfP-116 | 86.0 | YES | TP180 | 2.7 |
| 601020 | Month 3 | SP153 | 1.494 | 103.3 | POSITIVE | ConfP-116 | 98.9 | YES | TP180 | 3.7 |
| 601020 | Month 6 | SP153 | 0.243 | 14.2 | POSITIVE | ConfP-116 | 93.3 | YES | TP180 | 3.7 |
| 601020 | Month 9 | SP153 | 0.265 | 15.8 | POSITIVE | ConfP-116 | 96.9 | YES | TP180 | 2.7 |
| 601020 | Month 12 | SP153 | 0.249 | 14.7 | POSITIVE | ConfP-116 | 96.0 | YES | TP180 | 2.7 |
| 601020 | Month 18 | SP153 | 0.180 | 9.8 | POSITIVE | ConfP-117 | 92.1 | YES | TP180 | 2.7 |
| 601020 | Month 24 | SP153 | 0.145 | 7.3 | POSITIVE | ConfP-117 | 87.2 | YES | TP181 | 2.7 |
| 601022 | Baseline | SP153 | 0.048 | 0.4 | Negative | | | | | |
| 601022 | Month 1 | SP153 | 0.319 | 19.6 | POSITIVE | ConfP-117 | 97.0 | YES | TP181 | 3.7 |
| 601022 | Month 3 | SP153 | 1.013 | 69.0 | POSITIVE | ConfP-117 | 99.0 | YES | TP181 | 3.7 |
| 601022 | Month 6 | SP153 | 0.790 | 53.2 | POSITIVE | ConfP-117 | 97.5 | YES | TP181 | 3.7 |
| 601022 | Month 9 | SP154 | 0.998 | 70.9 | POSITIVE | ConfP-118 | 98.7 | YES | TP183 | 3.7 |
| 601022 | Month 12 | SP154 | 1.401 | 100.8 | POSITIVE | ConfP-118 | 98.9 | YES | TP183 | 3.7 |
| 601022 | Month 18 | SP154 | 1.831 | 132.7 | POSITIVE | ConfP-118 | 99.2 | YES | TP183 | 4.7 |
| 601022 | Month 24 | SP154 | 1.573 | 113.6 | POSITIVE | ConfP-118 | 98.6 | YES | TP183 | 3.7 |
| 601023 | Baseline | SP154 | 0.091 | 3.7 | POSITIVE | ConfP-117 | 90.0 | YES | TP181 | 2.7 |
| 601023 | Month 1 | SP154 | 1.359 | 97.7 | POSITIVE | ConfP-117 | 99.5 | YES | TP181 | 3.7 |
| 601023 | Month 3 | SP154 | 1.683 | 121.7 | POSITIVE | ConfP-117 | 99.5 | YES | TP182 | 4.7 |
| 601023 | Month 6 | SP154 | 1.603 | 115.8 | POSITIVE | ConfP-117 | 99.5 | YES | TP182 | 4.7 |
| 601023 | Month 9 | SP154 | 1.304 | 93.6 | POSITIVE | ConfP-117 | 99.2 | YES | TP182 | 3.7 |
| 601023 | Month 12 | SP154 | 1.170 | 83.7 | POSITIVE | ConfP-117 | 98.4 | YES | TP182 | 3.7 |
| 601023 | Month 18 | SP154 | 1.218 | 87.2 | POSITIVE | ConfP-117 | 96.4 | YES | TP182 | 3.7 |
| 601023 | Month 24 | SP154 | 1.084 | 77.3 | POSITIVE | ConfP-117 | 95.0 | YES | TP182 | 3.7 |
| 601025 | Baseline | SP155 | 0.049 | 0.5 | Negative | | | | | |
| 601025 | Month 1 | SP155 | 1.110 | 71.7 | POSITIVE | ConfP-118 | 98.9 | YES | TP183 | 3.7 |
| 601025 | Month 3 | SP155 | 2.021 | 132.8 | POSITIVE | ConfP-118 | 99.4 | YES | TP183 | 4.7 |
| 601025 | Month 6 | SP155 | 0.902 | 57.7 | POSITIVE | ConfP-118 | 98.7 | YES | TP184 | 3.7 |
| 601025 | Month 9 | SP155 | 0.467 | 28.5 | POSITIVE | ConfP-118 | 97.9 | YES | TP184 | 2.7 |
| 601025 | Month 12 | SP155 | 0.409 | 24.6 | POSITIVE | ConfP-118 | 98.0 | YES | TP184 | 2.7 |
| 601025 | Month 18 | SP155 | 0.668 | 42.0 | POSITIVE | ConfP-118 | 99.0 | YES | TP184 | 3.7 |
| 601025 | Month 24 | SP155 | 1.253 | 81.3 | POSITIVE | ConfP-118 | 98.2 | YES | TP184 | 3.7 |
| 601027 | Baseline | SP155 | 0.046 | 0.3 | Negative | | | | | |
| 601027 | Month 1 | SP155 | 0.714 | 45.1 | POSITIVE | ConfP-118 | 98.9 | YES | TP184 | 3.7 |
| 601027 | Month 3 | SP155 | 0.496 | 30.5 | POSITIVE | ConfP-118 | 99.0 | YES | TP185 | 3.7 |
| 601027 | Month 6 | SP155 | 0.345 | 20.3 | POSITIVE | ConfP-119 | 97.8 | YES | TP185 | 2.7 |
| 601027 | Month 9 | SP156 | 0.147 | 6.7 | POSITIVE | ConfP-119 | 90.8 | YES | TP186 | 2.7 |
| 601027 | Month 12 | SP156 | 0.170 | 8.4 | POSITIVE | ConfP-119 | 97.9 | YES | TP186 | 2.7 |
| 601027 | Month 18 | SP156 | 0.092 | 2.8 | POSITIVE | ConfP-119 | 94.2 | YES | TP186 | 2.7 |
| 601027 | Month 24 | SP156 | 0.119 | 4.8 | POSITIVE | ConfP-119 | 94.9 | YES | TP187 | 2.7 |
| 601027 | TERMOL/ | SP156 | 0.055 | 0.2 | Negative | | | | | |
| | ET OL | | | | | | | | | |
| 601030 | Baseline | SP192 | 0.080 | 0.3 | Negative | | | | | |
| 601030 | Month 1 | SP192 | 0.559 | 21.1 | POSITIVE | ConfP-141 | 98.4 | YES | TP309 | 2.7 |
| 601030 | Month 3 | SP192 | 1.213 | 49.5 | POSITIVE | ConfP-141 | 99.6 | YES | TP309 | 3.7 |
| 601030 | Month 6 | No sample received | | | | | | | | |
| 601030 | Month 9 | SP192 | 0.349 | 11.9 | POSITIVE | ConfP-141 | 99.4 | YES | TP309 | 2.7 |
| 601030 | Month 12 | SP192 | 0.405 | 14.4 | POSITIVE | ConfP-141 | 100.0 | YES | TP309 | 2.7 |
| 601030 | Month 18 | SP192 | 0.169 | 4.1 | POSITIVE | ConfP-141 | 97.8 | YES | TP309 | 2.7 |
| 601030 | Month 24 | SP192 | 0.277 | 8.8 | POSITIVE | ConfP-141 | 98.9 | YES | TP309 | 2.7 |
| 601033 | Baseline | SP156 | 0.069 | 1.2 | Negative | | | | | |
| 601033 | Month 1 | SP156 | 0.374 | 22.8 | POSITIVE | ConfP-119 | 97.2 | YES | TP185 | 2.7 |
| 601033 | Month 3 | SP156 | 0.355 | 21.5 | POSITIVE | ConfP-119 | 97.0 | YES | TP185 | 2.7 |
| 601033 | Month 6 | SP156 | 0.221 | 12.0 | POSITIVE | ConfP-119 | 95.4 | YES | TP185 | 2.7 |
| 601033 | Month 9 | SP156 | 0.243 | 13.5 | POSITIVE | ConfP-119 | 95.0 | YES | TP185 | 2.7 |
| 601033 | Month 12 | SP156 | 0.163 | 7.9 | POSITIVE | ConfP-119 | 92.9 | YES | TP186 | 3.7 |
| 601033 | Month 18 | SP156 | 0.113 | 4.3 | POSITIVE | ConfP-119 | 91.2 | YES | TP186 | 3.7 |
| 601033 | Month 24 | SP156 | 0.153 | 7.2 | POSITIVE | ConfP-119 | 90.7 | YES | TP186 | 2.7 |
| 601034 | Baseline | SP157 | 0.109 | 3.5 | POSITIVE | ConfP-119 | 95.5 | YES | TP187 | 2.7 |
| 601034 | Month 1 | SP157 | 1.117 | 58.0 | POSITIVE | ConfP-120 | 98.5 | YES | TP187 | 3.7 |
| 601034 | Month 3 | SP157 | 1.955 | 103.4 | POSITIVE | ConfP-120 | 98.4 | YES | TP187 | 3.7 |
| 601034 | Month 6 | SP157 | 1.303 | 68.1 | POSITIVE | ConfP-120 | 98.3 | YES | TP187 | 3.7 |
| 601034 | Month 9 | SP157 | 1.153 | 60.0 | POSITIVE | ConfP-120 | 98.7 | YES | TP187 | 3.7 |
| 601034 | Month 12 | SP157 | 1.326 | 69.3 | POSITIVE | ConfP-120 | 99.3 | YES | TP188 | 3.7 |
| 601034 | Month 18 | SP157 | 1.114 | 57.9 | POSITIVE | ConfP-120 | 98.7 | YES | TP188 | 3.7 |
| 601034 | Month 24 | SP157 | 0.862 | 44.2 | POSITIVE | ConfP-120 | 99.0 | YES | TP188 | 3.7 |
| 601205 | Baseline | SP46 | 0.050 | 0.3 | Negative | | | | | |
| 601205 | Month 1 | SP46 | 0.064 | 1.0 | Negative | | | | | |
| 601205 | Month 3 | SP46 | 0.198 | 7.8 | POSITIVE | ConfP-6 | 100.0 | YES | TP3 | 2.7 |
| 601205 | Month 6 | SP46 | 0.096 | 2.6 | POSITIVE | ConfP-36 | 97.6 | YES | TP274 | 2.7 |
| 601205 | Month 9 | SP46 | 0.060 | 0.8 | Negative | | | | | |
| 601205 | Month 12 | SP46 | 0.051 | 0.3 | Negative | | | | | |
| 601205 | Month 18 | SP46 | 0.046 | 0.1 | Negative | | | | | |
| 601205 | Month 24 | SP46 | 0.045 | 0.0 | Negative | | | | | |
| 601208 | Baseline | SP47 | 0.045 | 0.1 | Negative | | | | | |
| 601208 | Month 1 | SP47 | 1.225 | 59.8 | POSITIVE | ConfP-36 | 99.5 | YES | TP274 | 3.7 |
| 601208 | Month 3 | SP47 | 2.354 | 116.8 | POSITIVE | ConfP-6 | 99.7 | YES | TP3 | 3.7 |
| 601208 | Month 6 | SP47 | 0.632 | 29.8 | POSITIVE | ConfP-36 | 99.6 | YES | TP274 | 2.7 |
| 601208 | Month 9 | ConfP6 | 0.246 | 24.6 | POSITIVE | ConfP-36 | 97.7 | YES | TP274 | 2.7 |
| 601208 | Month 12 | SP47 | 1.359 | 66.5 | POSITIVE | ConfP-36 | 99.6 | YES | TP275 | 3.7 |
| 601208 | Month 18 | SP47 | 1.393 | 68.3 | POSITIVE | ConfP-36 | 99.5 | YES | TP275 | 3.7 |
| 601208 | Month 24 | SP47 | 0.804 | 38.5 | POSITIVE | ConfP-36 | 99.3 | YES | TP275 | 3.7 |
| 601209 | Baseline | SP158 | 0.044 | 0.1 | Negative | | | | | |
| 601209 | Month 1 | SP158 | 1.019 | 52.6 | POSITIVE | ConfP-120 | 98.5 | YES | TP188 | 3.7 |
| 601209 | Month 3 | SP158 | 1.735 | 91.2 | POSITIVE | ConfP-120 | 98.5 | YES | TP189 | 3.7 |
| 601209 | Month 6 | SP158 | 0.629 | 31.6 | POSITIVE | ConfP-120 | 96.9 | YES | TP189 | 3.7 |
| 601209 | Month 9 | SP158 | 0.356 | 16.9 | POSITIVE | ConfP-120 | 95.3 | YES | TP189 | 2.7 |
| 601209 | Month 12 | SP158 | 0.557 | 27.7 | POSITIVE | ConfP-121 | 96.6 | YES | TP189 | 3.7 |
| 601209 | Month 18 | SP158 | 0.308 | 14.3 | POSITIVE | ConfP-121 | 94.7 | YES | TP189 | 2.7 |
| 601209 | Month 24 | SP158 | 0.330 | 15.5 | POSITIVE | ConfP-121 | 94.2 | YES | TP189 | 3.7 |
| 601212 | Baseline | SP159 | 0.050 | 0.4 | Negative | | | | | |
| 601212 | Month 1 | SP159 | 1.749 | 92.0 | POSITIVE | ConfP-121 | 99.3 | YES | TP190 | 3.7 |
| 601212 | Month 3 | SP159 | 1.302 | 67.9 | POSITIVE | ConfP-121 | 99.3 | YES | TP190 | 3.7 |
| 601212 | Month 6 | SP159 | 0.779 | 39.7 | POSITIVE | ConfP-121 | 98.8 | YES | TP191 | 3.7 |
| 601212 | Month 9 | SP159 | 1.518 | 79.6 | POSITIVE | ConfP-121 | 99.2 | YES | TP191 | 3.7 |
| 601212 | Month 12 | SP159 | 1.061 | 54.9 | POSITIVE | ConfP-121 | 99.1 | YES | TP191 | 3.7 |
| 601212 | Month 18 | SP159 | 0.756 | 38.5 | POSITIVE | ConfP-121 | 99.1 | YES | TP191 | 3.7 |
| 601212 | Month 24 | SP159 | 1.228 | 63.9 | POSITIVE | ConfP-122 | 99.0 | YES | TP191 | 3.7 |
| 601301 | Baseline | SP47 | 0.044 | 0.1 | Negative | | | | | |
| 601301 | Month 1 | SP47 | 0.196 | 7.7 | POSITIVE | ConfP-36 | 99.4 | YES | TP275 | 3.7 |
| 601301 | Month 3 | SP47 | 2.516 | 125.0 | POSITIVE | ConfP-6 | 99.6 | YES | TP3 | 3.7 |
| 601301 | Month 6 | SP47 | 0.418 | 19.0 | POSITIVE | ConfP-36 | 99.7 | YES | TP275 | 3.7 |
| 601301 | Month 9 | SP48 | 0.303 | 13.8 | POSITIVE | ConfP-36 | 99.5 | YES | TP275 | 3.7 |
| 601301 | Month 12 | SP48 | 0.252 | 11.2 | POSITIVE | ConfP-37 | 99.5 | YES | TP276 | 2.7 |
| 601301 | Month 18 | SP48 | 0.205 | 8.7 | POSITIVE | ConfP-37 | 99.7 | YES | TP276 | 2.7 |
| 601301 | Month 24 | SP48 | 0.160 | 6.3 | POSITIVE | ConfP-37 | 98.8 | YES | TP276 | 2.7 |
| 601306 | Baseline | SP159 | 0.045 | 0.2 | Negative | | | | | |
| 601306 | Month 1 | SP159 | 0.738 | 37.5 | POSITIVE | ConfP-122 | 99.1 | YES | TP191 | 3.7 |
| 601306 | Month 3 | SP159 | 0.777 | 39.6 | POSITIVE | ConfP-122 | 99.1 | YES | TP192 | 3.7 |
| 601306 | Month 6 | SP159 | 0.317 | 14.8 | POSITIVE | ConfP-122 | 97.5 | YES | TP192 | 2.7 |
| 601306 | Month 9 | SP160 | 0.328 | 15.8 | POSITIVE | ConfP-122 | 97.5 | YES | TP193 | 2.7 |
| 601306 | Month 12 | SP160 | 0.262 | 12.2 | POSITIVE | ConfP-122 | 96.6 | YES | TP193 | 2.7 |
| 601306 | Month 18 | SP160 | 0.343 | 16.6 | POSITIVE | ConfP-123 | 95.1 | YES | TP193 | 2.7 |
| 601306 | Month 24 | SP160 | 0.475 | 24.0 | POSITIVE | ConfP-123 | 85.0 | YES | TP194 | 3.7 |
| 601309 | Baseline | SP160 | 0.048 | 0.3 | Negative | | | | | |
| 601309 | Month 1 | SP160 | 0.664 | 34.5 | POSITIVE | ConfP-122 | 99.8 | YES | TP192 | 2.7 |
| 601309 | Month 3 | SP160 | 0.499 | 25.3 | POSITIVE | ConfP-122 | 99.8 | YES | TP192 | 2.7 |
| 601309 | Month 6 | SP160 | 0.402 | 19.9 | POSITIVE | ConfP-122 | 99.3 | YES | TP192 | 2.7 |
| 601309 | Month 9 | SP160 | 0.322 | 15.5 | POSITIVE | ConfP-122 | 99.3 | YES | TP192 | 2.7 |
| 601309 | Month 12 | SP160 | 0.227 | 10.2 | POSITIVE | ConfP-122 | 98.9 | YES | TP193 | 2.7 |
| 601309 | Month 18 | SP160 | 0.113 | 3.9 | POSITIVE | ConfP-122 | 99.2 | YES | TP193 | 2.7 |
| 601309 | Month 24 | SP160 | 0.095 | 2.9 | POSITIVE | ConfP-122 | 99.0 | YES | TP193 | 2.7 |
| 601310 | Baseline | SP161 | 0.043 | 0.0 | Negative | | | | | |
| 601310 | Month 1 | SP161 | 0.053 | 0.6 | Negative | | | | | |
| 601310 | Month 3 | SP161 | 0.183 | 8.2 | POSITIVE | ConfP-123 | 97.9 | YES | TP194 | 2.7 |
| 601310 | Month 6 | SP161 | 0.066 | 1.3 | Negative | | | | | |
| 601310 | Month 9 | SP161 | 0.098 | 3.2 | POSITIVE | ConfP-123 | 93.2 | YES | TP194 | 2.7 |
| 601310 | Month 12 | SP161 | 0.071 | 1.6 | POSITIVE | ConfP-123 | 93.8 | YES | TP194 | 2.7 |
| 601310 | Month 18 | SP161 | 0.084 | 2.4 | POSITIVE | ConfP-123 | 95.1 | YES | TP194 | 2.7 |
| 601310 | Month 24 | SP161 | 0.098 | 3.2 | POSITIVE | ConfP-123 | 94.7 | YES | TP194 | 2.7 |
| 811002 | Baseline | SP48 | 0.046 | 0.3 | Negative | | | | | |
| 811002 | Month 1 | SP48 | 1.123 | 57.0 | POSITIVE | ConfP-6 | 99.6 | YES | TP3 | 3.7 |
| 811002 | Month 3 | SP48 | 0.048 | 0.4 | Negative | | | | | |
| 811002 | Month 6 | SP48 | 0.267 | 11.9 | POSITIVE | ConfP-37 | 99.8 | YES | TP276 | 3.7 |
| 811002 | Month 9 | SP48 | 0.107 | 3.5 | POSITIVE | ConfP-37 | 99.7 | YES | TP276 | 3.7 |
| 811002 | Month 12 | SP48 | 0.184 | 7.6 | POSITIVE | ConfP-37 | 94.3 | YES | TP276 | 3.7 |
| 811002 | Month 18 | SP48 | 0.146 | 5.6 | POSITIVE | ConfP-37 | 98.9 | YES | TP277 | 3.7 |
| 811002 | Month 24 | SP48 | 0.112 | 3.8 | POSITIVE | ConfP-37 | 98.2 | YES | TP277 | 2.7 |
| 811003 | Baseline | SP49 | 0.045 | 0.2 | Negative | | | | | |
| 811003 | Month 1 | SP49 | 0.410 | 20.9 | POSITIVE | ConfP-37 | 99.3 | YES | TP277 | 3.7 |
| 811003 | Month 3 | SP49 | 1.168 | 64.0 | POSITIVE | ConfP-6 | 99.3 | YES | TP4 | 3.7 |
| 811003 | Month 6 | SP49 | 0.564 | 29.7 | POSITIVE | ConfP-37 | 98.1 | YES | TP277 | 3.7 |
| 811003 | Month 9 | SP49 | 0.448 | 23.1 | POSITIVE | ConfP-37 | 97.3 | YES | TP277 | 3.7 |
| 811003 | Month 12 | SP49 | 0.200 | 9.0 | POSITIVE | ConfP-37 | 99.8 | YES | TP277 | 2.7 |
| 811003 | Month 18 | SP49 | 0.175 | 7.6 | POSITIVE | ConfP-37 | 84.5 | YES | TP278 | 2.7 |
| 811003 | Month 24 | SP49 | 0.146 | 5.9 | POSITIVE | ConfP-38 | 86.5 | YES | TP278 | 2.7 |
| 811004 | Baseline | SP49 | 0.050 | 0.5 | Negative | | | | | |
| 811004 | Month 1 | SP49 | 0.557 | 29.3 | POSITIVE | ConfP-38 | 99.3 | YES | TP278 | 3.7 |
| 811004 | Month 3 | SP49 | 0.762 | 40.9 | POSITIVE | ConfP-6 | 99.5 | YES | TP4 | 3.7 |
| 811004 | Month 6 | SP49 | 0.391 | 19.8 | POSITIVE | ConfP-38 | 99.1 | YES | TP278 | 3.7 |
| 811004 | Month 9 | SP50 | 0.354 | 17.5 | POSITIVE | ConfP-38 | 99.1 | YES | TP278 | 3.7 |
| 811004 | Month 12 | SP50 | 0.234 | 10.8 | POSITIVE | ConfP-38 | 99.0 | YES | TP278 | 3.7 |
| 811004 | Month 18 | SP50 | 0.347 | 17.1 | POSITIVE | ConfP-38 | 98.6 | YES | TP279 | 2.7 |
| 811004 | Month 24 | SP50 | 0.251 | 11.7 | POSITIVE | ConfP-33 | 98.9 | YES | TP279 | 2.7 |
| 811005 | Baseline | SP50 | 0.046 | 0.2 | Negative | | | | | |
| 811005 | Month 1 | SP50 | 1.232 | 66.7 | POSITIVE | ConfP-6 | 90.9 | YES | TP4 | 3.7 |
| 811005 | Month 3 | SP50 | 1.104 | 59.6 | POSITIVE | ConfP-38 | 97.7 | YES | TP279 | 3.7 |
| 811005 | Month 6 | SP50 | 0.726 | 38.4 | POSITIVE | ConfP-38 | 98.7 | YES | TP279 | 4.7 |
| 811005 | Month 9 | SP50 | 0.269 | 12.7 | POSITIVE | ConfP-38 | 99.0 | YES | TP279 | 2.7 |
| 811005 | Month 12 | SP50 | 0.161 | 6.7 | POSITIVE | ConfP-38 | 99.2 | YES | TP280 | 2.7 |
| 811005 | Month 18 | SP50 | 0.106 | 3.6 | POSITIVE | ConfP-38 | 98.3 | YES | TP280 | 2.7 |
| 811005 | Month 24 | SP50 | 0.154 | 6.3 | POSITIVE | ConfP-38 | 96.4 | YES | TP280 | 2.7 |
| 811011 | Baseline | SP161 | 0.043 | 0.0 | Negative | | | | | |
| 811011 | Month 1 | SP161 | 0.702 | 38.7 | POSITIVE | ConfP-123 | 99.6 | YES | TP195 | 3.7 |
| 811011 | Month 3 | SP161 | 0.451 | 23.9 | POSITIVE | ConfP-123 | 99.7 | YES | TP195 | 3.7 |
| 811011 | Month 6 | SP161 | 0.236 | 11.3 | POSITIVE | ConfP-123 | 99.1 | YES | TP195 | 3.7 |
| 811011 | Month 9 | SP162 | 0.170 | 7.7 | POSITIVE | ConfP-124 | 90.0 | YES | TP196 | 2.7 |
| 811011 | Month 12 | SP162 | 0.169 | 7.7 | POSITIVE | ConfP-124 | 87.0 | YES | TP196 | 2.7 |
| 811011 | Month 18 | SP162 | 0.145 | 6.2 | POSITIVE | ConfP-124 | 76.7 | YES | TP197 | 2.7 |
| 811011 | Month 24 | SP162 | 0.195 | 9.2 | POSITIVE | ConfP-124 | 97.9 | YES | TP197 | 2.7 |
| 811105 | Baseline | SP184 | 0.047 | 0.2 | Negative | | | | | |
| 811105 | Baseline | SP184 | 0.048 | 0.3 | Negative | | | | | |
| 811105 | Month 1 | SP162 | 1.048 | 60.4 | POSITIVE | ConfP-123 | 99.3 | YES | TP195 | 3.7 |
| 811105 | Month 3 | SP162 | 1.713 | 100.2 | POSITIVE | ConfP-123 | 99.5 | YES | TP195 | 4.7 |
| 811105 | Month 6 | SP162 | 1.329 | 77.2 | POSITIVE | ConfP-133 | 98.9 | YES | TP195 | 3.7 |
| 811105 | Month 9 | SP162 | 1.230 | 71.3 | POSITIVE | ConfP-124 | 92.0 | YES | TP196 | 3.7 |
| 811105 | Month 12 | SP162 | 1.175 | 68.0 | POSITIVE | ConfP-133 | 98.9 | YES | TP196 | 3.7 |
| 811105 | Month 18 | SP162 | 0.949 | 54.4 | POSITIVE | ConfP-124 | 91.9 | YES | TP196 | 3.7 |
| 811105 | Month 24 | SP162 | 0.826 | 47.1 | POSITIVE | ConfP-124 | 96.9 | YES | TP196 | 3.7 |
| 811106 | Baseline | SP163 | 0.043 | 0.1 | Negative | | | | | |
| 811106 | Month 1 | SP163 | 1.136 | 60.6 | POSITIVE | ConfP-124 | 98.7 | YES | TP197 | 3.7 |
| 811106 | Month 3 | SP163 | 1.515 | 81.6 | POSITIVE | ConfP-124 | 98.7 | YES | TP197 | 4.7 |
| 811106 | Month 6 | SP163 | 0.666 | 34.6 | POSITIVE | ConfP-124 | 97.2 | YES | TP197 | 3.7 |
| 811106 | Month 9 | SP163 | 0.676 | 35.1 | POSITIVE | ConfP-124 | 98.8 | YES | TP197 | 3.7 |
| 811106 | Month 12 | SP163 | 0.838 | 44.1 | POSITIVE | ConfP-125 | 99.3 | YES | TP198 | 3.7 |
| 811106 | Month 18 | SP163 | 0.566 | 29.0 | POSITIVE | ConfP-125 | 98.6 | YES | TP198 | 3.7 |
| 811106 | Month 24 | SP163 | 0.422 | 21.0 | POSITIVE | ConfP-125 | 98.5 | YES | TP198 | 2.7 |
| 811107 | Baseline | SP163 | 0.053 | 0.6 | Negative | | | | | |
| 811107 | Month 1 | SP163 | 0.801 | 42.0 | POSITIVE | ConfP-125 | 99.0 | YES | TP198 | 3.7 |
| 811107 | Month 3 | SP163 | 1.977 | 107.1 | POSITIVE | ConfP-125 | 99.1 | YES | TP198 | 4.7 |
| 811107 | Month 6 | SP163 | 1.602 | 86.4 | POSITIVE | ConfP-125 | 99.0 | YES | TP198 | 3.7 |
| 811107 | Month 9 | SP164 | 1.599 | 85.1 | POSITIVE | ConfP-126 | 99.2 | YES | TP200 | 3.7 |
| 811107 | Month 12 | SP164 | 1.510 | 80.3 | POSITIVE | ConfP-126 | 99.4 | YES | TP200 | 3.7 |
| 811107 | Month 18 | SP164 | 0.664 | 34.0 | POSITIVE | ConfP-126 | 98.8 | YES | TP200 | 3.7 |
| 811107 | Month 24 | SP164 | 0.777 | 40.2 | POSITIVE | ConfP-126 | 98.8 | YES | TP200 | 3.7 |
| 902201 | Baseline | SP164 | 0.044 | 0.1 | Negative | | | | | |
| 902201 | Month 1 | SP164 | 0.433 | 21.3 | POSITIVE | ConfP-125 | 98.9 | YES | TP199 | 3.7 |
| 902201 | Month 3 | SP164 | 2.434 | 130.8 | POSITIVE | ConfP-125 | 98.4 | YES | TP199 | 4.7 |
| 902201 | Month 6 | SP164 | 2.343 | 125.8 | POSITIVE | ConfP-125 | 99.3 | YES | TP199 | 4.7 |
| 902201 | Month 9 | SP164 | 2.300 | 123.5 | POSITIVE | ConfP-125 | 99.5 | YES | TP199 | 4.7 |
| 902201 | Month 12 | SP164 | 2.001 | 107.1 | POSITIVE | ConfP-125 | 99.5 | YES | TP199 | 4.7 |
| 902201 | Month 18 | SP164 | 0.495 | 24.7 | POSITIVE | ConfP-125 | 98.2 | YES | TP199 | 3.7 |
| 902201 | Month 24 | SP164 | 0.612 | 31.1 | POSITIVE | ConfP-125 | 99.1 | YES | TP200 | 3.7 |

The average anti-GA IgG response (Screening RA %) of the GA treatment group at Baseline was 0.456818182 having a standard deviation of 0.683615214.

The average anti-GA IgG response (Screening RA %) of the GA treatment group at Month 1 was 34.7515625 having a standard deviation of 35.27291909.

The average anti-GA IgG response (Screening RA %) of the GA treatment group at Month 3 was 67.7229572 having a standard deviation of 38.6096852.

The average anti-GA IgG response (Screening RA %) of the GA treatment group at Month 6 was 39.69111969 having a standard deviation of 32.3192211.

The average anti-GA IgG response (Screening RA %) of the GA treatment group at Month 9 was 30.60694981 having a standard deviation of 29.94922134.

The average anti-GA IgG response (Screening RA %) of the GA treatment group at Month 12 was 28.94263566 having a standard deviation of 29.45298377.

The average anti-GA IgG response (Screening RA %) of the GA treatment group at Month 18 was 24.8980695 having a standard deviation of 26.5184687.

The average anti-GA IgG response (Screening RA %) of the GA treatment group at Month 24 was 23.35938697 having a standard deviation of 26.93304872.

### CONCLUSION

Serum samples were obtained at various time points during treatment from patients who were treated with 40 mg/ml GA subcutaneous injection three times a week. The samples were tested for the presence of anti-GA IgG antibodies. In the screening tests, 1740 samples out of 1809 non-baseline samples were found to be positive and the positivity of 99.9% of the non-baseline samples was verified in the confirmatory test.

Every tested patients were confirmed positive for the presence of anti-GA IgG Abs. The total anti-GA IgG Abs titer was determined for all confirmed samples.

The immunological response profile of the averaged anti-GA IgG response (mean RA % values) in 40 mg/ml GA treatment group, was characterized by significant and fast increase of total anti-GA IgG Abs levels in the tested sera during the first treatment month followed by relatively slower additional increase recorded at the 3 months time point. Total Anti-GA IgG levels decreased by month 6 of the treatment but did not reach the baseline level at month 24. See FIG. 3.

### DISCUSSION

Daily subcutaneous (sc) Copaxone® 20 mg/mL is an approved drug product that contains the active ingredient, GA, the safety and efficacy of which are supported by over two decades of clinical research and over a decade of post-marketing experience. Numerous studies have been conducted for the clinical effects of daily sc Copaxone® 20 mg/mL, one of which is the antibody response to GA in MS patients (28).

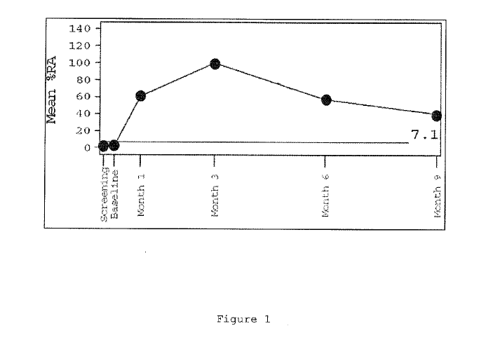

A controlled study of 90 MS patients who were selected from the pilot clinical trial and treated with sc Copaxone® 20 mg/ml every day was conducted and Ab levels were followed for 9 months. The total anti-GA specific antibody level peaked between about 3 and about 6 months, reaching at least 50 times baseline value. See FIG. 1. The rate of increase of anti-GA specific antibody level between initiation of treatment and 1 month was greater than that between 1 month and 3 months. Decrease of anti-GA antibody level was usually detected by 6 months from initiation of treatment. After 6 months, the antibody level declined and remained low, although at a higher level than at baseline.

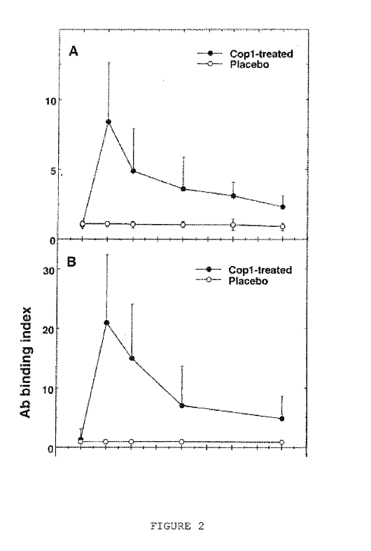

Consistent with the results of the 90 MS patients who were treated with sc Copaxone® 20 mg/ml every day in the pilot trial and whose Ab levels were followed for 9 months, in another controlled clinical trial of 217 patients who were treated also with Copaxone® 20 mg/mL everyday for 2 years, the anti-GA specific antibody level peaked between about 3 and about 6 months from initiation of treatment, reaching at least 8 times baseline value. See FIG. 2. By 6 months of treatment, however, decrease of anti-GA antibody level was observed. After 6 months, the antibody level declined and remained low, although at a somewhat higher level than at baseline. The antibodies were exclusively of the IgG subtype and not IgE or IgM, and IgG1 levels were 2-3 fold higher than those of IgG2 at all the time points examined (28).

These results together suggest that MS patients develop an immunological response to daily sc administration of Copaxone 20 mg/mL, which is part of an overall immunomodulatory activity which may be vital for the clinical efficacy of Copaxone 20 mg/mL.

Despite the clinical efficacy, a significant drawback to Copaxone®20 mg/mL therapy is the requirement of daily injections, which can be inconvenient. Moreover, in all clinical trials, injection-site reactions were seen to be the most frequent adverse reactions and were reported by the majority of patients receiving Copaxone® 20 mg/mL. In controlled studies, the proportion of patients reporting these reactions, at least once, was higher following treatment with Copaxone 20 mg/mL (70%) than placebo injections (37%). The most commonly reported injection-site reactions, which were more frequently reported in Copaxone® 20 mg/mL vs. placebo-treated patients, were erythema, pain, mass, pruritus, edema, inflammation and hypersensitivity.

However, several obstacles and limitations with potential approaches for addressing the drawbacks exist to current Copaxone® 20 mg/mL therapy. Subcutaneous drug delivery is limited, firstly, by the acceptable injection volume. Typically no more than 1 to 2 ml of solution is permitted (33). Secondly, the potential exists for drug degradation at the site of injection resulting in reduced bioavailability. Thirdly, based on the physiochemical properties of the drug, potent compounds may become locally trapped in the interstitial space which can lead to further localized irritation, precipitation of the drug and concentration-dependent adverse effects (34). Finally, due to the complex pharmacokinetic behavior of the drug, the efficacy and safety associated with any variation in the frequency of administration is unpredictable and requires empirical testing. For example, although controlled clinical trials have demonstrated the efficacy of IFNβ-1b in the treatment of MS, patient compliance, efficacy and tolerability are affected by the dosage regimen used. Merely increasing the dose of IFNβ-1b is insufficient to increase efficacy, the frequency of administration must also be increased (35).

The anti-GA specific antibody data of MS patients who were treated with Copaxone® 40 mg/ml three times a week for a year (described in Example 1) is surprisingly consistent with the two clinical studies discussed above, which were directed to 20 mg/ml GA daily. The newly developed formulation of the active ingredient of GA, i.e. Copaxone® 40 mg/ml three times a week, not only addresses the drawbacks of Copaxone® 20 mg/mL, e.g. by reducing frequency of injection and potentially the Injection Related Adverse Events and Injection Site Reactions (38), but also does not compromise the antibody response observed after the administration of Copaxone® 20 mg/mL represented in FIGS. 1 and 2.

### REFERENCES

1. Noseworthy J H, Lucchinetti C, Rodriguez M, Weinshenker B G. Multiple sclerosis. N Engl J Med 2000; 343:938-52.
2. Guideline on clinical investigation of medicinal products for the treatment of multiple sclerosis EMEA, London 16 Sep. 2006.
3. Bjartmar C, Fox R J. Pathological mechanisms and disease progression of multiple sclerosis: therapeutic implications. Drugs of Today 2002; 38:17-29.
4. Fleming J O. Diagnosis and management of multiple sclerosis. 1st ed. New York: Professional communications, Inc., 2002.
5. Anderson D W, Ellenberg J H, Leventhal C M et al. Revised estimate of the prevalence of multiple sclerosis in the United States. Ann Neurol 1992; 31:333-36.
6. Compston A, Lassmann H, McDonald I. The story of multiple sclerosis. In: Compston A, Confavreux C, Lassman H, Mcdonald I, Miller D, Noseworthy J H, Smith K, Wekerle H, editors. McAlpine's Multiple Sclerosis. London: Churchill Livingstone; 2006. p. 3-68.
7. Revel M., Pharmacal. Ther., 100(1):49-62 (2003).
8. Copaxone, Food and Drug Administration Approved Labeling (Reference ID: 3443331) [online], TEVA Pharmaceutical Industries Ltd., 2014 [retrieved on Dec. 24, 2014], Retrieved from the Internet: <URL: www.accessdata.fda.gov/drugsatfdadocs/label/2014/020622s0891b1.pdf>.
9. Filippi M, et al. (2001) Glatiramer acetate reduces the proportion of new MS lesions evolving into “black holes”. Neurology 57:731-733.
10. Kipnis J, Schwartz M (2002) Dual action of glatiramer acetate (Cop-1) in the treatment of CNS autoimmune and neurodegenerative disorders. TRENDS in Molecular Medicine 8:319-323.
11. Teitelbaum D, Aharoni R, Arnon R, Sela M (1988) Specific inhibition of the T-cell response to myelin basic protein by the synthetic copolymer Cop 1. Proc Natl Acad Sci USA 85:9724-9728.
12. Putheti P, Soderstrom M, Link H, Huang Y M (2003) Effect of glatiramer acetate (Copaxone®) on CD4+CD25high T regulatory cells and their IL-10 production in multiple sclerosis. J Neuroimmunol 144:125-131.
13. Stadelmann C, et al. (2002) BDNF and gp145trkB in multiple sclerosis brain lesions: neuroprotective interactions between immune and neuronal cells? Brain 125:75-85.
14. Ford C, et al. (2010) Continuous long-term immunomodulatory therapy in relapsing multiple sclerosis: results from the 15-year analysis of the US prospective open-label study of glatiramer acetate. Mult Scler 16:342-350.
15. Aharoni R, Teitelbaum D, Arnon R, Sela M (1999) Copolymer 1 acts against the immunodominant epitope 82-100 of myelin basic protein by T cell receptor antagonism in addition to major histocompatibility complex blocking. Proc Natl Acad Sci USA 96:634-639.
16. Arnon R, Aharoni R (2004) Mechanism of action of glatiramer acetate in multiple sclerosis and its potential for the development of new applications. Proc Natl Acad Sci USA 101 Suppl 2:14593-14598.
17. Teitelbaum D, Aharoni R, Sela M, Arnon R (1991) Cross-reactions and specificities of monoclonal antibodies against myelin basic protein and against the synthetic copolymer 1. Proc Natl Acad Sci USA 88:9528-9532.
18. Webb C, Teitelbaum D, Arnon R, Sela M (1973) In vivo and in vitro immunological cross-reactions between basic encephalitogen and synthetic basic polypeptides capable of suppressing experimental allergic encephalomyelitis. Eur J Immunol 3:279-286.
19. Duda P W, et al. (2000) Human and murine CD4 T cell reactivity to a complex antigen: recognition of the synthetic random polypeptide glatiramer acetate. J Immunol 165:7300-7307.
20. Duda P W, et al. (2000) Glatiramer acetate (Copaxone) induces degenerate, Th2-polarized immune responses in patients with multiple sclerosis. J Clin Invest 105:967-976.
21. Brenner T, et al. (2001) Humoral and cellular immune responses to Copolymer 1 in multiple sclerosis patients treated with Copaxone. J Neuroimmunol 115:152-160.
22. Aharoni R, Teitelbaum D, Sela M, Arnon R (1997) Copolymer 1 induces T cells of the T helper type 2 that crossreact with myelin basic protein and suppress experimental autoimmune encephalomyelitis. Proc Natl Acad Sci USA 94:10821-10826.
23. Wiesemann E, et al. (2003) Correlation of serum IL-13 and IL-5 levels with clinical response to Glatiramer acetate in patients with multiple sclerosis. Clin Exp Immunol 133:454-460.
24. Aharoni R, Teitelbaum D, Sela M, Arnon R (1998) Bystander suppression of experimental autoimmune encephalomyelitis by T cell lines and clones of the Th2 type induced by copolymer 1. J Neuroimmunol 91:135-146.
25. Aharoni R, at al. (2003) Glatiramer acetate-specific T cells in the brain express T helper ⅔ cytokines and brain-derived neurotrophic factor in situ. Proc Natl Acad Sci USA 100:14157-14162.
26. Miller A, at al. (1998) Treatment of multiple sclerosis with copolymer-1 (Copaxone): implicating mechanisms of Th1 to Th2/Th3 immune-deviation. J Neuroimmunol 92:113-121.
27. Neuhaus O, at al. (2000) Multiple Sclerosis: Comparison of copolymer-1-reactive T cell Lines from treated and untreated subjects reveals cytokine shift From T helper 1 to T helper 2 cells. Proceedings of the National Academy of Sciences 97:7452-7457.
28. Brenner T, et al. (2001) Humoral and cellular immune responses to Copolymer 1 in multiple sclerosis patients treated with Copaxone. J Neuroimmunol 115:152-160.
29. Venken K, et al. (2008) Natural naive CD4+CD25+CD127low regulatory T cell (Treg) development and function are disturbed in multiple sclerosis patients: recovery of memory Treg homeostasis during disease progression. J Immunol 180:6411-6420.
30. Haas J, et al. (2009) Glatiramer acetate improves regulatory T-cell function by expansion of naive CD4(+)CD25(+)FOXP3(+)CD31(+) T-cells in patients with multiple sclerosis. J Neuroimmunol 216:113-117.
31. Hong J, at al. (2005) Induction of CD4+CD25+ regulatory T cells by copolymer-I through activation of transcription factor Foxp3. Proc Natl Acad Sci USA 102:6449-6454.
32. Weber M S, et al. (2007) Type II monocytes modulate T cell-mediated central nervous system autoimmune disease. Nat Med 13:935-943.
33. Kansara V, Mitra A, Wu Y, Subcutaneous Delivery. Drug Deliv Technol, June 2009; 9(6):38-42.
34. Kansara V, Mitra A, Wu Y, Subcutaneous Delivery. Drug Deliv Technol, June 2009; 9(6):38-42.
35. Luca Durelli, (2003) Dose and frequency of interferon treatment matter—INCOMIN and OPTIMS. J Neurol. 250 Suppl 4:IV9-1V14.
36. Glacier, GA-MS-303, An Open-label, Randomized, Multicenter Study to Assess the Safety and Tolerability of Glatiramer Acetate 40 mg/l mL Three-times Weekly Versus 20 mg/l mL Daily in Patients With Relapsing-remitting Multiple Sclerosis by Jerry S. Wolinsky, T. Erik Borresen, Dennis W. Dietrich, Beverly F. Gilder, Daniel Wynn, Yulia Sidi, Abi Vainstein-Haras, Volker Knappertz, and Scott Kolodny [online]. Projects In Knowledge, 1997 [retrieved on 2015 Jan. 27]. Retrieved from the Internet: <URL: www.projectsinknowledge.com/Activity/pdfs/2181.07.pdf>.

What is claimed is:

1. A method of inducing anti-glatiramer acetate (GA) specific antibodies in a human subject afflicted with multiple sclerosis, comprising administration to the human subject of three subcutaneous injections of a 40 mg/ml dose of glatiramer acetate per week for at least 12 months, such that the level of anti-GA specific antibodies in the blood or serum of the human subject
i) increases for up to about 6 months after an initial 40 mg/ml dose of glatiramer acetate of the administration is administered to the human subject;
ii) increases at a greater rate during the first month after the initial 40 mg/ml dose of glatiramer acetate compared to the rate of increase after the first month to the third month after the initial 40 mg/ml dose of glatiramer acetate;
iii) peaks at about 3 or about 6 months or between about 3 and about 6 months after the initial 40 mg/ml dose of glatiramer acetate; and
iv) is higher than baseline at least about 12 months after the initial 40 mg/ml dose of glatiramer acetate,
wherein the 40 mg/ml dose of glatiramer acetate is in a prefilled syringe containing 1 ml of an aqueous pharmaceutical solution of 40 mg/ml of glatiramer acetate and 40 mg/ml mannitol, wherein the aqueous pharmaceutical solution has a pH in the range of 5.5 to 7.0.

2. The method of claim 1, wherein the level of anti-GA specific antibodies in the blood or serum of the human subject increases for about 3 to about 6 months after the initial 40 mg/ml dose of glatiramer acetate.

3. The method of claim 1, wherein three subcutaneous injections of a 40 mg/ml dose of glatiramer acetate per week are administered to the human subject for at least 18 months, and the level of anti-GA specific antibodies in the blood or serum of the human subject is higher than baseline about 18 months after the initial 40 mg/ml dose of glatiramer acetate.

4. The method of claim 3, wherein three subcutaneous injections of a 40 mg/ml dose of glatiramer acetate per week are administered to the human subject for at least 24 months, and the level of anti-GA specific antibodies in the blood or serum of the human subject is higher than baseline about 24 months after the initial 40 mg/ml dose of glatiramer acetate.

5. The method of claim 1, wherein the anti-GA specific antibodies are other than IgM or IgE antibodies.

6. The method of claim 1, wherein the level of anti-GA specific antibodies in the blood or serum of the human subject increases
i) to at least about 25, about 30, about 35, about 40, about 45, about 50, about 55, about 60, about 65, about 67.5, about 70, about 75, about 80, about 25 to about 50, about 29 to about 106, about 29 to about 60, about 31 to about 60, about 35 to about 70, about 39 to about 71, about 50 to about 75, about 75 to about 80, or about 50 to about 80 RA % within about 1, about 3, about 6, about 9, about 12, about 18, or about 24 months after the initial 40 mg/ml dose of glatiramer acetate, as measured by an enzyme-linked immunosorbent assay (ELISA);
ii) by at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 25.9 to about 975, or about 100 to about 200-fold increase in RA % over baseline within about 1, about 3, about 6, about 9, about 12, about 18, or about 24 months after the initial 40 mg/ml dose of glatiramer acetate, as measured by ELISA; or
iii) to at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150 or about 100 to about 200-fold over baseline.

7. A method of producing a glatiramer acetate response profile for a human subject afflicted with multiple sclerosis comprising the steps of:
a) obtaining blood or serum samples periodically collected from a human subject afflicted with multiple sclerosis who is administered three subcutaneous injections of a 40 mg/ml dose of glatiramer acetate per week for at least about 12 or at least about 24 months after the initial 40 mg/ml dose of glatiramer acetate is administered;
b) assaying whether the level of anti-glatiramer acetate (GA) specific antibodies in the blood or serum of the human subject
i) increases for up to about 6 months after the initial 40 mg/ml dose of glatiramer acetate;
ii) increases at a greater rate during the first month after the initial 40 mg/ml dose of glatiramer Acetate compared to the rate of increase after the first month to the third month after the initial 40 mg/ml dose of glatiramer acetate;
iii) peaks at about 3 or about 6 months or between about 3 and about 6 months after the initial 40 mg/ml dose of glatiramer acetate; and
iv) is higher than baseline about 12 and about 24 months after the initial 40 mg/ml dose of glatiramer acetate,
c) producing the glatiramer acetate response profile of the human subject comprising the level of anti-GA specific antibodies determined in step b).

8. The method of claim 7, comprising assaying whether the level of anti-GA specific antibodies in the blood or serum of the human subject increases for about 3 to about 6 months after the initial 40 mg/ml dose of glatiramer acetate.

9. The method of claim 7, further comprising assaying whether the anti-GA specific antibodies are other than IgM or IgE antibodies, and the glatiramer acetate response profile of the human subject produced in step c) identifies the anti-GA specific antibodies as other than IgM or IgG antibodies.

10. The method of claim 9, wherein the glatiramer acetate response profile is a written glatiramer acetate response profile report.

11. The method of claim 7, wherein step b) comprises assaying whether the level of anti-GA specific antibodies in the blood or serum of the human subject increases
i) to at least about 25, about 30, about 35, about 40, about 45, about 50, about 55, about 60, about 65, about 67.5, about 70, about 75, about 80, about 25 to about 50, about 29 to about 106, about 29 to about 60, about 31 to about 60, about 35 to about 70, about 39 to about 71, about 50 to about 75, about 75 to about 80, or about 50 to about 80 RA % within about 1, about 3, about 6, about 9, about 12, about 18, or about 24 months after the initial 40 mg/ml dose of glatiramer acetate, as measured by an enzyme-linked immunosorbent assay (ELISA);
ii) by at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 25.9 to about 975, or about 100 to about 200-fold increase in RA % over baseline within about 1, about 3, about 6, about 9, about 12, about 18, or about 24 months after the initial 40 mg/ml dose of glatiramer acetate, as measured by ELISA; or
iii) to at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150 or about 100 to about 200-fold over baseline.

12. The method of claim 1, wherein the three subcutaneous injections are on three days each week selected from the group consisting of day 1, day 3 and day 5; day 1, day 3 and day 6; day 1, day 4 and day 6; day 2, day 4 and day 6; day 2, day 4 and day 7; 2, day 5 and day 7; and day 3, day 5 and day 7.

13. The method of claim 1, wherein the aqueous pharmaceutical solution
a) has a viscosity in the range of 2.0-3.5 cPa; or
b) has an osmolality in the range of 270-330 mosmol/Kg.

14. The method of claim 13, wherein the aqueous pharmaceutical solution
a) has a viscosity in the range of 2.2-3.0 cPa; or
b) has an osmolality in the range of 275-325 mosmol/Kg.

15. The method of claim 1, wherein the 40 mg/ml dose of glatiramer acetate is an aqueous pharmaceutical solution comprising 40 mg/ml glatiramer acetate and 40 mg/ml mannitol, wherein the aqueous pharmaceutical solution
a) has a viscosity in the range of 2.0-3.5 cPa; or
b) has an osmolality in the range of 275-325 mosmol/Kg.

16. The method of claim 15, wherein the aqueous pharmaceutical solution has a viscosity in the range of 2.0-3.5 cPa.

17. The method of claim 16, wherein the aqueous pharmaceutical solution has a viscosity in the range of 2.61-2.92 cPa.

18. The method of claim 15, wherein the aqueous pharmaceutical solution has an osmolality in the range of 275-325 mosmol/Kg.

19. The method of claim 18, wherein the aqueous pharmaceutical solution has an osmolality in the range of 300-303 mosmol/Kg.

20. The method of claim 1, wherein the human subject is suffering from relapsing-remitting multiple sclerosis (RRMS).

21. The method of claim 1, wherein the human subject is suffering from multiple sclerosis other than RRMS.

22. The method of claim 21, wherein the human subject is suffering from progressive-relapsing multiple sclerosis, secondary progressive multiple sclerosis, or primary progressive multiple sclerosis.

23. The method of claim 1, wherein the human subject has previously received administration of a 20 mg/ml dose of glatiramer acetate.

24. The method of claim 1, wherein the human subject is a naïve subject or has been previously administered a multiple sclerosis drug other than glatiramer acetate.

25. The method of claim 24, wherein the multiple sclerosis drug other than glatiramer acetate is interferon β-1a, interferon β-1b, mitoxantrone, natalizumab, fingolimod, teriflunomide, or dimethyl fumarate.

26. The method of claim 1, wherein the human subject has a genotype comprising:
one or more A alleles at the location of one or more single nucleotide polymorphisms (SNPs) selected from the group consisting of: kgp10152733, kgp10224254, kgp10305127, kgp10351364, kgp10372946, kgp10404633, kgp10564659, kgp10591989, kgp10594414, kgp10619195, kgp10620244, kgp10633631, kgp10974833, kgp11002881, kgp11285862, kgp11328629, kgp11407560, kgp11514107, kgp11627530, kgp11702474, kgp11711524, kgp11768533, kgp11804835, kgp12083934, kgp12182745, kgp12230354, kgp1224440, kgp124162, kgp12557319, kgp1371881, kgp1699628, kgp1753445, kgp1779254, kgp1786079, kgp18379774, kgp18525257, kgp20163979, kgp2023214, kgp20478926, kgp21171930, kgp2262166, kgp22778566, kgp2465184, kgp24753470, kgp25191871, kgp25216186, kgp25952891, kgp26026546, kgp26533576, kgp27500525, kgp27571222, kgp28532436, kgp28586329, kgp28817122, kgp2958113, kgp29794723, kgp30282494, kgp304921, kgp3205849, kgp3218351, kgp3276689, kgp337461, kgp345301, kgp355027, kgp355723, kgp3593828, kgp3812034, kgp3951463, kgp4162414, kgp4223880, kgp4418535, kgp4543470, kgp4573213, kgp4634875, kgp4755147, kgp4842590, kgp485316, kgp5068397, kgp5334779, kgp5483926, kgp5564995, kgp5869992, kgp5908616, kgp6032617, kgp6038357, kgp6076976, kgp6091119, kgp6127371, kgp61811, kgp6214351, kgp6228750, kgp6236949, kgp6469620, kgp6505544, kgp6507761, kgp6666134, kgp6700691, kgp6772915, kgp6959492, kgp7077322, kgp7117398, kgp7178233, kgp7186699, kgp7506434, kgp759150, kgp7730397, kgp7802182, kgp7804623, kgp7924485, kgp8030775, kgp8036704, kgp8046214, kgp8106690, kgp8110667, kgp8178358, kgp8200264, kgp8372910, kgp841428, kgp8602316, kgp8615910, kgp8793915, kgp8796185, kgp8990121, kgp9018750, kgp9354462, kgp9368119, kgp9410843, kgp9450430, kgp9530088, kgp9627338, kgp9669946, kgp97310, kgp974569, kgp9806386, kgp9884626, rs10049206, rs10124492, rs10125298, rs10162089, rs10203396, rs10251797, rs10278591, rs10489312, rs10492882, rs10498793, rs10501082, rs10510774, rs10512340, rs10815160, rs10816302, rs10841337, rs11029892, rs11029928, rs11192469, rs11559024, rs11648129, rs12013377, rs13394010, rs13415334, rs1478682, rs1544352, rs1545223, rs1604169, rs1621509, rs1644418, rs17029538, rs17400875, rs17449018, rs17577980, rs1858973, rs1894406, rs1894407, rs197523, rs2058742, rs2071469, rs2071472, rs2139612, rs2241883, rs2309760, rs241440, rs241442, rs241444, rs241445, rs241446, rs241449, rs241453, rs241456, rs2453478, rs2660214, rs2824070, rs2845371, rs2857103, rs2926455, rs343087, rs343092, rs3767955, rs3792135, rs3829539, rs3899755, rs4075692, rs4143493, rs423239, rs4254166, rs4356336, rs4584668, rs4780822, rs4782279, rs5024722, rs6032209, rs6110157, rs623011, rs6497396, rs6845927, rs6895094, rs6899068, rs7024953, rs7028906, rs7029123, rs7062312, rs7187976, rs7191155, rs720176, rs7228827, rs7496451, rs7563131, rs759458, rs7666442, rs7670525, rs7677801, rs7725112, rs7850, rs7862565, rs7948420, rs8035826, rs8053136, rs8055485, rs823829, rs9315047, rs9501224, rs9508832, rs950928, rs9597498, rs9670531, rs9671124, rs9817308, rs9834010, rs9876830 or rs9931211;
one or more C alleles at the location of one or more SNPs selected from the group consisting of: kgp10910719, kgp11077373, kgp11453406, kgp12426624, kgp2045074, kgp22811918, kgp23298674, kgp2709692, kgp28687699, kgp3496814, kgp3669685, kgp3730395, kgp4056892, kgp4370912, kgp5053636, kgp5216209, kgp5292386, kgp6023196, kgp652534, kgp7059449, kgp7189498, kgp7521990, kgp7792268, kgp8303520, kgp9320791, kgp9795732, rs10201643, rs11022778, rs11136970, rs11147439, rs11691553, rs1579771, rs16901784, rs2136408, rs2325911, rs241443, rs2857104, rs3803277, rs3885907, rs4738738, rs4894701, rs502530, rs6032205, rs6687976, rs6718758, rs6835202, rs714342, rs7524868, rs7844274, rs9393727 or rs9671182; or
one or more G alleles at the location of one or more SNPs selected from the group consisting of: kgp10090631, kgp1009249, kgp10412303, kgp10523170, kgp1054273, kgp10558725, kgp10632945, kgp10679353, kgp10788130, kgp10826273, kgp10922969, kgp10948564, kgp10967046, kgp1098237, kgp11010680, kgp11141512, kgp11206453, kgp11210903, kgp1124492, kgp11281589, kgp11356379, kgp11467007, kgp11543962, kgp11580695, kgp11633966, kgp11686146, kgp11843177, kgp12008955, kgp12371757, kgp1285441, kgp13161760, kgp1355977, kgp15390522, kgp1683448, kgp1688752, kgp1912531, kgp19568724, kgp2092817, kgp2245775, kgp22793211, kgp22823022, kgp2282938, kgp2299675, kgp2356388, kgp23672937, kgp23737989, kgp2388352, kgp2391411, kgp24131116, kgp24415534, kgp2446153, kgp2451249, kgp24729706, kgp25543811, kgp25921291, kgp26271158, kgp2638591, kgp26528455, kgp2688306, kgp26995430, kgp270001, kgp2715873, kgp27640141, kgp2788291, kgp2923815, kgp29367521, kgp293787, kgp2959751, kgp297178, kgp3048169, kgp3182607, kgp3202939, kgp3267884, kgp3418770, kgp3450875, kgp3477351, kgp3598409, kgp3651767, kgp3854180, kgp3933330, kgp3984567, kgp4011779, kgp4096263, kgp4127859, kgp4155998, kgp4346717, kgp4420791, kgp4479467, kgp4524468, kgp4559907, kgp4705854, kgp4734301, kgp4812831, kgp487328, kgp4898179, kgp5002011, kgp5014707, kgp5017029, kgp512180, kgp5144181, kgp5159037, kgp5388938, kgp5409955, kgp5440506, kgp5441587, kgp55646, kgp5579170, kgp5680955, kgp6190988, kgp6539666, kgp6567154, kgp6599438, kgp6603796, kgp6737096, kgp6768546, kgp6835138, kgp6996560, kgp7063887, kgp7092772, kgp7121374, kgp7181058, kgp7331172, kgp7416024, kgp7481870, kgp767200, kgp7714238, kgp7747883, kgp8107491, kgp8169636, kgp8174785, kgp8183049, kgp8192546, kgp8335515, kgp8437961, kgp8440036, kgp85534, kgp8599417, kgp8767692, kgp8777935, kgp8817856, kgp8869954, kgp9071686, kgp9078300, kgp9354820, kgp9421884, kgp9551947, kgp9601362, kgp9627406, kgp9699754, kgp971582, kgp9854133, rs1079303, rs10841322, rs10954782, rs11002051, rs11029907, rs11083404, rs11085044, rs11192461, rs1157449, rs12494712, rs12943140, rs13002663, rs13419758, rs1380706, rs1387768, rs1410779, rs1508102, rs1532365, rs16886004, rs16895510, rs16927077, rs16930057, rs17224858, rs17238927, rs17329014, rs17638791, rs1886214, rs1894408, rs196295, rs196341, rs196343, rs1979992, rs1979993, rs2043136, rs2071470, rs2074037, rs2175121, rs241435, rs241447, rs241451, rs241452, rs241454, rs2598360, rs2621321, rs2621323, rs2816838, rs2839117, rs2857101, rs2934491, rs3135388, rs3218328, rs3799383, rs3815822, rs3818675, rs419132, rs4360791, rs4449139, rs4669694, rs4709792, rs4769060, rs4822644, rs484482, rs543122, rs6535882, rs6840089, rs7020402, rs7217872, rs7348267, rs7579987, rs7672014, rs7860748, rs7864679, rs7928078, rs8050872, rs858341, rs931570, rs9346979, rs9376361, rs9579566, rs9913349 or rs9931167, or one or more T alleles at the location of kgp18432055, kgp279772, kgp3991733 or kgp7242489.

27. The method of claim 1, wherein assaying the level of anti-GA specific antibodies comprises an enzyme-linked immunosorbent assay (ELISA), solid-phase ELISA or a radioimmunoassay.

28. The method of claim 1, wherein the baseline level of anti-GA specific antibodies is
i) the level of anti-GA specific antibodies in a corresponding naïve human subject afflicted with multiple sclerosis;
ii) the level of anti-GA specific antibodies in a corresponding naïve healthy human subject;
iii) the level of anti-GA specific antibodies in the human subject before the human subject has been administered the after the initial 40 mg/ml dose of glatiramer acetate; or
iv) the level of anti-GA specific antibodies in the human subject concurrently with administration of the initial 40 mg/ml dose of glatiramer acetate.

29. The method of claim 1, wherein the baseline level of anti-GA specific antibodies is the RA % for
i) the level of anti-GA specific antibodies in a corresponding naïve human subject afflicted with multiple sclerosis;
ii) the level of anti-GA specific antibodies in a corresponding naïve healthy human subject;
iii) the level of anti-GA specific antibodies in the human subject before the human subject has been administered the after the initial 40 mg/ml dose of glatiramer acetate; or
iv) the level of anti-GA specific antibodies in the human subject concurrently with administration of the initial 40 mg/ml dose of glatiramer acetate.

30. A method of inducing anti-glatiramer acetate (GA) specific antibodies in a human subject afflicted with multiple sclerosis, comprising administration to the human subject of three subcutaneous injections of a 40 mg/ml dose of glatiramer acetate per week for at least 12 months, such that the level of anti-GA specific antibodies in the blood or serum of the human subject
i) increases for up to 6 months after an initial 40 mg/ml dose of glatiramer acetate of the administration is administered to the human subject;
ii) increases at a greater rate during the first month after the initial 40 mg/ml dose of glatiramer acetate compared to the rate of increase after the first month to the third month after the initial 40 mg/ml dose of glatiramer acetate;
iii) peaks at 3 or 6 months or between 3 and 6 months after the initial 40 mg/ml dose of glatiramer acetate; and
iv) is higher than baseline at least 12 months after the initial 40 mg/ml dose of glatiramer acetate,
wherein the 40 mg/ml dose of glatiramer acetate is in a prefilled syringe containing 1 ml of an aqueous pharmaceutical solution of 40 mg/ml of glatiramer acetate and 40 mg/ml mannitol, and wherein the aqueous pharmaceutical solution
a) has a viscosity in the range of 2.0-3.5 cPa; or
b) has an osmolality in the range of 270-330 mosmol/Kg.
